SEQUENCE LISTING (205 sequences)

SEQ ID NO: 1 - DNA, 129 bp, Bacillus subtilis
atgaaaaaagctgtcattgtagaaaacaaaggttgtgcaacatgctcgatcggagccgcttgtctagtggacggtcctatccctgattttgaaattgccggtgcaacaggtctattcggtctatggggg

SEQ ID NO: 2 - protein, 43 aa, Bacillus subtilis
MKKAVIVENKGCATCSIGAACLVDGPIPDFEIAGATGLFGLWG

SEQ ID NO: 3 - DNA, 456 bp, Bacillus subtilis
atgattggaagaattatccgtttgtaccgtaaaagaaaaggctattctattaatcagctggctgttgagtcaggcgtatccaaatcctatttaagcaagattgaaagaggcgttcacacgaatccgtccgttcaatttttaaaaaaagtttctgccacactggaagttgaattaacagaattatttgacgcagaaacaatgatgtatgaaaaaatcagcggcggtgaagaagaatggcgcgtacatttagtgcaagccgtacaagccgggatggaaaaggaagaattgttcacttttacgaacagactcaagaaagaacagcctgaaactgcctcttaccgcaaccgcaaactgacggaatccaatatagaagaatggaaagcgctgatggcggaggcaagagaaatcggcttgtctgtccatgaagtcaaatcctttttaaaaacaaagggaaga

SEQ ID NO: 4 - protein, 152 aa, Bacillus subtilis
MIGRIIRLYRKRKGYSINQLAVESGVSKSYLSKIERGVHTNPSVQFLKKVSATLEVELTELFDAETMMYEKISGGEEEWRVHLVQAVQAGMEKEELFTFTNRLKKEQPETASYRNRKLTESNIEEWKALMAEAREIGLSVHEVKSFLKTKGR

SEQ ID NO: 5 - DNA, 165 bp, Bacillus subtilis
atgaaaagaaaccaaaaagaatgggaatctgtgagtaaaaaaggacttatgaagccgggaggtacttcgattgtgaaagctgctggctgcatgggctgttgggcctcgaagagtattgctatgacacgtgtttgtgcacttccgcatcctgctatgagagctatt

SEQ ID NO: 6 - protein, 55 aa, Bacillus subtilis
MKRNQKEWESVSKKGLMKPGGTSIVKAAGCMGCWASKSIAMTRVCALPHPAMRAI

SEQ ID NO: 7 - DNA, 831 bp, Bacillus subtilis
atgaaaatcagtatgcaaaaagcagatttttggaaaaaagcagcgatctcattacttgttttcaccatgttttttaccctgatgatgagcgaaacggtttttgcggcgggactgaataaagatcaaaagcgccgggcggaacagctgacaagtatctttgaaaacggcacaacggagatccaatatggatatgtagagcgattggatgacgggcgaggctatacatgcggacgggcaggctttacaacggctaccggggatgcattggaagtagtggaagtatacacaaaggcagttccgaataacaaactgaaaaagtatctgcctgaattgcgccgtctggccaaggaagaaagcgatgatacaagcaatctcaagggattcgcttctgcctggaagtcgcttgcaaatgataaggaatttcgcgccgctcaagacaaagtaaatgaccatttgtattatcagcctgccatgaaacgatcggataatgccggactaaaaacagcattggcaagagctgtgatgtacgatacggttattcagcatggcgatggtgatgaccctgactctttttatgccttgattaaacgtacgaacaaaaaagcgggcggatcacctaaagacggaatagacgagaagaagtggttgaataaattcttggacgtacgctatgacgatctgatgaatccggccaatcatgacacccgtgacgaatggagagaatcagttgcccgtgtggacgtgcttcgctctatcgccaaggagaacaactataatctaaacggaccgattcatgttcgttcaaacgagtacggtaattttgtaatcaaa

SEQ ID NO: 8 - protein, 277 aa, Bacillus subtilis
MKISMQKADFWKKAAISLLVFTMFFTLMMSETVFAAGLNKDQKRRAEQLTSIFENGTTEIQYGYVERLDDGRGYTCGRAGFTTATGDALEVVEVYTKAVPNNKLKKYLPELRRLAKEESDDTSNLKGFASAWKSLANDKEFRAAQDKVNDHLYYQPAMKRSDNAGLKTALARAVMYDTVIQHGDGDDPDSFYALIKRTNKKAGGSPKDGIDEKKWLNKFLDVRYDDLMNPANHDTRDEWRESVARVDVLRSIAKENNYNLNGPIHVRSNEYGNFVIK

SEQ ID NO: 9 - DNA, 792 bp, Bacillus subtilis
ttgatcatgacacaaccatcaaaaactacgaaactaactaaagatgaagtcgatcggctcataagcgattaccaaacaaagcaagatgaacaagcgcaggaaacgcttgtgcgggtgtatacaaatctggttgacatgcttgcgaaaaaatactcaaaaggcaaaagcttccacgaggatctccgccaggtcggcatgatcgggctgctaggcgcgattaagcgatacgatcctgttgtcggcaaatcgtttgaagcttttgcaatcccgacaatcatcggtgaaattaaacgtttcctcagagataaaacatggagcgttcatgtgccgagacgaattaaagaactcggtccaagaatcaaaatggcggttgatcagctgaccactgaaacacaaagatcgccgaaagtcgaagagattgccgaattcctcgatgtttctgaagaagaggttcttgaaacgatggaaatgggcaaaagctatcaagccttatccgttgaccacagcattgaagcggattcggacggaagcactgtcacgattcttgatatcgtcggatcacaggaggacggatatgagcgggtcaaccagcaattgatgctgcaaagcgtgcttcatgtcctttcagaccgtgagaaacaaatcatagaccttacgtatattcaaaacaaaagccaaaaagaaactggggacattctcggtatatctcaaatgcacgtctcgcgcttgcaacgcaaagctgtgaagaagctcagagaggccttgattgaagatccctcgatggagttaatg

SEQ ID NO: 10 - protein, 264 aa, Bacillus subtilis
MIMTQPSKTTKLTKDEVDRLISDYQTKQDEQAQETLVRVYTNLVDMLAKKYSKGKSFHEDLRQVGMIGLLGAIKRYDPVVGKSFEAFAIPTIIGEIKRFLRDKTWSVHVPRRIKELGPRIKMAVDQLTTETQRSPKVEEIAEFLDVSEEEVLETMEMGKSYQALSVDHSIEADSDGSTVTILDIVGSQEDGYERVNQQLMLQSVLHVLSDREKQIIDLTYIQNKSQKETGDILGISQMHVSRLQRKAVKKLREALIEDPSMELM

SEQ ID NO: 11 - DNA, 744 bp, Bacillus subtilis
atggttttattctttcagatcatggtctggtgcatcgtggccggactggggttatacgtgtatgccacgtggcgtttcgaagcgaaggtcaaagaaaaaatgtccgccattcggaaaacttggtatttgctgtttgttctgggcgctatggtatactggacatatgagcccacttccctatttacccactgggaacggtatctcattgtcgcagtcagttttgctttgattgatgcttttatcttcttaagtgcatatgtcaaaaaactggccggcagcgagcttgaaacagacacaagagaaattcttgaagaaaacaacgaaatgctccacatgtatctcaatcggctgaaaacataccaatacctattgaaaaacgaaccgatccatgtttattatggaagtatagatgcttatgctgaaggtattgataagctgctgaaaacctatgctgataaaatgaacttaacggcttctctttgccactattcgacacaggctgataaagaccggttaaccgagcatatggatgatccggcagatgtacaaacacggctcgatcgaaaggatgtttattacgaccaatacggaaaagtggttctcatcccttttaccatcgagacacagaactatgtcatcaagctgacgtctgacagcattgtcacggaatttgattatttgctatttacgtcattaacgagcatatatgatttggtgctgccaattgaggaggaaggtgaagga

SEQ ID NO: 12 - protein, 248 aa, Bacillus subtilis
MVLFFQIMVWCIVAGLGLYVYATWRFEAKVKEKMSAIRKTWYLLFVLGAMVYWTYEPTSLFTHWERYLIVAVSFALIDAFIFLSAYVKKLAGSELETDTREILEENNEMLHMYLNRLKTYQYLLKNEPIHVYYGSIDAYAEGIDKLLKTYADKMNLTASLCHYSTQADKDRLTEHMDDPADVQTRLDRKDVYYDQYGKVVLIPFTIETQNYVIKLTSDSIVTEFDYLLFTSLTSIYDLVLPIEEEGEG

SEQ ID NO: 13 - DNA, 120 bp, Bacillus subtilis
atgaaattgaaatctaagttgtttgttatttgtttggccgcagccgcgatttttacagcggctggcgtttctgctaatgcggaagcactcgactttcatgtgacagaaagaggaatgacg

SEQ ID NO: 14 - protein, 40 aa, Bacillus subtilis
MKLKSKLFVICLAAAAIFTAAGVSANAEALDFHVTERGMT

SEQ ID NO: 15 - DNA, 1134 bp, Bacillus subtilis
ttgaggatgaagcagacgattccgtcctcttatgtcgggcttaaaattaatgaatggtatactcatatccggcagttccacgtcgctgaagccgaacgggtcaagctcgaagtagaaagagaaattgaggatatggaagaagaccaagatttgctgctgtattattctttaatggagttcaggcaccgtgtcatgctggattacattaagccttttggagaggacacgtcgcagctagagttttcagaattgttagaagacatcgaagggaatcagtacaagctgacagggcttctcgaatattactttaatttttttcgaggaatgtatgaatttaagcagaagatgtttgtcagtgccatgatgtattataaacgggcagaaaagaatcttgccctcgtctcggatgatattgagaaagcagagtttgcttttaaaatggctgagattttttacaatttaaaacaaacctatgtttcgatgagctacgccgttcaggcattagaaacataccaaatgtatgaaacgtacaccgtccgcagaatccaatgtgaattcgttattgcaggtaattatgatgatatgcagtatccagaaagagcattgccccacttagaactggctttagatcttgcaaagaaagaaggcaatccccgcctgatcagttctgccctatataatctcggaaactgctatgagaaaatgggtgaactgcaaaaggcagccgaatactttgggaaatctgtttctatttgcaagtcggaaaagttcgataatcttccgcattctatctactctttaacacaagttctgtataaacaaaaaaatgacgccgaagcgcaaaaaaagtatcgtgaaggattggaaatcgcccgtcaatacagtgatgaattatttgtggagctttttcaatttttacatgcgttatacggaaaaaacattgacacagaatcagtctcacacacctttcaatttcttgaagaacatatgctgtatccttatattgaagagctggcgcatgatgctgcccaattctatatagaaaacggacagcccgaaaaagcactttcattttatgagaaaatggtgcacgcacaaaaacaaatccagagaggagattgtttatatgaaatc

SEQ ID NO: 16 - protein, 378 aa, Bacillus subtilis
MRMKQTIPSSYVGLKINEWYTHIRQFHVAEAERVKLEVEREIEDMEEDQDLLLYYSLMEFRHRVMLDYIKPFGEDTSQLEFSELLEDIEGNQYKLTGLLEYYFNFFRGMYEFKQKMFVSAMMYYKRAEKNLALVSDDIEKAEFAFKMAEIFYNLKQTYVSMSYAVQALETYQMYETYTVRRIQCEFVIAGNYDDMQYPERALPHLELALDLAKKEGNPRLISSALYNLGNCYEKMGELQKAAEYFGKSVSICKSEKFDNLPHSIYSLTQVLYKQKNDAEAQKKYREGLEIARQYSDELFVELFQFLHALYGKNIDTESVSHTFQFLEEHMLYPYIEELAHDAAQFYIENGQPEKALSFYEKMVHAQKQIQRGDCLYEI

SEQ ID NO: 17 - DNA, 1353 bp, Bacillus subtilis
atgaaaaacaagccgctcgcgtttcagatatgggttgtcatatccggcatcctgttagcgatatcgattttactgcttgtgttattttcaaacacgctgcgagattttttcactaatgaaacgtatacgacgattgaaaatgagcagcatgttctgacagagtaccgcctgccaggttcgattgaaaggcgctattacagcgaggaagcgacggcgccgacaactgtccgctccgtacagcacgtgctccttcctgaaaatgaagaggcttcttcagacaaggatttaagcattctgtcatcttcatttatccacaaggtgtacaagctggctgataagcaggaagctaaaaagaaacgttacagcgccgacgtcaatggagagaaagtgttttttgtcattaaaaagggactttccgtcaatggacaatcagcgatgatgctctcttacgcgcttgattcttatcgggacgatttggcctataccttgttcaaacagcttctgtttattatagctgtcgtcattttattaagctggattccggctatttggcttgcaaagtatttatcaaggcctcttgtatcatttgaaaaacacgtcaaacggatttctgaacaggattgggatgacccagtaaaagtggaccggaaagatgaaatcggcaaattgggccataccatcgaagagatgcgccaaaagcttgtgcaaaaggatgaaacagaaagaactctattgcaaaatatctctcatgatttaaaaacgccggtcatggtcatcagaggctatacacaatcaattaaagacgggatttttcctaaaggagaccttgaaaacactgtagatgttattgaatgcgaagctcttaagctggagaaaaaaataaaggatttattatatttaacgaagctggattatttagcgaagcaaaaagtgcagcacgacatgttcagtattgtggaagtgacagaagaagtcatcgaacgattgaagtgggcgcggaaagaactatcgtgggaaattgatgtagaagaggatattttgatgccgggcgatccggagcaatggaacaaactcctcgaaaacattttggaaaatcaaatccgctatgctgagacaaaaatagaaatcagcatgaaacaagatgatcgaaatatcgtgatcaccattaaaaatgacggtccgcatattgaagatgagatgctctccagcctctatgagccttttaataaagggaagaaaggcgaattcggcattggtctaagcatcgtaaaacgaattttaactcttcataaggcatctatctcaattgaaaatgacaaaacgggtgtatcataccgcatagcagtgccaaaa

SEQ ID NO: 18 - protein, 450 aa, Bacillus subtilis
MKNKPLAFQIWVVISGILLAISILLLVLFSNTLRDFFTNETYTTIENEQHVLTEYRLPGSIERRYYSEEATAPTTVRSVQHVLLPENEEASSDKDLSILSSSFIHKVYKLADKQEAKKKRYSADVNGEKVFFVIKKGLSVNGQSAMMLSYALDSYRDDLAYTLFKQLLFIIAVVILLSWIPAIWLAKYLSRPLVSFEKHVKRISEQDWDDPVKVDRKDEIGKLGHTIEEMRQKLVQKDETERTLLQNISHDLKTPVMVIRGYTQSIKDGIFPKGDLENTVDVIECEALKLEKKIKDLLYLTKLDYLAKQKVQHDMFSIVEVTEEVIERLKWARKELSWEIVEEDILMPGDPEQWNKLLENILENQIRYAETKIEISMKQDDRNIVITIKNDGPHIEDEMLSSLYEPFNKGKKGEFGIGLSIVKRILTLHKASISIENDKTGVSYRIAVPK

SEQ ID NO: 19 - DNA, 2013 bp, Bacillus subtilis
atgtttaaaaataatgtcatacttttaaattcaccttatcatgcacatgctcataaagaggggtttattctaaaaaggggatggacggttttggaaagcaagtacctagatctactcgcacaaaaatacgattgtgaagaaaaagtggtaacagaaatcatcaatttgaaagcgatattgaacctgccaaaaggcaccgagcattttgtcagtgatctgcacggagagtatcaggcattccagcacgtgttgcgcaatggttcaggacgagtcaaagagaagatacgcgacatcttcagcggtgtcatttacgatagagaaattgatgaattagcagcattggtctattatccggaagacaaactgaaattaatcaaacatgactttgatgcgaaagaagcgttaaacgagtggtataaagaaacgattcatcgaatgattaagctcgtttcatattgctcctctaagtatacccgctccaaattacgcaaagcactgcctgcccaatttgcttatattacggaggagctgttatacaaaacagaacaagctggcaacaaggagcaatattactccgaaatcattgatcagatcattgaacttggccaagccgataagctgatcaccggccttgcttacagcgttcagcgattggtggtcgaccatctgcatgtggtcggcgatatttatgaccgcggcccgcagccggatagaattatggaagaactgatcaactatcattctgtcgatattcagtggggaaatcacgatgtcctttggatcggcgcctattccggttccaaagtgtgcctggccaatattatccgcatctgtgcccgctacgacaacctggatattattgaggacgtgtacggcatcaacctgagaccgctgctgaacctggccgaaaaatattatgatgataatccagcgttccgtccaaaagcagacgaaaacaggccagaggatgagattaagcaaatcacaaaaatccatcaagcgattgccatgatccaattcaagcttgagagcccgattatcaagagacggccgaactttaatatggaagagcggctgttattagagaaaatagactatgacaaaaatgaaatcacgctgaacggaaaaacatatcaactggaaaacacctgctttgcgacgattaatccggagcagccagatcagctattagaagaagaagcagaagtcatagacaagctgctattctctgtccagcattccgaaaagctgggccgccatatgaattttatgatgaaaaaaggcagcctttatttaaaatataacggcaacctgttgattcacggctgtattccagttgatgaaaacggcaatatggaaacgatgatgattgaggataaaccgtatgcgggccgtgagctgctcgatgtatttgaacgattcttgcgggaagcctttgcccacccggaagaaaccgatgacctggcgacagatatggcttggtatttatggacaggcgaatactcctccctcttcggaaaacgcgccatgacgacatttgagcgctatttcatcaaagagaaggaaacgcataaagagaagaaaaacccgtattattatttacgagaagacgaggcaacctgccgaaacatcctggcagaattcggcctcaatccagatcacggccatatcatcaacggccatacacctgtaaaagaaatcgaaggagaagacccaatcaaagcaaacggaaaaatgatcgtcatcgacggcggcttctccaaagcctaccaatccacaacaggcatcgccggctacacgctgctatacaactcctacggcatgcagctcgtcgcccataaacacttcaattccaaggcagaagtcctaagcaccggaaccgacgtcttaacggtcaaacgattagtggacaaagagcttgagcggaagaaagtgaaggaaacgaatgtgggtgaggaattgttgcaggaagttgcgattttagagagtttgcgggagtatcggtatatgaag

SEQ ID NO: 20 - protein, 671 aa, Bacillus subtilis
MFKNNVILLNSPYHAHAHKEGFILKRGWTVLESKYLDLLAQKYDCEEKVVTEIINLKAILNLPKGTEHFVSDLHGEYQAFQHVLRNGSGRVKEKIRDIFSGVIYDREIDELAALVYYPEDKLKLIKHDFDAKEALNEWYKETIHRMIKLVSYCSSKYTRSKLRKALPAQFAYITEELLYKTEQAGNKEQYYSEIIDQIIELGQADKLITGLAYSVQRLVVDHLHVVGDIYDRGPQPDRIMEELINYHSVDIQWGNHDVLWIGAYSGSKVCLANIIRICARYDNLDIIEDVYGINLRPLLNLAEKYYDDNPAFRPKADENRPEDEIKQITKIHQAIAMIQFKLESPIIKRRPNFNMEERLLLEKIDYDKNEITLNGKTYQLENTCFATINPEQPDQLLEEEAEVIDKLLFSVQHSEKLGRHMNFMMKKGSLYLKYNGNLLIHGCIPVDENGNMETMMIEDKPYAGRELLDVFERFLREAFAHPEETDDLATDMAWYLWTGEYSSLFGKRAMTTFERYFIKEKETHKEKKNPYYYLREDEATCRNILAEFGLNPDHGHIINGHTPVKEIEGEDPIKANGKMIVIDGGFSKAYQSTTGIAGYTLLYNSYGMQLVAHKHFNSKAEVLSTGTDVLTVKRLVDKELERKKVKETNVGEELLQEVAILESLREYRYMK

SEQ ID NO: 21 - DNA, 765 bp, Bacillus subtilis
atgaaacgagaaagcaacattcaagtgctcagccgtggtcaaaaagatcagcctgtgagccagatttatcaagtatcaacaatgacttctctattagacggagtatatgacggagattttgaactgtcagagattccgaaatatggagacttcggtatcggaacctttaacaagcttgacggagagctgattgggtttgacggcgaattttaccgtcttcgctcagacggaaccgcgacaccggtccaaaatggagaccgttcaccgttctgttcatttacgttctttacaccggacatgacgcacaaaattgatgcgaaaatgacacgcgaagactttgaaaaagagatcaacagcatgctgccaagcagaaacttattttatgcaattcgcattgacggattgtttaaaaaggtgcagacaagaacagtagaacttcaagaaaaaccttacgtgccaatggttgaagcggtcaaaacacagccgattttcaacttcgacaacgtgagaggaacgattgtaggtttcttgacaccagcttatgcaaacggaatcgccgtttctggctatcacctgcacttcattgacgaaggacgcaattcaggcggacacgtttttgactatgtgcttgaggattgcacggttacgatttctcaaaaaatgaacatgaatctcagacttccgaacacagcggatttctttaatgcgaatctggataaccctgattttgcgaaagatatcgaaacaactgaaggaagccctgaa

SEQ ID NO: 22 - protein, 255 aa, Bacillus subtilis
MKRESNIQVLSRGQKDQPVSQIYQVSTMTSLLDGVYDGDFELSEIPKYGDFGIGTFNKLDGELIGFDGEFYRLRSDGTATPVQNGDRSPFCSFTFFTPDMTHKIDAKMTREDFEKEINSMLPSRNLFYAIRIDGLFKKVQTRTVELQEKPYVPMVEAVKTQPIFNFDNVRGTIVGFLTPAYANGIAVSGYHLHFIDEGRNSGGHVFDYVLEDCTVTISQKMNMNLRLPNTADFFNANLDNPDFAKDIETTEGSPE

SEQ ID NO: 23 - DNA, 1020 bp, Bacillus subtilis
atgaaggtaaaagtagcgatcaacgggtttggaagaatcggaagaatggtttttagaaaagcgatgttagacgatcaaattcaagtagtggccattaacgccagctattccgcagaaacgctggctcatttaataaagtatgacacaattcacggcagatacgacaaagaggttgtggctggtgaagatagcctgatcgtaaatggaaagaaagtgcttttgttaaacagccgtgatccaaaacagctgccttggcgggaatatgatattgacatagtcgtcgaagcaacagggaagtttaatgctaaagataaagcgatgggccatatagaagcaggtgcaaaaaaagtgattttgaccgctccgggaaaaaatgaagacgttaccattgtgatgggcgtaaatgaggaccaattcgacgctgagcgccatgtcattatttcaaatgcgtcatgcacgacaaattgccttgcgcctgttgtaaaagtgctggatgaagagtttggcattgagagcggtctgatgactacagttcatgcgtatacgaatgaccaaaaaaatattgataacccgcacaaagatttgcgccgggcgcgggcttgcggtgaatccatcattccaacaacaacaggagcggcaaaggcgctttcgcttgtgctgccgcatctgaaaggaaaacttcacggcctcgccttgcgtgtccctgttccgaacgtctcattggttgatctcgttgttgatctgaaaacggatgttacggctgaagaagtaaacgaggcatttaaacgcgctgccaaaacgtcgatgtacggtgtacttgattactcagatgaaccgctcgtttcgactgattataatacgaatccgcattcagcggtcattgacgggcttacaacaatggtaatggaagacaggaaagtaaaggtgctggcgtggtatgacaacgaatggggctactcctgcagagttgttgatctaatccgccatgtagcggcacgaatgaaacatccgtctgctgta

SEQ ID NO: 24 - protein, 340 aa, Bacillus subtilis
MKVKVAINGFGRIGRMVFRKAMLDDQIQVVAINASYSAETLAHLIKYDTIHGRYDKEVVAGEDSLIVNGKKVLLLNSRDPKQLPWREYDIDIVVEATGKFNAKDKAMGHIEAGAKKVILTAPGKNEDVTIVMGVNEDQFDAERHVIISNASCTTNCLAPVVKVLDEEFGIESGLMTTVHAYTNDQKNIDNPHKDLRRARACGESIIPTTTGAAKALSLVLPHLKGKLHGLALRVPVPNVSLVDLVVDLKTDVTAEEVNEAFKRAAKTSMYGVLDYSDEPLVSTDYNTNPHSAVIDGLTTMVMEDRKVKVLAWYDNEWGYSCRVVDLIRHVAARMKHPSAV

SEQ ID NO: 25 - DNA, 1176 bp, Bacillus subtilis
atgacgaaggaatttgagtttttaaaagcagagcttaatagtatgaaagaaaaccatacatggcaagacataaaacagcttgaatctatgcagggcccatctgtcacagtgaatcaccaaaaagtcattcagctatcttctaataattacctcggattcacttcacatcctagactcatcaacgccgcacaggaggccgttcagcagtatggagccggcaccggatcagtgagaacgattgcgggtacatttacaatgcatcaagagcttgagaaaaagctggcagcctttaaaaaaacggaggcggcacttgtattccaatcaggcttcacaacaaaccaaggcgtactttcaagtattctatcaaaagaggacattgtcatctcagatgaattgaaccatgcctctattattgacggaattcgactgacaaaggcggataaaaaggtgtatcagcacgtcaatatgagtgatttagagcgggtgctgagaaagtcaatgaattatcggatgcgtctgattgtgacagacggcgtattttccatggatggcaacatagctcctctgcctgatattgtagagctcgctgagaaatatgacgcatttgtgatggtggatgacgcccatgcatccggagtacttggcgaaaacggcaggggaacggtgaatcacttcggtcttgacggcagagtgcatattcaggtcggaacattaagcaaggcaatcggagtgctcggcggctacgctgcaggttcaaaggtgctgatcgattatttgcgccataaaggccgtccatttttattcagcacatctcatccgccggcagtcactgcagcttgtatggaagcgattgatgtcttgcttgaagagccggagcatatggagcgcttgtgggagaatactgcctattttaaagcaatgcttgtgaaaatgggtctgactctcacgaagagtgaaacgccgattcttcctattttaataggtgatgaaggtgtggcaaagcaattttcagatcagctcctttctcgcggtgtttttgcccaaagtatcgttttcccgactgtagcaaagggaaaagccagaattcgcacgattataacagcagagcacaccaaagatgaactggatcaggcgcttgatgtcatcgaaaagacggcaaaggagctccagctattg

SEQ ID NO: 26 - protein, 392 aa, Bacillus subtilis
MTKEFEFLKAELNSMKENHTWQDIKQLESMQGPSVTVNHQKVIQLSSNNYLGFTSHPRLINAAQEAVQQYGAGTGSVRTIAGTFTMHQELEKKLAAFKKTEAALVFQSGFTTNQGVLSSILSKEDIVISDELNHASIIDGIRLTKADKKVYQHVNMSDLERVLRKSMNYRMRLIVTDGVFSMDGNIAPLPDIVELAEKYDAFVMVDDAHASGVLGENGRGTVNHFGLDGRVHIQVGTLSKAIGVLGGYAAGSKVLIDYLRHKGRPFLFSTSHPPAVTAACMEAIDVLLEEPEHMERLWENTAYFKAMLVKMGLTLTKSETPILPILIGDEGVAKQFSDQLLSRGVFAQSIVFPTVAKGKARIRTIITAEHTKDELDQALDVIEKTAKELQLL

SEQ ID NO: 27 - DNA, 1581 bp, Bacillus subtilis
atgaactcagttgatttgaccgctgatttacaagccttattaacatgtccaaatgtgcgtcataatttatcagcagcacagctaacagaaaaagtcctctcccgaaacgaaggcattttaacatccacaggtgctgttcgcgcgacaacaggcgcttacacaggacgctcacctaaagataaattcatcgtggaggaagaaagcacgaaaaataagatcgattggggcccggtgaatcagccgatttcagaagaagcgtttgagcggctgtacacgaaagttgtcagctatttaaaggagcgagatgaactgtttgttttcgaaggatttgccggagcagacgagaaatacaggctgccgatcactgtcgtaaatgagttcgcatggcacaatttatttgcgcggcagctgtttatccgtccggaaggaaatgataagaaaacagttgagcagccgttcaccattctttctgctccgcatttcaaagcggatccaaaaacagacggcactcattccgaaacgtttattattgtctctttcgaaaagcggacaattttaatcggcggaactgagtatgccggtgaaatgaagaagtccattttctccattatgaatttcctgctgcctgaaagagatattttatctatgcactgctccgccaatgtcggtgaaaaaggcgatgtcgcccttttcttcggactgtcaggaacaggaaagaccaccctgtcggcagatgctgaccgcaagctgatcggtgacgatgaacatggctggtctgatacaggcgtctttaatattgaaggcggatgctacgctaagtgtattcatttaagcgaggaaaaggagccgcaaatctttaacgcgatccgcttcgggtctgttctcgaaaatgtcgttgtggatgaagatacacgcgaagccaattatgatgattccttctatactgaaaacacgcgggcagcttacccgattcatatgattaataacatcgtgactccaagcatggccggccatccgtcagccattgtatttttgacggctgatgccttcggagtcctgccgccgatcagcaaactaacgaaggagcaggtgatgtaccattttttgagcggttacacgagtaagcttgccggaaccgaacgtggtgtcacgtctcctgaaacgacgttttctacatgcttcggctcaccgttcctgccgcttcctgctcacgtctatgctgaaatgctcggcaaaaagatcgatgaacacggcgcagacgttttcttagtcaataccggatggaccgggggcggctacggcacaggcgaacgaatgaagctttcttacactagagcaatggtcaaagcagcgattgaaggcaaattagaggatgctgaaatgataactgacgatattttcggcctgcacattccggcccatgttcctggcgttcctgatcatatccttcagcctgaaaacacgtggaccaacaaggaagaatacaaagaaaaagcagtctaccttgcaaatgaattcaaagagaactttaaaaagttcgcacataccgatgccatcgcccaggcaggcggccctctcgta

SEQ ID NO: 28 - protein, 527 aa, Bacillus subtilis
MNSVDLTADLQALLTCPNVRHNLSAAQLTEKVLSRNEGILTSTGAVRATTGAYTGRSPKDKFIVEEESTKNKIDWGPVNQPISEEAFERLYTKVVSYLKERDELFVFEGFAGADEKYRLPITVVNEFAWHNLFARQLFIRPEGNDKKTVEQPFTILSAPHFKADPKTDGTHSETFIIVSFEKRTILIGGTEYAGEMKKSIFSIMNFLLPERDILSMHCSANVGEKGDVALFFGLSGTGKTTLSADADRKLIGDDEHGWSDTGVFNIEGGCYAKCIHLSEEKEPQIFNAIRFGSVLENVVVDEDTREANYDDSFYTENTRAAYPIHMINNIVTPSMAGHPSAIVFLTADAFGVLPPISKLTKEQVMYHFLSGYTSKLAGTERGVTSPETTFSTCFGSPFLPLPAHVYAEMLGKKIDEHGADVFLVNTGWTGGGYGTGERMKLSYTRAMVKAAIEGKLEDAEMITDDIFGLHIPAHVPGVPDHILQPENTWTNKEEYKEKAVYLANEFKENFKKFAHTDAIAQAGGPLV

SEQ ID NO: 29 - DNA, 762 bp, Bacillus subtilis
ttgttaacagccttaaaaacagatacaggaaaaatccgccagcataatgaagatgatgcggggatattcaaggggaaagatgaatttatattagcggttgtcgctgatggcatgggcggccatcttgctggagatgttgcgagcaagatggctgtgaaagccatgggggagaaatggaatgaagcagagacgattccaactgcgccctcggaatgtgaaaaatggctcattgaacagattctatcggtaaacagcaaaatatacgatcacgctcaagcccacgaagaatgccaaggcatggggacgacgattgtatgtgcactttttacggggaaaacggtttctgttgcccatatcggagacagcagatgctatttgcttcaggacgatgatttcgttcaagtgacagaagaccattcgcttgtaaatgaactggttcgcactggagagatttccagagaagacgctgaacatcatccgcgaaaaaatgtgttgacgaaggcgcttggaacagaccagttagtcagtattgacacccgttcctttgatatagaacccggagacaaactgcttctatgttctgacggactgacaaataaagtggaaggcactgagttaaaagacatcctgcaaagcgattcagctcctcaggaaaaagtaaacctgcttgtggacaaagccaatcagaatggcggagaagacaacattacagcagttttgcttgagcttgctttacaagttgaagagggtgaagatcagtgc

SEQ ID NO: 30 - protein, 254 aa, Bacillus subtilis
MLTALKTDTGKIRQHNEDDAGIFKGKDEFILAVVADGMGGHLAGDVASKMAVKAMGEKWNEAETIPTAPSECEKWLIEQILSVNSKIYDHAQAHEECQGMGTTIVCALFTGKTVSVAHIGDSRCYLLQDDDFVQVTEDHSLVNELVRTGEISREDAEHHPRKNVLTKALGTDQLVSIDTRSFDIEPGDKLLLCSDGLTNKVEGTELKDILQSDSAPQEKVNLLVDKANQNGGEDNITAVLLELALQVEEGEDQC

SEQ ID NO: 31 - DNA, 1545 bp, Bacillus subtilis
atgacagtcacatacgcgcacgaaccatttaccgattttacggaagcaaagaataaaactgcatttggggagtcattggcctttgtaaacactcagctcggcaagcattatccgcttgtcataaatggagaaaaaattgaaacggaccgcaaaatcatttctattaacccggcaaataaagaagagatcattgggtacgcgtctacagcggatcaagagcttgctgaaaaagcgatgcaagccgcattgcaggcatttgattcctggaaaaaacaaagaccggagcaccgcgcaaatattctctttaaggcagcggctattttgcgcagaagaaagcatgaattttcaagctatcttgtgaaggaagcaggaaaaccgtggaaggaagcagatgcggacacggctgaagcgatagactttttagagttctacgcgcgccaaatgttaaagctcaaggaaggggctccggtgaagagccgtgctggcgaggtcaatcaatatcattacgaagcgcttggcgtcggcatcgtcatttctccatttaacttcccgctcgcgattatggcgggaacagcggtggcagcgattgtgacaggaaatacgattctcttaaaaccggctgacgcagccccggtagtggcagcaaaatttgtcgaggtcatggaggaagcgggtctgccaaacggcgttctgaattacattccgggagatggtgcggagatcggtgatttcttagttgagcatccgaagacacggtttgtctcatttacaggttcccgtgcagtcggctgccggatttatgagcgagctgccaaagtgcagccgggccaaaaatggctcaaacgggtaattgcagaaatgggcggaaaagacacagtgcttgtcgacaaggacgctgatcttgaccttgctgcatcctctatcgtgtattcagcatttggatattcaggacagaagtgttctgcgggctcccgcgcggtcattcatcaggatgtgtatgatgaagtggtggaaaaagctgtggcgctgaccaaaacgctgactgtcggcaatccagaagatcctgatacgtatatgggtcccgtgattcatgaagcatcctacaacaaagtgatgaaatacattgaaatcggcaaatctgaaggcaagctattggccggcggagaaggcgatgattcaaaaggctactttattcagccgacgatctttgcagatgttgatgaaaacgcccgcttgatgcaggaagaaattttcggcccggttgttgcgatttgcaaagcgcgtgatttcgatcatatgctggagattgccaataacacggaatacggattaacaggtgcgcttctgacgaaaaaccgtgcgcacattgaacgggcgcgcgaggatttccatgtcggaaacctatattttaacagaggatgtaccggagcaattgtcggctatcagccgttcggcggttttaatatgtcaggaacagactcaaaagcaggcggtcccgattacttaattcttcatatgcaagccaaaacaacgtccgaagctttt

SEQ ID NO: 32 - protein, 515 aa, Bacillus subtilis
MTVTYAHEPFTDFTEAKNKTAFGESLAFVNTQLGKHYPLVINGEKIETDRKIISINPANKEEIIGYASTADQELAEKAMQAALQAFDSWKKQRPEHRANILFKAAAILRRRKHEFSSYLVKEAGKPWKEADADTAEAIDFLEFYARQMLKLKEGAPVKSRAGEVNQYHYEALGVGIVISPFNFPLAIMAGTAVAAIVTGNTILLKPADAAPVVAAKFVEVMEEAGLPNGVLNYIPGDGAEIGDFLVEHPKTRFVSFTGSRAVGCRIYERAAKVQPGQKWLKRVIAEMGGKDTVLVDKDADLDLAASSIVYSAFGYSGQKCSAGSRAVIHQDVYDEVVEKAVALTKTLTVGNPEDPDTYMGPVIHEASYNKVMKYIEIGKSEGKLLAGGEGDDSKGYFIQPTIFADVDENARLMQEEIFGPVVAICKARDFDHMLEIANNTEYGLTGALLTKNRAHIERAREDFHVGNLYFNRGCTGAIVGYQPFGGFNMSGTDSKAGGPDYLILHMQAKTTSEAF

SEQ ID NO: 33 - DNA, 1203 bp, Bacillus subtilis
atgacagctttatctaaatccaaagaaattattgatcagacgtctcattacggagccaacaattatcacccgctcccgattgttatttctgaagcgctgggtgcttgggtaaaggacccggaaggcaatgaatatatggatatgctgagtgcttactctgcggtaaaccaggggcacagacacccgaaaatcattcaggcattaaaggatcaggctgataaaatcaccctcacgtcacgcgcgtttcataacgatcagcttgggccgttttacgaaaaaacagctaaactgacaggcaaagagatgattctgccgatgaatacaggagccgaagcggttgaatccgcggtgaaagcggcgagacgctgggcgtatgaagtgaagggcgtagctgacaatcaagcggaaattatcgcatgtgtcgggaacttccacggccgcacgatgctggcggtatctctttcttctgaagaggaatataaacgaggattcggcccgatgcttccaggaatcaaactcattccttacggcgatgtggaagcgcttcgacaggccattacgccgaatacagcggcattcttgtttgaaccgattcaaggcgaagcgggcattgtgattccgcctgaaggatttttacaggaagcggcggcgatttgtaaggaagagaatgtcttgtttattgcggatgaaattcagacgggtctcggacgtacaggcaagacgtttgcctgtgactgggacggcattgttccggatatgtatatcttgggcaaagcgcttggcggcggtgtgttcccgatctcttgcattgcggcggaccgcgagatcctaggcgtgtttaaccctggctcacacggctcaacatttggtggaaacccgcttgcatgtgcagtgtctatcgcttcattagaagtgctggaggatgaaaagctggcggatcgttctcttgaacttggtgaatactttaaaagcgagcttgagagtattgacagccctgtcattaaagaagtccgcggcagagggctgtttatcggtgtggaattgactgaagcggcacgtccgtattgtgagcgtttgaaggaagagggacttttatgcaaggaaacgcatgatacagtcattcgttttgcaccgccattaatcatttccaaagaggacttggattgggcgatagagaaaattaagcacgtgctgcgaaacgca

SEQ ID NO: 34 - protein, 401 aa, Bacillus subtilis
MTALSKSKEIIDQTSHYGANNYHPLPIVISEALGAWVKDPEGNEYMDMLSAYSAVNQGHRHPKIIQALKDQADKITLTSRAFHNDQLGPFYEKTAKLTGKEMILPMNTGAEAVESAVKAARRWAYEVKGVADNQAEIIACVGNFHGRTMLAVSLSSEEEYKRGFGPMLPGIKLIPYGDVEALRQAITPNTAAFLFEPIQGEAGIVIPPEGFLQEAAAICKEENVLFIADEIQTGLGRTGKTFACDWDGIVPDMYILGKALGGGVFPISCIAADREILGVFNPGSHGSTFGGNPLACAVSIASLEVLEDEKLADRSLELGEYFKSELESIDSPVIKEVRGRGLFIGVELTEAARPYCERLKEEGLLCKETHDTVIRFAPPLIISKEDLDWAIEKIKHVLRNA

SEQ ID NO: 35 - DNA, 888 bp, Bacillus subtilis
atggataaaacgatttcggttattggaatgccaatggatttaggacaagcacgacgcggagtggatatgggcccgagtgccatccggtacgctcatctgatcgagaggctgtcagacatggggtatacggttgaagatctcggtgacattccgatcaatcgcgaaaaaatcaaaaatgacgaggaactgaaaaacctgaattccgttttggcgggaaatgaaaaactcgcgcaaaaggtcaacaaagtcattgaagagaaaaaattcccgcttgtcctgggcggtgaccacagtattgcgatcggcacgcttgcaggcacagcgaagcattacgataatctcggcgtcatctggtatgacgcgcacggcgatttgaatacacttgaaacttcaccatcgggcaatattcacggcatgccgctcgcggtcagcctaggcattggccacgagtcactggttaaccttgaaggctacgcgcctaaaatcaaaccggaaaacgtcgtcatcattggcgcccggtcacttgatgaaggggagcgcaagtacattaaggaaagcggcatgaaggtgtacacaatgcacgaaatcgatcgtcttggcatgacaaaggtcattgaagaaacccttgattatttatcagcatgtgatggcgtccatctgagccttgatctggacggacttgatccgaacgacgcaccgggtgtcggaacccctgtcgtcggcggcatcagctaccgggagagccatttggctatggaaatgctgtatgacgcaggcatcattacctcagccgaattcgttgaggttaacccgatccttgatcacaaaaacaaaacgggcaaaacagcagtagagctcgtagaatccctgttagggaagaagctgctg

SEQ ID NO: 36 - protein, 296 aa, Bacillus subtilis
MDKTISVIGMPMDLGQARRGVDMGPSAIRYAHLIERLSDMGYTVEDLGDIPINREKIKNDEELKNLNSVLAGNEKLAQKVNKVIEEKKFPLVLGGDHSIAIGTLAGTAKHYDNLGVIWYDAHGDLNTLETSPSGNIHGMPLAVSLGIGHESLVNLEGYAPKIKPENVVIIGARSLDEGERKYIKESGMKVYTMHEIDRLGMTKVIEETLDYLSACDGVHLSLDLDGLDPNDAPGVGTPVVGGISYRESHLAMEMLYDAGIITSAEFVEVNPILDHKNKTGKTAVELVESLLGKKLL

SEQ ID NO: 37 - DNA, 1041 bp, Bacillus subtilis
atgcagagtggaaagatgaaagctctaatgaaaaaggacggggcgttcggtgctgtgctgactgaagttcccattcctgagattgataaacatgaagtcctcataaaagtgaaagccgcttccatatgcggcacggatgtccacatttataattgggatcaatgggcacgtcagagaatcaaaacaccctatgttttcggccatgagttcagcggcatcgtagagggcgtgggagagaatgtcagcagtgtaaaagtgggagagtatgtgtctgcggaaacacacattgtctgtggtgaatgtgtcccttgcctaacaggaaaatctcatgtgtgtaccaatactgctataatcggagtggacacggcaggctgttttgcggagtatgtaaaagttccagctgataacatttggagaaatcccgctgatatggacccgtcgattgcttccattcaagagcctttaggaaatgcagttcataccgtactcgagagccagcctgcaggaggaacgactgcagtcattggatgcggaccgattggtcttatggctgttgcggttgcaaaagcagcaggagcttctcaggtgatagcgattgataagaatgaatacaggctgaggcttgcaaaacaaatgggagcgacttgtactgtttctattgaaaaagaagacccgctcaaaattgtaagcgctttaacgagtggagaaggagcagatcttgtttgtgagatgtcgggccatccctcagcgattgcccaaggtcttgcgatggctgcgaatggcggaagatttcatattctcagcttgccggaacatccggtgacaattgatttgacgaataaagtggtatttaaagggcttaccatccaaggaatcacaggaagaaaaatgttttcaacatggcgccaggtgtctcagttgatcagttcaaacatgatcgatcttgcacctgttattacccatcagtttccattagaggagtttgaaaaaggtttcgaactgatgagaagcgggcagtgcggaaaagtaattttaattcca

SEQ ID NO: 38 - protein, 347 aa, Bacillus subtilis
MQSGKMKALMKKDGAFGAVLTEVPIPEIDKHEVLIKVKAASICGTDVHIYNWDQWARQRIKTPYVFGHEFSGIVEGVGENVSSVKVGEYVSAETHIVCGECVPCLTGKSHVCTNTAIIGVDTAGCFAEYVKVPADNIWRNPADMDPSIASIQEPLGNAVHTVLESQPAGGTTAVIGCGPIGLMAVAVAKAAGASQVIAIDKNEYRLRLAKQMGATCTVSIEKEDPLKIVSALTSGEGADLVCEMSGHPSAIAQGLAMAANGGRFHILSLPEHPVTIDLTNKVVFKGLTIQGITGRKMFSTWRQVSQLISSNMIDLAPVITHQFPLEEFEKGFELMRSGQCGKVILIP

SEQ ID NO: 39 - DNA, 6127 bp, Bacillus subtilis
taatacgataagaacagcttagaaatacacaagagtgtgtataaagcaattagaatgagttgagttagagaatagggtagcagagaatgagtttagttgagctgagacattatgtttattctacccaaaagaagtctttcttttgggtttatttgttatatagtattttatcctctcatgccatcttctcattctccttgccataaggagtgagagcaatgaatttccaatcaaacatttccgcatttttagaggacagcttgtcccaccacacgataccgattgtggagaccttcacagtcgatacactgacacccattcaaatgatagagaagcttgacagggagattacgtatcttcttgaaagcaaggacgatacatccacttggtccagatattcgtttatcggcctgaatccatttctcacaattaaagaagagcagggccgtttttcggccgctgatcaggacagcaaatctctttacacaggaaatgaactaaaagaagtgctgaactggatgaataccacatacaaaatcaaaacacctgagcttggcattccttttgtcggcggagctgtcgggtacttaagctatgatatgatcccgctgattgagccttctgttccttcgcataccaaagaaacagacatggaaaagtgtatgctgtttgtttgccggacattaattgcgtatgatcatgaaaccaaaaacgtccactttatccaatatgcaaggctcactggagaggaaacaaaaaacgaaaaaatggatgtattccatcaaaatcatctggagcttcaaaatctcattgaaaaaatgatggaccaaaaaaacataaaagagctgtttctttctgctgattcatacaagacacccagctttgagacagtatcttctaattatgaaaaatcggcttttatggctgatgtagaaaaaatcaaaagctatataaaagcaggcgatatcttccagggtgttttatcacaaaaatttgaggtgccgataaaagcagatgcttttgagttataccgagtgcttaggatcgtcaatccttcgccgtatatgtattatatgaaactgctagacagagaaatagtcggcagctctccggaacggttaatacacgttcaagacgggcacttagaaatccatccgattgccggtacgagaaaacgcggtgcagacaaagctgaagatgagagactgaaggttgagctcatgaaggatgaaaaagaaaaagcggagcattacatgctcgttgatcttgcccgaaacgatatcggcagagtagcagagtatggttctgtttctgtgccggagttcacaaaaattgtttccttttcacatgtcatgcacattatctcggtggttacaggccgattgaaaaaaggggttcatcctgtcgatgcactgatgtctgctttcccggcggggactttaacaggcgcacccaaaatccgtgccatgcagcttttgcaagaactcgagccaacaccgagagagacatacggagggtgtattgcctacattgggtttgacgggaatatcgactcttgtattacgattcgcacgatgagtgtaaagaacggtgttgcatcgatacaggcaggtgctggcattgttgctgattctgttccggaagccgaatacgaagaaagctgtaataaagccggtgcgctgctgaaaacgattcatattgcagaagacatgtttcatagcaaggaggataaagctgatgaacagatttctacaattgtgcgttgacggaaaaacccttactgccggtgaggctgaaacgctgatgaatatgatgatggcagcggaaatgactccttctgaaatgggggggatattgtcaattcttgctcatcggggggagacgccagaagagcttgcgggttttgtgaaggcaatgcgggcacacgctcttacagtcgatggacttcctgatattgttgatacatgcggaacagggggagacggtatttccacttttaatatctcaacggcctcggcaattgttgcctcggcagctggtgcgaaaatcgctaagcatggcaatcgctctgtctcttctaaaagcggaagcgctgatgttttagaggagctagaggtttctattcaaaccactcccgaaaaggtcaaaagcagcattgaaacaaacaacatgggatttctttttgcgccgctttaccattcgtctatgaaacatgtagcaggtactagaaaagagctaggtttcagaacggtatttaatctgcttgggccgctcagcaatcctttacaggcgaagcgtcaggtgattggggtctattctgttgaaaaagctggactgatggcaagcgcactggagacgtttcagccgaagcacgttatgtttgtatcaagccgtgacggtttagatgagctttcaattacagcaccgaccgacgtgattgaattaaaggacggagagcgccgggagtataccgtttcacccgaagatttcggtttcacaaatggcagacttgaagatttacaggtgcagtctccgaaagagagcgcttatctcattcagaatatttttgaaaataaaagcagcagttccgctttatctattacggcttttaatgcgggtgctgcgatttacacggcgggaattaccgcctcactgaaggaaggaacggagctggcgttagagacgattacaagcggaggcgctgccgcgcagcttgaacgactaaagcagaaagaggaagagatctatgcttgaaaaaatcatcaaacaaaagaaagaagaagtgaaaacactggttctgccggtagagcagcctttcgagaaacgttcatttaaggaggcgccggcaagcccgaatcggtttatcgggttgattgccgaagtgaagaaagcatcgccgtcaaaagggcttattaaagaggattttgtacctgtgcagattgcaaaagactatgaggctgcgaaggcagatgcgatttccgttttaacagacaccccgttttttcaaggggaaaacagctatttatcagacgtaaagcgtgctgtttcgattcctgtacttagaaaagattttattattgattctcttcaagtagaggaatcaagaagaatcggagcggatgccatattgttaatcggcgaggtgcttgatcccttacaccttcatgaattatatcttgaagcaggtgaaaaggggatggacgtgttagtggaggttcatgatgcatcaacgctagaacaaatattgaaagtgttcacacccgacattctcggcgtaaataatcgaaacctaaaaacgtttgaaacatctgtaaagcagacagaacaaatcgcatctctcgttccgaaagaatccttgcttgtcagcgaaagcggaatcggttctttagaacatttaacatttgtcaatgaacatggggcgcgagctgtacttatcggtgaatcattgatgagacaaacttctcagcgtaaagcaatccatgctttgtttagggagtgaggttgtgaagaaaccggcattaaaatattgcggtattcggtcactaaaggatttgcagcttgcggcggaatcacaggctgattacctaggatttatttttgctgaaagcaaacgaaaagtatctccggaagatgtgaaaaaatggctgaaccaagttcgtgtcgaaaaacaggttgcaggtgtttttgttaatgaatcaatagagacgatgtcacgtattgccaagagcttgaagctcgacgtcattcagcttcacggtgatgaaaaaccggcggatgtcgctgctcttcgcaagctgacaggctgtgaaatatggaaggcgcttcaccatcaagataacacaactcaagaaatagcccgctttaaagataatgttgacggctttgtgattgattcatctgtaaaagggtctagaggcggaactggtgttgcattttcttgggactgtgtgccggaatatcagcaggcggctattggtaaacgctgctttatcgctggcggcgtgaatccggatagcatcacacgcctattgaaatggcagccagaaggaattgaccttgccagcggaattgaaaaaaacggacaaaaagatcagaatctgatgaggcttttagaagaaaggatgaaccgatatgtatccatatccgaatgaaataggcagatacggtgattttggcggaaagtttgttccggaaacactcatgcagccgttagatgaaatacaaacagcatttaaacaaatcaaggatgatcccgcttttcgtgaagagtattataagctgttaaaggactattccggacgcccgactgcattaacatacgctgatcgagtcactgaatacttaggcggcgcgaaaatctatttgaaacgagaagatttaaaccatacaggttctcataaaatcaataatgcgctaggtcaagcgctgcttgctaaaaaaatgggcaaaacgaaaatcattgctgaaaccggtgccggccagcatggtgttgccgctgcaacagttgcagccaaattcggcttttcctgtactgtgtttatgggtgaagaggatgttgcccgccagtctctgaacgttttccgcatgaagcttcttggagcggaggtagtgcctgtaacaagcggaaacggaacattgaaggatgccacaaatgaggcgatccggtactgggttcagcattgtgaggatcacttttatatgattggatcagttgtcggcccgcatccttatccgcaagtggtccgtgaatttcaaaaaatgatcggagaggaagcgaaggatcagttgaaacgtattgaaggcactatgcctgataaagtagtggcatgtgtaggcggaggaagcaatgcgatgggtatgtttcaggcatttttaaatgaagatgttgaactgatcggcgctgaagcagcaggaaaaggaattgatacacctcttcatgccgccactatttcgaaaggaaccgtaggggttattcacggttcattgacttatctcattcaggatgagttcgggcaaattattgagccctactctatttcagccggtctcgactatcctggaatcggtccggagcatgcatatttgcataaaagcggccgtgtcacttatgacagtataaccgatgaagaagcggtggatgcattaaagcttttgtcagaaaaagaggggattttgccggcaatcgaatctgcccatgcgttagcgaaagcattcaaactcgccaaaggaatggatcgcggtcaactcattctcgtctgtttatcaggccggggagacaaggatgtcaacacattaatgaatgtattggaagaagaggtgaaagcccatgtttaaattggatcttcaaccatcagaaaaattgtttatcccgtttattacggcgggcgatccagttcctgaggtttcgattgaactggcgaagtcactccaaaaagcaggcgccacagcattggagcttggtgttgcatactctgacccgcttgcagacggtccggtgatccagcgggcttcaaagcgggcgcttgatcaaggaatgaatatcgtaaaggcaatcgaattaggcggagaaatgaaaaaaaacggagtgaatattccgattatcctctttacgtattataatcctgtgttacaattgaacaaagaatactttttcgctttactgcgggaaaatcatattgacggtctgcttgttccggatctgccattagaagaaagcaacagccttcaagaggaatgtaaaagccatgaggtgacgtatatttctttagttgcgccgacaagcgaaagccgtttgaaaaccattattgaacaagccgaggggttcgtctactgtgtatcttctctgggtgtgaccggtgtccgcaatgagttcaattcatccgtgtacccgttcattcgtactgtgaagaatctcagcactgttccggttgctgtagggttcggtatatcaaaccgtgaacaggtcataaagatgaatgaaattagtgacggtgtcgtagtgggaagtgcgctcgtcagaaaaatagaagaattaaaggaccggctcatcagcgctgaaacgagaaatcaggcgctgcaggagtttgaggattatgcaatggcgtttagcggcttgtacagtttaaaa

SEQ ID NO: 40 - DNA, 801 bp, Bacillus subtilis
atgtttaaattggatcttcaaccatcagaaaaattgtttatcccgtttattacggcgggcgatccagttcctgaggtttcgattgaactggcgaagtcactccaaaaagcaggcgccacagcattggagcttggtgttgcatactctgacccgcttgcagacggtccggtgatccagcgggcttcaaagcgggcgcttgatcaaggaatgaatatcgtaaaggcaatcgaattaggcggagaaatgaaaaaaaacggagtgaatattccgattatcctctttacgtattataatcctgtgttacaattgaacaaagaatactttttcgctttactgcgggaaaatcatattgacggtctgcttgttccggatctgccattagaagaaagcaacagccttcaagaggaatgtaaaagccatgaggtgacgtatatttctttagttgcgccgacaagcgaaagccgtttgaaaaccattattgaacaagccgaggggttcgtctactgtgtatcttctctgggtgtgaccggtgtccgcaatgagttcaattcatccgtgtacccgttcattcgtactgtgaagaatctcagcactgttccggttgctgtagggttcggtatatcaaaccgtgaacaggtcataaagatgaatgaaattagtgacggtgtcgtagtgggaagtgcgctcgtcagaaaaatagaagaattaaaggaccggctcatcagcgctgaaacgagaaatcaggcgctgcaggagtttgaggattatgcaatggcgtttagcggcttgtacagtttaaaa

SEQ ID NO: 41 - protein, 267 aa, Bacillus subtilis
MFKLDLQPSEKLFIPFITAGDPVPEVSIELAKSLQKAGATALELGVAYSDPLADGPVIQRASKRALDQGMNIVKAIELGGEMKKNGVNIPIILFTYYNPVLQLNKEYFFALLRENHIDGLLVPDLPLEESNSLQEECKSHEVTYISLVAPTSESRLKTIIEQAEGFVYCVSSLGVTGVRNEFNSSVYPFIRTVKNLSTVPVAVGFGISNREQVIKMNEISDGVVVGSALVRKIEELKDRLISAETRNQALQEFEDYAMAFSGLYSLK

SEQ ID NO: 42 - DNA, 1195 bp, Bacillus subtilis
atgtatccatatccgaatgaaataggcagatacggtgattttggcggaaagtttgttccggaaacactcatgcagccgttagatgaaatacaaacagcatttaaacaaatcaaggatgatcccgcttttcgtgaagagtattataagctgttaaaggactattccggacgcccgactgcattaacatacgctgatcgagtcactgaatacttaggcggcgcgaaaatctatttgaaacgagaagatttaaaccatacaggttctcataaaatcaataatgcgctaggtcaagcgctgcttgctaaaaaaatgggcaaaacgaaaatcattgctgaaaccggtgccggccagcatggtgttgccgctgcaacagttgcagccaaattcggcttttcctgtactgtgtttatgggtgaagaggatgttgcccgccagtctctgaacgttttccgcatgaagcttcttggagcggaggtagtgcctgtaacaagcggaaacggaacattgaaggatgccacaaatgaggcgatccggtactgggttcagcattgtgaggatcacttttatatgattggatcagttgtcggcccgcatccttatccgcaagtggtccgtgaatttcaaaaaatgatcggagaggaagcgaaggatcagttgaaacgtattgaaggcactatgcctgataaagtagtggcatgtgtaggcggaggaagcaatgcgatgggtatgtttcaggcatttttaaatgaagatgttgaactgatcggcgctgaagcagcaggaaaaggaattgatacacctcttcatgccgccactatttcgaaaggaaccgtaggggttattcacggttcattgacttatctcattcaggatgagttcgggcaaattattgagccctactctatttcagccggtctcgactatcctggaatcggtccggagcatgcatatttgcataaaagcggccgtgtcacttatgacagtataaccgatgaagaagcggtggatgcattaaagcttttgtcagaaaaagaggggattttgccggcaatcgaatctgcccatgcgttagcgaaagcattcaaactcgccaaaggaatggatcgcggtcaactcattctcgtctgtttatcaggccggggagacaaggatgtcaacacattaatgaatgtattggaagaagaggtgaaagccc

SEQ ID NO: 43 - protein, 400 aa, Bacillus subtilis
MYPYPNEIGRYGDFGGKFVPETLMQPLDEIQTAFKQIKDDPAFREEYYKLLKDYSGRPTALTYADRVTEYLGGAKIYLKREDLNHTGSHKINNALGQALLAKKMGKTKIIAETGAGQHGVAAATVAAKFGFSCTVFMGEEDVARQSLNVFRMKLLGAEVVPVTSGNGTLKDATNEAIRYWVQHCEDHFYMIGSVVGPHPYPQVVREFQKMIGEEAKDQLKRIEGTMPDKVVACVGGGSNAMGMFQAFLNEDVELIGAEAAGKGIDTPLHAATISKGTVGVIHGSLTYLIQDEFGQIIEPYSISAGLDYPGIGPEHAYLHKSGRVTYDSITDEEAVDALKLLSEKEGILPAIESAHALAKAFKLAKGMDRGQLILVCLSGRGDKDVNTLMNVLEEEVKAHV

SEQ ID NO: 44 - DNA, 757 bp, Bacillus subtilis
atgcttgaaaaaatcatcaaacaaaagaaagaagaagtgaaaacactggttctgccggtagagcagcctttcgagaaacgttcatttaaggaggcgccggcaagcccgaatcggtttatcgggttgattgccgaagtgaagaaagcatcgccgtcaaaagggcttattaaagaggattttgtacctgtgcagattgcaaaagactatgaggctgcgaaggcagatgcgatttccgttttaacagacaccccgttttttcaaggggaaaacagctatttatcagacgtaaagcgtgctgtttcgattcctgtacttagaaaagattttattattgattctcttcaagtagaggaatcaagaagaatcggagcggatgccatattgttaatcggcgaggtgcttgatcccttacaccttcatgaattatatcttgaagcaggtgaaaaggggatggacgtgttagtggaggttcatgatgcatcaacgctagaacaaatattgaaagtgttcacacccgacattctcggcgtaaataatcgaaacctaaaaacgtttgaaacatctgtaaagcagacagaacaaatcgcatctctcgttccgaaagaatccttgcttgtcagcgaaagcggaatcggttctttagaacatttaacatttgtcaatgaacatggggcgcgagctgtacttatcggtgaatcattgatgagacaaacttctcagcgtaaagcaatccatgctttgtttagggagtgaggtt

SEQ ID NO: 45 - protein, 250 aa, Bacillus subtilis
MLEKIIKQKKEEVKTLVLPVEQPFEKRSFKEAPASPNRFIGLIAEVKKASPSKGLIKEDFVPVQIAKDYEAAKADAISVLTDTPFFQGENSYLSDVKRAVSIPVLRKDFIIDSLQVEESRRIGADAILLIGEVLDPLHLHELYLEAGEKGMDVLVEVHDASTLEQILKVFTPDILGVNNRNLKTFETSVKQTEQIASLVPKESLLVSESGIGSLEHLTFVNEHGARAVLIGESLMRQTSQRKAIHALFRE

SEQ ID NO: 46 - DNA, 1009 bp, Bacillus subtilis
atgaacagatttctacaattgtgcgttgacggaaaaacccttactgccggtgaggctgaaacgctgatgaatatgatgatggcagcggaaatgactccttctgaaatgggggggatattgtcaattcttgctcatcggggggagacgccagaagagcttgcgggttttgtgaaggcaatgcgggcacacgctcttacagtcgatggacttcctgatattgttgatacatgcggaacagggggagacggtatttccacttttaatatctcaacggcctcggcaattgttgcctcggcagctggtgcgaaaatcgctaagcatggcaatcgctctgtctcttctaaaagcggaagcgctgatgttttagaggagctagaggtttctattcaaaccactcccgaaaaggtcaaaagcagcattgaaacaaacaacatgggatttctttttgcgccgctttaccattcgtctatgaaacatgtagcaggtactagaaaagagctaggtttcagaacggtatttaatctgcttgggccgctcagcaatcctttacaggcgaagcgtcaggtgattggggtctattctgttgaaaaagctggactgatggcaagcgcactggagacgtttcagccgaagcacgttatgtttgtatcaagccgtgacggtttagatgagctttcaattacagcaccgaccgacgtgattgaattaaaggacggagagcgccgggagtataccgtttcacccgaagatttcggtttcacaaatggcagacttgaagatttacaggtgcagtctccgaaagagagcgcttatctcattcagaatatttttgaaaataaaagcagcagttccgctttatctattacggcttttaatgcgggtgctgcgatttacacggcgggaattaccgcctcactgaaggaaggaacggagctggcgttagagacgattacaagcggaggcgctgccgcgcagcttgaacgactaaagcagaaagaggaagagatct

SEQ ID NO: 47 - protein, 338 aa, Bacillus subtilis
MNRFLQLCVDGKTLTAGEAETLMNMMMAAEMTPSEMGGILSILAHRGETPEELAGFVKAMRAHALTVDGLPDIVDTCGTGGDGISTFNISTASAIVASAAGAKIAKHGNRSVSSKSGSADVLEELEVSIQTTPEKVKSSIETNNMGFLFAPLYHSSMKHVAGTRKELGFRTVFNLLGPLSNPLQAKRQVIGVYSVEKAGLMASALETFQPKHVMFVSSRDGLDELSITAPTDVIELKDGERREYTVSPEDFGFTNGRLEDLQVQSPKESAYLIQNIFENKSSSSALSITAFNAGAAIYTAGITASLKEGTELALETITSGGAAAQLERLKQKEEEIYA

SEQ ID NO: 48 - DNA, 1519 bp, Bacillus subtilis
atgaatttccaatcaaacatttccgcatttttagaggacagcttgtcccaccacacgataccgattgtggagaccttcacagtcgatacactgacacccattcaaatgatagagaagcttgacagggagattacgtatcttcttgaaagcaaggacgatacatccacttggtccagatattcgtttatcggcctgaatccatttctcacaattaaagaagagcagggccgtttttcggccgctgatcaggacagcaaatctctttacacaggaaatgaactaaaagaagtgctgaactggatgaataccacatacaaaatcaaaacacctgagcttggcattccttttgtcggcggagctgtcgggtacttaagctatgatatgatcccgctgattgagccttctgttccttcgcataccaaagaaacagacatggaaaagtgtatgctgtttgtttgccggacattaattgcgtatgatcatgaaaccaaaaacgtccactttatccaatatgcaaggctcactggagaggaaacaaaaaacgaaaaaatggatgtattccatcaaaatcatctggagcttcaaaatctcattgaaaaaatgatggaccaaaaaaacataaaagagctgtttctttctgctgattcatacaagacacccagctttgagacagtatcttctaattatgaaaaatcggcttttatggctgatgtagaaaaaatcaaaagctatataaaagcaggcgatatcttccagggtgttttatcacaaaaatttgaggtgccgataaaagcagatgcttttgagttataccgagtgcttaggatcgtcaatccttcgccgtatatgtattatatgaaactgctagacagagaaatagtcggcagctctccggaacggttaatacacgttcaagacgggcacttagaaatccatccgattgccggtacgagaaaacgcggtgcagacaaagctgaagatgagagactgaaggttgagctcatgaaggatgaaaaagaaaaagcggagcattacatgctcgttgatcttgcccgaaacgatatcggcagagtagcagagtatggttctgtttctgtgccggagttcacaaaaattgtttccttttcacatgtcatgcacattatctcggtggttacaggccgattgaaaaaaggggttcatcctgtcgatgcactgatgtctgctttcccggcggggactttaacaggcgcacccaaaatccgtgccatgcagcttttgcaagaactcgagccaacaccgagagagacatacggagggtgtattgcctacattgggtttgacgggaatatcgactcttgtattacgattcgcacgatgagtgtaaagaacggtgttgcatcgatacaggcaggtgctggcattgttgctgattctgttccggaagccgaatacgaagaaagctgtaataaagccggtgcgctgctgaaaacgattcatattgcagaagacatgtttcatagcaaggaggataaagctg

SEQ ID NO: 49 - protein, 515 aa, Bacillus subtilis
MNFQSNISAFLEDSLSHHTIPIVETFTVDTLTPIQMIEKLDREITYLLESKDDTSTWSRYSFIGLNPFLTIKEEQGRFSAADQDSKSLYTGNELKEVLNWMNTTYKIKTPELGIPFVGGAVGYLSYDMIPLIEPSVPSHTKETDMEKCMLFVCRTLIAYDHETKNVHFIQYARLTGEETKNEKMDVFHQNHLELQNLIEKMMDQKNIKELFLSADSYKTPSFETVSSNYEKSAFMADVEKIKSYIKAGDIFQGVLSQKFEVPIKADAFELYRVLRIVNPSPYMYYMKLLDREIVGSSPERLIHVQDGHLEIHPIAGTRKRGADKAEDERLKVELMKDEKEKAEHYMLVDLARNDIGRVAEYGSVSVPEFTKIVSFSHVMHIISVVTGRLKKGVHPVDALMSAFPAGTLTGAPKIRAMQLLQELEPTPRETYGGCIAYIGFDGNIDSCITIRTMSVKNGVASIQAGAGIVADSVPEAEYEESCNKAGALLKTIHIAEDMFHSKEDKADEQISTIVR

SEQ ID NO: 50 - DNA, 628 bp, Bacillus subtilis
gtgaagaaaccggcattaaaatattgcggtattcggtcactaaaggatttgcagcttgcggcggaatcacaggctgattacctaggatttatttttgctgaaagcaaacgaaaagtatctccggaagatgtgaaaaaatggctgaaccaagttcgtgtcgaaaaacaggttgcaggtgtttttgttaatgaatcaatagagacgatgtcacgtattgccaagagcttgaagctcgacgtcattcagcttcacggtgatgaaaaaccggcggatgtcgctgctcttcgcaagctgacaggctgtgaaatatggaaggcgcttcaccatcaagataacacaactcaagaaatagcccgctttaaagataatgttgacggctttgtgattgattcatctgtaaaagggtctagaggcggaactggtgttgcattttcttgggactgtgtgccggaatatcagcaggcggctattggtaaacgctgctttatcgctggcggcgtgaatccggatagcatcacacgcctattgaaatggcagccagaaggaattgaccttgccagcggaattgaaaaaaacggacaaaaagatcagaatctgatgaggcttttagaagaaaggatgaaccgat

SEQ ID NO: 51 - protein, 215 aa, Bacillus subtilis
MKKPALKYCGIRSLKDLQLAAESQADYLGFIFAESKRKVSPEDVKKWLNQVRVEKQVAGVFVNESIETMSRIAKSLKLDVIQLHGDEKPADVAALRKLTGCEIWKALHHQDNTTQEIARFKDNVDGFVIDSSVKGSRGGTGVAFSWDCVPEYQQAAIGKRCFIAGGVNPDSITRLLKWQPEGIDLASGIEKNGQKDQNLMRLLEERMNRYVSISE

SEQ ID NO: 52 - DNA, 909 bp, Bacillus subtilis
gtgatcacaagagattttttcttatttttatccaaaagcggctttctcaataaaatggcgaggaactggggaagtcgggtagcagcgggtaaaattatcggcgggaatgactttaacagttcaatcccgaccattcgacagcttaacagccaaggcttgtcagttactgtcgatcatttaggcgagtttgtgaacagcgccgaggtcgcacgggagcgtacggaagagtgcattcaaaccattgcgaccatcgcggatcaggagctgaactcacacgtttctttaaaaatgacgtctttaggtttggatatagatatggatttggtgtatgaaaatatgacaaaaatccttcagacggccgagaaacataaaatcatggtcaccattgacatggaggacgaagtcagatgccagaaaacgcttgatattttcaaagatttcagaaagaaatacgagcatgtgagcacagtgctgcaagcctatctgtaccggacggaaaaagacattgacgatttggattctttaaacccgttccttcgccttgtaaaaggagcttataaagaatcagaaaaagtagctttcccggagaaaagcgatgtcgatgaaaattacaaaaaaatcatccgaaagcagctcttaaacggtcactatacagcgattgccacacatgacgacaaaatgatcgactttacaaagcagcttgccaaggaacatggcattgccaatgacaagtttgaatttcagatgctgtacggcatgcggtcgcaaacccagctcagcctcgtaaaagaaggttataacatgagagtctacctgccatacggcgaggattggtacggctactttatgagacgccttgcagaacgtccgtcaaacattgcatttgctttcaaaggaatgacaaagaag

SEQ ID NO: 53 - protein, 303 aa, Bacillus subtilis
MITRDFFLFLSKSGFLNKMARNWGSRVAAGKIIGGNDFNSSIPTIRQLNSQGLSVTVDHLGEFVNSAEVARERTEECIQTIATIADQELNSHVSLKMTSLGLDIDMDLVYENMTKILQTAEKHKIMVTIDMEDEVRCQKTLDIFKDFRKKYEHVSTVLQAYLYRTEKDIDDLDSLNPFLRLVKGAYKESEKVAFPEKSDVDENYKKIIRKQLLNGHYTAIATHDDKMIDFTKQLAKEHGIANDKFEFQMLYGMRSQTQLSLVKEGYNMRVYLPYGEDWYGYFMRRLAERPSNIAFAFKGMTKK

SEQ ID NO: 54 - DNA, 1545 bp, Bacillus subtilis
atgacaacaccttacaaacacgagccattcacaaatttccaagatcaaaactacgtggaagcgtttaaaaaagcgcttgcgacagtaagcgaatatttaggaaaagactatccgcttgtcattaacggcgagagagtggaaacggaagcgaaaatcgtttcaatcaacccagctgataaagaagaagtcgtcggccgagtgtcaaaagcgtctcaagagcacgctgagcaagcgattcaagcggctgcaaaagcatttgaagagtggagatacacgtctcctgaagagagagcggctgtcctgttccgcgctgctgccaaagtccgcagaagaaaacatgaattctcagctttgcttgtgaaagaagcaggaaagccttggaacgaggcggatgccgatacggctgaagcgattgacttcatggagtattatgcacgccaaatgatcgaactggcaaaaggcaaaccggtcaacagccgtgaaggcgagaaaaaccaatatgtatacacgccgactggagtgacagtcgttatcccgccttggaacttcttgtttgcgatcatggcaggcacaacagtggcgccgatcgttactggaaacacagtggttctgaaacctgcgagtgctacacctgttattgcagcaaaatttgttgaggtgcttgaagagtccggattgccaaaaggcgtagtcaactttgttccgggaagcggatcggaagtaggcgactatcttgttgaccatccgaaaacaagccttatcacatttacgggatcaagagaagttggtacgagaattttcgaacgcgcggcgaaggttcagccgggccagcagcatttaaagcgtgtcatcgctgaaatgggcggtaaagatacggttgttgttgatgaggatgcggacattgaattagcggctcaatcgatctttacttcagcattcggctttgcgggacaaaaatgctctgcaggttcacgtgcagtagttcatgaaaaagtgtatgatcaagtattagagcgtgtcattgaaattacggaatcaaaagtaacagctaaacctgacagtgcagatgtttatatgggacctgtcattgaccaaggttcttatgataaaattatgagctatattgagatcggaaaacaggaagggcgtttagtaagcggcggtactggtgatgattcgaaaggatacttcatcaaaccgacgatcttcgctgaccttgatccgaaagcaagactcatgcaggaagaaattttcggacctgtcgttgcattttgtaaagtgtcagactttgatgaagctttagaagtggcaaacaatactgaatatggtttgacaggcgcggttatcacaaacaaccgcaagcacatcgagcgtgcgaaacaggaattccatgtcggaaacctatacttcaaccgcaactgtacaggtgctatcgtcggctaccatccgtttggcggcttcaaaatgtcgggaacggattcaaaagcaggcgggccggattacttggctctgcatatgcaagcaaaaacaatcagtgaaatgttc

SEQ ID NO: 55 - protein, 515 aa, Bacillus subtilis
MTTPYKHEPFTNFQDQNYVEAFKKALATVSEYLGKDYPLVINGERVETEAKIVSINPADKEEVVGRVSKASQEHAEQAIQAAAKAFEEWRYTSPEERAAVLFRAAAKVRRRKHEFSALLVKEAGKPWNEADADTAEAIDFMEYYARQMIELAKGKPVNSREGEKNQYVYTPTGVTVVIPPWNFLFAIMAGTTVAPIVTGNTVVLKPASATPVIAAKFVEVLEESGLPKGVVNFVPGSGSEVGDYLVDHPKTSLITFTGSREVGTRIFERAAKVQPGQQHLKRVIAEMGGKDTVVVDEDADIELAAQSIFTSAFGFAGQKCSAGSRAVVHEKVYDQVLERVIEITESKVTAKPDSADVYMGPVIDQGSYDKIMSYIEIGKQEGRLVSGGTGDDSKGYFIKPTIFADLDPKARLMQEEIFGPVVAFCKVSDFDEALEVANNTEYGLTGAVITNNRKHIERAKQEFHVGNLYFNRNCTGAIVGYHPFGGFKMSGTDSKAGGPDYLALHMQAKTISEMF

SEQ ID NO: 56 - DNA, 762 bp, Bacillus subtilis
atgcaatccttgaattatgaagatcaggtgctttggacgcgctggaaagagtggaaagatcctaaagccggtgacgacttaatgcgccgttacatgccgcttgtcacatatcatgtaggcagaatttctgtcggactgccgaaatcagtgcataaagacgatcttatgagccttggtatgcttggtttatatgatgcccttgaaaaatttgaccccagccgggacttaaaatttgatacctacgcctcgtttagaattcgcggcgcaatcatagacgggcttcgtaaagaagattggctgcccagaacctcgcgcgaaaaaacaaaaaaggttgaagcagcaattgaaaagcttgaacagcggtatcttcggaatgtatcgcccgcggaaattgcagaggaactcggaatgacggtacaggatgtcgtgtcaacaatgaatgaaggtttttttgcaaatctgctgtcaattgatgaaaagctccatgatcaagatgacggggaaaacattcaagtcatgatcagagatgacaaaaatgttccgcctgaagaaaagattatgaaggatgaactgattgcacagcttgcggaaaaaattcacgaactctctgaaaaagaacagctggttgtcagtttgttctacaaagaggagttgacactgacagaaatcggacaagtattaaatctttctacgtcccgcatatctcagatccattcaaaggcattatttaaattaaagaatctgctggaaaaagtgatacaa

SEQ ID NO: 57 - protein, 254 aa, Bacillus subtilis
MQSLNYEDQVLWTRWKEWKDPKAGDDLMRRYMPLVTYHVGRISVGLPKSVHKDDLMSLGMLGLYDALEKFDPSRDLKFDTYASFRIRGAIIDGLRKEDWLPRTSREKTKKVEAAIEKLEQRYLRNVSPAEIAEELGMTVQDVVSTMNEGFFANLLSIDEKLHDQDDGENIQVMIRDDKNVPPEEKIMKDELIAQLAEKIHELSEKEQLVVSLFYKEELTLTEIGQVLNLSTSRISQIHSKALFKLKNLLEKVIQ

SEQ ID NO: 58 - DNA, 602 bp, Bacillus licheniformis
atgaattttcaaacaatcgagcttgacacatggtatagaaaatcttattttgaccattacatgaaggaagcgaaatgttctttcagcatcacggcaaacgtcaatgtgacaaatttgctcgccgtgctcaagaaaaagaagctcaagctgtatccggcttttatttatatcgtatcaagggtcattcattcgcgccctgagtttagaacaacgtttgatgacaaaggaagctgggttattgggaacaaatgcatccgtgctatgcgatttttcatcaggacgaccaaacgttttccgccctctggacggaatactcagacgatttttcgcagttttatcatcaatatcttctggacgccgagcgctttggagacaaaaggggcctttgggctaagccggacatcccgcccaatacgttttcagtttcttctattccatgggtgcgcttttcaacattcaatttaaaccttgataacagcgaacacttgctgccgattattacaaacgggaaatacttttcagaaggcagggaaacatttttgcccgtttcctgcaagttcaccatgcagtgtgtgacggctatcatgccggcgcttttataa

SEQ ID NO: 59 - protein, 200 aa, Bacillus licheniformis
MNFQTIELDTWYRKSYFDHYMKEAKCSFSITANVNVTNLLAVLKKKKLKLYPAFIYIVSRVIHSRPEFRTTFDDKGQLGYWEQMHPCYAIFHQDDQTFSALWTEYSDDFSQFYHQYLLDAERFGDKRGLWAKPDIPPNTFSVSSIPWVRFSTFNLNLDNSEHLLPIITNGKYFSEGRETFLPVSCKFTMQCVTAIMPALL

SEQ ID NO: 60 - DNA, 39 bp, Artificial Sequence
  synthetic primer
ctacattctagacgatttgtttgatcgatatgtggaagc

SEQ ID NO: 61 - DNA, 37 bp, Artificial Sequence
  synthetic primer
ggctgaggatccattcctcagcccagaagagaaccta

SEQ ID NO: 62 - DNA, 38 bp, Artificial Sequence
  synthetic primer
tccctcggatccgaaataggttctgcttattgtattcg

SEQ ID NO: 63 - DNA, 38 bp, Artificial Sequence
  synthetic primer
agcgttgagctcgcgccatgccattatattggctgctg

SEQ ID NO: 64 - DNA, 38 bp, Artificial Sequence
  synthetic primer
gtgacggaattccacgtgcgtcttatattgctgagctt

SEQ ID NO: 65 - DNA, 38 bp, Artificial Sequence
  synthetic primer
cgttttggatccaaaaacacccctttagataatcttat

SEQ ID NO: 66 - DNA, 40 bp, Artificial Sequence
  synthetic primer
atcaaaggatccgctatgctccaaatgtacacctttccgt

SEQ ID NO: 67 - DNA, 39 bp, Artificial Sequence
  synthetic primer
atatttctgcaggctgatataaataatactgtgtgttcc

SEQ ID NO: 68 - DNA, 37 bp, Artificial Sequence
  synthetic primer
catcttgaattcaaagggtacaagcacagagacagag

SEQ ID NO: 69 - DNA, 37 bp, Artificial Sequence
  synthetic primer
tgacttggatccggtaagtgggcagtttgtgggcagt

SEQ ID NO: 70 - DNA, 37 bp, Artificial Sequence
  synthetic primer
tagataggatcctattgaaaactgtttaagaagagga

SEQ ID NO: 71 - DNA, 37 bp, Artificial Sequence
  synthetic primer
ctgattctgcaggagtgtttttgaaggaagcttcatt

SEQ ID NO: 72 - DNA, 37 bp, Artificial Sequence
  synthetic primer
ctccgcggtaccgtcacgaatgcgcctcttattctat

SEQ ID NO: 73 - DNA, 38 bp, Artificial Sequence
  synthetic primer
tcgctgggatccttggcgccgtggaatcgattttgtcc

SEQ ID NO: 74 - DNA, 37 bp, Artificial Sequence
  synthetic primer
gcaatgggatcctatatcaacggttatgaattcacaa

SEQ ID NO: 75 - DNA, 37 bp, Artificial Sequence
  synthetic primer
ccagaactgcaggagcgaggcgtctcgctgcctgaaa

SEQ ID NO: 76 - DNA, 41 bp, Artificial Sequence
  synthetic primer
gacaaggagctcatgaaaaaaagcataaagctttatgttgc

SEQ ID NO: 77 - DNA, 38 bp, Artificial Sequence
  synthetic primer
gacaagggatcccggcatgtccgttattacttaatttc

SEQ ID NO: 78 - DNA, 32 bp, Artificial Sequence
  synthetic primer
gacaagggatcctgccgcttaccggaaacgga

SEQ ID NO: 79 - DNA, 36 bp, Artificial Sequence
  synthetic primer
gacaagtctagattatcgtttgtgcagtattacttg

SEQ ID NO: 80 - DNA, 36 bp, Artificial Sequence
  synthetic primer
actgatgagctctgcctaaacagcaaacagcagaac

SEQ ID NO: 81 - DNA, 39 bp, Artificial Sequence
  synthetic primer
acgaatggatccatcataaagccgcagcagattaaatat

SEQ ID NO: 82 - DNA, 36 bp, Artificial Sequence
  synthetic primer
actgatggatccatcttcgataaatatgaaagtggc

SEQ ID NO: 83 - DNA, 38 bp, Artificial Sequence
  synthetic primer
actgattctagagcctttttctcttgatgcaattcttc

SEQ ID NO: 84 - DNA, 31 bp, Artificial Sequence
  synthetic primer
gagcctctagagcccattgaatcatttgttt

SEQ ID NO: 85 - DNA, 34 bp, Artificial Sequence
  synthetic primer
gagccggatccttaaggatgtcgtttttgtgtct

SEQ ID NO: 86 - DNA, 32 bp, Artificial Sequence
  synthetic primer
gagccggatccatttcggggttctcaaaaaaa

SEQ ID NO: 87 - DNA, 31 bp, Artificial Sequence
  synthetic primer
gagccgagctcatgcaaatggaaaaattgat

SEQ ID NO: 88 - DNA, 35 bp, Artificial Sequence
  synthetic primer
gaagttctagagattgtaattacaaaaggggggtg

SEQ ID NO: 89 - DNA, 32 bp, Artificial Sequence
  synthetic primer
gaagtggatcctttcaccgatcataaaagccc

SEQ ID NO: 90 - DNA, 33 bp, Artificial Sequence
  synthetic primer
tgaaaggatccatttttcattgattgttaagtc

SEQ ID NO: 91 - DNA, 33 bp, Artificial Sequence
  synthetic primer
gaagttagagctcgggggggcataaatttcccg

SEQ ID NO: 92 - DNA, 32 bp, Artificial Sequence
  synthetic primer
gcttatggatccgatacaagagaggtctctcg

SEQ ID NO: 93 - DNA, 31 bp, Artificial Sequence
  synthetic primer
gcttatggatccctgtcatggcgcattaacg

SEQ ID NO: 94 - DNA, 37 bp, Artificial Sequence
  synthetic primer
actgatggatccatcgattttcgttcgtgaatacatg

SEQ ID NO: 95 - DNA, 36 bp, Artificial Sequence
  synthetic primer
actgatggatcccatatgcaagggtttattgttttc

SEQ ID NO: 96 - DNA, 37 bp, Artificial Sequence
  synthetic primer
gcacgttctagaccaccgtcccctgtgttgtatccac

SEQ ID NO: 97 - DNA, 37 bp, Artificial Sequence
  synthetic primer
aggaagggatccagagcgaggaagatgtaggatgatc

SEQ ID NO: 98 - DNA, 36 bp, Artificial Sequence
  synthetic primer
tgacaaggatcctgtatcataccgcatagcagtgcc

SEQ ID NO: 99 - DNA, 37 bp, Artificial Sequence
  synthetic primer
ttccgcgagctcggcgagagcttcagactccgtcaga

SEQ ID NO: 100 - DNA, 31 bp, Artificial Sequence
  synthetic primer
gagcctctagatcagcgatttgacgcggcgc

SEQ ID NO: 101 - DNA, 38 bp, Artificial Sequence
  synthetic primer
ttatctggatccctgatgagcaatgatggtaagataga

SEQ ID NO: 102 - DNA, 37 bp, Artificial Sequence
  synthetic primer
gggtaaggatcccccaaaagggcatagtcattctact

SEQ ID NO: 103 - DNA, 36 bp, Artificial Sequence
  synthetic primer
gagatcggtacccttttgggccatatcgtggatttc

SEQ ID NO: 104 - DNA, 36 bp, Artificial Sequence
  synthetic primer
gagccaagcttcattgacagcaaccaggcagatctc

SEQ ID NO: 105 - DNA, 32 bp, Artificial Sequence
  synthetic primer
gcttataagcttgatacaagagaggtctctcg

SEQ ID NO: 106 - DNA, 31 bp, Artificial Sequence
  synthetic primer
gcttataagcttctgtcatggcgcattaacg

SEQ ID NO: 107 - DNA, 36 bp, Artificial Sequence
  synthetic primer
gagccgagctccatgccgatgaagtcatcgtcgagc

SEQ ID NO: 108 - DNA, 37 bp, Artificial Sequence
  synthetic primer
cgtgaaaagctttcgcgggatgtatgaatttgataag

SEQ ID NO: 109 - DNA, 37 bp, Artificial Sequence
  synthetic primer
tgtagggagctcgatgcgccacaatgtcggtacaacg

SEQ ID NO: 110 - DNA, 36 bp, Artificial Sequence
  synthetic primer
cgagaatctagaacaggatgaatcatctgtggcggg

SEQ ID NO: 111 - DNA, 56 bp, Artificial Sequence
  synthetic primer
cgactgtccagccgctcggcacatcggatccgcttaccgaaagccagactcagcaa

SEQ ID NO: 112 - DNA, 56 bp, Artificial Sequence
  synthetic primer
ttgctgagtctggctttcggtaagcggatccgatgtgccgagcggctggacagtcg

SEQ ID NO: 113 - DNA, 56 bp, Artificial Sequence
  synthetic primer
cgttaatgcgccatgacagccatgaggatcccacaagcccgcacgccttgccacac

SEQ ID NO: 114 - DNA, 56 bp, Artificial Sequence
  synthetic primer
gtgtggcaaggcgtgcgggcttgtgggatcctcatggctgtcatggcgcattaacg

SEQ ID NO: 115 - DNA, 37 bp, Artificial Sequence
  synthetic primer
gacttcgtcgacgagtgcggacggccagcatcaccac

SEQ ID NO: 116 - DNA, 34 bp, Artificial Sequence
  synthetic primer
ggcatatctagagacatgaagcgggaaacagatg

SEQ ID NO: 117 - DNA, 34 bp, Artificial Sequence
  synthetic primer
ggtgcggagctcgacagtatcacagccagcgctg

SEQ ID NO: 118 - DNA, 37 bp, Artificial Sequence
  synthetic primer
aagcgttctagactgcggatgcagatcgatctcggga

SEQ ID NO: 119 - DNA, 56 bp, Artificial Sequence
  synthetic primer
aaccttccgctcacatgtgagcaggggatccgcttaccgaaagccagactcagcaa

SEQ ID NO: 120 - DNA, 56 bp, Artificial Sequence
  synthetic primer
ttgctgagtctggctttcggtaagcggatcccctgctcacatgtgagcggaaggtt

SEQ ID NO: 121 - DNA, 56 bp, Artificial Sequence
  synthetic primer
cgttaatgcgccatgacagccatgaggatccgccttcagccttcccgcggctggct

SEQ ID NO: 122 - DNA, 56 bp, Artificial Sequence
  synthetic primer
agccagccgcgggaaggctgaaggcggatcctcatggctgtcatggcgcattaacg

SEQ ID NO: 123 - DNA, 37 bp, Artificial Sequence
  synthetic primer
caagcactgcagcccacacttcaggcggctcaggtcg

SEQ ID NO: 124 - DNA, 34 bp, Artificial Sequence
  synthetic primer
gagatatctagaatggtatgaagcggaattcccg

SEQ ID NO: 125 - DNA, 37 bp, Artificial Sequence
  synthetic primer
ataaacggtacccccctatagatgcgaacgttagccc

SEQ ID NO: 126 - DNA, 37 bp, Artificial Sequence
  synthetic primer
aaggaggaattccatcttgaggtatacaaacagtcat

SEQ ID NO: 127 - DNA, 56 bp, Artificial Sequence
  synthetic primer
tctccgagaaagacaggcaggatcgggatccgcttaccgaaagccagactcagcaa

SEQ ID NO: 128 - DNA, 56 bp, Artificial Sequence
  synthetic primer
ttgctgagtctggctttcggtaagcggatcccgatcctgcctgtctttctcggaga

SEQ ID NO: 129 - DNA, 37 bp, Artificial Sequence
  synthetic primer
aaggacggtaccggctcattaccctcttttcaagggt

SEQ ID NO: 130 - DNA, 56 bp, Artificial Sequence
  synthetic primer
accaaagccggactcccccgcgagaggatccgcttaccgaaagccagactcagcaa

SEQ ID NO: 131 - DNA, 56 bp, Artificial Sequence
  synthetic primer
ttgctgagtctggctttcggtaagcggatcctctcgcgggggagtccggctttggt

SEQ ID NO: 132 - DNA, 56 bp, Artificial Sequence
  synthetic primer
cgttaatgcgccatgacagccatgaggatcccatacggggtacacaatgtaccata

SEQ ID NO: 133 - DNA, 56 bp, Artificial Sequence
  synthetic primer
tatggtacattgtgtaccccgtatgggatcctcatggctgtcatggcgcattaacg

SEQ ID NO: 134 - DNA, 37 bp, Artificial Sequence
  synthetic primer
gtcaacctgcagagcggcccaggtacaagttggggaa

SEQ ID NO: 135 - DNA, 37 bp, Artificial Sequence
  synthetic primer
ggatcagagctcgcttgtcctcctgggaacagccgga

SEQ ID NO: 136 - DNA, 38 bp, Artificial Sequence
  synthetic primer
tatatgctgcagggctcagacggtaccggttgttccta

SEQ ID NO: 137 - DNA, 37 bp, Artificial Sequence
  synthetic primer
ctgaactctagaccttcaccaggcacagaggaggtga

SEQ ID NO: 138 - DNA, 56 bp, Artificial Sequence
  synthetic primer
gccaataagttctctttagagaacaggatccgcttaccgaaagccagactcagcaa

SEQ ID NO: 139 - DNA, 57 bp, Artificial Sequence
  synthetic primer
ttgctgagtctggctttcggtaagcggatccttgttctctaaagagaacttattggc

SEQ ID NO: 140 - DNA, 56 bp, Artificial Sequence
  synthetic primer
cgttaatgcgccatgacagccatgaggatccgggctaacgttcgcatctatagggg

SEQ ID NO: 141 - DNA, 56 bp, Artificial Sequence
  synthetic primer
cccctatagatgcgaacgttagcccggatcctcatggctgtcatggcgcattaacg

SEQ ID NO: 142 - DNA, 37 bp, Artificial Sequence
  synthetic primer
tgagacgagctcgatgcataggcgacggcagggcgcc

SEQ ID NO: 143 - DNA, 35 bp, Artificial Sequence
  synthetic primer
cgaaattctagatcccgcgattccgccctttgtgg

SEQ ID NO: 144 - DNA, 34 bp, Artificial Sequence
  synthetic primer
ttccaagagctcgcggaataccggaagcagcccc

SEQ ID NO: 145 - DNA, 37 bp, Artificial Sequence
  synthetic primer
caattctctagagcggtcggcgcaggtataggagggg

SEQ ID NO: 146 - DNA, 56 bp, Artificial Sequence
  synthetic primer
gaaaagaaaccaaaaagaatgggaaggatccgcttaccgaaagccagactcagcaa

SEQ ID NO: 147 - DNA, 56 bp, Artificial Sequence
  synthetic primer
ttgctgagtctggctttcggtaagcggatccttcccattctttttggtttcttttc

SEQ ID NO: 148 - DNA, 56 bp, Artificial Sequence
  synthetic primer
cgttaatgcgccatgacagccatgaggatccgctatttaacatttgagaataggga

SEQ ID NO: 149 - DNA, 56 bp, Artificial Sequence
  synthetic primer
tccctattctcaaatgttaaatagcggatcctcatggctgtcatggcgcattaacg

SEQ ID NO: 150 - DNA, 37 bp, Artificial Sequence
  synthetic primer
caggcggagctcccatttatgacgtgcttccctaagc

SEQ ID NO: 151 - DNA, 37 bp, Artificial Sequence
  synthetic primer
tacgaatctagagatcattgcggaagtagaagtggaa

SEQ ID NO: 152 - DNA, 56 bp, Artificial Sequence
  synthetic primer
tttagattgagttcatctgcagcggggatccgcttaccgaaagccagactcagcaa

SEQ ID NO: 153 - DNA, 56 bp, Artificial Sequence
  synthetic primer
ttgctgagtctggctttcggtaagcggatccccgctgcagatgaactcaatctaaa

SEQ ID NO: 154 - DNA, 56 bp, Artificial Sequence
  synthetic primer
cgttaatgcgccatgacagccatgaggatccgccaatcagccttagcccctctcac

SEQ ID NO: 155 - DNA, 56 bp, Artificial Sequence
  synthetic primer
gtgagaggggctaaggctgattggcggatcctcatggctgtcatggcgcattaacg

SEQ ID NO: 156 - DNA, 37 bp, Artificial Sequence
  synthetic primer
atactcgtcgacatacgttgaattgccgagaagccgc

SEQ ID NO: 157 - DNA, 25 bp, Artificial Sequence
  synthetic primer
ctggagtacctggatctggatctcc

SEQ ID NO: 158 - DNA, 25 bp, Artificial Sequence
  synthetic primer
gctcggcttgtttcagctcatttcc

SEQ ID NO: 159 - DNA, 37 bp, Artificial Sequence
  synthetic primer
cggtttgagctcgcgtcctgatctgcagaagctcatt

SEQ ID NO: 160 - DNA, 56 bp, Artificial Sequence
  synthetic primer
ctaaagatgaagtcgatcggctcatggatccgcttaccgaaagccagactcagcaa

SEQ ID NO: 161 - DNA, 56 bp, Artificial Sequence
  synthetic primer
ttgctgagtctggctttcggtaagcggatccatgagccgatcgacttcatctttag

SEQ ID NO: 162 - DNA, 56 bp, Artificial Sequence
  synthetic primer
cgttaatgcgccatgacagccatgaggatccgaagatccctcgatggagttaatgt

SEQ ID NO: 163 - DNA, 56 bp, Artificial Sequence
  synthetic primer
acattaactccatcgagggatcttcggatcctcatggctgtcatggcgcattaacg

SEQ ID NO: 164 - DNA, 38 bp, Artificial Sequence
  synthetic primer
gcttcggtcgactttgccgtctggatatgcgtctctcg

SEQ ID NO: 165 - DNA, 37 bp, Artificial Sequence
  synthetic primer
gtcaaagagctctatgacagcctcctcaaattgcagg

SEQ ID NO: 166 - DNA, 37 bp, Artificial Sequence
  synthetic primer
ttccatgtcgacgctgtgcaaaaccgccggcagcgcc

SEQ ID NO: 167 - DNA, 37 bp, Artificial Sequence
  synthetic primer
acattcgaattcagcaggtcaatcagctcgctgacgc

SEQ ID NO: 168 - DNA, 56 bp, Artificial Sequence
  synthetic primer
ccagcactgcgctccctcacccgaaggatccgcttaccgaaagccagactcagcaa

SEQ ID NO: 169 - DNA, 56 bp, Artificial Sequence
  synthetic primer
ttgctgagtctggctttcggtaagcggatccttcgggtgagggagcgcagtgctgg

SEQ ID NO: 170 - DNA, 56 bp, Artificial Sequence
  synthetic primer
cgttaatgcgccatgacagccatgaggatcctcgagagatccggatggttttcctg

SEQ ID NO: 171 - DNA, 56 bp, Artificial Sequence
  synthetic primer
caggaaaaccatccggatctctcgaggatcctcatggctgtcatggcgcattaacg

SEQ ID NO: 172 - DNA, 37 bp, Artificial Sequence
  synthetic primer
agtcataagctttctggcgtttgatttcatcaacggg

SEQ ID NO: 173 - DNA, 25 bp, Artificial Sequence
  synthetic primer
cagcgcgacttgttaagggacaata

SEQ ID NO: 174 - DNA, 25 bp, Artificial Sequence
  synthetic primer
ggctgctgtgatgaactttgtcgga

SEQ ID NO: 175 - DNA, 30 bp, Artificial Sequence
  synthetic primer
ctcagttcatccatcaaatcaccaagtccg

SEQ ID NO: 176 - DNA, 46 bp, Artificial Sequence
  synthetic primer
tacacgttagaagacggctagatgcgtctgattgtgacagacggcg

SEQ ID NO: 177 - DNA, 26 bp, Artificial Sequence
  synthetic primer
aaccttccagtccggtttactgtcgc

SEQ ID NO: 178 - DNA, 47 bp, Artificial Sequence
  synthetic primer
gtaccataagaagacggagcttgccgtgtccactccgattatagcag

SEQ ID NO: 179 - DNA, 43 bp, Artificial Sequence
  synthetic primer
ccttgtcttgaagacggagctggatccataacttcgtataatg

SEQ ID NO: 180 - DNA, 41 bp, Artificial Sequence
  synthetic primer
gtaccataagaagacggctagaggatgcatatggcggccgc

SEQ ID NO: 181 - DNA, 25 bp, Artificial Sequence
  synthetic primer
catatgctccggctcttcaagcaag

SEQ ID NO: 182 - DNA, 27 bp, Artificial Sequence
  synthetic primer
cctgagattgataaacatgaagtcctc

SEQ ID NO: 183 - DNA, 24 bp, Artificial Sequence
  synthetic primer
atattgaagtcggctggattgtgg

SEQ ID NO: 184 - DNA, 29 bp, Artificial Sequence
  synthetic primer
gcggcagatctcggcgcattaagtcgtca

SEQ ID NO: 185 - DNA, 32 bp, Artificial Sequence
  synthetic primer
gcggcgaattctctgctggaaaaagtgataca

SEQ ID NO: 186 - DNA, 19 bp, Artificial Sequence
  synthetic primer
ttcgctgggataacaacat

SEQ ID NO: 187 - DNA, 32 bp, Artificial Sequence
  synthetic primer
gcggcagatcttaagctggatccataacttcg

SEQ ID NO: 188 - DNA, 32 bp, Artificial Sequence
  synthetic primer
gcggcgaattcatatggcggccgcataacttc

SEQ ID NO: 189 - DNA, 19 bp, Artificial Sequence
  synthetic primer
caatttacgcggggtggtg

SEQ ID NO: 190 - DNA, 21 bp, Artificial Sequence
  synthetic primer
gaataggttacgcagttgttg

SEQ ID NO: 191 - DNA, 21 bp, Artificial Sequence
  synthetic primer
ctcctgatccaaacatgtaag

SEQ ID NO: 192 - DNA, 19 bp, Artificial Sequence
  synthetic primer
aacccttgcatatgtctag

SEQ ID NO: 193 - DNA, 28 bp, Artificial Sequence
  synthetic primer
gcgcccttgatcctaagtcagatgaaac

SEQ ID NO: 194 - DNA, 28 bp, Artificial Sequence
  synthetic primer
cgggtccgatactgactgtaagtttgac

SEQ ID NO: 195 - DNA, 42 bp, Artificial Sequence
  synthetic primer
gtaccataaccatgccttggttaggatgcatatggcggccgc

SEQ ID NO: 196 - DNA, 47 bp, Artificial Sequence
  synthetic primer
ccttgtcttccatcttgctggagctggatccataacttcgtataatg

SEQ ID NO: 197 - DNA, 25 bp, Artificial Sequence
  synthetic primer
gagagcaaggacatgacattgacgc

SEQ ID NO: 198 - DNA, 27 bp, Artificial Sequence
  synthetic primer
gatcttcaccctcttcaacttgtaaag

SEQ ID NO: 199 - DNA, 25 bp, Artificial Sequence
  synthetic primer
tttgcttcctcctgcacaaggcctc

SEQ ID NO: 200 - DNA, 26 bp, Artificial Sequence
  synthetic primer
cgttattgtgtgtgcatttccattgt

SEQ ID NO: 201 - DNA, 39 bp, Artificial Sequence
  synthetic primer
caatggaaatgcacacacaataacgtgactggcaagaga

SEQ ID NO: 202 - DNA, 25 bp, Artificial Sequence
  synthetic primer
gtaatggccctctcgtataaaaaac

SEQ ID NO: 203 - DNA, 46 bp, Artificial Sequence
  synthetic primer
gttttttatacgagagggccattaccaattagaatgaatatttccc

SEQ ID NO: 204 - DNA, 28 bp, Artificial Sequence
  synthetic primer
gaccaaaatgtttcgattcagcattcct

SEQ ID NO: 205 - DNA, 8142 bp, Artificial Sequence
  synthetic pCRM_TSpleo plasmid
ggggatctctgcagtgagatctggtaatgactctctagcttgaggcatcaaataaaacgaaaggctcagtcgaaagactgggcctttcgttttatctgttgtttgtcggtgaacgctctcctgagtaggacaaatccgccgctctagctaagcagaaggccatcctgacggatggcctttttgcgtttctacaaactcttgttaactctagagctgcctgccgcgtttcggtgatgaagatcttcccgatgattaattaattcagaacgctcggttgccgccgggcgttttttatgcagcaatggcaagaacgttgctctagaataattctacacagcccagtccagactattcggcactgaaattatgggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggaattgtgagcggataacaattaagcttaggagggagtgttaaatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaggagctcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatagttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattaacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactggagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggccatgtctgcccgtatttcgcgtaaggaaatccattatgtactatttcaagctaattccggtggaaacgaggtcatcatttccttccgaaaaaacggttgcatttaaatcttacatatgtaatactttcaaagactacatttgtaagatttgatgtttgagtcggctgaaagatcgtacgtaccaattattgtttcgtgattgttcaagccataacactgtagggatagtggaaagagtgcttcatctggttacgatcaatcaaatattcaaacggagggagacgattttgatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttaggcatcgcatcctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtccaatagaccagttgcaatccaaacgagagtctaatagaatgaggtcgaaaagtaaatcgcgtaataaggtaatagatttacattagaaaatgaaaggggattttatgcgtgagaatgttacagtctatcccggcattgccagtcggggatattaaaaagagtataggtttttattgcgataaactaggtttcactttggttcaccatgaagatggattcgcagttctaatgtgtaatgaggttcggattcatctatgggaggcaagtgatgaaggctggcgctctcgtagtaatgattcaccggtttgtacaggtgcggagtcgtttattgctggtactgctagttgccgcattgaagtagagggaattgatgaattatatcaacatattaagcctttgggcattttgcaccccaatacatcattaaaagatcagtggtgggatgaacgagactttgcagtaattgatcccgacaacaatttgattacaaataaaaagctaaaatctattattaatctgttcctgcaggagagaccg

(19) United States
(12) Patent Application Publication
(10) Pub. No.: US 2009/0011463 A1
(43) Pub. Date: Jan. 8, 2009

(54) Pcka Modifications and Enhanced Protein Expression in Bacillus

(76) Inventors: Walter Weyler, San Francisco, CA (US); Amy Kuang-Hua Hsu, Redwood City, CA (US)

Correspondence Address: GENENCOR INTERNATIONAL, INC.;ATTENTION: LEGAL DEPARTMENT, 925 PAGE MILL ROAD, PALO ALTO, CA, 94304, US

(21) Appl. No.: 10/591,852

(22) PCT Filed: Apr. 7, 2005
(86) PCT No.: PCT/US05/11821

§ 371 Date: Jul. 24, 2008

Related U.S. Application Data
(60) Provisional application No. 60/561,110, filed on Apr. 9, 2004.

(51) Int. Cl. C12P 21/04; C12N 1/20; C12N 9/56; C12N 15/74; C12N 15/11; C12N 15/00

(52) U.S. Cl. 435/69.4; 435/252.3; 435/252.31; 435/222; 536/23.2; 435/320.1; 435/252.33; 435/69.1; 435/69.6; 435/471

(57) ABSTRACT

The present invention provides cells that have been genetically manipulated to have an altered capacity to produce expressed proteins, wherein the pckA gene has been modified or deleted. In particular, the present invention relates to Gram-positive microorganisms, such as Bacillus species having enhanced expression of a protein of interest, wherein one or more chromosomal genes have been modified and/or inactivated (e.g., pckA), and preferably wherein one or more chromosomal genes (e.g., pckA) have been modified and/or deleted from the Bacillus chromosome. In some further embodiments, one or more indigenous chromosomal regions have been modified and/or deleted from a corresponding wild-type Bacillus host chromosome.

### FIELD OF THE INVENTION

The present invention provides cells that have been genetically manipulated to have an altered capacity to produce expressed proteins, wherein the pckA gene has been modified or deleted. In particular, the present invention relates to Gram-positive microorganisms, such as Bacillus species having enhanced expression of a protein of interest, wherein one or more chromosomal genes have been modified and/or inactivated (e.g., pckA), and preferably wherein one or more chromosomal genes (e.g., pckA) have been modified and/or deleted from the Bacillus chromosome. In some further embodiments, one or more indigenous chromosomal regions have been modified and/or deleted from a corresponding wild-type Bacillus host chromosome.

### BACKGROUND OF THE INVENTION

Genetic engineering has allowed the improvement of microorganisms used as industrial bioreactors, cell factories and in food fermentations. In particular, Bacillus species produce and secrete a large number of useful proteins and metabolites (Zukowski, “Production of commercially valuable products,” In: Doi and McGlouglin (eds.) Biology of Bacilli: Applications to Industry, Butterworth-Heinemann, Stoneham. Mass pp 311-337 [1992]). The most common Bacillus species used in industry are B. licheniformis, B. amyloliquefaciens and B. subtilis. Because of their GRAS (generally recognized as safe) status, strains of these Bacillus species are natural candidates for the production of proteins utilized in the food and pharmaceutical industries. Important production enzymes include α-amylases, neutral proteases, and alkaline (or serine) proteases. However, in spite of advances in the understanding of production of proteins in Bacillus host cells, there remains a need for methods to increase expression of these proteins.

### SUMMARY OF THE INVENTION

The present invention provides cells that have been genetically manipulated to have an altered capacity to produce expressed proteins, wherein the pckA gene has been modified or deleted. In particular, the present invention relates to Gram-positive microorganisms, such as Bacillus species having enhanced expression of a protein of interest, wherein one or more chromosomal genes have been modified and/or inactivated (e.g., pckA), and preferably wherein one or more chromosomal genes (e.g., pckA) have been modified and/or deleted from the Bacillus chromosome. In some further embodiments, one or more indigenous chromosomal regions have been modified and/or deleted from a corresponding wild-type Bacillus host chromosome. In some preferred embodiments, the present invention provides methods and compositions for the improved expression and/or secretion of at least one protein of interest in Bacillus.

In particularly preferred embodiments, the present invention provides means for improved expression and/or secretion of at least one protein of interest in Bacillus. More particularly, in these embodiments, the present invention involves modification and/or inactivation of one or more chromosomal genes in a Bacillus host strain, wherein the modified and/or inactivated genes are not necessary for strain viability. One result of modifying and/or inactivating one or more of the chromosomal genes is the production of an altered Bacillus strain that is able to express a higher level of a protein of interest over a corresponding non-altered Bacillus host strain.

Furthermore, in alternative embodiments, the present invention provides means for removing large regions of chromosomal DNA in a Bacillus host strain, wherein the deleted indigenous chromosomal region is not necessary for strain viability. One result of removing one or more indigenous chromosomal regions is the production of an altered Bacillus strain that is able to express a higher level of a protein of interest over a corresponding unaltered Bacillus strain. In some preferred embodiments, the Bacillus host strain is a recombinant host strain comprising a polynucleotide encoding a protein of interest. In some particularly preferred embodiments, the altered Bacillus strain is a B. subtilis strain. As explained in detail below, deleted indigenous chromosomal regions include, but are not limited to prophage regions, antimicrobial (e.g., antibiotic) regions, regulator regions, multi-contiguous single gene regions and operon regions.

In some embodiments, the present invention provides methods and compositions for enhancing expression of a protein of interest from a Bacillus cell. In some preferred embodiments, the methods comprise inactivating the pckA gene in a Bacillus host strain to produce an altered Bacillus strain; growing the altered Bacillus strain under suitable growth conditions; and allowing a protein of interest to be expressed in the altered Bacillus, wherein the expression of the protein is enhanced, compared to the corresponding unaltered Bacillus host strain. In alternative embodiments, one or more additional chromosomal genes selected from the group consisting of sbo, sir, ybcO, csn, spolISA, sigB, phrC, rapA, CssS trpA, trpB, trpC, trpD, trpE, trpF, tdh/kbl, alsD, sigD, prpC, gapB, fbp, rocA, ycgN, ycgM, rocF, and rocD are inactivated in a Bacillus host strain to produce an altered Bacillus strain; growing the altered Bacillus strain under suitable growth conditions; and allowing at least one protein of interest to be expressed in the altered Bacillus, wherein the expression of the protein is enhanced, compared to the corresponding unaltered Bacillus host strain. In some embodiments, the protein of interest is a homologous protein, while in other embodiments, the protein of interest is a heterologous protein. In some embodiments, more than one protein of interest is produced. In some preferred embodiments, the Bacillus species is a B. subtilis strain. In yet further embodiments, inactivation of a chromosomal gene comprises the deletion of a gene to produce the altered Bacillus strain. In additional embodiments, inactivation of a chromosomal gene comprises insertional inactivation. In some preferred embodiments, the protein of interest is an enzyme. In some embodiments, the protein of interest is selected from proteases, cellulases, amylases, carbohydrases, lipases, isomerases, transferases, kinases and phosphatases, while in other embodiments, the protein of interest is selected from the group consisting of antibodies, hormones and growth factors.

In yet additional embodiments, the present invention provides altered Bacillus strains comprising the deletion of the pckA gene. While in other embodiments, the altered Bacillus strains further comprise deletions in one or more chromosomal genes selected from the group of sbo, sir, ybcO, csn, spolISA, sigB, phrC, rapA, CssS, trpA, trpB, trpC, trpD, trpE, trpF, tdh/kbl, alsD, sigD, prpC, gapB, fbp, rocA, ycgN, ycgM, rocF, and rocD. In some embodiments, the altered strain is a protease producing Bacillus strain. In an alternative embodiment, the altered Bacillus strain is a subtilisin producing strain. In yet other embodiments, the altered Bacillus strain further, comprises a mutation in a gene selected from the group consisting of degU, degQ, degS, scoC4, spolIE, and oppA.

In further embodiments, the present invention provides DNA constructs comprising an incoming sequence. In some embodiments, the incoming sequence includes a selective marker and a gene and/or gene fragment comprised of the pckA gene. In further embodiments, the incoming sequence further comprises a gene and/or gene fragment selected from the group consisting of sbo, sir, ybcO, csn, spolISA, sigB, phrC, rapA, CssS, trpA, trpB, trpC, trpD, trpE, trpF, tdh/kbl, alsD, sigD, prpC, gapB, fbp, rocA, ycgN, ycgM, rocF, and rocD. In alternative embodiments, the selective marker is located in between two fragments of the gene. In other embodiments, the incoming sequence comprises a selective marker and a homology box, wherein the homology box flanks the 5′ and/or 3′ end of the marker. In additional embodiments, a host cell is transformed with the DNA construct. In further embodiments, the host cell is an E. coli or a Bacillus cell. In some preferred embodiments, the DNA construct is chromosomally integrated into the host cell.

The present invention also provides methods for obtaining an altered Bacillus strain expressing a protein of interest which comprises transforming a Bacillus host cell with the DNA construct of the present invention, wherein the DNA construct is integrated into the chromosome of the Bacillus host cell; producing an altered Bacillus strain, wherein one or more chromosomal genes have been inactivated; and growing the altered Bacillus strain under suitable growth conditions for the expression of a at least one protein of interest. In some embodiments, the (protein of interest is selected from proteases, cellulases, amylases, carbohydrases, lipases, isomerases, transferases, kinases and phosphatases, while in other embodiments, the protein of interest is selected from the group consisting of antibodies, hormones and growth factors. In yet additional embodiments, the Bacillus host strain is selected from the group consisting of B. licheniformis, B. lentus, B. subtilis, B. amyloliquefaciens B. brevis, B. stearothermophilus, B. alkalophilus, B. coagulans, B. circulans, B. pumilus, B. thuringiensis, B. clausii, B. megaterium, and preferably, B. subtilis. In some embodiments, the Bacillus host strain is a recombinant host. In yet additional embodiments, the protein of interest is recovered. In further embodiments, the selective marker is excised from the altered Bacillus.

The present invention further provides methods for obtaining an altered Bacillus strain expressing a protein of interest. In some embodiments, the method comprises transforming a Bacillus host cell with a DNA construct comprising an incoming sequence wherein the incoming sequence comprises a selective marker and pckA. In further embodiments, the incoming sequence further comprises at least one gene selected from the group consisting of sbo, sir, ybcO, csn, spolISA, sigB, phrC, rapA, CssS, trpA, trpB, trpC, trpD, trpE, trpF, tdh/kbl, alsD, sigD, prpC, gapB, fbp, rocA, ycgN, ycgM, rocF, and rocD, wherein the DNA construct is integrated into the chromosome of the Bacillus host cell and results in the deletion of one or more gene(s); obtaining an altered Bacillus strain, and growing the altered Bacillus strain under suitable growth conditions for the expression of the protein of interest.

In some alternative embodiments, the present invention provides a DNA construct comprising an incoming sequence, wherein the incoming sequence includes a selective marker and a cssS gene, a cssS gene fragment or a homologous sequence thereto. In some embodiments, the selective marker is located between two fragments of the gene. In alternative embodiments, the incoming sequence comprises a selective marker and a homology box wherein the homology box flanks the 5′ and/or 3′ end of the marker. In yet other embodiments, a host cell is transformed with the DNA construct. In additional embodiments, the host cell is an E. coli or a Bacillus cell. In still further embodiments, the DNA construct is chromosomally integrated into the host cell.

The present invention also provides methods for obtaining Bacillus subtilis strains that demonstrate enhanced protease production. In some embodiments, the methods comprise the steps of transforming a Bacillus subtilis host cell with a DNA construct according to the invention; allowing homologous recombination of the DNA construct and a homologous region of the Bacillus chromosome wherein pckA is deleted from the Bacillus chromosome; obtaining an altered Bacillus subtilis strain; and growing the altered Bacillus strain under conditions suitable for the expression of a protease. In further embodiments, at least one of the following genes, sbo, sir, ybcO, csn, spolISA, sigB, phrC, rapA, CssS, trpA, trpB, trpC, trpD, trpE, trpF, tdh/kbl, alsD, sigD, prpC, gapB, fbp, rocA, ycgN, ycgM, rocF, and rocD, is deleted from the Bacillus chromosome; obtaining an altered Bacillus subtilis strain; and growing the altered Bacillus strain under conditions suitable for the expression of a protease. In some embodiments, the protease producing Bacillus is a subtilisin producing strain. In alternative embodiments, the protease is a heterologous protease. In additional embodiments, the protease producing strain further includes a mutation in a gene selected from the group consisting of degU, degQ, degS, scoC4, spolIE, and oppA. In some embodiments, the inactivation comprises the insertional inactivation of the gene.

The present invention further provides altered Bacillus subtilis strains comprising a deletion of one pckA, wherein the altered B. subtilis strain is capable of expressing at least one protein of interest. In further embodiments, the altered B. subtilis strains comprise a deletion of or more chromosomal genes selected from the group consisting of sbo, slr, ybcO, csn, spolISA, sigB, phrC, rapA, CssS, trpA, trpB, trpC, trpD, trpE, trpF, tdh/kbl, alsD, sigD, prpC, gapB, fbp, rocA, ycgN, ycgM, rocF, and rocD, wherein the altered Bacillus subtilis strain is capable of expressing at least one protein of interest. In some embodiments, the protein of interest is an enzyme. In some additional embodiments, the protein of interest is a heterologous protein.

In some embodiments, the present invention provides altered Bacillus strains comprising a deletion of one or more indigenous chromosomal regions or fragments thereof, wherein the indigenous chromosomal region includes about 0.5 to 500 kilobases (kb) and wherein the altered Bacillus strains have an enhanced level of expression of a protein of interest compared to the corresponding unaltered Bacillus strains when grown under essentially the same growth conditions. In some preferred embodiments, these altered Bacillus strains comprise a deletion of the pckA gene.

In yet additional embodiments, the present invention provides protease-producing Bacillus strains which comprise at least one deletion of an indigenous chromosomal region selected from the group consisting of a PBSX region, a skin region, a prophage 7 region, a SPβ region, a prophage 1 region, a prophage 2 region, a prophage 3 region, a prophage 4 region, a prophage 5 region, a prophage 6 region, a PPS region, a PKS region, a yvfF-yveK region, a DHB region, and fragments thereof.

In further embodiments, the present invention provides methods for enhancing the expression of at least one protein of interest in Bacillus comprising: obtaining an altered Bacillus strain produced by introducing a DNA construct including a selective marker and an inactivating chromosomal segment into a Bacillus host strain, wherein the DNA construct is integrated into the Bacillus chromosome resulting in the deletion of an indigenous chromosomal region or fragment thereof from the Bacillus host cell; and growing the altered Bacillus strain under suitable growth conditions, wherein expression of a protein of interest is greater in the altered Bacillus strain compared to the expression of the protein of interest is the corresponding unaltered Bacillus host cell.

The present invention also provides methods for obtaining at least one protein of interest from a Bacillus strain comprising the steps of: transforming a Bacillus host cell with a DNA construct which comprises a selective marker and an inactivating chromosomal segment, wherein the DNA construct is integrated into the chromosome of the Bacillus strain and results in deletion of an indigenous chromosomal region or fragment thereof to form an altered Bacillus strain; culturing the altered Bacillus strain under suitable growth conditions to allow the expression of a protein of interest; and recovering the protein of interest.

The present invention also provides a means for the use of DNA microarray data to screen and/or identify beneficial mutations. In some particularly preferred embodiments, these mutations involve the pckA gene. In further embodiments, the mutations involve genes selected from the group consisting of trpA, trpB, trpC, trpD, trpE, trpF, tdh/kbl, rocA, ycgN, ycgM, rocF, and rocD. In some preferred embodiments, these beneficial mutations are based on transcriptome evidence for the simultaneous expression of a given amino acid biosynthetic pathway and biodegradative pathway, and/or evidence that deletion of the degradative pathway results in a better performing strain and/or evidence that overexpression of the biosynthetic pathway results in a better performing strain. In additional embodiments, the present invention provides means for the use of DNA microarray data to provide beneficial mutations. In some preferred embodiments, these mutations involve the pckA gene, while in further embodiments, these mutations involve genes selected from the group consisting of trpA, trpB, trpC, trpD, trpE, trpF, tdh/kbl rocA, ycgN, ycgM, rocF, and rocD, when the expression of mRNA from genes comprising an amino acid biosynthetic pathway is not balanced and overexpression of the entire pathway provides a better performing strain than the parent (i.e., wild-type and/or originating) strain. Furthermore, the present invention provides means to improve production strains through the inactivation of gluconeogenic genes. In some of these preferred embodiments, the inactivated gluconeogenic genes are selected from the group consisting of pckA, gapB, and fbp.

The present invention provides methods for enhancing expression of at least one protein of interest from Bacillus comprising the steps of obtaining an altered Bacillus strain capable of producing a protein of interest, wherein the altered Bacillus strain has an inactivated pckA chromosomal gene and growing the altered Bacillus strain under conditions such that the protein of interest is expressed by the altered Bacillus strain, wherein the expression of the protein of interest is enhanced, compared to the expression of the protein of interest in an unaltered Bacillus host strain. In further embodiments, the altered Bacillus strain further comprises at least one inactivated chromosomal gene selected from the group consisting of sbo, sir, ybcO, csn, spolISA, sigB, phrC, rapA, CssS, trpA, trpB, trpC, trpD, trpE, trpF, tdh/kbl, alsD, sigD, prpC, gapB, fbp, rocA, ycgN, ycgM, rocF, and rocD, and growing the altered Bacillus strain under conditions such that the protein of interest is expressed by the altered Bacillus strain, wherein the expression of the protein of interest is enhanced, compared to the expression of the protein of interest in an unaltered Bacillus host strain. In some embodiments, the protein of interest is selected from the group consisting of homologous proteins and heterologous proteins. In some embodiments, the protein of interest is selected from proteases, cellulases, amylases, carbohydrases, lipases, isomerases, transferases, kinases and phosphatases, while in other embodiments, the protein of interest is selected from the group consisting of antibodies, hormones and growth factors. In some particularly preferred embodiments, the protein of interest is a protease. In some additional embodiments, the altered Bacillus strain is obtained by deleting the pckA region, while in alternative embodiments, the altered Bacillus strain is further obtained by deleting one or more chromosomal genes selected from the group consisting of sbo, sir, ybcO, csn, spolISA, sigB, phrC, rapA, CssS, trpA, trpB, trpC, trpD, trpE, trpF, tdh/kbl, alsD, sigD, prpC, gapB, fbp, rocA, ycgN, ycgM, rocF, and rocD.

The present invention also provides altered Bacillus strains obtained using the method described herein. In some preferred embodiments, the altered Bacillus strains comprise a chromosomal deletion of the pckA gene, while in other embodiments, the altered Bacillus strains further comprises chromosomal deletions of one or more genes selected from the group consisting of sbo, sir, ybcO, csn, spolISA, sigB, phrC, rapA, CssS, trpA, trpB, trpC, trpD, trpE, trpF, tdh/kbl, alsD, sigD, prpC, gapB, fbp, rocA, ycgN, ycgM, rocF, and rocD. In some embodiments, more than one of these chromosomal genes have been deleted. In some particularly preferred embodiments, the altered strains are B. subtilis strains. In additional preferred embodiments, the altered Bacillus strains are protease producing strains. In some particularly preferred embodiments, the protease is a subtilisin. In yet additional embodiments, the subtilisin is selected from the group consisting of subtilisin 168, subtilisin BPN′, subtilisin Carlsberg, subtilisin DY, subtilisin 147, subtilisin 309 and variants thereof. In yet further embodiments, altered Bacillus strains further comprise mutation(s) in at least one gene selected from the group consisting of degU, degQ, degS, scoC4, spolIE, and oppA. In some particularly preferred embodiments, the altered Bacillus strains further comprise a heterologous protein of interest.

The present invention also provides DNA constructs comprising the pckA gene. In additional embodiments, the present invention provides DNA constructs further comprising at least one gene selected from the group consisting of sbo, sir, ybcO, csn, spolISA, sigB, phrC, rapA, CssS, trpA, trpB, trpC, trpD, trpE, trpF, tdh/kbl, alsD, sigD, prpC, gapB, fbp, rocA, ycgN, ycgM, rocF, and rocD, gene fragments thereof, and homologous sequences thereto. In some preferred embodiments, the DNA constructs comprise at least one nucleic acid sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO:17, SEQ ID NO:39, SEQ ID NO:40, SEQ ID NO:42, SEQ ID NO:44, SEQ ID NO:46, SEQ ID NO:48, SEQ ID NO:50, SEQ ID NO:37, SEQ ID NO:25, SEQ ID NO:21, SEQ ID NO:50, SEQ ID NO:29, SEQ ID NO:23, SEQ ID NO:27, SEQ ID NO:19, SEQ ID NO:31, SEQ ID NO:48, SEQ ID NO:46, SEQ ID NO:35, and SEQ ID NO:33. In some embodiments, the DNA constructs further comprise at least one polynucleotide sequence encoding at least one protein of interest.

The present invention also provides plasmids comprising the DNA constructs. In further embodiments, the present invention provides host cells comprising the plasmids comprising the DNA constructs. In some embodiments, the host cells are selected from the group consisting of Bacillus cells and E. coli cells. In some preferred embodiments, the host cell is B. subtilis. In some particularly preferred embodiments, the DNA construct is integrated into the chromosome of the host cell. In alternative embodiments, the DNA construct comprises at least one gene that encodes at least one amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO:18, SEQ ID NO:41, SEQ ID NO:43, SEQ ID NO:45, SEQ ID NO:47, SEQ ID NO:49, SEQ ID NO:51, SEQ ID NO:38, SEQ ID NO:26, SEQ ID NO:22, SEQ ID NO:57, SEQ ID NO:30, SEQ ID NO:24, SEQ ID NO:28, SEQ ID NO:20, SEQ ID NO:32, SEQ ID NO:55, SEQ ID NO:53, SEQ ID NO:36, and SEQ ID NO:34. In additional embodiments, the DNA constructs further comprise at least one selective marker, wherein the selective marker is flanked on each side by a fragment of the gene or homologous gene sequence thereto.

The present invention also provides DNA constructs comprising an incoming sequence, wherein the incoming sequence comprises a nucleic acid encoding a protein of interest, and a selective marker flanked on each side with a homology box, wherein the homology box includes nucleic acid sequences having 80 to 100% sequence identity to the sequence immediately flanking the coding regions of the pckA gene. In additional embodiments, the incoming sequence further comprises at least one gene selected from the group consisting of sbo, sir, ybcO, csn, spolISA, sigB, phrC, rapA, CssS, trpA, trpB, trpC, trpD, trpE, trpF, tdh/kbl, alsD, sigD, prpC, gapB, fbp, rocA, ycgN, ycgM, rocF, and rocD. In some embodiments, the DNA constructs further comprise at least one nucleic acid which flanks the coding sequence of the gene. The present invention also provides plasmids comprising the DNA constructs. In further embodiments, the present invention provides host cells comprising the plasmids comprising the DNA constructs. In some embodiments, the host cells are selected from the group consisting of Bacillus cells and E. coli cells. In some preferred embodiments, the host cell is B. subtilis. In some particularly preferred embodiments, the DNA construct is integrated into the chromosome of the host cell. In additional preferred embodiments, the selective marker has been excised from the host cell chromosome.

The present invention further provides methods for obtaining an altered Bacillus strain with enhanced protease production comprising: transforming a Bacillus host cell with at least one DNA construct of the present invention, wherein the protein of interest in the DNA construct is a protease, and wherein the DNA construct is integrated into the chromosome of the Bacillus host cell under conditions such that at least one gene is inactivated to produce an altered Bacillus strain; and growing the altered Bacillus strain under conditions such that enhanced protease production is obtained. In some particularly preferred embodiments, the method further comprises recovering the protease. In alternative preferred embodiments, at least one inactivated gene is deleted from the chromosome of the altered Bacillus strain. The present invention also provides altered Bacillus strains produced using the methods described herein. In some embodiments, the Bacillus host strain is selected from the group consisting of B. licheniformis, B. lentus, B. subtilis, B. amyloliquefaciens, B. brevis, B. stearothermophilus, B. alkalophilus, B. coagulans, B. circulans, B. pumilus, B. lautus, B. clausii, B. megaterium, and B. thuringiensis. In some preferred embodiments, the Bacillus host cell is B. subtilis.

The present invention also provides methods for enhancing expression of a protease in an altered Bacillus comprising: transforming a Bacillus host cell with a DNA construct of the present invention; allowing homologous recombination of the DNA construct and a region of the chromosome of the Bacillus host cell, wherein at least one gene of the chromosome of the Bacillus host cell is inactivated, to produce an altered Bacillus strain; and growing the altered Bacillus strain under conditions suitable for the expression of the protease, wherein the production of the protease is greater in the altered Bacillus subtilis strain compared to the Bacillus subtilis host prior to transformation. In some preferred embodiments, the protease is subtilisin. In additional embodiments, the protease is a recombinant protease. In yet further embodiments, inactivation is achieved by deletion of at least one gene. In still further embodiments, inactivation is by insertional inactivation of at least one gene. The present invention also provides altered Bacillus strains obtained using the methods described herein. In some embodiments, altered Bacillus strain comprises an inactivated pckA gene. In additional embodiments, the altered Bacillus strain further comprises at least one inactivated gene selected from the group consisting of sbo, sir, ybcO, csn, spolISA, sigB, phrC, rapA, CssS, trpA, trpB, trpC, trpD, trpE, trpF, tdh/kbl, alsD, sigD, prpC, gapB, fbp, rocA, ycgN, ycgM, rocF, and rocD. In some preferred embodiments, the inactivated gene has been inactivated by deletion. In additional embodiments, the altered Bacillus strains further comprise at least one mutation in a gene selected from the group consisting of degU, degS, degQ, scoC4, spolIE, and oppA. In some preferred embodiments, the mutation is degU(Hy)32. In still further embodiments, the strain is a recombinant protease producing strain. In some preferred embodiments, the altered Bacillus strains are selected from the group consisting of B. licheniformis, B. lentus, B. subtilis, B. amyloliquefaciens, B. brevis, B. stearothermophilus, B. alkalophilus, B. coagulans, B. circulans, B. pumilus, B. lautus, B. clausii, B. megaterium, and B. thuringiensis.

The present invention also provides altered Bacillus strains comprising a deletion of one or more indigenous chromosomal regions or fragments thereof, wherein the indigenous chromosomal region includes about 0.5 to 500 kb, and wherein the altered Bacillus strain has an enhanced level of expression of a protein of interest compared to a corresponding unaltered Bacillus strain when the altered and unaltered Bacillus strains are grown under essentially the same growth conditions. In preferred embodiments, the altered Bacillus strain is selected from the group consisting of B. licheniformis, B. lentus, B. subtilis, B. amyloliquefaciens, B. brevis, B. stearothermophilus, B. alkalophilus, B. coagulans, B. circulans, B. pumilus, B. lautus, B. clausii, B. megaterium, and B. thuringiensis. In some preferred embodiments, the altered Bacillus strain is selected from the group consisting of B. subtilis, B. licheniformis, and B. amyloliquefaciens. In some particularly preferred embodiments, the altered Bacillus strain is a B. subtilis strain. In yet further embodiments, the indigenous chromosomal region is selected from the group consisting of a PBSX region, a skin region, a prophage 7 region, a SPβ region, a prophage 1 region, a prophage 2 region, a prophage 4 region, a prophage 3 region, a prophage 4 region, a prophage 5 region, a prophage 6, region, a PPS region, a PKS region, a YVFF-YVEK region, a DHB region, and fragments thereof. In some preferred embodiments, two indigenous chromosomal regions or fragments thereof have been deleted. In some embodiments, the at least one protein of interest is selected from proteases, cellulases, amylases, carbohydrases, lipases, isomerases, transferases, kinases and phosphatases, while in other embodiments, the protein of interest is selected from the group consisting of antibodies, hormones and growth factors. In yet additional embodiments, the protein of interest is a protease. In some preferred embodiments, the protease is a subtilisin. In some particularly preferred embodiments, the subtilisin is selected from the group consisting of subtilisin 168, subtilisin BPN′, subtilisin Carlsberg, subtilisin DY, subtilisin 147 and subtilisin 309 and variants thereof. In further preferred embodiments, the Bacillus host is a recombinant strain. In some particularly preferred embodiments, the altered Bacillus strains further comprise at least one mutation in a gene selected from the group consisting of degU, degQ, degS, sco4, spolIE and oppA. In some preferred embodiments, the mutation is degU(Hy)32.

The present invention further provides protease producing Bacillus strains comprising a deletion of an indigenous chromosomal region selected from the group consisting of a PBSX region, a skin region, a prophage 7 region, a SPβ region, a prophage 1 region, a prophage 2 region, a prophage 3 region, a prophage 4 region, a prophage 5 region, a prophage 6 region, a PPS region, a PKS region, a YVFF-YVEK region, a DHB region, and fragments thereof. In some preferred embodiments, the protease is a subtilisin. In some embodiments, the protease is a heterologous protease. In some preferred embodiments, the altered Bacillus strain is selected from the group consisting of B. licheniformis, B. lentus, B. subtilis, B. amyloliquefaciens, B. brevis, B. stearothermophilus, B. alkalophilus, B. coagulans, B. circulans, B. pumilus, B. lautus, B. clausii, B. megaterium, and B. thuringiensis. In additional embodiments, the Bacillus strain is a B. subtilis strain.

The present invention also provides methods for enhancing the expression of a protein of interest in Bacillus comprising: introducing a DNA construct including a selective marker and an inactivating chromosomal segment into a Bacillus host strain, wherein the DNA construct is integrated into the chromosome of the Bacillus host strain, resulting in the deletion of an indigenous chromosomal region or fragment thereof from the Bacillus host cell to produce an altered Bacillus strain; and growing the altered Bacillus strain under suitable conditions, wherein expression of a protein of interest is greater in the altered Bacillus strain compared to the expression of the protein of interest in a Bacillus host cell that has not been altered. In some preferred embodiments, the methods further comprise the step of recovering the protein of interest. In some embodiments, the methods further comprise the step of excising the selective marker from the altered Bacillus strain. In additional embodiments, the indigenous chromosomal region is selected from the group of regions consisting of PBSX, skin, prophage 7, SPβ, prophage 1, prophage 2, prophage 3, prophage 4, prophage 5, prophage 6, PPS, PKS, YVFF-YVEK, DHB, and fragments thereof. In further embodiments, the altered Bacillus strain comprises deletion of at least two indigenous chromosomal regions. In some preferred embodiments, the protein of interest is an enzyme. In some embodiments, the protein of interest is selected from proteases, cellulases, amylases, carbohydrases, lipases, isomerases, transferases, kinases and phosphatases, while in other embodiments, the protein of interest is selected from the group consisting of antibodies, hormones and growth factors. In some embodiments, the Bacillus host strain is selected from the group consisting of B. licheniformis, B. lentus, B. subtilis, B. amyloliquefaciens, B. brevis, B. stearothermophilus, B. clausii, B. alkalophilus, B. coagulans, B. circulans, B. pumilus and B. thuringiensis. The present invention also provides altered Bacillus strains produced using the methods described herein.

The present invention also provides methods for obtaining a protein of interest from a Bacillus strain comprising: transforming a Bacillus host cell with a DNA construct comprising a selective marker and an inactivating chromosomal segment, wherein the DNA construct is integrated into the chromosome of the Bacillus strain resulting in deletion of an indigenous chromosomal region or fragment thereof, to produce an altered Bacillus strain, culturing the altered Bacillus strain under suitable growth conditions to allow the expression of a protein of interest, and recovering the protein of interest. In some preferred embodiments, the protein of interest is an enzyme. In some particularly preferred embodiments, the Bacillus host comprises a heterologous gene encoding a protein of interest. In additional embodiments, the Bacillus host cell is selected from the group consisting of B. licheniformis, B. lentus, B. subtilis, B. amyloliquefaciens, B. brevis, B. stearothermophilus, B. clausii, B. alkalophilus, B. coagulans, B. circulans, B. pumilus and B. thuringiensis. In some preferred embodiments, the indigenous chromosomal region is selected from the group of regions consisting of PBSX, skin, prophage 7, SPβ, prophage 1, prophage 2, prophage 3, prophage 4, prophage 5, prophage 6, PPS, PKS, YVFF-YVEK, DHB, and fragments thereof. In some particularly preferred embodiments the altered Bacillus strains further comprise at least one mutation in a gene selected from the group consisting of degU, degQ, degS, sco4, spolIE and oppA. In some embodiments, the protein of interest is an enzyme selected from the group consisting of proteases, cellulases, amylases, carbohydrases, lipases, isomerases, transferases, kinases, and phosphatases. In some particularly preferred embodiments, the enzyme is a protease. In some preferred embodiments, the protein of interest is an enzyme. In other embodiments, the protein of interest is selected from the group consisting of antibodies, hormones and growth factors.

The present invention further provides methods for enhancing the expression of a protein of interest in Bacillus comprising: obtaining nucleic acid from at least one Bacillus cell; performing transcriptome DNA array analysis on the nucleic acid from said Bacillus cell to identify at least one gene of interest; modifying at least one gene of interest to produce a DNA construct; introducing the DNA construct into a Bacillus host cell to produce an altered Bacillus strain, wherein the altered Bacillus strain is capable of producing a protein of interest, under conditions such that expression of the protein of interest is enhanced as compared to the expression of the protein of interest in a Bacillus that has not been altered. In some embodiments, the protein of interest is associated with at least one biochemical pathway selected from the group consisting of amino acid biosynthetic pathways and biodegradative pathways. In some embodiments, the methods involve disabling at least one biodegradative pathway. In some embodiments, the biodegradative pathway is disabled due to the transcription of the gene of interest. However, it is not intended that the present invention be limited to these pathways, as it is contemplated that the methods will find use in the modification of other biochemical pathways within cells such that enhanced expression of a protein of interest results. In some particularly preferred embodiments, the Bacillus host comprises a heterologous gene encoding a protein of interest. In additional embodiments, the Bacillus host cell is selected from the group consisting of B. licheniformis, B. lentus, B. subtilis, B. amyloliquefaciens, B. brevis, B. stearothermophilus, B. clausii, B. alkalophilus, B. coagulans, B. circulans, B. pumilus and B. thuringiensis. In some embodiments, the protein of interest is an enzyme. In some preferred embodiments, the protein of interest is selected from proteases, cellulases, amylases, carbohydrases, lipases, isomerases, transferases, kinases and phosphatases, while in other embodiments, the protein of interest is selected from the group consisting of antibodies, hormones and growth factors.

The present invention further provides methods for enhancing the expression of a protein of interest in Bacillus, comprising: obtaining nucleic acid containing at least one gene of interest from at least one Bacillus cell; fragmenting said nucleic acid; amplifying said fragments to produce a pool of amplified fragments comprising said at least one gene of interest; ligating said amplified fragments to produce a DNA construct; directly transforming said DNA construct into a Bacillus host cell to produce an altered Bacillus strain; culturing said altered Bacillus strain under conditions such that expression of said protein of interest is enhanced as compared to the expression of said protein of interest in a Bacillus that has not been altered. In some preferred embodiments, said amplifying comprises using the polymerase chain reaction. In some embodiments, the altered Bacillus strain comprises modified gene selected from the group consisting of prpC, sigD and tdh/kbl. In some particularly preferred embodiments, the Bacillus host comprises a heterologous gene encoding a protein of interest. In additional embodiments, the Bacillus host cell is selected from the group consisting of B. licheniformis, B. lentus, B. subtilis, B. amyloliquefaciens, B. brevis, B. stearothermophilus, B. clausii, B. alkalophilus, B. coagulans, B. circulans, B. pumilus and B. thuringiensis. In some embodiments, the protein of interest is an enzyme. In some preferred embodiments, the protein of interest is selected from proteases, cellulases, amylases, carbohydrases, lipases, isomerases, transferases, kinases and phosphatases, while in other embodiments, the protein of interest is selected from the group consisting of antibodies, hormones and growth factors.

The present invention further provides isolated nucleic acids comprising the sequences set forth in nucleic acid sequences selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO:39, SEQ ID NO:40, SEQ ID NO:42, SEQ ID NO:44, SEQ ID NO:46, SEQ ID NO:48, SEQ ID NO:50, SEQ ID NO:37, SEQ ID NO:25, SEQ ID NO:21, SEQ ID NO:50, SEQ ID NO:23, SEQ ID NO:27, SEQ ID NO:19, SEQ ID NO:31, SEQ ID NO:48, SEQ ID NO:46, SEQ ID NO:35, and SEQ ID NO:33.

The present invention also provides isolated nucleic acid sequences encoding amino acids, wherein the amino acids are selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 41, SEQ ID NO:43, SEQ ID NO:45, SEQ ID NO:47, SEQ ID NO:49, SEQ ID NO:51, SEQ ID NO:38, SEQ ID NO:26, SEQ ID NO:22, SEQ ID NO:57, SEQ ID NO:24, SEQ ID NO:28, SEQ ID NO:20, SEQ ID NO:32, SEQ ID NO:55, SEQ ID NO:53, SEQ ID NO:36, and SEQ ID NO:34.

The present invention further provides isolated amino acid sequences, wherein the amino acid sequences are selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 41, SEQ ID NO: 43, SEQ ID NO:45, SEQ ID NO:47, SEQ ID NO:49, SEQ ID NO:51, SEQ ID NO:38, SEQ ID NO:26, SEQ ID NO:22, SEQ ID NO:57, SEQ ID NO:24, SEQ ID NO:28, SEQ ID NO:20, SEQ ID NO:32, SEQ ID NO:55, SEQ ID NO:53, SEQ ID NO:36, and SEQ ID NO:34.

### BRIEF DESCRIPTION OF THE DRAWINGS

FIG. 1, Panels A and B illustrate a general schematic diagram of one method (“Method 1” See, Example 1) provided by the present invention. In this method, flanking regions of a gene and/or an indigenous chromosomal region are amplified out of a wild-type Bacillus chromosome, cut with restriction enzymes (including at least BamHI) and ligated into pJM102. The construct is cloned through E. coli and the plasmid is isolated, linearized with BamHI and ligated to an antimicrobial marker with complementary ends. After cloning again in E. coli, a liquid culture is grown and used to isolate plasmid DNA for use in transforming a Bacillus host strain (preferably, a competent Bacillus host strain).

FIG. 2 illustrates the location of primers used in the construction of a DNA cassette according to some embodiments of the present invention. The diagram provides an explanation of the primer naming system used herein. Primers 1 and 4 are used for checking the presence of the deletion. These primers are referred to as “DeletionX-UF-chk” and “DeletionX-UR-chk-del.” DeletionX-UF-chk is also used in a PCR reaction with a reverse primer inside the antimicrobial marker (Primer 11: called for example PBSX-UR-chk-Del) for a positive check of the cassette's presence in the chromosome. Primers 2 and 6 are used to amplify the upstream flanking region. These primers are referred to as “DeletionX-UF” and “DeletionX-UR,” and contain engineered restriction sites at the black vertical bars. Primers 5 and 8 are used to amplify the downstream flanking region. These primers are referred to as “DeletionX-DF” and “DeletionX-DR.” These primers may either contain engineered BamHI sites for ligation and cloning, or 25 base pair tails homologous to an appropriate part of the Bacillus subtilis chromosome for use in PCR fusion. In some embodiments, primers 3 and 7 are used to fuse the cassette together in the case of those cassettes created by PCR fusion, while in other embodiments, they are used to check for the presence of the insert. These primers are referred to as “DeletionX-UF-nested” and “DeletionX-DR-nested.” In some embodiments, the sequence corresponding to an “antibiotic marker” is a Spc resistance marker and the region to be deleted is the cssS gene.

FIG. 3 is a general schematic diagram of one method (“Method 2”; See Example 2) of the present invention. Flanking regions are engineered to include 25 bp of sequence complementary to a selective marker sequence. The selective marker sequence also includes 25 bp tails that complement DNA of one flanking region. Primers near the ends of the flanking regions are used to amplify all three templates in a single reaction tube, thereby creating a fusion fragment. This fusion fragment or DNA construct is directly transformed into a competent Bacillus host strain.

FIG. 4 provides an electrophoresis gel of Bacillus DHB deletion clones. Lanes 1 and 2 depict two strains carrying the DHB deletion amplified with primers 1 and 11, and illustrate a 1.2 kb band amplified from upstream of the inactivating chromosomal segments into the phleomycin marker. Lane 3 depicts the wild-type control for this reaction. Only non-specific amplification is observed. Lanes 4 and 5 depict the DHB deleted strains amplified with primers 9 and 12. This 2 kb band amplifies through the antibiotic region to below the downstream section of the inactivated chromosomal segment. Lane 6 is the negative control for this reaction and a band is not illustrated. Lanes 7 and 8 depict the deletion strains amplified with primers 1 and 4 and the illustration confirms that the DHB region is missing. Lane 9 is the wild-type control.

FIG. 5 illustrates gel electrophoresis of two clones of a production strain of Bacillus subtilis (wild-type) wherein sir is replaced with a phleomycin (phleo) marker which results in a deletion of the slr gene. Lanes 1 and 2 represent the clones amplified with primers at locations 1 and 11. Lane 3 is the wild-type chromosomal DNA amplified with the same primers. A 1.2 kb band is observed for the insert. Lanes 4 and 5 represent the clones amplified with primers at locations 9 and 12. Lane 6 is the wild-type chromosomal DNA amplified with the same primers. Correct transformants include a 2 kb band. Lanes 7 and 8 represent the clones amplified with primers at locations 2 and 4. Lane 9 is the wild-type chromosomal DNA amplified with the same primers. No band is observed for the deletion strains, but a band around 1 kb is observed in the wild-type. Reference is made to FIG. 2 for an explanation of primer locations.

FIG. 6 provides an electrophoresis gel of a clone of a production strain of Bacillus subtilis (wild-type) wherein cssS is inactivated by the integration of a spectinomycin marker into the chromosome. Lane 1 is a control without the integration and is approximately 1.5 kb smaller.

FIG. 7 provides a bar graph showing improved subtilisin secretion measured from shake flask cultures with Bacillus subtilis wild-type strain (unaltered) and corresponding altered Bacillus subtilis strains having various deletions. Protease activity (g/L) was measured after 17, 24 and 40 hours or was measured at 24 and 40 hours.

FIG. 8 provides a bar graph showing improved protease secretion as measured from shake flask cultures in Bacillus subtilis wild-type strain (unaltered) and corresponding altered deletion strains (-sbo) and (-sir). Protease activity (g/L) was measured after 17, 24 and 40 hours.

FIG. 9 provides a photograph showing the halo produced by a control strain and a pckA-deletion strain.

FIG. 10, Panel A provides a graph showing the optical density of the parent strain and the pckA-strain grown in minimal medium over time (“EFT” refers to the elapsed fermentation time). As indicated by this graph, the pckA-deletion strain produced more growth in a shorter time period than the parent strain. FIG. 10, Panel B provides a graph showing the titer of the parent strain and the pckA-deletion strain grown in a rich medium expressed in g/liter over time. FIG. 10, Panel C provides a graph showing the carbon yield of the parent strain and the pckA-deletion strain grown in a rich medium. As indicated in this Panel, the pckA-deletion strain was more efficient at carbon utilization.

### DESCRIPTION OF THE INVENTION

The present invention provides cells that have been genetically manipulated to have an altered capacity to produce expressed proteins. In particular, the present invention relates to Gram-positive microorganisms, such as Bacillus species having enhanced expression of at least one protein of interest, wherein one or more chromosomal genes have been inactivated or otherwise modified. In particularly preferred embodiments, the pckA gene is inactivated or otherwise modified. In some preferred embodiments, one or more chromosomal genes have been modified and/or deleted from the Bacillus chromosome. In some further embodiments, one or more indigenous chromosomal regions have been deleted from a corresponding wild-type Bacillus host chromosome. In preferred embodiments, the region comprising at least the pckA gene is deleted from the Bacillus chromosome.

### DEFINITIONS

All patents and publications, including all sequences disclosed within such patents and publications, referred to herein are expressly incorporated by reference. Unless defined otherwise herein, all technical and scientific terms used herein have the same meaning as commonly understood by one of ordinary skill in the art to which this invention belongs (See e.g., Singleton et al., DICTIONARY OF MICROBIOLOGY AND MOLECULAR BIOLOGY, 2D ED., John Wiley and Sons, New York [1994]; and Hale and Marham, THE HARPER COLLINS DICTIONARY OF BIOLOGY, Harper Perennial, NY [1991], both of which provide one of skill with a general dictionary of many of the terms used herein). Although any methods and materials similar or equivalent to those described herein can be used in the practice or testing of the present invention, the preferred methods and materials are described. Numeric ranges are inclusive of the numbers defining the range. As used herein and in the appended claims, the singular “a,” “an,” and “the” include the plural reference unless the context clearly dictates otherwise. Thus, for example, reference to a “host cell” includes a plurality of such host cells.

Unless otherwise indicated, nucleic acids are written left to right in 5′ to 3′ orientation; amino acid sequences are written left to right in amino to carboxy orientation, respectively. The headings provided herein are not limitations of the various aspects or embodiments of the invention that can be had by reference to the specification as a whole. Accordingly, the terms defined immediately below are more fully defined by reference to the Specification as a whole.

As used herein, “host cell” refers to a cell that has the capacity to act as a host or expression vehicle for a newly introduced DNA sequence. In preferred embodiments of the present invention, the host cells are Bacillus sp. or E. coli cells.

As used herein, “the genus Bacillus” includes all species within the genus “Bacillus,” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus.” The production of resistant endospores in the presence of oxygen is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

As used herein, “nucleic acid” refers to a nucleotide or polynucleotide sequence, and fragments or portions thereof, as well as to DNA, cDNA, and RNA of genomic or synthetic origin which may be double-stranded or single-stranded, whether representing the sense or antisense strand. It will be understood that as a result of the degeneracy of the genetic code, a multitude of nucleotide sequences may encode a given protein.

As used herein the term “gene” means a chromosomal segment of DNA involved in producing a polypeptide chain that may or may not include regions preceding and following the coding regions (e.g. 5′ untranslated (5′ UTR) or leader sequences and 3′ untranslated (3′ UTR) or trailer sequences, as well as intervening sequence (introns) between individual coding segments (exons)).

In some embodiments, the gene encodes therapeutically significant proteins or peptides, such as growth factors, cytokines, ligands, receptors and inhibitors, as well as vaccines and antibodies. The gene may encode commercially important industrial proteins or peptides, such as enzymes (e.g., proteases, carbohydrases such as amylases and glucoamylases, cellulases, oxidases and lipases). However, it is not intended that the present invention be limited to any particular enzyme or protein. In some embodiments, the gene of interest is a naturally-occurring gene, while in other embodiments, it is a mutated gene or a synthetic gene.

As used herein, the term “vector” refers to any nucleic acid that can be replicated in cells and can carry new genes or DNA segments into cells. Thus, the term refers to a nucleic acid construct designed for transfer between different host cells. An “expression vector” refers to a vector that has the ability to incorporate and express heterologous DNA fragments in a foreign cell. Many prokaryotic and eukaryotic expression vectors are commercially available. Selection of appropriate expression vectors is within the knowledge of those having skill in the art.

As used herein, the terms “DNA construct,” “expression cassette,” and “expression vector,” refer to a nucleic acid construct generated recombinantly or synthetically, with a series of specified nucleic acid elements that permit transcription of a particular nucleic acid in a target cell (i.e., these are vectors or vector elements, as described above). The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments, DNA constructs also include a series of specified nucleic acid elements that permit transcription of a particular nucleic acid in a target cell. In one embodiment, a DNA construct of the invention comprises a selective marker and an inactivating chromosomal segment as defined herein.

As used herein, “transforming DNA,” “transforming sequence,” and “DNA construct” refer to DNA that is used to introduce sequences into a host cell or organism. Transforming DNA is DNA used to introduce sequences into a host cell or organism. The DNA may be generated in vitro by PCR or any other suitable techniques. In some preferred embodiments, the transforming DNA comprises an incoming sequence, while in other preferred embodiments it further comprise an incoming sequence flanked by homology boxes. In yet a further embodiment, the transforming DNA comprises other non-homologous sequences, added to the ends (i.e., stuffer sequences or flanks). The ends can be closed such that the transforming DNA forms a closed circle, such as, for example, insertion into a vector.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell.

As used herein, the terms “isolated” and “purified” refer to a nucleic acid or amino acid (or other component) that is removed from at least one component with which it is naturally associated.

As used herein, the term “enhanced expression” is broadly construed to include enhanced production of a protein of interest. Enhanced expression is that expression above the normal level of expression in the corresponding host strain that has not been altered according to the teachings herein but has been grown under essentially the same growth conditions.

In some preferred embodiments, “enhancement” is achieved by any modification that results in an increase in a desired property. For example, in some particularly preferred embodiments, the present invention provides means for enhancing protein production, such that the enhanced strains produced a greater quantity and/or quality of a protein of interest than the parental strain (e.g., the wild-type and/or originating strain).

As used herein the term “expression” refers to a process by which a polypeptide is produced based on the nucleic acid sequence of a gene. The process includes both transcription and translation.

As used herein in the context of introducing a nucleic acid sequence into a cell, the term “introduced” refers to any method suitable for transferring the nucleic acid sequence into the cell. Such methods for introduction include but are not limited to protoplast fusion, transfection, transformation, conjugation, and transduction (See e.g., Ferrari et al., “Genetics,” in Hardwood et al, (eds.), Bacillus, Plenum Publishing Corp., pages 57-72, [1989]).

As used herein, the terms “transformed” and “stably transformed” refers to a cell that has a non-native (heterologous) polynucleotide sequence integrated into its genome or as an episomal plasmid that is maintained for at least two generations.

As used herein “an incoming sequence” refers to a DNA sequence that is introduced into the Bacillus chromosome. In some preferred embodiments, the incoming sequence is part of a DNA construct. In preferred embodiments, the incoming sequence encodes one or more proteins of interest. In some embodiments, the incoming sequence comprises a sequence that may or may not already be present in the genome of the cell to be transformed (i.e., it may be either a homologous or heterologous sequence). In some embodiments, the incoming sequence encodes one or more proteins of interest, a gene, and/or a mutated or modified gene. In alternative embodiments, the incoming sequence encodes a functional wild-type gene or operon, a functional mutant gene or operon, or a non-functional gene or operon. In some embodiments, the non-functional sequence may be inserted into a gene to disrupt function of the gene. In some embodiments, the incoming sequence encodes one or more functional wild-type genes, while in other embodiments, the incoming sequence encodes one or more functional mutant genes, and in yet additional embodiments, the incoming sequence encodes one or more non-functional genes. In another embodiment, the incoming sequence encodes a sequence that is already present in the chromosome of the host cell to be transformed. In a preferred embodiment, the incoming sequence comprises the pckA gene, while in alternative preferred embodiments, the incoming sequence further comprises at least one gene selected from the group consisting of sbo, sir, ybcO, csn, spolISA, phrC, sigB, rapA, CssS, trpA, trpB, trpC, trpD, trpE, trpF, tdh/kbl, alsD, sigD, prpC, gapB, fbp, rocA, ycgN, ycgM, rocF, and rocD, and fragments thereof. In yet another embodiment, the incoming sequence includes a selective marker. In a further embodiment the incoming sequence includes two homology boxes.

In some embodiments, the incoming sequence encodes at least one heterologous protein including, but not limited to hormones, enzymes, and growth factors. In another embodiment, the enzyme includes, but is not limited to hydrolases, such as protease, esterase, lipase, phenol oxidase, permease, amylase, pullulanase, cellulase, glucose isomerase, laccase and protein disulfide isomerase.

As used herein, “homology box” refers to a nucleic acid sequence, which is homologous to a sequence in the Bacillus chromosome. More specifically, a homology box is an upstream or downstream region having between about 80 and 100% sequence identity, between about 90 and 100% sequence identity, or between about 95 and 100% sequence identity with the immediate flanking coding region of a gene or part of a gene to be inactivated according to the invention. These sequences direct where in the Bacillus chromosome a DNA construct is integrated and directs what part of the Bacillus chromosome is replaced by the incoming sequence. While not meant to limit the invention, a homology box may include about between 1 base pair (bp) to 200 kilobases (kb). Preferably, a homology box includes about between 1 bp and 10.0 kb; between 1 bp and 5.0 kb; between 1 bp and 2.5 kb; between 1 bp and 1.0 kb, and between 0.25 kb and 2.5 kb. A homology box may also include about 10.0 kb, 5.0 kb, 2.5 kb, 2.0 kb, 1.5 kb, 1.0 kb, 0.5 kb, 0.25 kb and 0.1 kb. In some embodiments, the 5′ and 3′ ends of a selective marker are flanked by a homology box wherein the homology box comprises nucleic acid sequences immediately flanking the coding region of the gene.

As used herein, the term “selectable marker-encoding nucleotide sequence” refers to a nucleotide sequence which is capable of expression in the host cells and where expression of the selectable marker confers to cells containing the expressed gene the ability to grow in the presence of a corresponding selective agent or lack of an essential nutrient.

As used herein, the terms “selectable marker” and “selective marker” refer to a nucleic acid (e.g., a gene) capable of expression in host cell which allows for ease of selection of those hosts containing the vector. Examples of such selectable markers include but are not limited to antimicrobials. Thus, the term “selectable marker” refers to genes that provide an indication that a host cell has taken up an incoming DNA of interest or some other reaction has occurred. Typically, selectable markers are genes that confer antimicrobial resistance or a metabolic advantage on the host cell to allow cells containing the exogenous DNA to be distinguished from cells that have not received any exogenous sequence during the transformation. A “residing selectable marker” is one that is located on the chromosome of the microorganism to be transformed. A residing selectable marker encodes a gene that is different from the selectable marker on the transforming DNA construct. Selective markers are well known to those of skill in the art. As indicated above, preferably the marker is an antimicrobial resistant marker (e.g., amp^{R}; phleo^{R}; spec^{R}; kan^{R}; ery^{R}; tet^{R}; cmp^{R}; and neo^{R}; See e.g., Guerot-Fleury, Gene, 167:335-337 [1995]; Palmeros et al., Gene 247:255-264 [2000]; and Trieu-Cuot et al., Gene, 23:331-341 [1983]). In some particularly preferred embodiments, the present invention provides a chloramphenicol resistance gene (e.g., the gene present on pC194, as well as the resistance gene present in the Bacillus licheniformis genome). This resistance gene is particularly useful in the present invention, as well as in embodiments involving chromosomal amplification of chromosomally integrated cassettes and integrative plasmids (See e.g., Albertini and Galizzi, Bacteriol., 162:1203-1211 [1985]; and Stahl and Ferrari, J. Bacteriol., 158:411-418 [1984]). The DNA sequence of this naturally-occurring chloramphenicol resistance gene is shown below:

(SEQ ID NO:58)

ATGAATTTTCAAACAATCGAGCTTGACACATGGTATAGAAAATCTTATTT
TGACCATTACATGAAGGAAGCGAAATGTTCTTTCAGCATCACGGCAAACG
TCAATGTGACAAATTTGCTCGCCGTGCTCAAGAAAAAGAAGCTCAAGCTG
TATCCGGCTTTTATTTATATCGTATCAAGGGTCATTCATTCGCGCCCTGA
GTTTAGAACAACGTTTGATGACAAAGGAAGCTGGGTTATTGGGAACAAAT
GCATCCGTGCTATGCGATTTTTCATCAGGACGACCAAACGTTTTCCGCCC
TCTGGACGGAATACTCAGACGATTTTTCGCAGTTTTATCATCAATATCTT
CTGGACGCCGAGCGCTTTGGAGACAAAAGGGGCCTTTGGGCTAAGCCGGA
CATCCCGCCCAATACGTTTTCAGTTTCTTCTATTCCATGGGTGCGCTTTT
CAACATTCAATTTAAACCTTGATAACAGCGAACACTTGCTGCCGATTATT
ACAAACGGGAAATACTTTTCAGAAGGCAGGGAAACATTTTTGCCCGTTTC
CTGCAAGTTCACCATGCAGTGTGTGACGGCTATCATGCCGGCGCTTTTAT
AA.

The deduced amino acid sequence of this chloramphenicol resistance protein is:

(SEQ ID NO:59)

MNFQTIELDTWYRKSYFDHYMKEAKCSFSITANVNVTNLLAVLKKKKLKL
YPAFIYIVSRVIHSRPEFRTTFDDKGQLGYWEQMHPCYAIFHQDDQTFSA
LWTEYSDDFSQFYHQYLLDAERFGDKRGLWAKPDIPPNTFSVSSIPWVRF
STFNLNLDNSEHLLPIITNGKYFSEGRETFLPVSCKFTMQCVTAIMPAL
L.

Other markers useful in accordance with the invention include, but are not limited to auxotrophic markers, such as tryptophan; and detection markers, such as β-galactosidase.

As used herein, the term “promoter” refers to a nucleic acid sequence that functions to direct transcription of a downstream gene. In preferred embodiments, the promoter is appropriate to the host cell in which the target gene is being expressed. The promoter, together with other transcriptional and translational regulatory nucleic acid sequences (also termed “control sequences”) is necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

A nucleic acid is “operably linked” when it is placed into a functional relationship with another nucleic acid sequence. For example, DNA encoding a secretory leader (i.e., a signal peptide), is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “inactivation” includes any method that prevents the functional expression of the pckA gene, alone or in combination with one or more of the sbo, sir, ybcO, csn, spolISA, sigB, phrC, rapA, CssS, trpA, trpB, trpC, trpD, trpE, trpF, tdh/kbl, alsD, sigD, prpC, gapB, fbp, rocA, ycgN, ycgM, rocF, and rocD chromosomal genes, wherein the gene or gene product is unable to exert its known function. Inactivation or enhancement occurs via any suitable means, including deletions, substitutions (e.g., mutations), interruptions, and/or insertions in the nucleic acid gene sequence. In one embodiment, the expression product of an inactivated gene is a truncated protein with a corresponding change in the biological activity of the protein. In some embodiments, the change in biological activity is an increase in activity, while in preferred embodiments, the change is results in the loss of biological activity. In some embodiments, an altered Bacillus strain comprises inactivation of one or more genes that results preferably in stable and non-reverting inactivation.

In some preferred embodiments, inactivation is achieved by deletion. In some preferred embodiments, the gene is deleted by homologous recombination. For example, in some embodiments when sbo is the gene to be deleted, a DNA construct comprising an incoming sequence having a selective marker flanked on each side by a homology box is used. The homology box comprises nucleotide sequences homologous to nucleic acids flanking regions of the chromosomal sbo gene. The DNA construct aligns with the homologous sequences of the Bacillus host chromosome and in a double crossover event the sbo gene is excised out of the host chromosome.

As used herein, “deletion” of a gene refers to deletion of the entire coding sequence, deletion of part of the coding sequence, or deletion of the coding sequence including flanking regions. The deletion may be partial as long as the sequences left in the chromosome provides the desired biological activity of the gene. The flanking regions of the coding sequence may include from about 1 bp to about 500 bp at the 5′ and 3′ ends. The flanking region may be larger than 500 bp but will preferably not include other genes in the region which may be inactivated or deleted according to the invention. The end result is that the deleted gene is effectively non-functional. In simple terms, a “deletion” is defined as a change in either nucleotide or amino acid sequence in which one or more nucleotides or amino acid residues, respectively, have been removed (i.e., are absent). Thus, a “deletion mutant” has fewer nucleotides or amino acids than the respective wild-type organism.

In still another embodiment of the present invention, deletion of a gene active at an inappropriate time as determined by DNA array analysis (e.g., transcriptome analysis, as described herein) provides enhanced expression of a product protein. In some preferred embodiments, the present invention provides deletion of the pckA gene, while in alternative preferred embodiments, deletion of one or more of genes selected from the group consisting of, gapB, fbp, and/or alsD, provides an improved strain for the improved efficiency of feed utilization. As used herein, “transcriptome analysis” refers to the analysis of gene transcription.

In another embodiment of the present invention, a gene is considered to be “optimized” by the deletion of a regulatory sequence in which this deletion results in increased expression of a desired product. In some preferred embodiments of the present invention, the tryptophan operon (i.e., comprising genes trpA trpB, trpC, trpD, trpE, trpF) is optimized by the deletion of the DNA sequence coding for the TRAP binding RNA sequence (See, Yang, et al., J. Mol. Biol., 270:696-710 [1997]). This deletion is contemplated to increase expression of the desired product from the host strain.

In another preferred embodiment, inactivation is by insertion. For example, in some embodiments, when pckA is the gene to be inactivated, a DNA construct comprises an incoming sequence having the pckA gene interrupted by a selective marker. The selective marker will be flanked on each side by sections of the pckA coding sequence. The DNA construct aligns with essentially identical sequences of the pckA gene in the host chromosome and in a double crossover event the pckA gene is inactivated by the insertion of the selective marker. In simple terms, an “insertion” or “addition” is a change in a nucleotide or amino acid sequence which has resulted in the addition of one or more nucleotides or amino acid residues, respectively, as compared to the naturally occurring sequence.

In another embodiment, activation is by insertion in a single crossover event with a plasmid as the vector. For example, a pckA chromosomal gene is aligned with a plasmid comprising the gene or part of the gene coding sequence and a selective marker. In some embodiments, the selective marker is located within the gene coding sequence or on a part of the plasmid separate from the gene. The vector is integrated into the Bacillus chromosome, and the gene is inactivated by the insertion of the vector in the coding sequence.

In alternative embodiments, inactivation results due to mutation of the gene. Methods of mutating genes are well known in the art and include but are not limited to site-directed mutation, generation of random mutations, and gapped-duplex approaches (See e.g., U.S. Pat. No. 4,760,025; Moring et al., Biotech. 2:646 [1984]; and Kramer et al., Nucleic Acids Res., 12:9441 [1984]).

As used herein, a “substitution” results from the replacement of one or more nucleotides or amino acids by different nucleotides or amino acids, respectively.

As used herein, “homologous genes” refers to a pair of genes from different, but usually related species, which correspond to each other and which are identical or very similar to each other. The term encompasses genes that are separated by speciation (i.e., the development of new species) (e.g., orthologous genes), as well as genes that. have been separated by genetic duplication (e.g., paralogous genes).

As used herein, “ortholog” and “orthologous genes” refer to genes in different species that have evolved from a common ancestral gene (i.e., a homologous gene) by speciation. Typically, orthologs retain the same function in during the course of evolution. Identification of orthologs finds use in the reliable prediction of gene function in newly sequenced genomes.

As used herein, “paralog” and “paralogous genes” refer to genes that are related by duplication within a genome. While orthologs retain the same function through the course of evolution, paralogs evolve new functions, even though some functions are often related to the original one. Examples of paralogous genes include, but are not limited to genes encoding trypsin, chymotrypsin, elastase, and thrombin, which are all serine proteinases and occur together within the same species.

As used herein, “homology” refers to sequence similarity or identity, with identity being preferred. This homology is determined using standard techniques known in the art (See e.g., Smith and Waterman, Adv. Appl. Math., 2:482 [1981]; Needleman and Wunsch, J. Mol. Biol., 48:443 [1970]; Pearson and Lipman, Proc. Natl. Acad. Sci. USA 85:2444 [1988]; programs such as GAP, BESTFIT, FASTA, and TFASTA in the Wisconsin Genetics Software Package (Genetics Computer Group, Madison, Wis.); and Devereux et al., Nucl. Acid Res., 12:387-395 [1984]).

As used herein, an “analogous sequence” is one wherein the function of the gene is essentially the same as the gene designated from Bacillus subtilis strain 168. Additionally, analogous genes include at least 60%, 65%, 70%, 75%, 80%, 85%, 90%, 95%, 97%, 98%, 99% or 100% sequence identity with the sequence of the Bacillus subtilis strain 168 gene. Alternately, analogous sequences have an alignment of between 70 to 100% of the genes found in the B. subtilis 168 region and/or have at least between 5-10 genes found in the region aligned with the genes in the B. subtilis 168 chromosome. In additional embodiments more than one of the above properties applies to the sequence. Analogous sequences are determined by known methods of sequence alignment. A commonly used alignment method is BLAST, although as indicated above and below, there are other methods that also find use in aligning sequences.

One example of a useful algorithm is PILEUP. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle (Feng and Doolittle, J. Mol. Evol., 35:351-360 [1987]). The method is similar to that described by Higgins and Sharp (Higgins and Sharp, CABIOS 5:151-153 [1989]). Useful PILEUP parameters including a default gap weight of 3.00, a default gap length weight of 0.10, and weighted end gaps.

Another example of a useful algorithm is the BLAST algorithm, described by Altschul et al., (Altschul et al., J. Mol. Biol., 215:403-410, [1990]; and Karlin et al., Proc. Natl. Acad. Sci. USA 90:5873-5787 [1993]). A particularly useful BLAST program is the WU-BLAST-2 program (See, Altschul et al., Meth. Enzymol.,, 266:460-480 [1996]). WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched. However, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

Thus, “percent (%) nucleic acid sequence identity” is defined as the percentage of nucleotide residues in a candidate sequence that are identical with the nucleotide residues of the sequence shown in the nucleic acid figures. A preferred method utilizes the BLASTN module of WU-BLAST-2 set to the default parameters, with overlap span and overlap fraction set to 1 and 0.125, respectively.

The alignment may include the introduction of gaps in the sequences to be aligned. In addition, for sequences which contain either more or fewer nucleosides than those of the nucleic acid figures, it is understood that the percentage of homology will be determined based on the number of homologous nucleosides in relation to the total number of nucleosides. Thus, for example, homology of sequences shorter than those of the sequences identified herein and as discussed below, will be determined using the number of nucleosides in the shorter sequence.

As used herein, the term “hybridization” refers to the process by which a strand of nucleic acid joins with a complementary strand through base pairing, as known in the art.

A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm-5° C. (5° below the Tm of the probe); “high stringency” at about 5-10° C. below the Tm; “intermediate stringency” at about 10-20° C. below the Tm of the probe; and “low stringency” at about 20-25° C. below the Tm. Functionally, maximum stringency conditions may be used to identify sequences having strict identity or near-strict identity with the hybridization probe; while an intermediate or low stringency hybridization can be used to identify or detect polynucleotide sequence homologs.

Moderate and high stringency hybridization conditions are well known in the art. An example of high stringency conditions includes hybridization at about 42° C. in 50% formamide, 5×SSC, 5×Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C. An example of moderate stringent conditions include an overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate and 20 mg/ml denaturated sheared salmon sperm DNA, followed by washing the filters in 1×SSC at about 37-50° C. Those of skill in the art know how to adjust the temperature, ionic strength, etc. as necessary to accommodate factors such as probe length and the like.

As used herein, “recombinant” includes reference to a cell or vector, that has been modified by the introduction of a heterologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention. “Recombination,” “recombining,” and generating a “recombined” nucleic acid are generally the assembly of two or more nucleic acid fragments wherein the assembly gives rise to a chimeric gene.

In a preferred embodiment, mutant DNA sequences are generated with site saturation mutagenesis in at least one codon. In another preferred embodiment, site saturation mutagenesis is performed for two or more codons. In a further embodiment, mutant DNA sequences have more than 40%, more than 45%, more than 50%, more than 55%, more than 60%, more than 65%, more than 70%, more than 75%, more than 80%, more than 85%, more than 90%, more than 95%, or more than 98% homology with the wild-type sequence. In alternative embodiments, mutant DNA is generated in vivo using any known mutagenic procedure such as, for example, radiation, nitrosoguanidine and the like. The desired DNA sequence is then isolated and used in the methods provided herein.

In an alternative embodiment, the transforming DNA sequence comprises homology boxes without the presence of an incoming sequence. In this embodiment, it is desired to delete the endogenous DNA sequence between the two homology boxes. Furthermore, in some embodiments, the transforming sequences are wild-type, while in other embodiments, they are mutant or modified sequences. In addition, in some embodiments, the transforming sequences are homologous, while in other embodiments, they are heterologous.

As used herein, the term “target sequence” refers to a DNA sequence in the host cell that encodes the sequence where it is desired for the incoming sequence to be inserted into the host cell genome. In some embodiments, the target sequence encodes a functional wild-type gene or operon, while in other embodiments the target sequence encodes a functional mutant gene or operon, or a non-functional gene or operon.

As used herein, a “flanking sequence” refers to any sequence that is either upstream or downstream of the sequence being discussed (e.g., for genes A-B-C, gene B is flanked by the A and C gene sequences). In a preferred embodiment, the incoming sequence is flanked by a homology box on each side. In another embodiment, the incoming sequence and the homology boxes comprise a unit that is flanked by stuffer sequence on each side. In some embodiments, a flanking sequence is present on only a single side (either 3′ or 5′), but in preferred embodiments, it is on each side of the sequence being flanked. The sequence of each homology box is homologous to a sequence in the Bacillus chromosome. These sequences direct where in the Bacillus chromosome the new construct gets integrated and what part of the Bacillus chromosome will be replaced by the incoming sequence. In a preferred embodiment, the 5′ and 3′ ends of a selective marker are flanked by a polynucleotide sequence comprising a section of the inactivating chromosomal segment. In some embodiments, a flanking sequence is present on only a single side (either 3′ or 5′), while in preferred embodiments, it is present on each side of the sequence being flanked.

As used herein, the term “stuffer sequence” refers to any extra DNA that flanks homology boxes (typically vector sequences). However, the term encompasses any non-homologous DNA sequence. Not to be limited by any theory, a stuffer sequence provides a noncritical target for a cell to initiate DNA uptake.

As used herein, the term “library of mutants” refers to a population of cells which are identical in most of their genome but include different homologues of one or more genes. Such libraries find use for example, in methods to identify genes or operons with improved traits.

As used herein, the terms “hypercompetent” and “super competent” mean that greater than 1% of a cell population is transformable with chromosomal DNA (e.g., Bacillus DNA). Alternatively, the terms are used in reference to cell populations in which greater than 10% of a cell population is transformable with a self-replicating plasmid (e.g., a Bacillus plasmid). Preferably, the super competent cells are transformed at a rate greater than observed for the wild-type or parental cell population. Super competent and hypercompetent are used interchangeably herein.

As used herein, the terms “amplification” and “gene amplification” refer to a process by which specific DNA sequences are disproportionately replicated such that the amplified gene becomes present in a higher copy number than was initially present in the genome. In some embodiments, selection of cells by growth in the presence of a drug (e.g., an inhibitor of an inhibitable enzyme) results in the amplification of either the endogenous gene encoding the gene product required for growth in the presence of the drug or by amplification of exogenous (i.e., input) sequences encoding this gene product, or both.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

As used herein, the term “co-amplification” refers to the introduction into a single cell of an amplifiable marker in conjunction with other gene sequences (i.e., comprising one or more non-selectable genes such as those contained within an expression vector) and the application of appropriate selective pressure such that the cell amplifies both the amplifiable marker and the other, non-selectable gene sequences. The amplifiable marker may be physically linked to the other gene sequences or alternatively two separate pieces of DNA, one containing the amplifiable marker and the other containing the non-selectable marker, may be introduced into the same cell.

As used herein, the terms “amplifiable marker,” “amplifiable gene,” and “amplification vector” refer to a gene or a vector encoding a gene which permits the amplification of that gene under appropriate growth conditions.

“Template specificity” is achieved in most amplification techniques by the choice of enzyme. Amplification enzymes are enzymes that, under conditions they are used, will process only specific sequences of nucleic acid in a heterogeneous mixture of nucleic acid. For example, in the case of Qβ replicase, MDV-1 RNA is the specific template for the replicase (See e.g., Kacian et al., Proc. Natl. Acad. Sci. USA 69:3038 [1972]). Other nucleic acids are not replicated by this amplification enzyme. Similarly, in the case of T7 RNA polymerase, this amplification enzyme has a stringent specificity for its own promoters (See, Chamberlin et al., Nature 228:227 [1970]). In the case of T4 DNA ligase, the enzyme will not ligate the two oligonucleotides or polynucleotides, where there is a mismatch between the oligonucleotide or polynucleotide substrate and the template at the ligation junction (See, Wu and Wallace, Genomics 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences.

As used herein, the term “amplifiable nucleic acid” refers to nucleic acids which may be amplified by any amplification method. It is contemplated that “amplifiable nucleic acid” will usually comprise “sample template.”

As used herein, the term “sample template” refers to nucleic acid originating from a sample which is analyzed for the presence of “target” (defined below). In contrast, “background template” is used in reference to nucleic acid other than sample template which may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target,” when used in reference to the polymerase chain reaction, refers to the region of nucleic acid bounded by the primers used for polymerase chain reaction. Thus, the “target” is sought to be sorted out from other nucleic acid sequences. A “segment” is defined as a region of nucleic acid within the target sequence.

As used herein, the term “polymerase chain reaction” (“PCR”) refers to the methods of U.S. Pat. Nos. 4,683,195 4,683,202, and 4,965,188, hereby incorporated by reference, which include methods for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

As used herein, the term “amplification reagents” refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “RT-PCR” refers to the replication and amplification of RNA sequences. In this method, reverse transcription is coupled to PCR, most often using a one enzyme procedure in which a thermostable polymerase is employed, as described in U.S. Pat. No. 5,322,770, herein incorporated by reference. In RT-PCR, the RNA template is converted to cDNA due to the reverse transcriptase activity of the polymerase, and then amplified using the polymerizing activity of the polymerase (i.e., as in other PCR methods).

As used herein, the terms “restriction endonucleases” and “restriction enzymes” refer to bacterial enzymes, each of which cut double-stranded DNA at or near a specific nucleotide sequence.

A “restriction site” refers to a nucleotide sequence recognized and cleaved by a given restriction endonuclease and is frequently the site for insertion of DNA fragments. In certain embodiments of the invention restriction sites are engineered into the selective marker and into 5′ and 3′ ends of the DNA construct.

As used herein “an inactivating chromosomal segment” comprises two sections. Each section comprises polynucleotides that are homologous with the upstream or downstream genomic chromosomal DNA that immediately flanks an indigenous chromosome region as defined herein. “Immediately flanks” means the nucleotides comprising the inactivating chromosomal segment do not include the nucleotides defining the indigenous chromosomal region. The inactivating chromosomal segment directs where in the Bacillus chromosome the DNA construct gets integrated and what part of the Bacillus chromosome will be replaced.

As used herein, “indigenous chromosomal region” and “a fragment of an indigenous chromosomal region” refer to a segment of the Bacillus chromosome which is deleted from a Bacillus host cell in some embodiments of the present invention. In general, the terms “segment,” “region,” “section,” and “element” are used interchangeably herein. In some embodiments, deleted segments include one or more genes with known functions, while in other embodiments, deleted segments include one or more genes with unknown functions, and in other embodiments, the deleted segments include a combination of genes with known and unknown functions. In some embodiments, indigenous chromosomal regions or fragments thereof include as many as 200 genes or more.

In some embodiments, an indigenous chromosomal region or fragment thereof has a necessary function under certain conditions, but the region is not necessary for Bacillus strain viability under laboratory conditions. Preferred laboratory conditions include but are not limited to conditions such as growth in a fermenter, in a shake flask on plated media, etc., at standard temperatures and atmospheric conditions (e.g., aerobic).

An indigenous chromosomal region or fragment thereof may encompass a range of about 0.5 kb to 500 kb; about 1.0 kb to 500 kb; about 5 kb to 500 kb; about 10 kb to 500 kb; about 10 kb to 200 kb; about 10 kb to 100 kb; about 10 kb to 50 kb; about 100 kb to 500 kb; and about 200 kb to 500 kb of the Bacillus chromosome. In another aspect, when an indigenous chromosomal region or fragment thereof has been deleted, the chromosome of the altered Bacillus strain may include 99%, 98%, 97%, 96%, 95%, 94%, 93%, 92%, 91%, 90%, 85%, 80%, 75% or 70% of the corresponding unaltered Bacillus host chromosome. Preferably, the chromosome of an altered Bacillus strain according to the invention will include about 99 to 90%; 99 to 92%; and 98 to 94% of the corresponding unaltered Bacillus host strain chromosome genome.

As used herein, “strain viability” refers to reproductive viability. The deletion of an indigenous chromosomal region or fragment thereof, does not deleteriously affect division and survival of the altered Bacillus strain under laboratory conditions.

As used herein, “altered Bacillus strain” refers to a genetically engineered Bacillus sp. wherein a protein of interest has an enhanced level of expression and/or production as compared to the expression and/or production of the same protein of interest in a corresponding unaltered Bacillus host strain grown under essentially the same growth conditions. In some embodiments, the enhanced level of expression results from the inactivation of one or more chromosomal genes. In one embodiment, the enhanced level of expression results from the deletion of one or more chromosomal genes. In some embodiments, the altered Bacillus strains are genetically engineered Bacillus sp. having one or more deleted indigenous chromosomal regions or fragments thereof, wherein a protein of interest has an enhanced level of expression or production, as compared to a corresponding unaltered Bacillus host strain grown under essentially the same growth conditions. In an alternative embodiment, the enhanced level of expression results from the insertional inactivation of one or more chromosomal genes. In some alternate embodiments, enhanced level of expression results due to increased activation or an otherwise optimized gene. In some preferred embodiments, the inactivated gene is the pckA gene, while in alternative preferred embodiments, the inactivated genes are further selected from the group consisting of sbo, sir, ybcO, csn, spolISA, phrC, sigB, rapA, CssS, trpA, trpB, trpC, trpD, trpE, trpF, tdh/kbl, alsD, sigD, prpC, gapB, fbp, rocA, ycgN, ycgM, rocF, and rocD.

In certain embodiments, the altered Bacillus strain comprises two inactivated genes, while in other embodiments, there are three inactivated genes, four inactivated genes, five inactivated genes, six inactivated genes, or more. Thus, it is not intended that the number of inactivated genes be limited to an particular number of genes. In some embodiments, the inactivated genes are contiguous to each another, while in other embodiments, they are located in separate regions of the Bacillus chromosome. In some embodiments, an inactivated chromosomal gene has a necessary function under certain conditions, but the gene is not necessary for Bacillus strain viability under laboratory conditions. Preferred laboratory conditions include but are not limited to conditions such as growth in a fermenter, in a shake flask, plated media, etc., suitable for the growth of the microorganism.

As used herein, a “corresponding unaltered Bacillus strain” is the host strain (e.g., the originating and/or wild-type strain) from which the indigenous chromosomal region or fragment thereof is deleted or modified and from which the altered strain is derived.

As used herein, the term “chromosomal integration” refers to the process whereby the incoming sequence is introduced into the chromosome of a host cell (e.g., Bacillus). The homologous regions of the transforming DNA align with homologous regions of the chromosome. Subsequently, the sequence between the homology boxes is replaced by the incoming sequence in a double crossover (i.e., homologous recombination). In some embodiments of the present invention, homologous sections of an inactivating chromosomal segment of a DNA construct align with the flanking homologous regions of the indigenous chromosomal region of the Bacillus chromosome. Subsequently, the indigenous chromosomal region is deleted by the DNA construct in a double crossover (i.e., homologous recombination).

“Homologous recombination” means the exchange of DNA fragments between two DNA molecules or paired chromosomes at the site of identical or nearly identical nucleotide sequences. In a preferred embodiment, chromosomal integration is homologous recombination.

“Homologous sequences” as used herein means a nucleic acid or polypeptide sequence having 100%, 99%, 98%, 97%, 96%, 95%, 94%, 93%, 92%, 91%, 90%, 88%, 85%, 80%, 75%, or 70% sequence identity to another nucleic acid or polypeptide sequence when optimally aligned for comparison. In some embodiments, homologous sequences have between 85% and 100% sequence identity, while in other embodiments there is between 90% and 100% sequence identity, and in more preferred embodiments, there is 95% and 100% sequence identity.

As used herein “amino acid” refers to peptide or protein sequences or portions thereof. The terms “protein”, “peptide” and “polypeptide” are used interchangeably.

As used herein, “protein of interest” and “polypeptide of interest” refer to a protein/polypeptide that is desired and/or being assessed. In some embodiments, the protein of interest is intracellularly expressed, while in other embodiments, it is a secreted polypeptide. Particularly preferred polypeptides include enzymes, including, but not limited to those selected from amylolytic enzymes, proteolytic enzymes, cellulytic enzymes, oxidoreductase enzymes and plant cell-wall degrading enzymes. More particularly, these enzyme include, but are not limited to amylases, proteases, xylanases, lipases, laccases, phenol oxidases, oxidases, cutinases, cellulases, hemicellulases, esterases, perioxidases, catalases, glucose oxidases, phytases, pectinases, glucosidases, isomerases, transferases, galactosidases and chitinases. In some particularly preferred embodiments of the present invention, the polypeptide of interest is a protease. In some embodiments, the protein of interest is a secreted polypeptide which is fused to a signal peptide (i.e., an amino-terminal extension on a protein to be secreted). Nearly all secreted proteins use an amino-terminal protein extension which plays a crucial role in the targeting to and translocation of precursor proteins across the membrane. This extension is proteolytically removed by a signal peptidase during or immediately following membrane transfer.

In some embodiments of the present invention, the polypeptide of interest is selected from hormones, antibodies, growth factors, receptors, etc. Hormones encompassed by the present invention include but are not limited to, follicle-stimulating hormone, luteinizing hormone, corticotropin-releasing factor, somatostatin, gonadotropin hormone, vasopressin, oxytocin, erythropoietin, insulin and the like. Growth factors include, but are not limited to platelet-derived growth factor, insulin-like growth factors, epidermal growth factor, nerve growth factor, fibroblast growth factor, transforming growth factors, cytokines, such as interleukins (e.g., IL-1 through IL-13), interferons, colony stimulating factors, and the like. Antibodies include but are not limited to immunoglobulins obtained directly from any species from which it is desirable to produce antibodies. In addition, the present invention encompasses modified antibodies. Polyclonal and monoclonal antibodies are also encompassed by the present invention. In particularly preferred embodiments, the antibodies are human antibodies.

As used herein, the term “heterologous protein” refers to a protein or polypeptide that does not naturally occur in the host cell. Examples of heterologous proteins include enzymes such as hydrolases including proteases, cellulases, amylases, carbohydrases, and lipases; isomerases such as racemases, epimerases, tautomerases, or mutases; transferases, kinases and phophatases. In some embodiments, the proteins are therapeutically significant proteins or peptides, including but not limited to growth factors, cytokines, ligands, receptors and inhibitors, as well as vaccines and antibodies. In additional embodiments, the proteins are commercially important industrial proteins/peptides (e.g., proteases, carbohydrases such as amylases and glucoamylases, cellulases, oxidases and lipases). In some embodiments, the gene encoding the proteins are naturally occurring genes, while in other embodiments, mutated and/or synthetic genes are used.

As used herein, “homologous protein” refers to a protein or polypeptide native or naturally occurring in a cell. In preferred embodiments, the cell is a Gram-positive cell, while in particularly preferred embodiments, the cell is a Bacillus host cell. In alternative embodiments, the homologous protein is a native protein produced by other organisms, including but not limited to E. coli. The invention encompasses host cells producing the homologous protein via recombinant DNA technology.

As used herein, an “operon region” comprises a group of contiguous genes that are transcribed as a single transcription unit from a common promoter, and are thereby subject to co-regulation. In some embodiments, the operon includes a regulator gene. In most preferred embodiments, operons that are highly expressed as measured by RNA levels, but have an unknown or unnecessary function are used.

As used herein, a “multi-contiguous single gene region” is a region wherein at least the coding regions of two genes occur in tandem and in some embodiments, include intervening sequences preceding and following the coding regions. In some embodiments, an antimicrobial region is included.

As used herein, an “antimicrobial region” is a region containing at least one gene that encodes an antimicrobial protein.

### DETAILED DESCRIPTION OF THE INVENTION

The present invention provides cells that have been genetically manipulated to have an altered capacity to produce expressed proteins, wherein the pckA gene has been modified or deleted. In particular, the present invention relates to Gram-positive microorganisms, such as Bacillus species having enhanced expression of a protein of interest, wherein one or more chromosomal genes have been modified and/or inactivated (e.g., pckA), and preferably wherein one or more chromosomal genes (e.g., pckA) have been modified and/or deleted from the Bacillus chromosome. In some further embodiments, one or more indigenous chromosomal regions have been modified and/or deleted from a corresponding wild-type Bacillus host chromosome. In preferred embodiments, such deletions provide advantages such as improved production of a protein of interest.

A. Gene Deletions

As indicated above, the present invention includes embodiments that involve singe or multiple gene deletions and/or mutations, as well as large chromosomal deletions.

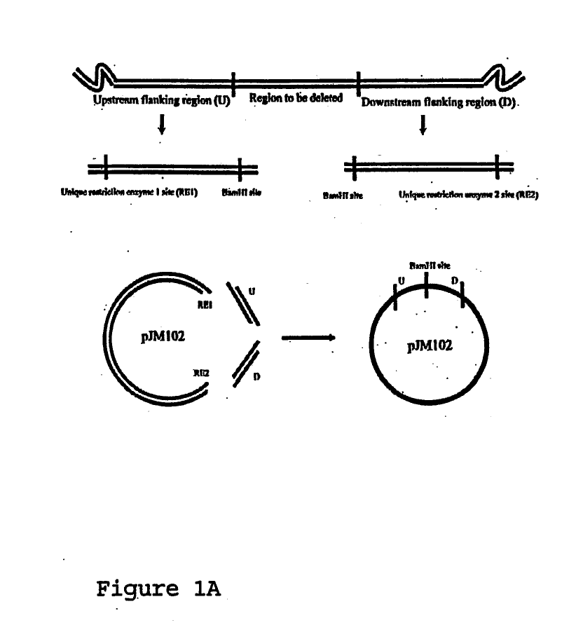

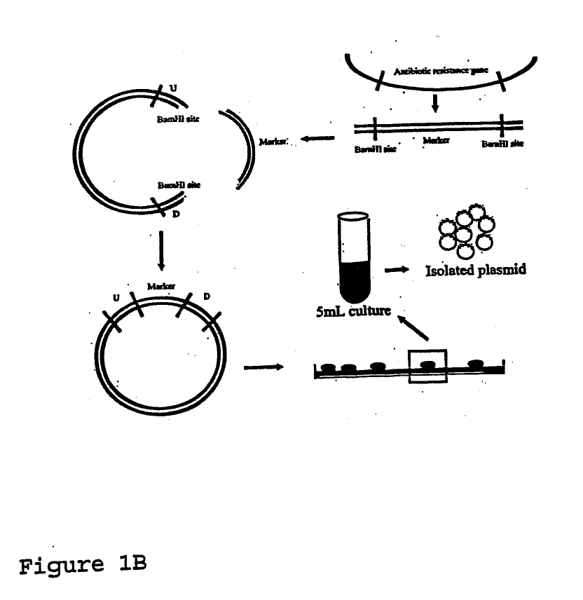

In some preferred embodiments, the present invention includes a DNA construct comprising an incoming sequence. The DNA construct is assembled in vitro, followed by direct cloning of the construct into a competent Bacillus host, such that the DNA construct becomes integrated into the Bacillus chromosome. For example, PCR fusion and/or ligation can be employed to assemble a DNA construct in vitro. In some embodiments, the DNA construct is a non-plasmid construct, while in other embodiments it is incorporated into a vector (e.g., a plasmid). In some embodiments, circular plasmids are used. In preferred embodiments, circular plasmids are designed to use an appropriate restriction enzyme (i.e., one that does not disrupt the DNA construct). However, linear plasmids also find use in the present invention (See, FIG. 1). However, other methods are suitable for use in the present invention, as known to those in the art (See e.g., Perego, “Integrational Vectors for Genetic Manipulation in Bacillus subtilis,” in (Sonenshein et al. (eds.), Bacillus subtilis and Other Gram-Positive Bacteria, American Society for Microbiology, Washington, D.C. [1993]).

In some embodiments, the incoming sequence includes a selective marker. In some preferred embodiments, the incoming sequence includes the chromosomal pckA gene, while in alternative preferred embodiments, the incoming sequence further comprises a chromosomal gene selected from the group consisting of sbo, sir, ybcO, csn, spolISA, phrC, sigB, rapA, CssS, trpA, trpB, trpC, trpD, trpE, trpF, tdh/kbl, alsD, sigD, prpC, gapB, fbp, rocA, ycgN, ycgM, rocF, and rocD, or fragments of any of these genes (alone or in combination). In additional embodiments, the incoming sequence includes a homologous pckA gene sequence, while in other embodiments, the incoming sequence further comprises at least one additional homologous sequence selected from the group consisting of sbo, sir, ybcO, csn, spolISA, phrC, sigB, rapA, CssS trpA, trpB, trpC, trpD, trpE, trpF, tdh/kbl, alsD, sigD, prpC, gapB, fbp, rocA, ycgN, ycgM, rocF, and/or rocD gene sequence. A homologous sequence is a nucleic acid sequence having at least 99%, 98%, 97%, 96%, 95%, 94% 93%, 92%, 91%, 90%, 88%, 85% or 80% sequence identity to a sbo, sir, ybcO, csn, spolISA, phrC, sigB, rapA, CssS trpA, trpB, trpC, trpD, trpE, trpF, tdh/kbl, alsD, sigD, prpC, gapB, pckA, fbp, rocA, ycgN, ycgM, rocF, and rocD gene or gene fragment thereof, which may be included in the incoming sequence. In preferred embodiments, the incoming sequence comprising a homologous sequence comprises at least 95% sequence identity to a sbo, sir, ybcO, csn, spolISA, phrC, sigB, rapA, CssS trpA, trpB, trpC, trpD, trpE, trpF, tdh/kbl, alsD, sigD, prpC, gapB, pckA, fbp, rocA, ycgN, ycgM, rocF, or rocD gene or gene fragment of any of these genes. In yet other embodiments, the incoming sequence comprises a selective marker flanked on the 5′ and 3′ ends with a fragment of the gene sequence. In some embodiments, when the DNA construct comprising the selective marker and gene, gene fragment or homologous sequence thereto is transformed into a host cell, the location of the selective marker renders the gene non-functional for its intended purpose. In some embodiments, the incoming sequence comprises the selective marker located in the promoter region of the gene. In other embodiments, the incoming sequence comprises the selective marker located after the promoter region of gene. In yet other embodiments, the incoming sequence comprises the selective marker located in the coding region of the gene. In further embodiments, the incoming sequence comprises a selective marker flanked by a homology box on both ends. In still further embodiments, the incoming sequence includes a sequence that interrupts the transcription and/or translation of the coding sequence. In yet additional embodiments, the DNA construct includes restriction sites engineered at the upstream and downstream ends of the construct.

Whether the DNA construct is incorporated into a vector or used without the presence of plasmid DNA, it is used to transform microorganisms. It is contemplated that any suitable method for transformation will find use with the present invention. In preferred embodiments, at least one copy of the DNA construct is integrated into the host Bacillus chromosome. In some embodiments, one or more DNA constructs of the invention are used to transform host cells. For example, one DNA construct may be used to inactivate a sir gene and another construct may be used to inactivate a phrC gene. Of course, additional combinations are contemplated and provided by the present invention.

In some preferred embodiments, the DNA construct also includes a polynucleotide encoding a protein of interest. In some of these preferred embodiments, the DNA construct also includes a constitutive or inducible promoter that is operably linked to the sequence encoding the protein of interest. In some preferred embodiments in which the protein of interest is a protease, the promoter is selected from the group consisting of a tac promoter, a β-lactamase promoter, or an aprE promoter (DeBoer et al., Proc. Natl. Acad. Sci. USA 80:21-25 [1983]). However, it is not intended that the present invention be limited to any particular promoter, as any suitable promoter known to those in the art finds use with the present invention. Nonetheless, in particularly preferred embodiments, the promoter is the B. subtilis aprE promoter.

Various methods are known for the transformation of Bacillus species. Indeed, methods for altering the chromosome of Bacillus involving plasmid constructs and transformation of the plasmids into E. coli are well known. In most methods, plasmids are subsequently isolated from E. coli and transformed into Bacillus. However, it is not essential to use such intervening microorganisms such as E. coli, and in some preferred embodiments, the DNA construct is directly transformed into a competent Bacillus host.

In some embodiments, the well-known Bacillus subtilis strain 168 finds use in the present invention. Indeed, the genome of this strain has been well-characterized (See, Kunst et al., Nature 390:249-256 [1997]; and Henner et al., Microbiol. Rev., 44:57-82 [1980]). The genome is comprised of one 4215 kb chromosome. While the coordinates used herein refer to the 168 strain, the invention encompasses analogous sequences from Bacillus strains.

In some embodiments, the incoming chromosomal sequence includes the pckA gene, while in alternative embodiments, the incoming chromosomal sequence further comprises one or more genes selected from the group consisting of sbo, sir, ybcO, csn, spolISA, sigB, phrC, rapA, CssS, trpA, trpB, trpC, trpD, trpE, trpF, tdh/kbl, alsD, sigD, prpC, gapB, fbp, rocA, ycgN, ycgM, rocF, and rocD gene fragments thereof and homologous sequences thereto. The DNA coding sequences of these genes from B. subtilis 168 are provided in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO:17, SEQ ID NO:39, SEQ ID NO:40, SEQ ID NO:42, SEQ ID NO:44, SEQ ID NO:46, SEQ ID NO:48, SEQ ID NO:50, SEQ ID NO:37, SEQ ID NO:25, SEQ ID NO:21, SEQ ID NO:50, SEQ ID NO:29, SEQ ID NO:23, SEQ ID NO:27, SEQ ID NO:19, SEQ ID NO:31, SEQ ID NO:48, SEQ ID NO:46, SEQ ID NO:35, and SEQ ID NO:33.

As mentioned above, in some embodiments, the incoming sequence which comprises pckA, alone or in combination with sbo, sir, ybcO, csn, spolISA, sigB, phrC, rapA, CssS, trpA, trpB, trpC, trpD, trpE, trpF, tdh, kbl, alsD, sigD, prpC, gapB, fbp, rocA, ycgN, ycgM, rocF, and rocD gene, a gene fragment thereof, or a homologous sequence thereto includes the coding region and may further include immediate chromosomal coding region flanking sequences. In some embodiments the coding region flanking sequences include a range of about 1 bp to 2500 bp; about 1 bp to 1500 bp, about 1 bp to 1000 bp, about 1 bp to 500 bp, and 1 bp to 250 bp. The number of nucleic acid sequences comprising the coding region flanking sequence may be different on each end of the gene coding sequence. For example, in some embodiments, the 5′ end of the coding sequence includes less than 25 bp and the 3′ end of the coding sequence includes more than 100 bp. Sequences of these genes and gene products are provided below. The numbering used herein is that used in subtilist (See e.g., Moszer et al., Microbiol, 141:261-268 [1995]).

The sbo coding sequence of B. subtilis 168 is shown below:

(SEQ ID NO:1)

ATGAAAAAAGCTGTCATTGTAGAAAACAAAGGTTGTGCAACATGCTCGAT
CGGAGCCGCTTGTCTAGTGGACGGTCCTATCCCTGATTTTGAAATTGCCG
GTGCAACAGGTCTATTCGGTCTATGGGGG.

The deduced amino acid sequence for Sbo is:

(SEQ ID NO:2)

MKKAVIVENKGCATCSIGAACLVDGPIPDFEIAGATGLFGLWG.

In one embodiment, the gene region found at about 3834868 to 3835219 bp of the B. subtilis 168 chromosome was deleted using the present invention. The sbo coding region found at about 3835081 to 3835209 produces subtilisin A, an antimicrobial that has activity against some Gram-positive bacteria. (See, Zheng et al., J. Bacteriol., 181:7346-7355 [1994]).

The sir coding sequence of B. subtilis 168 is shown below:

(SEQ ID NO: 3)

ATGATTGGAAGAATTATCCGTTTGTACCGTAAAAGAAAAGGCTATTCTAT
TAATCAGCTGGCTGTTGAGTCAGGCGTATCCAAATCCTATTTAAGCAAGA
TTGAAAGAGGCGTTCACACGAATCCGTCCGTTCAATTTTTAAAAAAAGTT
TCTGCCACACTGGAAGTTGAATTAACAGAATTATTTGACGCAGAAACAAT
GATGTATGAAAAAATCAGCGGCGGTGAAGAAGAATGGCGCGTACATTTAG
TGCAAGCCGTACAAGCCGGGATGGAAAAGGAAGAATTGTTCACTTTTACG
AACAGACTCAAGAAAGAACAGCCTGAAACTGCCTCTTACCGCAACCGCAA
ACTGACGGAATCCAATATAGAAGAATGGAAAGCGCTGATGGCGGAGGCAA
GAGAAATCGGCTTGTCTGTCCATGAAGTCAAATCCTTTTTAAAAACAAAG
GGAAGA.

The deduced amino acid sequence for Slr is:

(SEQ ID NO: 4)

MIGRIIRLYRKRKGYSINQLAVESGVSKSYLSKIERGVHTNPSVQFLKKV
SATLEVELTELFDAETMMYEKISGGEEEWRVHLVQAVQAGMEKEELFTFT
NRLKKEQPETASYRNRKLTESNIEEWKALMAEAREIGLSVHEVKSFLKTK
GR.

In one embodiment, the sequence found at about 3529014-3529603 bp of the B. subtilis 168 chromosome was deleted using the present invention. The slr coding sequence is found at about 3529131 to 3529586 of the chromosome.

The phrC coding sequence of B. subtilis 168 is provided below:

(SEQ ID NO: 13)

ATGAAATTGAAATCTAAGTTGTTTGTTATTTGTTTGGCCGCAGCCGCGAT
TTTTACAGCGGCTGGCGTTTCTGCTAATGCGGAAGCACTCGACTTTCATG
TGACAGAAAGAGGAATGACG.

The deduced amino acid sequence for PhrC is:

(SEQ ID NO: 14)

MKLKSKLFVICLAAAAIFTAAGVSANAEALDFHVTERGMT

Additionally, the coding region found at about 429531 to 429650 bp of the B. subtilis 168 chromosome was inactivated by an insertion of a selective marker at 429591 of the coding sequence.

The sigB coding sequence of B. subtilis 168 is shown below:

(SEQ ID NO: 9)

TTGATCATGACACAACCATCAAAAACTACGAAACTAACTAAAGATGAAGT
CGATCGGCTCATAAGCGATTACCAAACAAAGCAAGATGAACAAGCGCAGG
AAACGCTTGTGCGGGTGTATACAAATCTGGTTGACATGCTTGCGAAAAAA
TACTCAAAAGGCAAAAGCTTCCACGAGGATCTCCGCCAGGTCGGCATGAT
CGGGCTGCTAGGCGCGATTAAGCGATACGATCCTGTTGTCGGCAAATCGT
TTGAAGCTTTTGCAATCCCGACAATCATCGGTGAAATTAAACGTTTCCTC
AGAGATAAAACATGGAGCGTTCATGTGCCGAGACGAATTAAAGAACTCGG
TCCAAGAATCAAAATGGCGGTTGATCAGCTGACCACTGAAACACAAAGAT
CGCCGAAAGTCGAAGAGATTGCCGAATTCCTCGATGTTTCTGAAGAAGAG
GTTCTTGAAACGATGGAAATGGGCAAAAGCTATCAAGCCTTATCCGTTGA
CCACAGCATTGAAGCGGATTCGGACGGAAGCACTGTCACGATTCTTGATA
TCGTCGGATCACAGGAGGACGGATATGAGCGGGTCAACCAGCAATTGATG
CTGCAAAGCGTGCTTCATGTCCTTTCAGACCGTGAGAAACAAATCATAGA
CCTTACGTATATTCAAAACAAAAGCCAAAAAGAAACTGGGGACATTCTCG
GTATATCTCAAATGCACGTCTCGCGCTTGCAACGCAAAGCTGTGAAGAAG
CTCAGAGAGGCCTTGATTGAAGATCCCTCGATGGAGTTAATG.

The deduced amino acid sequence for SigB is:

(SEQ ID NO: 10)

MIMTQPSKTTKLTKDEVDRLISDYQTKQDEQAQETLVRVYTNLVDMLAKK
YSKGKSFHEDLRQVGMIGLLGAIKRYDPVVGKSFEAFAIPTIIGEIKRFL
RDKTWSVHVPRRIKELGPRIKMAVDQLTTETQRSPKVEEIAEFLDVSEEE
VLETMEMGKSYQALSVDHSIEADSDGSTVTILDIVGSQEDGYERVNQQLM
LQSVLHVLSDREKQIIDLTYIQNKSQKETGDILGISQMHVSRLQRKAVKK
LREALIEDPSMELM.

Additionally, the coding sequence is found at about 522417 to 5232085 bp of the B. subtilis 168 chromosome.

The spolISA coding sequence of B. subtilis 168 is shown below:

(SEQ ID NO: 11)

ATGGTTTTATTCTTTCAGATCATGGTCTGGTGCATCGTGGCCGGACTGGG
GTTATACGTGTATGCCACGTGGCGTTTCGAAGCGAAGGTCAAAGAAAAAA
TGTCCGCCATTCGGAAAACTTGGTATTTGCTGTTTGTTCTGGGCGCTATG
GTATACTGGACATATGAGCCCACTTCCCTATTTACCCACTGGGAACGGTA
TCTCATTGTCGCAGTCAGTTTTGCTTTGATTGATGCTTTTATCTTCTTAA
GTGCATATGTCAAAAAACTGGCCGGCAGCGAGCTTGAAACAGACACAAGA
GAAATTCTTGAAGAAAACAACGAAATGCTCCACATGTATCTCAATCGGCT
GAAAACATACCAATACCTATTGAAAAACGAACCGATCCATGTTTATTATG
GAAGTATAGATGCTTATGCTGAAGGTATTGATAAGCTGCTGAAAACCTAT
GCTGATAAAATGAACTTAACGGCTTCTCTTTGCCACTATTCGACACAGGC
TGATAAAGACCGGTTAACCGAGCATATGGATGATCCGGCAGATGTACAAA
CACGGCTCGATCGAAAGGATGTTTATTACGACCAATACGGAAAAGTGGTT
CTCATCCCTTTTACCATCGAGACACAGAACTATGTCATCAAGCTGACGTC
TGACAGCATTGTCACGGAATTTGATTATTTGCTATTTACGTCATTAACGA
GCATATATGATTTGGTGCTGCCAATTGAGGAGGAAGGTGAAGGA.

The deduced amino acid sequence for SpolISA is:

(SEQ ID NO: 12)

MVLFFQIMVWCIVAGLGLYVYATWRFEAKVKEKMSAIRKTWYLLFVLGAM
VYWTYEPTSLFTHWERYLIVAVSFALIDAFIFLSAYVKKLAGSELETDTR
EILEENNEMLHMYLNRLKTYQYLLKNEPIHVYYGSIDAYAEGIDKLLKTY
ADKMNLTASLCHYSTQADKDRLTEHMDDPADVQTRLDRKDVYYDQYGKVV
LIPFTIETQNYVIKLTSDSIVTEFDYLLFTSLTSIYDLVLPIEEEGEG.

Additionally, the coding region is found at about 1347587 to 1348714 bp of the B. subtilis 168 chromosome.

The csn coding sequence of B. subtilis 168 is shown below:

(SEQ ID NO: 7)

ATGAAAATCAGTATGCAAAAAGCAGATTTTTGGAAAAAAGCAGCGATCTC
ATTACTTGTTTTCACCATGTTTTTTACCCTGATGATGAGCGAAACGGTTT
TTGCGGCGGGACTGAATAAAGATCAAAAGCGCCGGGCGGAACAGCTGACA
AGTATCTTTGAAAACGGCACAACGGAGATCCAATATGGATATGTAGAGCG
ATTGGATGACGGGCGAGGCTATACATGCGGACGGGCAGGCTTTACAACGG
CTACCGGGGATGCATTGGAAGTAGTGGAAGTATACACAAAGGCAGTTCCG
AATAACAAACTGAAAAAGTATCTGCCTGAATTGCGCCGTCTGGCCAAGGA
AGAAAGCGATGATACAAGCAATCTCAAGGGATTCGCTTCTGCCTGGAAGT
CGCTTGCAAATGATAAGGAATTTCGCGCCGCTCAAGACAAAGTAAATGAC
CATTTGTATTATCAGCCTGCCATGAAACGATCGGATAATGCCGGACTAAA
AACAGCATTGGCAAGAGCTGTGATGTACGATACGGTTATTCAGCATGGCG
ATGGTGATGACCCTGACTCTTTTTATGCCTTGATTAAACGTACGAACAAA
AAAGCGGGCGGATCACCTAAAGACGGAATAGACGAGAAGAAGTGGTTGAA
TAAATTCTTGGACGTACGCTATGACGATCTGATGAATCCGGCCAATCATG
ACACCCGTGACGAATGGAGAGAATCAGTTGCCCGTGTGGACGTGCTTCGC
TCTATCGCCAAGGAGAACAACTATAATCTAAACGGACCGATTCATGTTCG
TTCAAACGAGTACGGTAATTTTGTAATCAAA.

The deduced amino acid sequence for Csn is:

(SEQ ID NO: 8)

MKISMQKADFWKKAAISLLVFTMFFTLMMSETVFAAGLNKDQKRRAEQLT
SIFENGTTEIQYGYVERLDDGRGYTCGRAGFTTATGDALEVVEVYTKAVP
NNKLKKYLPELRRLAKEESDDTSNLKGFASAWKSLANDKEFRAAQDKVND
HLYYQPAMKRSDNAGLKTALARAVMYDTVIQHGDGDDPDSFYALIKRTNK
KAGGSPKDGIDEKKWLNKFLDVRYDDLMNPANHDTRDEWRESVARVDVLR
SIAKENNYNLNGPIHVRSNEYGNFVIK.

Additionally, the coding region is found at about 2747213 to 2748043 bp of the B. subtilis 168 chromosome.

The ybcO coding sequence of B. subtilis 168 is shown below:

(SEQ ID NO: 5)

ATGAAAAGAAACCAAAAAGAATGGGAATCTGTGAGTAAAAAAGGACTTAT
GAAGCCGGGAGGTACTTCGATTGTGAAAGCTGCTGGCTGCATGGGCTGTT
GGGCCTCGAAGAGTATTGCTATGACACGTGTTTGTGCACTTCCGCATCCT
GCTATGAGAGCTATT.

The deduced amino acid sequence for YbcO is:

(SEQ ID NO: 6)

MKRNQKEWESVSKKGLMKPGGTSIVKAAGCMGCWASKSIAMTRVCALPHP
AMRAI.

Additionally, the coding region is found at about 213926 to 214090 bp of the B. subtilis 168 chromosome.

The rapA coding sequence of B. subtilis 168 is shown below:

(SEQ ID NO: 15)

TTGAGGATGAAGCAGACGATTCCGTCCTCTTATGTCGGGCTTAAAATTAA
TGAATGGTATACTCATATCCGGCAGTTCCACGTCGCTGAAGCCGAACGGG
TCAAGCTCGAAGTAGAAAGAGAAATTGAGGATATGGAAGAAGACCAAGAT
TTGCTGCTGTATTATTCTTTAATGGAGTTCAGGCACCGTGTCATGCTGGA
TTACATTAAGCCTTTTGGAGAGGACACGTCGCAGCTAGAGTTTTCAGAAT
TGTTAGAAGACATCGAAGGGAATCAGTACAAGCTGACAGGGCTTCTCGAA
TATTACTTTAATTTTTTTCGAGGAATGTATGAATTTAAGCAGAAGATGTT
TGTCAGTGCCATGATGTATTATAAACGGGCAGAAAAGAATCTTGCCCTCG
TCTCGGATGATATTGAGAAAGCAGAGTTTGCTTTTAAAATGGCTGAGATT
TTTTACAATTTAAAACAAACCTATGTTTCGATGAGCTACGCCGTTCAGGC
ATTAGAAACATACCAAATGTATGAAACGTACACCGTCCGCAGAATCCAAT
GTGAATTCGTTATTGCAGGTAATTATGATGATATGCAGTATCCAGAAAGA
GCATTGCCCCACTTAGAACTGGCTTTAGATCTTGCAAAGAAAGAAGGCAA
TCCCCGCCTGATCAGTTCTGCCCTATATAATCTCGGAAACTGCTATGAGA
AAATGGGTGAACTGCAAAAGGCAGCCGAATACTTTGGGAAATCTGTTTCT
ATTTGCAAGTCGGAAAAGTTCGATAATCTTCCGCATTCTATCTACTCTTT
AACACAAGTTCTGTATAAACAAAAAAATGACGCCGAAGCGCAAAAAAAGT
ATCGTGAAGGATTGGAAATCGCCCGTCAATACAGTGATGAATTATTTGTG
GAGCTTTTTCAATTTTTACATGCGTTATACGGAAAAAACATTGACACAGA
ATCAGTCTCACACACCTTTCAATTTCTTGAAGAACATATGCTGTATCCTT
ATATTGAAGAGCTGGCGCATGATGCTGCCCAATTCTATATAGAAAACGGA
CAGCCCGAAAAAGCACTTTCATTTTATGAGAAAATGGTGCACGCACAAAA
ACAAATCCAGAGAGGAGATTGTTTATATGAAATC.

The deduced amino acid sequence for RapA is:

(SEQ ID NO: 16)

MRMKQTIPSSYVGLKINEWYTHIRQFHVAEAERVKLEVEREIEDMEEDQD
LLLYYSLMEFRHRVMLDYIKPFGEDTSQLEFSELLEDIEGNQYKLTGLLE
YYFNFFRGMYEFKQKMFVSAMMYYKRAEKNLALVSDDIEKAEFAFKMAEI
FYNLKQTYVSMSYAVQALETYQMYETYTVRRIQCEFVIAGNYDDMQYPER
ALPHLELALDLAKKEGNPRLISSALYNLGNCYEKMGELQKAAEYFGKSVS
ICKSEKFDNLPHSIYSLTQVLYKQKNDAEAQKKYREGLEIARQYSDELFV
ELFQFLHALYGKNIDTESVSHTFQFLEEHMLYPYIEELAHDAAQFYIENG
QPEKALSFYEKMVHAQKQIQRGDCLYEI

Additionally, the coding region is found at about 1315179 to 1316312 bp of the B. subtilis 168 chromosome.

The Css coding sequence of B. subtilis 168 is shown below:

(SEQ ID NO: 17)

ATGAAAAACAAGCCGCTCGCGTTTCAGATATGGGTTGTCATATCCGGCAT
CCTGTTAGCGATATCGATTTTACTGCTTGTGTTATTTTCAAACACGCTGC
GAGATTTTTTCACTAATGAAACGTATACGACGATTGAAAATGAGCAGCAT
GTTCTGACAGAGTACCGCCTGCCAGGTTCGATTGAAAGGCGCTATTACAG
CGAGGAAGCGACGGCGCCGACAACTGTCCGCTCCGTACAGCACGTGCTCC
TTCCTGAAAATGAAGAGGCTTCTTCAGACAAGGATTTAAGCATTCTGTCA
TCTTCATTTATCCACAAGGTGTACAAGCTGGCTGATAAGCAGGAAGCTAA
AAAGAAACGTTACAGCGCCGACGTCAATGGAGAGAAAGTGTTTTTTGTCA
TTAAAAAGGGACTTTCCGTCAATGGACAATCAGCGATGATGCTCTCTTAC
GCGCTTGATTCTTATCGGGACGATTTGGCCTATACCTTGTTCAAACAGCT
TCTGTTTATTATAGCTGTCGTCATTTTATTAAGCTGGATTCCGGCTATTT
GGCTTGCAAAGTATTTATCAAGGCCTCTTGTATCATTTGAAAAACACGTC
AAACGGATTTCTGAACAGGATTGGGATGACCCAGTAAAAGTGGACCGGAA
AGATGAAATCGGCAAATTGGGCCATACCATCGAAGAGATGCGCCAAAAGC
TTGTGCAAAAGGATGAAACAGAAAGAACTCTATTGCAAAATATCTCTCAT
GATTTAAAAACGCCGGTCATGGTCATCAGAGGCTATACACAATCAATTAA
AGACGGGATTTTTCCTAAAGGAGACCTTGAAAACACTGTAGATGTTATTG
AATGCGAAGCTCTTAAGCTGGAGAAAAAAATAAAGGATTTATTATATTTA
ACGAAGCTGGATTATTTAGCGAAGCAAAAAGTGCAGCACGACATGTTCAG
TATTGTGGAAGTGACAGAAGAAGTCATCGAACGATTGAAGTGGGCGCGGA
AAGAACTATCGTGGGAAATTGATGTAGAAGAGGATATTTTGATGCCGGGC
GATCCGGAGCAATGGAACAAACTCCTCGAAAACATTTTGGAAAATCAAAT
CCGCTATGCTGAGACAAAAATAGAAATCAGCATGAAACAAGATGATCGAA
ATATCGTGATCACCATTAAAAATGACGGTCCGCATATTGAAGATGAGATG
CTCTCCAGCCTCTATGAGCCTTTTAATAAAGGGAAGAAAGGCGAATTCGG
CATTGGTCTAAGCATCGTAAAACGAATTTTAACTCTTCATAAGGCATCTA
TCTCAATTGAAAATGACAAAACGGGTGTATCATACCGCATAGCAGTGCCA
AAA.

The deduced amino acid sequence for Css (GenBank Accession No. 032193) is:

(SEQ ID NO: 18)

MKNKPLAFQIWVVISGILLAISILLLVLFSNTLRDFFTNETYTTIENEQH
VLTEYRLPGSIERRYYSEEATAPTTVRSVQ HVLLPENEEASSDKDLSIL
S SSFIHKVYKLADKQEAKKKRYSADVNGEKVFFVIKKGLSVNGQSAMML
SYALDSYRDDLAYTLFKQLLFIIAVVILLSWIPAIWLAKYLSRPLVSFEK
HVKRISEQDWDDPVKVDRKDEIGKLGHTIEEMRQKLVQKDETERTLLQNI
SHDLKTPVMVIRGYTQSIKDGIFPKGDLENTVDVIECEALKLEKKIKDLL
YLTKLDYLAKQKVQHDMFSIVEVTEEVIERLKWARKELSWEIVEEDILMP
GDPEQWNKLLENILENQIRYAETKIEISMKQDDRNIVITIKNDGPHIEDE
MLSSLYEPFNKGKKGEFGIGLSIVKRILTLHKASISIENDKTGVSYRIAV
PK.

Additionally, the gene region is found at about 3384612 to 3386774 bp of the B. subtilis 168 chromosome.

The fbp coding sequence of the Fbp protein (fructose-1,6-biophosphatase) of B. subtilis 168 is shown below:

(SEQ ID NO: 19)

ATGTTTAAAAATAATGTCATACTTTTAAATTCACCTTATCATGCACATGC
TCATAAAGAGGGGTTTATTCTAAAAAGGGGATGGACGGTTTTGGAAAGCA
AGTACCTAGATCTACTCGCACAAAAATACGATTGTGAAGAAAAAGTGGTA
ACAGAAATCATCAATTTGAAAGCGATATTGAACCTGCCAAAAGGCACCGA
GCATTTTGTCAGTGATCTGCACGGAGAGTATCAGGCATTCCAGCACGTGT
TGCGCAATGGTTCAGGACGAGTCAAAGAGAAGATACGCGACATCTTCAGC
GGTGTCATTTACGATAGAGAAATTGATGAATTAGCAGCATTGGTCTATTA
TCCGGAAGACAAACTGAAATTAATCAAACATGACTTTGATGCGAAAGAAG
CGTTAAACGAGTGGTATAAAGAAACGATTCATCGAATGATTAAGCTCGTT
TCATATTGCTCCTCTAAGTATACCCGCTCCAAATTACGCAAAGCACTGCC
TGCCCAATTTGCTTATATTACGGAGGAGCTGTTATACAAAACAGAACAAG
CTGGCAACAAGGAGCAATATTACTCCGAAATCATTGATCAGATCATTGAA
CTTGGCCAAGCCGATAAGCTGATCACCGGCCTTGCTTACAGCGTTCAGCG
ATTGGTGGTCGACCATCTGCATGTGGTCGGCGATATTTATGACCGCGGCC
CGCAGCCGGATAGAATTATGGAAGAACTGATCAACTATCATTCTGTCGAT
ATTCAGTGGGGAAATCACGATGTCCTTTGGATCGGCGCCTATTCCGGTTC
CAAAGTGTGCCTGGCCAATATTATCCGCATCTGTGCCCGCTACGACAACC
TGGATATTATTGAGGACGTGTACGGCATCAACCTGAGACCGCTGCTGAAC
CTGGCCGAAAAATATTATGATGATAATCCAGCGTTCCGTCCAAAAGCAGA
CGAAAACAGGCCAGAGGATGAGATTAAGCAAATCACAAAAATCCATCAAG
CGATTGCCATGATCCAATTCAAGCTTGAGAGCCCGATTATCAAGAGACGG
CCGAACTTTAATATGGAAGAGCGGCTGTTATTAGAGAAAATAGACTATGA
CAAAAATGAAATCACGCTGAACGGAAAAACATATCAACTGGAAAACACCT
GCTTTGCGACGATTAATCCGGAGCAGCCAGATCAGCTATTAGAAGAAGAA
GCAGAAGTCATAGACAAGCTGCTATTCTCTGTCCAGCATTCCGAAAAGCT
GGGCCGCCATATGAATTTTATGATGAAAAAAGGCAGCCTTTATTTAAAAT
ATAACGGCAACCTGTTGATTCACGGCTGTATTCCAGTTGATGAAAACGGC
AATATGGAAACGATGATGATTGAGGATAAACCGTATGCGGGCCGTGAGCT
GCTCGATGTATTTGAACGATTCTTGCGGGAAGCCTTTGCCCACCCGGAAG
AAACCGATGACCTGGCGACAGATATGGCTTGGTATTTATGGACAGGCGAA
TACTCCTCCCTCTTCGGAAAACGCGCCATGACGACATTTGAGCGCTATTT
CATCAAAGAGAAGGAAACGCATAAAGAGAAGAAAAACCCGTATTATTATT
TACGAGAAGACGAGGCAACCTGCCGAAACATCCTGGCAGAATTCGGCCTC
AATCCAGATCACGGCCATATCATCAACGGCCATACACCTGTAAAAGAAAT
CGAAGGAGAAGACCCAATCAAAGCAAACGGAAAAATGATCGTCATCGACG
GCGGCTTCTCCAAAGCCTACCAATCCACAACAGGCATCGCCGGCTACACG
CTGCTATACAACTCCTACGGCATGCAGCTCGTCGCCCATAAACACTTCAA
TTCCAAGGCAGAAGTCCTAAGCACCGGAACCGACGTCTTAACGGTCAAAC
GATTAGTGGACAAAGAGCTTGAGCGGAAGAAAGTGAAGGAAACGAATGTG
GGTGAGGAATTGTTGCAGGAAGTTGCGATTTTAGAGAGTTTGCGGGAGTA
TCGGTATATGAAG.

The deduced amino acid sequence of the Fbp protein is:

(SEQ ID NO: 20)

MFKNNVILLNSPYHAHAHKEGFILKRGWTVLESKYLDLLAQKYDCEEKVV
TEIINLKAILNLPKGTEHFVSDLHGEYQAFQHVLRNGSGRVKEKIRDIFS
GVIYDREIDELAALVYYPEDKLKLIKHDFDAKEALNEWYKETIHRMIKLV
SYCSSKYTRSKLRKALPAQFAYITEELLYKTEQAGNKEQYYSEIIDQIIE
LGQADKLITGLAYSVQRLVVDHLHVVGDIYDRGPQPDRIMEELINYHSVD
IQWGNHDVLWIGAYSGSKVCLANIIRICARYDNLDIIEDVYGINLRPLLN
LAEKYYDDNPAFRPKADENRPEDEIKQITKIHQAIAMIQFKLESPIIKRR
PNFNMEERLLLEKIDYDKNEITLNGKTYQLENTCFATINPEQPDQLLEEE
AEVIDKLLFSVQHSEKLGRHMNFMMKKGSLYLKYNGNLLIHGCIPVDENG
NMETMMIEDKPYAGRELLDVFERFLREAFAHPEETDDLATDMAWYLWTGE
YSSLFGKRAMTTFERYFIKEKETHKEKKNPYYYLREDEATCRNILAEFGL
NPDHGHIINGHTPVKEIEGEDPIKANGKMIVIDGGFSKAYQSTTGIAGYT
LLYNSYGMQLVAHKHFNSKAEVLSTGTDVLTVKRLVDKELERKKVKETNV
GEELLQEVAILESLREYRYMK.

Additionally, the coding region is found at about 4127053 to 4129065 bp of the B. subtilis 168 chromosome.

The alsD coding sequence of the alsD protein (alpha-acetolactate decarboxylase) of B. subtilis 168 is shown below:

(SEQ ID NO: 21)

ATGAAACGAGAAAGCAACATTCAAGTGCTCAGCCGTGGTCAAAAAGATCA
GCCTGTGAGCCAGATTTATCAAGTATCAACAATGACTTCTCTATTAGACG
GAGTATATGACGGAGATTTTGAACTGTCAGAGATTCCGAAATATGGAGAC
TTCGGTATCGGAACCTTTAACAAGCTTGACGGAGAGCTGATTGGGTTTGA
CGGCGAATTTTACCGTCTTCGCTCAGACGGAACCGCGACACCGGTCCAAA
ATGGAGACCGTTCACCGTTCTGTTCATTTACGTTCTTTACACCGGACATG
ACGCACAAAATTGATGCGAAAATGACACGCGAAGACTTTGAAAAAGAGAT
CAACAGCATGCTGCCAAGCAGAAACTTATTTTATGCAATTCGCATTGACG
GATTGTTTAAAAAGGTGCAGACAAGAACAGTAGAACTTCAAGAAAAACCT
TACGTGCCAATGGTTGAAGCGGTCAAAACACAGCCGATTTTCAACTTCGA
CAACGTGAGAGGAACGATTGTAGGTTTCTTGACACCAGCTTATGCAAACG
GAATCGCCGTTTCTGGCTATCACCTGCACTTCATTGACGAAGGACGCAAT
TCAGGCGGACACGTTTTTGACTATGTGCTTGAGGATTGCACGGTTACGAT
TTCTCAAAAAATGAACATGAATCTCAGACTTCCGAACACAGCGGATTTCT
TTAATGCGAATCTGGATAACCCTGATTTTGCGAAAGATATCGAAACAACT
GAAGGAAGCCCTGAA.

The deduced amino acid sequence AlsD protein sequence is:

(SEQ ID NO: 22)

MKRESNIQVLSRGQKDQPVSQIYQVSTMTSLLDGVYDGDFELSEIPKYGD
FGIGTFNKLDGELIGFDGEFYRLRSDGTATPVQNGDRSPFCSFTFFTPDM
THKIDAKMTREDFEKEINSMLPSRNLFYAIRIDGLFKKVQTRTVELQEKP
YVPMVEAVKTQPIFNFDNVRGTIVGFLTPAYANGIAVSGYHLHFIDEGRN
SGGHVFDYVLEDCTVTISQKMNMNLRLPNTADFFNANLDNPDFAKDIETT
EGSPE.

Additionally, the coding region is found at about 3707829-3708593 bp of the B. subtilis 168 chromosome.

The gapB coding sequence of the gapB protein (glyceraldehyde-3-phosphate dehydrogenase) of B. subtilis 168 is shown below:

(SEQ ID NO: 23)

ATGAAGGTAAAAGTAGCGATCAACGGGTTTGGAAGAATCGGAAGAATGGT
TTTTAGAAAAGCGATGTTAGACGATCAAATTCAAGTAGTGGCCATTAACG
CCAGCTATTCCGCAGAAACGCTGGCTCATTTAATAAAGTATGACACAATT
CACGGCAGATACGACAAAGAGGTTGTGGCTGGTGAAGATAGCCTGATCGT
AAATGGAAAGAAAGTGCTTTTGTTAAACAGCCGTGATCCAAAACAGCTGC
CTTGGCGGGAATATGATATTGACATAGTCGTCGAAGCAACAGGGAAGTTT
AATGCTAAAGATAAAGCGATGGGCCATATAGAAGCAGGTGCAAAAAAAGT
GATTTTGACCGCTCCGGGAAAAAATGAAGACGTTACCATTGTGATGGGCG
TAAATGAGGACCAATTCGACGCTGAGCGCCATGTCATTATTTCAAATGCG
TCATGCACGACAAATTGCCTTGCGCCTGTTGTAAAAGTGCTGGATGAAGA
GTTTGGCATTGAGAGCGGTCTGATGACTACAGTTCATGCGTATACGAATG
ACCAAAAAAATATTGATAACCCGCACAAAGATTTGCGCCGGGCGCGGGCT
TGCGGTGAATCCATCATTCCAACAACAACAGGAGCGGCAAAGGCGCTTTC
GCTTGTGCTGCCGCATCTGAAAGGAAAACTTCACGGCCTCGCCTTGCGTG
TCCCTGTTCCGAACGTCTCATTGGTTGATCTCGTTGTTGATCTGAAAACG
GATGTTACGGCTGAAGAAGTAAACGAGGCATTTAAACGCGCTGCCAAAAC
GTCGATGTACGGTGTACTTGATTACTCAGATGAACCGCTCGTTTCGACTG
ATTATAATACGAATCCGCATTCAGCGGTCATTGACGGGCTTACAACAATG
GTAATGGAAGACAGGAAAGTAAAGGTGCTGGCGTGGTATGACAACGAATG
GGGCTACTCCTGCAGAGTTGTTGATCTAATCCGCCATGTAGCGGCACGAA
TGAAACATCCGTCTGCTGTA.

The deduced amino acid sequence of the GapB protein is:

(SEQ ID NO: 24)

MKVKVAINGFGRIGRMVFRKAMLDDQIQVVAINASYSAETLAHLIKYDTI
HGRYDKEVVAGEDSLIVNGKKVLLLNSRDPKQLPWREYDIDIVVEATGKF
NAKDKAMGHIEAGAKKVILTAPGKNEDVTIVMGVNEDQFDAERHVIISNA
SCTTNCLAPVVKVLDEEFGIESGLMTTVHAYTNDQKNIDNPHKDLRRARA
CGESIIPTTTGAAKALSLVLPHLKGKLHGLALRVPVPNVSLVDLVVDLKT
DVTAEEVNEAFKRAAKTSMYGVLDYSDEPLVSTDYNTNPHSAVIDGLTTM
VMEDRKVKVLAWYDNEWGYSCRVVDLIRHVAARMKHPSAV.

Additionally, the coding region is found at about 2966075-2967094 bp of the B. subtilis 168 chromosome.

The Kbl coding sequence of the Kbl protein (2-amino-3-ketobutyrate CoA ligase) is shown below:

(SEQ ID NO: 25)

ATGACGAAGGAATTTGAGTTTTTAAAAGCAGAGCTTAATAGTATGAAAGA
AAACCATACATGGCAAGACATAAAACAGCTTGAATCTATGCAGGGCCCAT
CTGTCACAGTGAATCACCAAAAAGTCATTCAGCTATCTTCTAATAATTAC
CTCGGATTCACTTCACATCCTAGACTCATCAACGCCGCACAGGAGGCCGT
TCAGCAGTATGGAGCCGGCACCGGATCAGTGAGAACGATTGCGGGTACAT
TTACAATGCATCAAGAGCTTGAGAAAAAGCTGGCAGCCTTTAAAAAAACG
GAGGCGGCACTTGTATTCCAATCAGGCTTCACAACAAACCAAGGCGTACT
TTCAAGTATTCTATCAAAAGAGGACATTGTCATCTCAGATGAATTGAACC
ATGCCTCTATTATTGACGGAATTCGACTGACAAAGGCGGATAAAAAGGTG
TATCAGCACGTCAATATGAGTGATTTAGAGCGGGTGCTGAGAAAGTCAAT
GAATTATCGGATGCGTCTGATTGTGACAGACGGCGTATTTTCCATGGATG
GCAACATAGCTCCTCTGCCTGATATTGTAGAGCTCGCTGAGAAATATGAC
GCATTTGTGATGGTGGATGACGCCCATGCATCCGGAGTACTTGGCGAAAA
CGGCAGGGGAACGGTGAATCACTTCGGTCTTGACGGCAGAGTGCATATTC
AGGTCGGAACATTAAGCAAGGCAATCGGAGTGCTCGGCGGCTACGCTGCA
GGTTCAAAGGTGCTGATCGATTATTTGCGCCATAAAGGCCGTCCATTTTT
ATTCAGCACATCTCATCCGCCGGCAGTCACTGCAGCTTGTATGGAAGCGA
TTGATGTCTTGCTTGAAGAGCCGGAGCATATGGAGCGCTTGTGGGAGAAT
ACTGCCTATTTTAAAGCAATGCTTGTGAAAATGGGTCTGACTCTCACGAA
GAGTGAAACGCCGATTCTTCCTATTTTAATAGGTGATGAAGGTGTGGCAA
AGCAATTTTCAGATCAGCTCCTTTCTCGCGGTGTTTTTGCCCAAAGTATC
GTTTTCCCGACTGTAGCAAAGGGAAAAGCCAGAATTCGCACGATTATAAC
AGCAGAGCACACCAAAGATGAACTGGATCAGGCGCTTGATGTCATCGAAA
AGACGGCAAAGGAGCTCCAGCTATTG.

The deduced amino acid sequence of the Kbl protein is:

(SEQ ID NO: 26)

MTKEFEFLKAELNSMKENHTWQDIKQLESMQGPSVTVNHQKVIQLSSNNY
LGFTSHPRLINAAQEAVQQYGAGTGSVRTIAGTFTMHQELEKKLAAFKKT
EAALVFQSGFTTNQGVLSSILSKEDIVISDELNHASIIDGIRLTKADKKV
YQHVNMSDLERVLRKSMNYRMRLIVTDGVFSMDGNIAPLPDIVELAEKYD
AFVMVDDAHASGVLGENGRGTVNHFGLDGRVHIQVGTLSKAIGVLGGYAA
GSKVLIDYLRHKGRPFLFSTSHPPAVTAACMEAIDVLLEEPEHMERLWEN
TAYFKAMLVKMGLTLTKSETPILPILIGDEGVAKQFSDQLLSRGVFAQSI
VFPTVAKGKARIRTIITAEHTKDELDQALDVIEKTAKELQLL.

Additionally, the coding region is found at about 1770787-1771962 bp of the B. subtilis 168 chromosome.

The pckA coding sequence of the PckA (phosphoenolpyruvate carboxykinase) of B. subtilis 168 is shown below:

(SEQ ID NO: 27)

ATGAACTCAGTTGATTTGACCGCTGATTTACAAGCCTTATTAACATGTCC
AAATGTGCGTCATAATTTATCAGCAGCACAGCTAACAGAAAAAGTCCTCT
CCCGAAACGAAGGCATTTTAACATCCACAGGTGCTGTTCGCGCGACAACA
GGCGCTTACACAGGACGCTCACCTAAAGATAAATTCATCGTGGAGGAAGA
AAGCACGAAAAATAAGATCGATTGGGGCCCGGTGAATCAGCCGATTTCAG
AAGAAGCGTTTGAGCGGCTGTACACGAAAGTTGTCAGCTATTTAAAGGAG
CGAGATGAACTGTTTGTTTTCGAAGGATTTGCCGGAGCAGACGAGAAATA
CAGGCTGCCGATCACTGTCGTAAATGAGTTCGCATGGCACAATTTATTTG
CGCGGCAGCTGTTTATCCGTCCGGAAGGAAATGATAAGAAAACAGTTGAG
CAGCCGTTCACCATTCTTTCTGCTCCGCATTTCAAAGCGGATCCAAAAAC
AGACGGCACTCATTCCGAAACGTTTATTATTGTCTCTTTCGAAAAGCGGA
CAATTTTAATCGGCGGAACTGAGTATGCCGGTGAAATGAAGAAGTCCATT
TTCTCCATTATGAATTTCCTGCTGCCTGAAAGAGATATTTTATCTATGCA
CTGCTCCGCCAATGTCGGTGAAAAAGGCGATGTCGCCCTTTTCTTCGGAC
TGTCAGGAACAGGAAAGACCACCCTGTCGGCAGATGCTGACCGCAAGCTG
ATCGGTGACGATGAACATGGCTGGTCTGATACAGGCGTCTTTAATATTGA
AGGCGGATGCTACGCTAAGTGTATTCATTTAAGCGAGGAAAAGGAGCCGC
AAATCTTTAACGCGATCCGCTTCGGGTCTGTTCTCGAAAATGTCGTTGTG
GATGAAGATACACGCGAAGCCAATTATGATGATTCCTTCTATACTGAAAA
CACGCGGGCAGCTTACCCGATTCATATGATTAATAACATCGTGACTCCAA
GCATGGCCGGCCATCCGTCAGCCATTGTATTTTTGACGGCTGATGCCTTC
GGAGTCCTGCCGCCGATCAGCAAACTAACGAAGGAGCAGGTGATGTACCA
TTTTTTGAGCGGTTACACGAGTAAGCTTGCCGGAACCGAACGTGGTGTCA
CGTCTCCTGAAACGACGTTTTCTACATGCTTCGGCTCACCGTTCCTGCCG
CTTCCTGCTCACGTCTATGCTGAAATGCTCGGCAAAAAGATCGATGAACA
CGGCGCAGACGTTTTCTTAGTCAATACCGGATGGACCGGGGGCGGCTACG
GCACAGGCGAACGAATGAAGCTTTCTTACACTAGAGCAATGGTCAAAGCA
GCGATTGAAGGCAAATTAGAGGATGCTGAAATGATAACTGACGATATTTT
CGGCCTGCACATTCCGGCCCATGTTCCTGGCGTTCCTGATCATATCCTTC
AGCCTGAAAACACGTGGACCAACAAGGAAGAATACAAAGAAAAAGCAGTC
TACCTTGCAAATGAATTCAAAGAGAACTTTAAAAAGTTCGCACATACCGA
TGCCATCGCCCAGGCAGGCGGCCCTCTCGTA.

The deduced amino acid sequence of the PckA protein is:

(SEQ ID NO: 28)

MNSVDLTADLQALLTCPNVRHNLSAAQLTEKVLSRNEGILTSTGAVRATT
GAYTGRSPKDKFIVEEESTKNKIDWGPVNQPISEEAFERLYTKVVSYLKE
RDELFVFEGFAGADEKYRLPITVVNEFAWHNLFARQLFIRPEGNDKKTVE
QPFTILSAPHFKADPKTDGTHSETFIIVSFEKRTILIGGTEYAGEMKKSI
FSIMNFLLPERDILSMHCSANVGEKGDVALFFGLSGTGKTTLSADADRKL
IGDDEHGWSDTGVFNIEGGCYAKCIHLSEEKEPQIFNAIRFGSVLENVVV
DEDTREANYDDSFYTENTRAAYPIHMINNIVTPSMAGHPSAIVFLTADAF
GVLPPISKLTKEQVMYHFLSGYTSKLAGTERGVTSPETTFSTCFGSPFLP
LPAHVYAEMLGKKIDEHGADVFLVNTGWTGGGYGTGERMKLSYTRAMVKA
AIEGKLEDAEMITDDIFGLHIPAHVPGVPDHILQPENTWTNKEEYKEKAV
YLANEFKENFKKFAHTDAIAQAGGPLV.

Additionally, the coding region is found at about 3128579-3130159 bp of the B. subtilis 168 chromosome.

The prpC coding sequence of the prpC protein (protein phosphatase) of B. subtilis 168 is shown below:

(SEQ ID NO: 29)

TTGTTAACAGCCTTAAAAACAGATACAGGAAAAATCCGCCAGCATAATGA
AGATGATGCGGGGATATTCAAGGGGAAAGATGAATTTATATTAGCGGTTG
TCGCTGATGGCATGGGCGGCCATCTTGCTGGAGATGTTGCGAGCAAGATG
GCTGTGAAAGCCATGGGGGAGAAATGGAATGAAGCAGAGACGATTCCAAC
TGCGCCCTCGGAATGTGAAAAATGGCTCATTGAACAGATTCTATCGGTAA
ACAGCAAAATATACGATCACGCTCAAGCCCACGAAGAATGCCAAGGCATG
GGGACGACGATTGTATGTGCACTTTTTACGGGGAAAACGGTTTCTGTTGC
CCATATCGGAGACAGCAGATGCTATTTGCTTCAGGACGATGATTTCGTTC
AAGTGACAGAAGACCATTCGCTTGTAAATGAACTGGTTCGCACTGGAGAG
ATTTCCAGAGAAGACGCTGAACATCATCCGCGAAAAAATGTGTTGACGAA
GGCGCTTGGAACAGACCAGTTAGTCAGTATTGACACCCGTTCCTTTGATA
TAGAACCCGGAGACAAACTGCTTCTATGTTCTGACGGACTGACAAATAAA
GTGGAAGGCACTGAGTTAAAAGACATCCTGCAAAGCGATTCAGCTCCTCA
GGAAAAAGTAAACCTGCTTGTGGACAAAGCCAATCAGAATGGCGGAGAAG
ACAACATTACAGCAGTTTTGCTTGAGCTTGCTTTACAAGTTGAAGAGGGT
GAAGATCAGTGC.

The deduced amino acid sequence of the prpC protein is:

(SEQ ID NO:30)

MLTALKTDTGKIRQHNEDDAGIFKGKDEFILAVVADGMGGHLAGDVASKM
AVKAMGEKWNEAETIPTAPSECEKWLIEQILSVNSKIYDHAQAHEECQGM
GTTIVCALFTGKTVSVAHIGDSRCYLLQDDDFVQVTEDHSLVNELVRTGE
ISREDAEHHPRKNVLTKALGTDQLVSIDTRSFDIEPGDKLLLCSDGLTNK
VEGTELKDILQSDSAPQEKVNLLVDKANQNGGEDNITAVLLELALQVEEG
EDQC.

Additionally, the coding region is found at about 1649684-1650445 bp of the B. subtilis 168 chromosome.

The rocA coding sequence of the rocA protein (pyrroline-5 carboxylate dehydrogenase) of B. subtilis 168 is shown below:

(SEQ ID NO:31)

ATGACAGTCACATACGCGCACGAACCATTTACCGATTTTACGGAAGCAAA
GAATAAAACTGCATTTGGGGAGTCATTGGCCTTTGTAAACACTCAGCTCG
GCAAGCATTATCCGCTTGTCATAAATGGAGAAAAAATTGAAACGGACCGC
AAAATCATTTCTATTAACCCGGCAAATAAAGAAGAGATCATTGGGTACGC
GTCTACAGCGGATCAAGAGCTTGCTGAAAAAGCGATGCAAGCCGCATTGC
AGGCATTTGATTCCTGGAAAAAACAAAGACCGGAGCACCGCGCAAATATT
CTCTTTAAGGCAGCGGCTATTTTGCGCAGAAGAAAGCATGAATTTTCAAG
CTATCTTGTGAAGGAAGCAGGAAAACCGTGGAAGGAAGCAGATGCGGACA
CGGCTGAAGCGATAGACTTTTTAGAGTTCTACGCGCGCCAAATGTTAAAG
CTCAAGGAAGGGGCTCCGGTGAAGAGCCGTGCTGGCGAGGTCAATCAATA
TCATTACGAAGCGCTTGGCGTCGGCATCGTCATTTCTCCATTTAACTTCC
CGCTCGCGATTATGGCGGGAACAGCGGTGGCAGCGATTGTGACAGGAAAT
ACGATTCTCTTAAAACCGGCTGACGCAGCCCCGGTAGTGGCAGCAAAATT
TGTCGAGGTCATGGAGGAAGCGGGTCTGCCAAACGGCGTTCTGAATTACA
TTCCGGGAGATGGTGCGGAGATCGGTGATTTCTTAGTTGAGCATCCGAAG
ACACGGTTTGTCTCATTTACAGGTTCCCGTGCAGTCGGCTGCCGGATTTA
TGAGCGAGCTGCCAAAGTGCAGCCGGGCCAAAAATGGCTCAAACGGGTAA
TTGCAGAAATGGGCGGAAAAGACACAGTGCTTGTCGACAAGGACGCTGAT
CTTGACCTTGCTGCATCCTCTATCGTGTATTCAGCATTTGGATATTCAGG
ACAGAAGTGTTCTGCGGGCTCCCGCGCGGTCATTCATCAGGATGTGTATG
ATGAAGTGGTGGAAAAAGCTGTGGCGCTGACCAAAACGCTGACTGTCGGC
AATCCAGAAGATCCTGATACGTATATGGGTCCCGTGATTCATGAAGCATC
CTACAACAAAGTGATGAAATACATTGAAATCGGCAAATCTGAAGGCAAGC
TATTGGCCGGCGGAGAAGGCGATGATTCAAAAGGCTACTTTATTCAGCCG
ACGATCTTTGCAGATGTTGATGAAAACGCCCGCTTGATGCAGGAAGAAAT
TTTCGGCCCGGTTGTTGCGATTTGCAAAGCGCGTGATTTCGATCATATGC
TGGAGATTGCCAATAACACGGAATACGGATTAACAGGTGCGCTTCTGACG
AAAAACCGTGCGCACATTGAACGGGCGCGCGAGGATTTCCATGTCGGAAA
CCTATATTTTAACAGAGGATGTACCGGAGCAATTGTCGGCTATCAGCCGT
TCGGCGGTTTTAATATGTCAGGAACAGACTCAAAAGCAGGCGGTCCCGAT
TACTTAATTCTTCATATGCAAGCCAAAACAACGTCCGAAGCTTTT.

The deduced amino acid sequence of the RocA protein is:

(SEQ ID NO:32)

MTVTYAHEPFTDFTEAKNKTAFGESLAFVNTQLGKHYPLVINGEKIETDR
KIISINPANKEEIIGYASTADQELAEKAMQAALQAFDSWKKQRPEHRANI
LFKAAAILRRRKHEFSSYLVKEAGKPWKEADADTAEAIDFLEFYARQMLK
LKEGAPVKSRAGEVNQYHYEALGVGIVISPFNFPLAIMAGTAVAAIVTGN
TILLKPADAAPVVAAKFVEVMEEAGLPNGVLNYIPGDGAEIGDFLVEHPK
TRFVSFTGSRAVGCRIYERAAKVQPGQKWLKRVIAEMGGKDTVLVDKDAD
LDLAASSIVYSAFGYSGQKCSAGSRAVIHQDVYDEVVEKAVALTKTLTVG
NPEDPDTYMGPVIHEASYNKVMKYIEIGKSEGKLLAGGEGDDSKGYFIQP
TIFADVDENARLMQEEIFGPVVAICKARDFDHMLEIANNTEYGLTGALLT
KNRAHIERAREDFHVGNLYFNRGCTGAIVGYQPFGGFNMSGTDSKAGGPD
YLILHMQAKTTSEAF.

Additionally, the coding region is found at about 3877991-3879535 bp of the B. subtilis 168 chromosome.

The rocD coding sequence of the rocD protein (ornithine aminotransferase) of B. subtilis 168 is shown below:

(SEQ ID NO:33)

ATGACAGCTTTATCTAAATCCAAAGAAATTATTGATCAGACGTCTCATTA
CGGAGCCAACAATTATCACCCGCTCCCGATTGTTATTTCTGAAGCGCTGG
GTGCTTGGGTAAAGGACCCGGAAGGCAATGAATATATGGATATGCTGAGT
GCTTACTCTGCGGTAAACCAGGGGCACAGACACCCGAAAATCATTCAGGC
ATTAAAGGATCAGGCTGATAAAATCACCCTCACGTCACGCGCGTTTCATA
ACGATCAGCTTGGGCCGTTTTACGAAAAAACAGCTAAACTGACAGGCAAA
GAGATGATTCTGCCGATGAATACAGGAGCCGAAGCGGTTGAATCCGCGGT
GAAAGCGGCGAGACGCTGGGCGTATGAAGTGAAGGGCGTAGCTGACAATC
AAGCGGAAATTATCGCATGTGTCGGGAACTTCCACGGCCGCACGATGCTG
GCGGTATCTCTTTCTTCTGAAGAGGAATATAAACGAGGATTCGGCCCGAT
GCTTCCAGGAATCAAACTCATTCCTTACGGCGATGTGGAAGCGCTTCGAC
AGGCCATTACGCCGAATACAGCGGCATTCTTGTTTGAACCGATTCAAGGC
GAAGCGGGCATTGTGATTCCGCCTGAAGGATTTTTACAGGAAGCGGCGGC
GATTTGTAAGGAAGAGAATGTCTTGTTTATTGCGGATGAAATTCAGACGG
GTCTCGGACGTACAGGCAAGACGTTTGCCTGTGACTGGGACGGCATTGTT
CCGGATATGTATATCTTGGGCAAAGCGCTTGGCGGCGGTGTGTTCCCGAT
CTCTTGCATTGCGGCGGACCGCGAGATCCTAGGCGTGTTTAACCCTGGCT
CACACGGCTCAACATTTGGTGGAAACCCGCTTGCATGTGCAGTGTCTATC
GCTTCATTAGAAGTGCTGGAGGATGAAAAGCTGGCGGATCGTTCTCTTGA
ACTTGGTGAATACTTTAAAAGCGAGCTTGAGAGTATTGACAGCCCTGTCA
TTAAAGAAGTCCGCGGCAGAGGGCTGTTTATCGGTGTGGAATTGACTGAA
GCGGCACGTCCGTATTGTGAGCGTTTGAAGGAAGAGGGACTTTTATGCAA
GGAAACGCATGATACAGTCATTCGTTTTGCACCGCCATTAATCATTTCCA
AAGAGGACTTGGATTGGGCGATAGAGAAAATTAAGCACGTGCTGCGAAAC
GCA.

The deduced amino acid sequence of the RocD protein is:

(SEQ ID NO:34)

MTALSKSKEIIDQTSHYGANNYHPLPIVISEALGAWVKDPEGNEYMDMLS
AYSAVNQGHRHPKIIQALKDQADKITLTSRAFHNDQLGPFYEKTAKLTGK
EMILPMNTGAEAVESAVKAARRWAYEVKGVADNQAEIIACVGNFHGRTML
AVSLSSEEEYKRGFGPMLPGIKLIPYGDVEALRQAITPNTAAFLFEPIQG
EAGIVIPPEGFLQEAAAICKNEENVLFIADEIQTGLGRTGKTFACDWDGI
VPDMYILGKALGGGVFPISCIAADREILGVFNPGSHGSTFGGNPLACAVS
IASLEVLEDEKLADRSLELGEYFKSELESIDSPVIKEVRGRGLFIGVELT
EAARPYCERLKEEGLLCKETHDTVIRFAPPLIISKEDLDWAIEKIKHVLR
NA.

Additionally, the coding region is found at about 4143328-4144530 bp of the B. subtilis 168 chromosome.

The rocF coding sequence of the rocF protein (arginase) of B. subtilis 168 is shown below:

(SEQ ID NO:35)

ATGGATAAAACGATTTCGGTTATTGGAATGCCAATGGATTTAGGACAAGC
ACGACGCGGAGTGGATATGGGCCCGAGTGCCATCCGGTACGCTCATCTGA
TCGAGAGGCTGTCAGACATGGGGTATACGGTTGAAGATCTCGGTGACATT
CCGATCAATCGCGAAAAAATCAAAAATGACGAGGAACTGAAAAACCTGAA
TTCCGTTTTGGCGGGAAATGAAAAACTCGCGCAAAAGGTCAACAAAGTCA
TTGAAGAGAAAAAATTCCCGCTTGTCCTGGGCGGTGACCACAGTATTGCG
ATCGGCACGCTTGCAGGCACAGCGAAGCATTACGATAATCTCGGCGTCAT
CTGGTATGACGCGCACGGCGATTTGAATACACTTGAAACTTCACCATCGG
GCAATATTCACGGCATGCCGCTCGCGGTCAGCCTAGGCATTGGCCACGAG
TCACTGGTTAACCTTGAAGGCTACGCGCCTAAAATCAAACCGGAAAACGT
CGTCATCATTGGCGCCCGGTCACTTGATGAAGGGGAGCGCAAGTACATTA
AGGAAAGCGGCATGAAGGTGTACACAATGCACGAAATCGATCGTCTTGGC
ATGACAAAGGTCATTGAAGAAACCCTTGATTATTTATCAGCATGTGATGG
CGTCCATCTGAGCCTTGATCTGGACGGACTTGATCCGAACGACGCACCGG
GTGTCGGAACCCCTGTCGTCGGCGGCATCAGCTACCGGGAGAGCCATTTG
GCTATGGAAATGCTGTATGACGCAGGCATCATTACCTCAGCCGAATTCGT
TGAGGTTAACCCGATCCTTGATCACAAAAACAAAACGGGCAAAACAGCAG
TAGAGCTCGTAGAATCCCTGTTAGGGAAGAAGCTGCTG.

The deduced amino acid sequence of the RocF protein:

(SEQ ID NO:36)

MDKTISVIGMPMDLGQARRGVDMGPSAIRYAHLIERLSDMGYTVEDLGDI
PINREKIKNDEELKNLNSVLAGNEKLAQKVNKVIEEKKFPLVLGGDHSIA
IGTLAGTAKHYDNLGVIWYDAHGDLNTLETSPSGNIHGMPLAVSLGIGHE
SLVNLEGYAPKIKPENVVIIGARSLDEGERKYIKESGMKVYTMHEIDRLG
MTKVIEETLDYLSACDGVHLSLDLDGLDPNDAPGVGTPVVGGISYRESHL
AMEMLYDAGIITSAEFVEVNPILDHKNKTGKTAVELVESLLGKKLL.

Additionally, the coding region is found at about 4140738-4141625 bp of the B. subtilis 168 chromosome.

The Tdh coding sequence of the Tdh protein (threonine 3-dehydrogenase) of B. subtilis 168 is shown below:

(SEQ ID NO:37)

ATGCAGAGTGGAAAGATGAAAGCTCTAATGAAAAAGGACGGGGCGTTCGG
TGCTGTGCTGACTGAAGTTCCCATTCCTGAGATTGATAAACATGAAGTCC
TCATAAAAGTGAAAGCCGCTTCCATATGCGGCACGGATGTCCACATTTAT
AATTGGGATCAATGGGCACGTCAGAGAATCAAAACACCCTATGTTTTCGG
CCATGAGTTCAGCGGCATCGTAGAGGGCGTGGGAGAGAATGTCAGCAGTG
TAAAAGTGGGAGAGTATGTGTCTGCGGAAACACACATTGTCTGTGGTGAA
TGTGTCCCTTGCCTAACAGGAAAATCTCATGTGTGTACCAATACTGCTAT
AATCGGAGTGGACACGGCAGGCTGTTTTGCGGAGTATGTAAAAGTTCCAG
CTGATAACATTTGGAGAAATCCCGCTGATATGGACCCGTCGATTGCTTCC
ATTCAAGAGCCTTTAGGAAATGCAGTTCATACCGTACTCGAGAGCCAGCC
TGCAGGAGGAACGACTGCAGTCATTGGATGCGGACCGATTGGTCTTATGG
CTGTTGCGGTTGCAAAAGCAGCAGGAGCTTCTCAGGTGATAGCGATTGAT
AAGAATGAATACAGGCTGAGGCTTGCAAAACAAATGGGAGCGACTTGTAC
TGTTTCTATTGAAAAAGAAGACCCGCTCAAAATTGTAAGCGCTTTAACGA
GTGGAGAAGGAGCAGATCTTGTTTGTGAGATGTCGGGCCATCCCTCAGCG
ATTGCCCAAGGTCTTGCGATGGCTGCGAATGGCGGAAGATTTCATATTCT
CAGCTTGCCGGAACATCCGGTGACAATTGATTTGACGAATAAAGTGGTAT
TTAAAGGGCTTACCATCCAAGGAATCACAGGAAGAAAAATGTTTTCAACA
TGGCGCCAGGTGTCTCAGTTGATCAGTTCAAACATGATCGATCTTGCACC
TGTTATTACCCATCAGTTTCCATTAGAGGAGTTTGAAAAAGGTTTCGAAC
TGATGAGAAGCGGGCAGTGCGGAAAAGTAATTTTAATTCCA.

The deduced amino acid sequence of the Tdh protein is:

(SEQ ID NO:38)

MQSGKMKALMKKDGAFGAVLTEVPIPEIDKHEVLIKVKAASICGTDVHIY
NWDQWARQRIKTPYVFGHEFSGIVEGVGENVSSVKVGEYVSAETHIVCGE
CVPCLTGKSHVCTNTAIIGVDTAGCFAEYVKVPADNIWRNPADMDPSIAS
IQEPLGNAVHTVLESQPAGGTTAVIGCGPIGLMAVAVAKAAGASQVIAID
KNEYRLRLAKQMGATCTVSIEKEDPLKIVSALTSGEGADLVCEMSGHPSA
IAQGLAMAANGGRFHILSLPEHPVTIDLTNKVVFKGLTIQGITGRKMFST
WRQVSQLISSNMIDLAPVITHQFPLEEFEKGFELMRSGQCGKVILIP.

Additionally, the coding region is found at about 1769731-1770771 bp of the B. subtilis 168 chromosome.

The coding sequences for the tryptophan operon regulatory region and genes trpE (SEQ ID NO:48), trpD (SEQ ID NO:46), trpC (SEQ ID NO:44), trpF (SEQ ID NO:50), trpB (SEQ ID NO:42), and trpA (SEQ ID NO:40) are shown below. The operon regulatory region is underlined. The trpE start (ATG) is shown in bold, followed as Well by the trpD, trpC trpF, trpB, and trpA starts (also indicated in bold, in the order shown).

(SEQ ID NO:39)

TAATACGATAAGAACAGCTTAGAAATACACAAGAGTGTGTATAAAGCAAT
TAGAATGAGTTGAGTTAGAGAATAGGGTAGCAGAGAATGAGTTTAGTTGA
GCTGAGACATTATGTTTATTCTACCCAAAAGAAGTCTTTCTTTTGGGTTT
ATTTGTTATATAGTATTTTATCCTCTCATGCCATCTTCTCATTCTCCTTG
CCATAAGGAGTGAGAGCAATGAATTTCCAATCAAACATTTCCGCATTTTT
AGAGGACAGCTTGTCCCACCACACGATACCGATTGTGGAGACCTTCACAG
TCGATACACTGACACCCATTCAAATGATAGAGAAGCTTGACAGGGAGATT
ACGTATCTTCTTGAAAGCAAGGACGATACATCCACTTGGTCCAGATATTC
GTTTATCGGCCTGAATCCATTTCTCACAATTAAAGAAGAGCAGGGCCGTT
TTTCGGCCGCTGATCAGGACAGCAAATCTCTTTACACAGGAAATGAACTA
AAAGAAGTGCTGAACTGGATGAATACCACATACAAAATCAAAACACCTGA
GCTTGGCATTCCTTTTGTCGGCGGAGCTGTCGGGTACTTAAGCTATGATA
TGATCCCGCTGATTGAGCCTTCTGTTCCTTCGCATACCAAAGAAACAGAC
ATGGAAAAGTGTATGCTGTTTGTTTGCCGGACATTAATTGCGTATGATCA
TGAAACCAAAAACGTCCACTTTATCCAATATGCAAGGCTCACTGGAGAGG
AAACAAAAAACGAAAAAATGGATGTATTCCATCAAAATCATCTGGAGCTT
CAAAATCTCATTGAAAAAATGATGGACCAAAAAAACATAAAAGAGCTGTT
TCTTTCTGCTGATTCATACAAGACACCCAGCTTTGAGACAGTATCTTCTA
ATTATGAAAAATCGGCTTTTATGGCTGATGTAGAAAAAATCAAAAGCTAT
ATAAAAGCAGGCGATATCTTCCAGGGTGTTTTATCACAAAAATTTGAGGT
GCCGATAAAAGCAGATGCTTTTGAGTTATACCGAGTGCTTAGGATCGTCA
ATCCTTCGCCGTATATGTATTATATGAAACTGCTAGACAGAGAAATAGTC
GGCAGCTCTCCGGAACGGTTAATACACGTTCAAGACGGGCACTTAGAAAT
CCATCCGATTGCCGGTACGAGAAAACGCGGTGCAGACAAAGCTGAAGATG
AGAGACTGAAGGTTGAGCTCATGAAGGATGAAAAAGAAAAAGCGGAGCAT
TACATGCTCGTTGATCTTGCCCGAAACGATATCGGCAGAGTAGCAGAGTA
TGGTTCTGTTTCTGTGCCGGAGTTCACAAAAATTGTTTCCTTTTCACATG
TCATGCACATTATCTCGGTGG1TACAGGCCGATTGAAAAAAGGGGTTCAT
CCTGTCGATGCACTGATGTCTGCTTTCCCGGCGGGGACTTTAACAGGCGC
ACCCAAAATCCGTGCCATGCAGCTTTTGCAAGAACTCGAGCCAACACCGA
GAGAGACATACGGAGGGTGTATTGCCTACATTGGGTTTGACGGGAATATC
GACTCTTGTATTACGATTCGCACGATGAGTGTAAAGAACGGTGTTGCATC
GATACAGGCAGGTGCTGGCATTGTTGCTGATTCTGTTCCGGAAGCCGAAT
ACGAAGAAAGCTGTAATAAAGCCGGTGCGCTGCTGAAAACGATTCATATT
GCAGAAGACATGTTTCATAGCAAGGAGGATAAAGCTGATGAACAGATTTC
TACAATTGTGCGTTGACGGAAAAACCCTTACTGCCGGTGAGGCTGAAACG
CTGATGAATATGATGATGGCAGCGGAAATGACTCCTTCTGAAATGGGGGG
GATATTGTCAATTCTTGCTCATCGGGGGGAGACGCCAGAAGAGCTTGCGG
GTTTTGTGAAGGCAATGCGGGCACACGCTCTTACAGTCGATGGACTTCCT
GATATTGTTGATAAAATGCGGAACAGGGGGAGACGGTATTTCCACTTTTA
ATATCTCAACGGCCTCGGCAATTGTTGCCTCGGCAGCTGGTGCGAAAATC
GCTAAGCATGGCAATCGCTCTGTCTCTTCTAAAAGCGGAAGCGCTGATGT
TTTAGAGGAGCTAGAGGTTTCTATTCAAACCACTCCCGAAAAGGTCAAAA
GCAGCATTGAAACAAACAACATGGGATTTCTTTTTGCGCCGCTTTACCAT
TCGTCTATGAAACATGTAGCAGGTACTAGAAAAGAGCTAGGTTTCAGAAC
GGTATTTAATCTGCTTGGGCCGCTCAGCAATCCTTTACAGGCGAAGCGTC
AGGTGATTGGGGTCTATTCTGTTGAAAAAGCTGGACTGATGGCAAGCGCA
CTGGAGACGTTTCAGCCGAAGCACGTTATGTTTGTATCAAGCCGTGACGG
TTTAGATGAGCTTTCAATTACAGCACCGACCGACGTGATTGAATTAAAGG
ACGGAGAGCGCCGGGAGTATACCGTTTCACCCGAAGATTTCGGTTTCACA
AATGGCAGACTTGAAGATTTACAGGTGCAGTCTCCGAAAGAGAGCGCTTA
TCTCATTCAGAATATTTTTGAAAATAAAAGCAGCAGTTCCGCTTTATCTA
TTACGGCTTTTAATGCGGGTGCTGCGATTTACACGGCGGGAATTACCGCC
TCACTGAAGGAAGGAACGGAGCTGGCGTTAGAGACGATTACAAGCGGAGG
CGCTGCCGCGCAGCTTGAACGACTAAAGCAGAAAGAGGAAGAGATCTATG
CTTGAAAAAATCATCAAACAAAAGAAAGAAGAAGTGAAAACACTGGTTCT
GCCGGTAGAGCAGCCTTTCGAGAAACGTTCATTTAAGGAGGCGCCGGCAA
GCCCGAATCGGTTTATCGGGTTGATTGCCGAAGTGAAGAAAGCATCGCCG
TCAAAAGGGCTTATTAAAGAGGATTTTGTACCTGTGCAGATTGCAAAAGA
CTATGAGGCTGCGAAGGCAGATGCGATTTCCGTTTTAACAGACACCCCGT
TTTTTCAAGGGGAAAACAGCTATTTATCAGACGTAAAGCGTGCTGTTTCG
ATTCCTGTACTTAGAAAAGATTTTATTATTGATTCTCTTCAAGTAGAGGA
ATCAAGAAGAATCGGAGCGGATGCCATATTGTTAATCGGCGAGGTGCTTG
ATCCCTTACACCTTCATGAATTATATCTTGAAGCAGGTGAAAAGGGGATG
GACGTGTTAGTGGAGGTTCATGATGCATCAACGCTAGAACAAATATTGAA
AGTGTTCACACCCGACATTCTCGGCGTAAATAATCGAAACCTAAAAACGT
TTGAAACATCTGTAAAGCAGACAGAACAAATCGCATCTCTCGTTCCGAAA
GAATCCTTGCTTGTCAGCGAAAGCGGAATCGGTTCTTTAGAACATTTAAC
ATTTGTCAATGAACATGGGGCGCGAGCTGTACTTATCGGTGAATCATTGA
TGAGACAAACTTCTCAGCGTAAAGCAATCCATGCTTTGTTTAGGGAGTGA
GGTTGTGAAGAAACCGGCATTAAAATATTGCGGTATTCGGTCACTAAAGG
ATTTGCAGCTTGCGGCGGAATCACAGGCTGATTACCTAGGATTTATTTTT
GCTGAAAGCAAACGAAAAGTATCTCCGGAAGATGTGAAAAAATGGCTGAA
CCAAGTTCGTGTCGAAAAACAGGTTGCAGGTGTTTTTGTTAATGAATCAA
TAGAGACGATGTCACGTATTGCCAAGAGCTTGAAGCTCGACGTCATTCAG
CTTCACGGTGATGAAAAACCGGCGGATGTCGCTGCTCTTCGCAAGCTGAC
AGGCTGTGAAATATGGAAGGCGCTTCACCATCAAGATAACACAACTCAAG
AAATAGCCCGCTTTAAAGATAATGTTGACGGCTTTGTGATTGATTCATCT
GTAAAAGGGTCTAGAGGCGGAACTGGTGTTGCATTTTCTTGGGACTGTGT
GCCGGAATATCAGCAGGCGGCTATTGGTAAACGCTGCTTTATCGCTGGCG
GCGTGAATCCGGATAGCATCACACGCCTATTGAAATGGCAGCCAGAAGGA
ATTGACCTTGCCAGCGGAATTGAAAAAAACGGACAAAAAGATCAGAATCT
GATGAGGCTTTTAGAAGAAAGGATGAACCGATATGTATCCATATCCGAAT
GAAATAGGCAGATACGGTGATTTTGGCGGAAAGTTTGTTCCGGAAACACT
CATGCAGCCGTTAGATGAAATACAAACAGCATTTAAACAAATCAAGGATG
ATCCCGCTTTTCGTGAAGAGTATTATAAGCTGTTAAAGGACTATTCCGGA
CGCCCGACTGCATTAACATACGCTGATCGAGTCACTGAATACTTAGGCGG
CGCGAAAATCTATTTGAAACGAGAAGATTTAAACCATACAGGTTCTCATA
AAATCAATAATGCGCTAGGTCAAGCGCTGCTTGCTAAAAAAATGGGCAAA
ACGAAAATCATTGCTGAAACCGGTGCCGGCCAGCATGGTGTTGCCGCTGC
AACAGTTGCAGCCAAATTCGGCTTTTCCTGTACTGTGTTTATGGGTGAAG
AGGATGTTGCCCGCCAGTCTCTGAACGTTTTCCGCATGAAGCTTCTTGGA
GCGGAGGTAGTGCCTGTAACAAGCGGAAACGGAACATTGAAGGATGCCAC
AAATGAGGCGATCCGGTACTGGGTTCAGCATTGTGAGGATCACTTTTATA
TGATTGGATCAGTTGTCGGCCCGCATCCTTATCCGCAAGTGGTCCGTGAA
TTTCAAAAAATGATCGGAGAGGAAGCGAAGGATCAGTTGAAACGTATTGA
AGGCACTATGCCTGATAAAGTAGTGGCATGTGTAGGCGGAGGAAGCAATG
CGATGGGTATGTTTCAGGCATTTTTAAATGAAGATGTTGAACTGATCGGC
GCTGAAGCAGCAGGAAAAGGAATTGATACACCTCTTCATGCCGCCACTAT
TTCGAAAGGAACCGTAGGGGTTATTCACGGTTCATTGACTTATCTCATTC
AGGATGAGTTCGGGCAAATTATTGAGCCCTACTCTATTTCAGCCGGTCTC
GACTATCCTGGAATCGGTCCGGAGCATGCATATTTGCATAAAAGCGGCCG
TGTCACTTATGACAGTATAACCGATGAAGAAGCGGTGGATGCATTAAAGC
TTTTGTCAGAAAAAGAGGGGATTTTGCCGGCAATCGAATCTGCCCATGCG
TTAGCGAAAGCATTCAAACTCGCCAAAGGAATGGATCGCGGTCAACTCAT
TCTCGTCTGTTTATCAGGCCGGGGAGACAAGGATGTCAACACATTAATGA
ATGTATTGGAAGAAGAGGTGAAAGCCCATGTTTAAATTGGATCTTCAACC
ATCAGAAAAATTGTTTATCCCGTTTATTACGGCGGGCGATCCAGTTCCTG
AGGTTTCGATTGAACTGGCGAAGTCACTCCAAAAAGCAGGCGCCACAGCA
TTGGAGCTTGGTGTTGCATACTCTGACCCGCTTGCAGACGGTCCGGTGAT
CCAGCGGGCTTCAAAGCGGGCGCTTGATCAAGGAATGAATATCGTAAAGG
CAATCGAATTAGGCGGAGAAATGAAAAAAAACGGAGTGAATATTCCGATT
ATCCTCTTTACGTATTATAATCCTGTGTTACAATTGAACAAAGAATACTT
TTTCGCTTTACTGCGGGAAAATCATATTGACGGTCTGCTTGTTCCGGATC
TGCCATTAGAAGAAAGCAACAGCCTTCAAGAGGAATGTAAAAGCCATGAG
GTGACGTATATTTCTTTAGTTGCGCCGACAAGCGAAAGCCGTTTGAAAAC
CATTATTGAACAAGCCGAGGGGTTCGTCTACTGTGTATCTTCTCTGGGTG
TGACCGGTGTCCGCAATGAGTTCAATTCATCCGTGTACCCGTTCATTCGT
ACTGTGAAGAATCTCAGCACTGTTCCGGTTGCTGTAGGGTTCGGTATATC
AAACCGTGAACAGGTCATAAAGATGAATGAAATTAGTGACGGTGTCGTAG
TGGGAAGTGCGCTCGTCAGAAAAATAGAAGAATTAAAGGACCGGCTCATC
AGCGCTGAAACGAGAAATCAGGCGCTGCAGGAGTTTGAGGATTATGCAAT
GGCGTTTAGCGGCTTGTACAGTTTAAAA.

The deduced TrpA protein (tryptophan synthase (alpha subunit)) sequence is:

(SEQ ID NO:41)

MFKLDLQPSEKLFIPFITAGDPVPEVSIELAKSLQKAGATALELGVAYSD
PLADGPVIQRASKRALDQGMNIVKAIELGGEMKKNGVNIPIILFTYYNPV
LQLNKEYFFALLRENHIDGLLVPDLPLEESNSLQEECKSHEVTYISLVAP
TSESRLKTIIEQAEGFVYCVSSLGVTGVRNEFNSSVYPFIRTVKNLSTVP
VAVGFGISNREQVIKMNEISDGVVVGSALVRKIEELKDRLISAETRNQAL
QEFEDYAMAFSGLYSLK.

The deduced TrpB protein (tryptophan synthase (beta subunit)) sequence is:

(SEQ ID NO:43)

MYPYPNEIGRYGDFGGKFVPETLMQPLDEIQTAFKQIKDDPAFREEYYKL
LKDYSGRPTALTYADRVTEYLGGAKIYLKREDLNHTGSHKINNALGQALL
AKKMGKTKIIAETGAGQHGVAAATVAAKFGFSCTVFMGEEDVARQSLNVF
RMKLLGAEVVPVTSGNGTLKDATNEAIRYWVQHCEDHFYMIGSVVGPHPY
PQVVREFQKMIGEEAKDQLKRIEGTMPDKVVACVGGGSNAMGMFQAFLNE
DVELIGAEAAGKGIDTPLHAATISKGTVGVIHGSLTYLIQDEFGQIIEPY
SISAGLDYPGIGPEHAYLHKSGRVTYDSITDEEAVDALKLLSEKEGILPA
IESAHALAKAFKLAKGMDRGQLILVCLSGRGDKDVNTLMNVLEEEVKAH
V.

The deduced TrpC protein indol-3-glycerol phosphate synthase) sequence is:

(SEQ ID NO:45)

MLEKIIKQKKEEVKTLVLPVEQPFEKRSFKEAPASPNRFIGLIAEVKKAS
PSKGLIKEDFVPVQIAKDYEAAKADAISVLTDTPFFQGENSYLSDVKRAV
SIPVLRKDFIIDSLQVEESRRIGADAILLIGEVLDPLHLHELYLEAGEKG
MDVLVEVHDASTLEQILKVFTPDILGVNNRNLKTFETSVKQTEQIASLVP
KESLLVSESGIGSLEHLTFVNEHGARAVLIGESLMRQTSQRKAIHALFR
E.

The deduced TrpD protein (anthranilate phosphoribosyltransferase) sequence is:

(SEQ ID NO:47)

MNRFLQLCVDGKTLTAGEAETLMNMMMAAEMTPSEMGGILSILAHRGETP
EELAGFKAMRAHALTVDGLPDIVDTCGTGGDGISTFNISTASAIVASAAG
AKIAKHGNRSVSSKSGSADVLEELEVSIQTTPEKVKSSIETNNMGFLFAP
LYHSSMKHVAGTRKELGFRTVFNLLGPLSNPLQAKRQVIGVYSVEKAGLM
ASALETFQPKHVMFVSSRDGLDELSITAPTDVIELKDGERREYTVSPEDF
GFTNGRLEDLQVQSPKESAYLIQNIFENKSSSSALSITAFNAGAAIYTAG
ITASLKEGTELALETITSGGAAAQLERLKQKEEEIYA.

The deduced TrpE protein (anthranilate synthase) sequence is:

(SEQ ID NO:49)

MNFQSNISAFLEDSLSHHTIPIVETFTVDTLTPIQMIEKLDREITYLLES
KDDTSTWSRYSFIGLNPFLTIKEEQGRFSAADQDSKSLYTGNELKEVLNW
MNTTYKIKTPELGIPFVGGAVGYLSYDMIPLIEPSVPSHTKETDMEKCML
FVCRTLIAYDHETKNVHFIQYARLTGEETKNEKMDVFHQNHLELQNLIEK
MMDQKNIKELFLSADSYKTPSFETVSSNYEKSAFMADVEKIKSYIKAGDI
FQGVLSQKFEVPIKADAFELYRVLRIVNPSPYMYYMKLLDREIVGSSPER
LIHVQDGHLEIHPIAGTRKRGADKAEDERLKVELMKDEKEKAEHYMLVDL
ARNDIGRVAEYGSVSVPEFTKIVSFSHVMHIISVVTGRLKKGVHPVDALM
SAFPAGTLTGAPKIRAMQLLQELEPTPRETYGGCIAYIGFDGNIDSCITI
RTMSVKNGVASIQAGAGIVADSVPEAEYEESCNKAGALLKTIHIAEDMFH
SKEDKADEQISTIVR.

The deduced TrpF protein (phosphoribosyl anthranilate isomerase) sequence is:

(SEQ ID NO:51)

MKKPALKYCGIRSLKDLQLAAESQADYLGFIFAESKRKVSPEDVKKWLNQ
VRVEKQVAGVFVNESIETMSRIAKSLKLDVIQLHGDEKPADVAALRKLTG
CEIWKALHHQDNTTQEIARFKDNVDGFVIDSSVKGSRGGTGVAFSWDCVP
EYQQAAIGKRCFIAGGVNPDSITRLLKWQPEGIDLASGIEKNGQKDQNLM
RLLEERMNRYVSISE.

Additionally, the coding region is found at about 2370707 bp to 2376834 bp (first bp=2376834; last bp=2370707) bp of the B. subtilis 168 chromosome.

The ycgM coding sequence of the ycgM protein (similar to proline oxidase) of B. subtilis 168 is shown below:

(SEQ ID NO:52)

GTGATCACAAGAGATTTTTTCTTATTTTTATCCAAAAGCGGCTTTCTCAA
TAAAATGGCGAGGAACTGGGGAAGTCGGGTAGCAGCGGGTAAAATTATCG
GCGGGAATGACTTTAACAGTTCAATCCCGACCATTCGACAGCTTAACAGC
CAAGGCTTGTCAGTTACTGTCGATCATTTAGGCGAGTTTGTGAACAGCGC
CGAGGTCGCACGGGAGCGTACGGAAGAGTGCATTCAAACCATTGCGACCA
TCGCGGATCAGGAGCTGAACTCACACGTTTCTTTAAAAATGACGTCTTTA
GGTTTGGATATAGATATGGATTTGGTGTATGAAAATATGACAAAAATCCT
TCAGACGGCCGAGAAACATAAAATCATGGTCACCATTGACATGGAGGACG
AAGTCAGATGCCAGAAAACGCTTGATATTTTCAAAGATTTCAGAAAGAAA
TACGAGCATGTGAGCACAGTGCTGCAAGCCTATCTGTACCGGACGGAAAA
AGACATTGACGATTTGGATTCTTTAAACCCGTTCCTTCGCCTTGTAAAAG
GAGCTTATAAAGAATCAGAAAAAGTAGCTTTCCCGGAGAAAAGCGATGTC
GATGAAAATTACAAAAAAATCATCCGAAAGCAGCTCTTAAACGGTCACTA
TACAGCGATTGCCACACATGACGACAAAATGATCGACTTTACAAAGCAGC
TTGCCAAGGAACATGGCATTGCCAATGACAAGTTTGAATTTCAGATGCTG
TACGGCATGCGGTCGCAAACCCAGCTCAGCCTCGTAAAAGAAGGTTATAA
CATGAGAGTCTACCTGCCATACGGCGAGGATTGGTACGGCTACTTTATGA
GACGCCTTGCAGAACGTCCGTCAAACATTGCATTTGCTTTCAAAGGAATG
ACAAAGAAG.

The deduced amino acid sequence of the YcgM protein is:

(SEQ ID NO:53)

MITRDFFLFLSKSGFLNKMARNWGSRVAAGKIIGGNDFNSSIPTIRQLNS
QGLSVTVDHLGEFVNSAEVARERTEECIQTIATIADQELNSHVSLKMTSL
GLDIDMDLVYENMTKILQTAEKHKIMVTIDMEDEVRCQKTLDIFKDFRKK
YEHVSTVLQAYLYRTEKDIDDLDSLNPFLRLVKGAYKESEKVAFPEKSDV
DENYKKIIRKQLLNGHYTAIATHDDKMIDFTKQLAKEHGIANDKFEFQML
YGMRSQTQLSLVKEGYNMRVYLPYGEDWYGYFMRRLAERPSNIAFAFKGM
TKK.

Additionally, the coding region is found at about 344111-345019 bp of the B. subtilis 168 chromosome.

The ycgN coding sequence of the ycgN protein (similar to 1-pyrroline-5-carboxylate dehydrogenase) of B. subtilis 168 is shown below:

(SEQ ID NO:54)

ATGACAACACCTTACAAACACGAGCCATTCACAAATTTCCAAGATCAAAA
CTACGTGGAAGCGTTTAAAAAAGCGCTTGCGACAGTAAGCGAATATTTAG
GAAAAGACTATCCGCTTGTCATTAACGGCGAGAGAGTGGAAACGGAAGCG
AAAATCGTTTCAATCAACCCAGCTGATAAAGAAGAAGTCGTCGGCCGAGT
GTCAAAAGCGTCTCAAGAGCACGCTGAGCAAGCGATTCAAGCGGCTGCAA
AAGCATTTGAAGAGTGGAGATACACGTCTCCTGAAGAGAGAGCGGCTGTC
CTGTTCCGCGCTGCTGCCAAAGTCCGCAGAAGAAAACATGAATTCTCAGC
TTTGCTTGTGAAAGAAGCAGGAAAGCCTTGGAACGAGGCGGATGCCGATA
CGGCTGAAGCGATTGACTTCATGGAGTATTATGCACGCCAAATGATCGAA
CTGGCAAAAGGCAAACCGGTCAACAGCCGTGAAGGCGAGAAAAACCAATA
TGTATACACGCCGACTGGAGTGACAGTCGTTATCCCGCCTTGGAACTTCT
TGTTTGCGATCATGGCAGGCACAACAGTGGCGCCGATCGTTACTGGAAAC
ACAGTGGTTCTGAAACCTGCGAGTGCTACACCTGTTATTGCAGCAAAATT
TGTTGAGGTGCTTGAAGAGTCCGGATTGCCAAAAGGCGTAGTCAACTTTG
TTCCGGGAAGCGGATCGGAAGTAGGCGACTATCTTGTTGACCATCCGAAA
ACAAGCCTTATCACATTTACGGGATCAAGAGAAGTTGGTACGAGAATTTT
CGAACGCGCGGCGAAGGTTCAGCCGGGCCAGCAGCATTTAAAGCGTGTCA
TCGCTGAAATGGGCGGTAAAGATACGGTTGTTGTTGATGAGGATGCGGAC
ATTGAATTAGCGGCTCAATCGATCTTTACTTCAGCATTCGGCTTTGCGGG
ACAAAAATGCTCTGCAGGTTCACGTGCAGTAGTTCATGAAAAAGTGTATG
ATCAAGTATTAGAGCGTGTCATTGAAATTACGGAATCAAAAGTAACAGCT
AAACCTGACAGTGCAGATGTTTATATGGGACCTGTCATTGACCAAGGTTC
TTATGATAAAATTATGAGCTATATTGAGATCGGAAAACAGGAAGGGCGTT
TAGTAAGCGGCGGTACTGGTGATGATTCGAAAGGATACTTCATCAAACCG
ACGATCTTCGCTGACCTTGATCCGAAAGCAAGACTCATGCAGGAAGAAAT
TTTCGGACCTGTCGTTGCATTTTGTAAAGTGTCAGACTTTGATGAAGCTT
TAGAAGTGGCAAACAATACTGAATATGGTTTGACAGGCGCGGTTATCACA
AACAACCGCAAGCACATCGAGCGTGCGAAACAGGAATTCCATGTCGGAAA
CCTATACTTCAACCGCAACTGTACAGGTGCTATCGTCGGCTACCATCCGT
TTGGCGGCTTCAAAATGTCGGGAACGGATTCAAAAGCAGGCGGGCCGGAT
TACTTGGCTCTGCATATGCAAGCAAAAACAATCAGTGAAATGTTC.

The deduced amino acid sequence of YcgN protein is:

(SEQ ID NO:55)

MTTPYKHEPFTNFQDQNYVEAFKKALATVSEYLGKDYPLVINGERVETEA
KIVSINPADKEEVVGRVSKASQEHAEQAIQAAAKAFEEWRYTSPEERAAV
LFRAAAKVRRRKHEFSALLVKEAGKPWNEADADTAEAIDFMEYYARQMIE
LAKGKPVNSREGEKNQYVYTPTGVTVVIPPWNFLFAIMAGTTVAPIVTGN
TVVLKPASATPVIAAKFVEVLEESGLPKGVVNFVPGSGSEVGDYLVDHPK
TSLITFTGSREVGTRIFERAAKVQPGQQHLKRVIAEMGGKDTVVVDEDAD
IELAAQSIFTSAFGFAGQKCSAGSRAVVHEKVYDQVLERVIEITESKVTA
KPDSADVYMGPVIDQGSYDKIMSYIEIGKQEGRLVSGGTGDDSKGYFIKP
TIFADLDPKARLMQEEIFGPVVAFCKVSDFDEALEVANNTEYGLTGAVIT
NNRKHIERAKQEFHVGNLYFNRNCTGAIVGYHPFGGFKMSGTDSKAGGPD
YLALHMQAKTISEMF.

Additionally, the coding region is found at about 345039-346583 bp of the B. subtilis 168 chromosome.

The sigD coding sequence of the sigD protein (RNA polymerase flagella, motility, chemotaxis and autolysis sigma factor) of B. subtilis 168 is shown below:

(SEQ ID NO:56)

ATGCAATCCTTGAATTATGAAGATCAGGTGCTTTGGACGCGCTGGAAAGA
GTGGAAAGATCCTAAAGCCGGTGACGACTTAATGCGCCGTTACATGCCGC
TTGTCACATATCATGTAGGCAGAATTTCTGTCGGACTGCCGAAATCAGTG
CATAAAGACGATCTTATGAGCCTTGGTATGCTTGGTTTATATGATGCCCT
TGAAAAATTTGACCCCAGCCGGGACTTAAAATTTGATACCTACGCCTCGT
TTAGAATTCGCGGCGCAATCATAGACGGGCTTCGTAAAGAAGATTGGCTG
CCCAGAACCTCGCGCGAAAAAACAAAAAAGGTTGAAGCAGCAATTGAAAA
GCTTGAACAGCGGTATCTTCGGAATGTATCGCCCGCGGAAATTGCAGAGG
AACTCGGAATGACGGTACAGGATGTCGTGTCAACAATGAATGAAGGTTTT
TTTGCAAATCTGCTGTCAATTGATGAAAAGCTCCATGATCAAGATGACGG
GGAAAACATTCAAGTCATGATCAGAGATGACAAAAATGTTCCGCCTGAAG
AAAAGATTATGAAGGATGAACTGATTGCACAGCTTGCGGAAAAAATTCAC
GAACTCTCTGAAAAAGAACAGCTGGTTGTCAGTTTGTTCTACAAAGAGGA
GTTGACACTGACAGAAATCGGACAAGTATTAAATCTTTCTACGTCCCGCA
TATCTCAGATCCATTCAAAGGCATTATTTAAATTAAAGAATCTGCTGGAA
AAAGTGATACAA.

The deduced amino acid sequence of the SigD is:

(SEQ ID NO:57)

MQSLNYEDQVLWTRWKEWKDPKAGDDLMRRYMPLVTYHVGRISVGLPKSV
HKDDLMSLGMLGLYDALEKFDPSRDLKFDTYASFRIRGAIIDGLRKEDWL
PRTSREKTKKVEAAIEKLEQRYLRNVSPAEIAEELGMTVQDVVSTMNEGF
FANLLSIDEKLHDQDDGENIQVMIRDDKNVPPEEKIMKDELIAQLAEKIH
ELSEKEQLVVSLFYKEELTLTEIGQVLNLSTSRISQIHSKALFKLKNLLE
KVIQ.

Additionally, the coding region is found at about 1715786-1716547 bp of the B. subtilis 168 chromosome.

As indicated above, it is contemplated that inactivated analogous genes found in other Bacillus hosts will find use in the present invention.

In some preferred embodiments, the host cell is a member of the genus Bacillus, while in some embodiments, the Bacillus strain of interest is alkalophilic. Numerous alkalophilic Bacillus strains are known (See e.g., U.S. Pat. No. 5,217,878; and Aunstrup et al., Proc IV IFS: Ferment. Technol. Today, 299-305 [1972]). In some preferred embodiments, the Bacillus strain of interest is an industrial Bacillus strain. Examples of industrial Bacillus strains include, but are not limited to B. licheniformis, B. lentus, B. subtilis, and B. amyloliquefaciens. In additional embodiments, the Bacillus host strain is selected from the group consisting of B. lentus, B. brevis, B. stearothermophilus, B. alkalophilus, B. coagulans, B. circulans, B. pumilus, B. thuringiensis, B. clausii, and B. megaterium, as well as other organisms within the genus Bacillus, as discussed above. In some particularly preferred embodiments, B. subtilis is used. For example, U.S. Pat. Nos. 5,264,366 and 4,760,025 (RE 34,606) describe various Bacillus host strains that find use in the present invention, although other suitable strains are contemplated for use in the present invention.

An industrial strain may be a non-recombinant strain of a Bacillus sp., a mutant of a naturally occurring strain or a recombinant strain. Preferably, the host strain is a recombinant host strain wherein a polynucleotide encoding a polypeptide of interest has been introduced into the host. A further preferred host strain is a Bacillus subtilis host strain and particularly a recombinant Bacillus subtilis host strain. Numerous B. subtilis strains are known, including but not limited to 1A6 (ATCC 39085), 168 (1A01), SB19, W23, Ts85, B637, PB1753 through PB1758, PB3360, JH642, 1A243 (ATCC 39,087), ATCC 21332, ATCC 6051, MI113, DE100 (ATCC 39,094), GX4931, PBT 110, and PEP 211strain (See e.g., Hoch et al., Genetics, 73:215-228 [1973]; U.S. Pat. No. 4,450,235; U.S. Pat. No. 4,302,544; and EP 0134048). The use of B. subtilis as an expression host is further described by Palva et al. and others (See, Palva et al., Gene 19:81-87 [1982]; also see Fahnestock and Fischer, J. Bacteriol., 165:796-804 [1986]; and Wang et al., Gene 69:39-47 [1988]).

Industrial protease producing Bacillus strains provide particularly preferred expression hosts. In some preferred embodiments, use of these strains in the present invention provides further enhancements in efficiency and protease production. Two general types of proteases are typically secreted by Bacillus sp., namely neutral (or “metalloproteases”) and alkaline (or “serine”) proteases. Serine proteases are enzymes which catalyze the hydrolysis of peptide bonds in which there is an essential serine residue at the active site. Serine proteases have molecular weights in the 25,000 to 30,000 range (See, Priest, Bacteriol. Rev., 41:711-753 [1977]). Subtilisin is a preferred serine protease for use in the present invention. A wide variety of Bacillus subtilisins have been identified and sequenced, for example, subtilisin 168, subtilisin BPN′, subtilisin Carlsberg, subtilisin DY, subtilisin 147 and subtilisin 309 (See e.g., EP 414279 B; WO 89/06279; and Stahl et al., J. Bacteriol., 159:811-818 [1984]). In some embodiments of the present invention, the Bacillus host strains produce mutant (e.g., variant) proteases. Numerous references provide examples of variant proteases and reference (See e.g., WO 99/20770; WO 99/20726; WO 99/20769; WO 89/06279; RE 34,606; U.S. Pat. No. 4,914,031; U.S. Pat. No. 4,980,288; U.S. Pat. No. 5,208,158; U.S. Pat. No. 5,310,675; U.S. Pat. No. 5,336,611; U.S. Pat. No. 5,399,283; U.S. Pat. No. 5,441,882; U.S. Pat. No. 5,482,849; U.S. Pat. No. 5,631,217; U.S. Pat. No. 5,665,587; U.S. Pat. No. 5,700,676; U.S. Pat. No. 5,741,694; U.S. Pat. No. 5,858,757; U.S. Pat. No. 5,880,080; U.S. Pat. No. 6,197,567; and U.S. Pat. No. 6,218,165).

In yet another embodiment, a preferred Bacillus host is a Bacillus sp. that includes a mutation or deletion in at least one of the following genes, degU, degS, degR and degQ. Preferably the mutation is in a degU gene, and more preferably the mutation is degU(Hy)32. (See, Msadek et al., J. Bacteriol., 172:824-834 [1990]; and Olmos et al., Mol. Gen. Genet., 253:562-567 [1997]). A most preferred host strain is a Bacillus subtilis carrying a degU32(Hy) mutation. In a further embodiment, the Bacillus host comprises a mutation or deletion in scoC4, (See, Caldwell et al., J. Bacteriol., 183:7329-7340 [2001]); spolIE (See, Arigoni et al., Mol. Microbiol., 31:1407-1415 [1999]); oppA or other genes of the opp operon (See, Perego et al., Mol. Microbiol., 5:173-185 [1991]). Indeed, it is contemplated that any mutation in the opp operon that causes the same phenotype as a mutation in the oppA gene will find use in some embodiments of the altered Bacillus strain of the present invention. In some embodiments, these mutations occur alone, while in other embodiments, combinations of mutations are present. In some embodiments, an altered Bacillus of the invention is obtained from a Bacillus host strain that already includes a mutation to one or more of the above-mentioned genes. In alternate embodiments, an altered Bacillus of the invention is further engineered to include mutation of one or more of the above-mentioned genes.

In yet another embodiment, the incoming sequence comprises a selective marker located between two loxP sites (See, Kuhn and Torres, Meth. Mol. Biol., 180:175-204 [2002]), and the antimicrobial is then deleted by the action of Cre protein. In some embodiments, this results in the insertion of a single loxP site, as well as a deletion of native DNA, as determined by the primers used to construct homologous flanking DNA and antimicrobial-containing incoming DNA.

Those of skill in the art are well aware of suitable methods for introducing polynucleotide sequences into Bacillus cells (See e.g., Ferrari et al., “Genetics,” in Harwood et al. (ed.), Bacillus, Plenum Publishing Corp. [1989], pages 57-72; See also, Saunders et al., J. Bacteriol., 157:718-726 [1984]; Hoch et al., J. Bacteriol., 93:1925-1937 [1967]; Mann et al., Current Microbiol., 13:131-135 [1986]; and Holubova, Folia Microbiol., 30:97 [1985]; for B. subtilis, Chang et al., Mol. Gen. Genet., 168:11-115 [1979]; for B. megaterium, Vorobjeva et al., FEMS Microbiol. Lett., 7:261-263 [1980]; for B amyloliquefaciens, Smith et al., Appl. Env. Microbiol., 51:634 (1986); for B. thuringiensis, Fisher et al., Arch. Microbiol., 139:213-217 [1981]; and for B. sphaericus, McDonald, J. Gen. Microbiol., 130:203 [1984]). Indeed, such methods as transformation including protoplast transformation and congression, transduction, and protoplast fusion are known and suited for use in the present invention. Methods of transformation are particularly preferred to introduce a DNA construct provided by the present invention into a host cell.

In addition to commonly used methods, in some embodiments, host cells are directly transformed (i.e., an intermediate cell is not used to amplify, or otherwise process, the DNA construct prior to introduction into the host cell). Introduction of the DNA construct into the host cell includes those physical and chemical methods known in the art to introduce DNA into a host cell without insertion into a plasmid or vector. Such methods include, but are not limited to calcium chloride precipitation, electroporation, naked DNA, liposomes and the like. In additional embodiments, DNA constructs are co-transformed with a plasmid, without being inserted into the plasmid. In further embodiments, a selective marker is deleted from the altered Bacillus strain by methods known in the art (See, Stahl et al., J. Bacteriol., 158:411-418 [1984]; and Palmeros et al., Gene 247:255-264 [2000]).

In some embodiments, host cells are transformed with one or more DNA constructs according to the present invention to produce an altered Bacillus strain wherein two or more genes have been inactivated in the host cell. In some embodiments, two or more genes are deleted from the host cell chromosome. In alternative embodiments, two or more genes are inactivated by insertion of a DNA construct. In some embodiments, the inactivated genes are contiguous (whether inactivated by deletion and/or insertion), while in other embodiments, they are not contiguous genes.

There are various assays known to those of ordinary skill in the art for detecting and measuring activity of intracellularly and extracellularly expressed polypeptides. In particular, for proteases, there are assays based on the release of acid-soluble peptides from casein or hemoglobin measured as absorbance at 280 nm or calorimetrically using the Folin method (See e.g., Bergmeyer et al., “Methods of Enzymatic Analysis” vol. 5, Pentidases, Proteinases and their Inhibitors, Verlag Chemie, Weinheim [1984]). Other assays involve the solubilization of chromogenic substrates (See e.g., Ward, “Proteinases,” in Fogarty (ed.)., Microbial Enzymes and Biotechnology, Applied Science, London, [1983], pp 251-317). Other exemplary assays include succinyl-Ala-Ala-Pro-Phe-para nitroanilide assay (SAAPFpNA) and the 2,4,6-trinitrobenzene sulfonate sodium salt assay (TNBS assay). Numerous additional references known to those in the art provide suitable methods (See e.g., Wells et al., Nucleic Acids Res. 11:7911-7925 [1983]; Christianson et al., Anal. Biochem., 223:119-129 [1994]; and Hsia et al., Anal Biochem., 242:221-227 [1999]).

Means for determining the levels of secretion of a protein of interest in a host cell and detecting expressed proteins include the use of immunoassays with either polyclonal or monoclonal antibodies specific for the protein. Examples include enzyme-linked immunosorbent assay (ELISA), radioimmunoassay (RIA), fluorescence immunoassay (FIA), and fluorescent activated cell sorting (FACS). However, other methods are known to those in the art and find use in assessing the protein of interest (See e.g., Hampton et al., Serological Methods, A Laboratory Manual, APS Press, St. Paul, Minn. [1990]; and Maddox et al., J. Exp. Med., 158:1211 [1983]). In some preferred embodiments, secretion of a protein of interest is higher in the altered strain obtained using the present invention than in a corresponding unaltered host. As known in the art, the altered Bacillus cells produced using the present invention are maintained and grown under conditions suitable for the expression and recovery of a polypeptide of interest from cell culture (See e.g., Hardwood and Cutting (eds.) Molecular Biological Methods for Bacillus, John Wiley & Sons [1990]).

B. Large Chromosomal Deletions

As indicated above, in addition to single and multiple gene deletions, the present invention provides large chromosomal deletions. In some preferred embodiments of the present invention, an indigenous chromosomal region or fragment thereof is deleted from a Bacillus host cell to produce an altered Bacillus strain. In some embodiments, the indigenous chromosomal region includes prophage regions, antimicrobial regions, (e.g., antibiotic regions), regulator regions, multi-contiguous single gene regions and/or operon regions. The coordinates delineating indigenous chromosomal regions referred to herein are specified according to the Bacillus subtilis strain 168 chromosome map. Numbers generally relate to the beginning of the ribosomal binding site, if present, or the end of the coding region, and generally do not include a terminator that might be present. The Bacillus subtilis genome of strain 168 is well known (See, Kunst et al., Nature 390:249-256 [1997]; and Henner et al., Microbiol. Rev., 44:57-82 [1980]), and is comprised of one 4215 kb chromosome. However, the present invention also includes analogous sequences from any Bacillus strain. Particularly preferred are other B. subtilis strains, B. licheniformis strains and B. amyloliquefaciens strains.

In some embodiments, the indigenous chromosomal region includes prophage segments and fragments thereof. A “prophage segment” is viral DNA that has been inserted into the bacterial chromosome wherein the viral DNA is effectively indistinguishable from normal bacterial genes. The B. subtilis genome is comprised of numerous prophage segments; these segments are not infective. (Seaman et al., Biochem., 3:607-613 [1964]; and Stickler et al., Virol., 26:142-145 [1965]). Although any one of the Bacillus subtilis prophage regions may be deleted, reference is made to the following non-limiting examples.

One prophage region that is deleted in some embodiments of the present invention is a sigma K intervening “skin” element. This region is found at about 2652600 bp (spolVCA) to 2700579 bp (yqaB) of the B. subtilis 168 chromosome. Using the present invention, about a 46 kb segment was deleted, corresponding to 2653562 bp to 2699604 bp of the chromosome. This element is believed to be a remnant of an ancestral temperate phage which is position within the SIGK ORF, between the genes spolVCB and spolIIC. However, it is not intended that the present invention be limited to any particular mechanism or mode of action involving the deleted region. The element has been shown to contain 57 open reading frames with putative ribosome binding sites (See, Takemaru et al., Microbiol., 141:323-327 [1995]). During spore formation in the mother cell, the skin element is excised leading to the reconstruction of the sigK gene.

Another region suitable for deletion is a prophage 7 region. This region is found at about 2701208 bp (yrkS) to 2749572 bp (yraK) of the B. subtilis 168 chromosome. Using the present invention, about a 48.5 kb segment was deleted, corresponding to 2701087 bp to 2749642 bp of the chromosome.

A further region is a skin+prophage 7 region. This region is found at about 2652151 bp to 2749642 bp of the B. subtilis 168 chromosome. Using the present invention, a segment of about 97.5 kb was deleted. This region also includes the intervening spolIIC gene. The skin/prophage 7 region includes but is not limited to the following genes: spoIVCA-DNA recombinase, bit (multidrug resistance), cypA (cytochrome P450-like enzyme), czcD (cation-efflux system membrane protein), and rapE (response regulator aspartate phosphatase).

Yet another region is the PBSX region. This region is found at about 1319884 bp (xkdA) to 1347491 bp (xlyA) of the B. subtilis 168 chromosome. Using the present invention, a segment of about 29 kb was deleted, corresponding to 1319663 to 1348691 bp of the chromosome. Under normal non-induced conditions this prophage element is non-infective and is not bactericidal (except for a few sensitive strains such as W23 and S31). It is inducible with mitomycin C and activated by the SOS response and results in cell lysis with the release of phage-like particles. The phage particles contain bacterial chromosomal DNA and kill sensitive bacteria without injecting DNA. (Canosi et al., J. Gen. Virol. 39: 81-90 [1978]). This region includes the following non-limiting list of genes: xtmA-B; xkdA-K and M-X, xre, xtrA, xpf, xep, xhlA-B and xlyA.

A further region is the SPβ region. This region is found at about 2150824 bp (yodU) to 2286246 bp (ypqP) of the B. subtilis 168 chromosome. Using the present invention, a segment of about 133.5 kb was deleted, corresponding to 2151827 to 2285246 bp of the chromosome. This element is a temperate prophage whose function has not yet been characterized. However, genes in this region include putative spore coat proteins (yodU, sspC, yokH), putative stress response proteins (yorD, yppQ, ypnP) and other genes that have homology to genes in the spore coat protein and stress response genes such as members of the yom operon. Other genes is this region include: yot; yos, yoq, yop, yon, yom, yoz, yol, yok, ypo, and ypm.

An additional region is the prophage 1 region. This region is found at about 202098 bp (ybbU) to 220015 bp (ybdE) of the B. subtilis 168 chromosome. Using the present invention, a segment of about 18.0 kb was deleted, corresponding to 202112 to 220141 bp of the chromosome. Genes in this region include the AdaA/B operon which provides an adaptive response to DNA alkylation and ndhF which codes for NADH dehydrogenase, subunit 5.

A further region is the prophage 2 region. This region is found at about 529069 bp (ydcL) to 569493 bp (ydeJ) of the B. subtilis 168 chromosome. Using the present invention, a segment of about 40.5 kb was deleted, corresponding to 529067 to 569578 bp of the chromosome. Genes in this region include rapI/phrI (response regulator asparate phosphatase), sacV (transcriptional regulator of the levansucrase) and cspC.

Another region is the prophage 3 region. Using the present invention, a segment of about 50.7 kb segment was deleted, corresponding to about 652000 to 664300 bp of the B. subtilis 168 chromosome.

Yet another region is the prophage 4 region. This region is found at about 1263017 bp (yjcM) to 1313627 bp (yjoA) of the B. subtilis 168 chromosome. Using the present invention, a segment of about 2.3 kb was deleted, corresponding to 1262987 to 1313692 bp of the chromosome.

An additional region is the prophage 5 region. Using the present invention a segment of about 20.8 kb segment was deleted, corresponding to about 1879200 to 1900000 bp of the B. subtilis 168 chromosome.

Another region is the prophage 6 region. Using the present invention a segment of about a 31.9 kb segment was deleted, corresponding to about 2046050 to 2078000 bp in the B. subtilis 168 chromosome.

In further embodiments, the indigenous chromosomal region includes one or more operon regions, multi-contiguous single gene regions, and/or anti-microbial regions. In some embodiments, these regions include the following:

1) The PPS Operon Region:

This region is found at about 1959410 bp (ppsE) to 1997178 bp (ppsA) of the Bacillus subtilis 168 chromosome. Using the present invention, a segment of about 38.6 kb was deleted, corresponding to about 1960409 to 1998026 bp of the chromosome. This operon region is involved in antimicrobial synthesis and encodes plipastatin synthetase;

2) The PKS Operon Region:

This region is found at about 1781110 bp (pksA) to 1857712 bp (pksR) of the B. subtilis 168 chromosome. Using the present invention, a segment of about 76.2 kb was deleted, corresponding to about 1781795 to 1857985 bp of the chromosome. This region encodes polyketide synthase and is involved in anti-microbial synthesis. (Scotti et al., Gene, 130:65-71 [1993]);

3) The yvfF-yveK Operon Region:

This region is found at about 3513149 bp (yvfF) to 3528184 bp (yveK) of the B. subtilis 168 chromosome. Using the present invention, a segment of about 15.8 kb was deleted, corresponding to about 3513137 to 3528896 bp of the chromosome. This region codes for a putative polysaccharide (See, Dartois et al., Seventh International Conference on Bacillus (1993) Institute Pasteur [1993], page 56). This region includes the following genes; yvfA-F, yveK-T and sir. The sir gene region which is found at about 3529014-3529603 bp of the B. subtilis 168 chromosome encompasses about a 589 bp segment. This region is the regulator region of the yvfF-yveK operon;

4) The DHB Operon Region:
This region is found at about 3279750 bp (yukL) to 3293206 bp (yuiH) of the B. subtilis 168 chromosome. Using the present invention, a segment of about 13.0 kb was deleted, corresponding to 3279418-3292920 bp of the chromosome. This region encodes the biosynthetic template for the catecholic siderophone 2,3-dihydroxy benzoate-glycine-threonine trimeric ester bacilibactin. (See, May et al., J. Biol. Chem., 276:7209-7217 [2001]). This region includes the following genes: yukL, yukM, dhbA-C, E and F, and yuiI-H.

While the regions, as described above, are examples of preferred indigenous chromosomal regions to be deleted, in some embodiments of the present invention, a fragment of the region is also deleted. In some embodiments, such fragments include a range of about 1% to 99% of the indigenous chromosomal region. In other embodiments, fragments include a range of about 5% to 95% of the indigenous chromosomal region. In yet additional embodiments, fragments comprise at least 99%, 98%, 97%, 96%, 95%, 94%, 93%, 92%, 90%, 88%, 85%, 80%, 75%, 70%, 65%, 50%, 40%, 30%, 25%, 20% and 10% of the indigenous chromosomal region.

Further non-limiting examples of fragments of indigenous chromosomal regions to be deleted with reference to the chromosomal location, in the B. subtilis 168 chromosome include the following:

a) for the skin region:
i) a coordinate location of about 2666663 to 2693807, which includes yqcC to yqaM, and
ii) a coordinate location of about 2658440 to 2659688, which includes rapE to phrE;

b) for the PBSX prophage region:
i) a coordinate location of about 1320043 to 1345263, which includes xkdA to xkdX, and
ii) a coordinate location of about 1326662 to 1345102, which includes xkdE to xkdW;

c) for the SPβ region:
i) a coordinate location of about 2149354 to 2237029, which includes yodV to yonA;

d) for the DHB region:
i) a coordinate location of about 3282879 to 3291353, which includes dhbF to dhbA;

e) for the yvfF-yveK region:
i) a coordinate location of about 3516549 to 3522333, which includes yvfB to yveQ,
ii) a coordinate location of about 3513181 to 3528915, which includes yvfF to yveK, and
iii) a coordinate location of about 3521233 to 3528205, which includes yveQ to yveL;

f) for the prophage 1 region:
i) a coordinate location of about 213926 to 220015, which includes ybcO to ybdE, and
ii) a coordinate location of about 214146 to 220015, which includes ybcP to ybdE;

g) for the prophage 2 region:
i) a coordinate location of about 546867 to 559005, which includes rapI to cspC; and

h) for the prophage 4 region:
i) a coordinate location of about 1263017 to 675421, which includes yjcM to ydjJ.

The number of fragments of indigenous chromosomal regions which are suitable for deletion are numerous, because a fragment may be comprised of only a few bps less than the identified indigenous chromosomal region. Furthermore, many of the identified indigenous chromosomal regions encompass a large number of genes. Those of skill in the art are capable of easily determining which fragments of the indigenous chromosomal regions are suitable for deletion for use in a particular application.

The definition of an indigenous chromosomal region is not so strict as to exclude a number of adjacent nucleotides to the defined segment. For example, while the SPβ region is defined herein as located at coordinates 2150824 to 2286246 of the B. subtilis 168 chromosome, an indigenous chromosomal region may include a further 10 to 5000 bp, a further 100 to 4000 bp, or a further 100 to 1000 bp on either side of the region. The number of bp on either side of the region is limited by the presence of another gene not included in the indigenous chromosomal region targeted for deletion.

As stated above, the location of specified regions herein disclosed are in reference to the B. subtilis 168 chromosome. Other analogous regions from Bacillus strains are included in the definition of an indigenous chromosomal region. While the analogous region may be found in any Bacillus strain, particularly preferred analogous regions are regions found in other Bacillus subtilis strains, Bacillus licheniformis strains and Bacillus amyloliquefaciens strains.

In certain embodiments, more than one indigenous chromosomal region or fragment thereof is deleted from a Bacillus strain. However, the deletion of one or more indigenous chromosomal regions or fragments thereof does not deleteriously affect reproductive viability of the strain which includes the deletion. In some embodiments, two indigenous chromosomal regions or fragments thereof are deleted. In additional embodiments, three indigenous chromosomal regions or fragments thereof are deleted. In yet another embodiment, four indigenous chromosomal regions or fragments thereof are deleted. In a further embodiment, five indigenous chromosomal regions or fragments thereof are deleted. In another embodiment, as many as 14 indigenous chromosomal regions or fragments thereof are deleted. In some embodiments, the indigenous chromosomal regions or fragments thereof are contiguous, while in other embodiments, they are located on separate regions of the Bacillus chromosome.

A strain of any member of the genus Bacillus comprising a deleted indigenous chromosomal region or fragment thereof finds use in the present invention. In some preferred embodiments, the Bacillus strain is selected from the group consisting of B. subtilis strains, B. amyloliquefaciens strains, B. lentus strains, and B. licheniformis strains. In some preferred embodiments, the strain is an industrial Bacillus strain, and most preferably an industrial B. subtilis strain. In a further preferred embodiment, the altered Bacillus strain is a protease-producing strain. In some particularly preferred embodiments, it is a B. subtilis strain that has been previously engineered to include a polynucleotide encoding a protease enzyme.

As indicated above, a Bacillus strain in which an indigenous chromosomal region or fragment thereof has been deleted is referred to herein as “an altered Bacillus strain.” In preferred embodiments of the present invention, the altered Bacillus strain has an enhanced level of expression of a protein of interest (i.e., the expression of the protein of interest is enhanced, compared to a corresponding unaltered Bacillus strain grown under the same growth conditions).

One measure of enhancement is the secretion of the protein of interest. In some embodiments, production of the protein of interest is enhanced by at least 0.5%, 1.0%, 1.5%, 2.0%, 2.5%, 3.0%, 4.0%, 5.0%, 8.0%,10%,15%, 20% and 25% or more, compared to the corresponding unaltered Bacillus strain. In other embodiments, production of the protein of interest is enhanced by between about 0.25% to 20%; 0.5% to 15% and 1.0% to 10%, compared to the corresponding unaltered Bacillus strain as measured in grams of protein produced per liter.

The altered Bacillus strains provided by the present invention comprising a deletion of an indigenous chromosomal region or fragment thereof are produced using any suitable methods, including but not limited to the following means. In one general embodiment, a DNA construct is introduced into a Bacillus host. The DNA construct comprises an inactivating chromosomal segment, and in some embodiments, further comprises a selective marker. Preferably, the selective marker is flanked on both the 5′ and 3′ ends by one section of the inactivating chromosomal segment.

In some embodiments, the inactivating chromosomal segment, while preferably having 100% sequence identity to the immediate upstream and downstream nucleotides of an indigenous chromosomal region to be deleted (or a fragment of said region), has between about 70 to 100%, about 80 to 100%, about 90 to 100%, and about 95 to 100% sequence identity to the upstream and downstream nucleotides of the indigenous chromosomal region. Each section of the inactivating chromosomal segment must include sufficient 5′ and 3′ flanking sequences of the indigenous chromosomal region to provide for homologous recombination with the indigenous chromosomal region in the unaltered host.

In some embodiments, each section of the inactivating chromosomal segment comprises about 50 to 10,000 base pairs (bp). However, lower or higher bp sections find use in the present invention. Preferably, each section is about 50 to 5000 bp, about 100 to 5000 bp, about 100 to 3000 bp; 100 to 2000 bp; about 100 to 1000 bp; about 200 to 4000 bp, about 400 to 3000 bp, about 500 to 2000 bp, and also about 800 to 1500 bp.

In some embodiments, a DNA construct comprising a selective marker and an inactivating chromosomal segment is assembled in vitro, followed by direct cloning of said construct into a competent Bacillus host, such that the DNA construct becomes integrated into the Bacillus chromosome. For example, PCR fusion and/or ligation are suitable for assembling a DNA construct in vitro. In some embodiments, the DNA construct is a non-plasmid construct, while in other embodiments, it is incorporated into a vector (i.e., a plasmid). In some embodiments, a circular plasmid is used, and the circular plasmid is cut using an appropriate restriction enzyme (i.e., one that does not disrupt the DNA construct). Thus, linear plasmids find use in the present invention (See e.g., FIG. 1; and Perego, “Integrational Vectors for Genetic Manipulation in Bacillus subtilis,” in Bacillus subtilis and other Gram-Positive Bacteria, Sonenshein. et al., Eds., Am. Soc. Microbiol., Washington, D.C. [1993]).

In some embodiments, a DNA construct or vector, preferably a plasmid including an inactivating chromosomal segment includes a sufficient amount of the 5′ and 3′ flanking sequences (seq) of the indigenous chromosomal segment or fragment thereof to provide for homologous recombination with the indigenous chromosomal region or fragment thereof in the unaltered host. In another embodiment, the DNA construct includes restriction sites engineered at upstream and downstream ends of the construct. Non-limiting examples of DNA constructs useful according to the invention and identified according to the coordinate location include:

1. A DNA construct for deleting a PBSX region: [5′ flanking seq 1318874-1319860 bp which includes the end of yjqB and the entire yjpC including the ribosome binding site (RBS)]-marker gene-[3′ flanking seq1348691-1349656 bp which includes a terminator and upstream section of the pit].

2. A DNA construct for deleting a prophage 1 region: [5′ flanking seq 201248-202112 bp which contains the entire gimS including the RBS and terminator and the ybbU RBS]-marker gene-[3′ flanking seq 220141-221195 bp which includes the entire ybgd including the RBS].

3. A DNA construct for deleting a prophage 2 region: [5′ flanking seq 527925-529067 bp which contains the end of ydcK, the entire tRNAs as follows: trnS-Asn, trnS-Ser, trnS-Glu, trnS-Gln, trnS-Lys, trnS-Leu1 and trnS-leu2]-marker gene-[3′ flanking seq 569578-571062 bp which contains the entire ydeK and upstream part of ydeL].

4. A DNA construct for deleting a prophage 4 region: [5′ flanking seq 1263127-1264270 bp which includes part of yjcM]-marker gene-[3′ flanking seq 1313660-1314583 bp which contains part of yjoB including the RBS].

5. A DNA construct for deleting a yvfF-yveK region: [5′ flanking seq 3512061-3513161 bp which includes part of sigL, the entire yvfG and the start of yvfF]-marker gene-[3′ flanking seq 3528896-3529810 bp which includes the entire sir and the start of pnbA.

6. A DNA construct for deleting a DHB operon region: [5′ flanking seq 3278457-3280255 which includes the end of ald including the terminator, the entire yuxI including the RBS, the entire yukJ including the RBS and terminator and the end of yukL]-marker gene-[3′ flanking seq 3292919-3294076 which includes the end of yuiH including the RBS, the entire yuiG including the RBS and terminator and the upstream end of yuiF including the terminator.

Whether the DNA construct is incorporated into a vector or used without the presence of plasmid DNA, it is introduced into a microorganism, preferably an E. coli cell or a competent Bacillus cell.

Methods for introducing DNA into Bacillus cells involving plasmid constructs and transformation of plasmids into E. coli are well known. The plasmids are subsequently isolated from E. coli and transformed into Bacillus. However, it is not essential to use intervening microorganisms such as E. coli, and in some embodiments, a DNA construct or vector is directly introduced into a Bacillus host.

In a preferred embodiment, the host cell is a Bacillus sp. (See e.g., U.S. Pat. No. 5,264,366, U.S. Pat. No. 4,760,025, and RE 34,6060). In some embodiments, the Bacillus strain of interest is an alkalophilic Bacillus. Numerous alkalophilic Bacillus strains are known (See e.g., U.S. Pat. No. 5,217,878; and Aunstrup et al., Proc IV IFS: Ferment. Tech. Today, 299-305 [1972]). Another type of Bacillus strain of particular interest is a cell of an industrial Bacillus strain. Examples of industrial Bacillus strains include, but are not limited to B. licheniformis, B. lentus, B. subtilis, and B. amyloliquefaciens. In additional embodiments, the Bacillus host strain is selected from the group consisting of B. licheniformis, B. subtilis, B. lentus, B. brevis, B. stearothermophilus, B. alkalophilus, B. amyloliquefaciens, B. coagulans, B. circulans, B. pumilus, B. thuringiensis, B. clausii, and B. megaterium. In particularly preferred embodiments, B. subtilis cells are used.

In some embodiments, the industrial host strains are selected from the group consisting of non-recombinant strains of Bacillus sp., mutants of a naturally-occurring Bacillus strain, and recombinant Bacillus host strains. Preferably, the host strain is a recombinant host strain, wherein a polynucleotide encoding a polypeptide of interest has been previously introduced into the host. A further preferred host strain is a Bacillus subtilis host strain, and particularly a recombinant Bacillus subtilis host strain. Numerous B. subtilis strains are known and suitable for use in the present invention (See e.g., 1A6 (ATCC 39085),168 (1A01), SB19, W23, Ts85, B637, PB1753 through PB1758, PB3360, JH642, 1A243 (ATCC 39,087), ATCC 21332, ATCC 6051, MI113, DE100 (ATCC 39,094), GX4931, PBT 110, and PEP 211strain; Hoch et al., Genetics, 73:215-228 [1973]; U.S. Pat. No. 4,450,235; U.S. Pat. No. 4,302,544; EP 0134048; Palva et al., Gene, 19:81-87 [1982]; Fahnestock and Fischer, J. Bacteriol., (1986) 165:796-804 [1986]; and Wang et al., Gene 69:39-47 [1988]). Of particular interest as expression hosts are industrial protease-producing Bacillus strains. By using these strains, the high efficiency seen for production of the protease is further enhanced by the altered Bacillus strain of the present invention.

Industrial protease producing Bacillus strains provide particularly preferred expression hosts. In some preferred embodiments, use of these strains in the present invention provides further enhancements in efficiency and protease production. As indicated above, there are two general types of proteases are typically secreted by Bacillus sp., namely neutral (or “metalloproteases”) and alkaline (or “serine”) proteases. Also as indicated above, subtilisin is a preferred serine protease for use in the present invention. A wide variety of Bacillus subtilisins have been identified and sequenced, for example, subtilisin 168, subtilisin BPN′, subtilisin Carlsberg, subtilisin DY, subtilisin 147 and subtilisin 309 (See e.g., EP 414279 B; WO 89/06279; and Stahl et al., J. Bacteriol., 159:811-818 [1984]). In some embodiments of the present invention, the Bacillus host strains produce mutant (e.g., variant) proteases. Numerous references provide examples of variant proteases and reference (See e.g., WO 99/20770; WO 99/20726; WO 99/20769; WO 89/06279; RE 34,606; U.S. Pat. No. 4,914,031; U.S. Pat. No. 4,980,288; U.S. Pat. No. 5,208,158; U.S. Pat. No. 5,310,675; U.S. Pat. No. 5,336,611; U.S. Pat. No. 5,399,283; U.S. Pat. No. 5,441,882; U.S. Pat. No. 5,482,849; U.S. Pat. No. 5,631,217; U.S. Pat. No. 5,665,587; U.S. Pat. No. 5,700,676; U.S. Pat. No. 5,741,694; U.S. Pat. No. 5,858,757; U.S. Pat. No. 5,880,080; U.S. Pat. No. 6,197,567; and U.S. Pat. No. 6,218,165.

In yet another embodiment, a preferred Bacillus host is a Bacillus sp. that includes a mutation or deletion in at least one of the following genes, degU, degS, degR and degQ. Preferably the mutation is in a degU gene, and more preferably the mutation is degU(Hy)32. (See, Msadek et al., J. Bacteriol., 172:824-834 [1990]; and Olmos et al., Mol. Gen. Genet., 253:562-567 [1997]). A most preferred host strain is a Bacillus subtilis carrying a degU32(Hy) mutation. In a further embodiment, the Bacillus host comprises a mutation or deletion in scoC4, (See, Caldwell et al., J. Bacteriol., 183:7329-7340 [2001]); spolIE (See, Arigoni et al., Mol. Microbiol., 31:1407-1415 [1999]); oppA or other genes of the opp operon (See, Perego et al., Mol. Microbiol., 5:173-185 [1991]). Indeed, it is contemplated that any mutation in the opp operon that causes the same phenotype as a mutation in the oppA gene will find use in some embodiments of the altered Bacillus strain of the present invention. In some embodiments, these mutations occur alone, while in other embodiments, combinations of mutations are present. In some embodiments, an altered Bacillus of the invention is obtained from a Bacillus host strain that already includes a mutation in one or more of the above-mentioned genes. In alternate embodiments, an altered Bacillus of the invention is further engineered to include mutation in one or more of the above-mentioned genes.

In some embodiments, two or more DNA constructs are introduced into a Bacillus host cell, resulting in the deletion of two or more indigenous chromosomal regions in an altered Bacillus. In some embodiments, these regions are contiguous, (e.g., the skin plus prophage 7 region), while in other embodiments, the regions are separated (e.g., the PBSX region and the PKS region; the skin region and the DHB region; or the PKS region, the SPβ region and the yvfF-yveK region).

Those of skill in the art are well aware of suitable methods for introducing polynucleotide sequences into bacterial (e.g., E. coli and Bacillus) cells (See e.g., Ferrari et al., “Genetics,” in Harwood et al. (ed.), Bacillus, Plenum Publishing Corp. [1989], pages 57-72; See also, Saunders et al., J. Bacteriol., 157:718-726 [1984]; Hoch et al., J. Bacteriol., 93:1925-1937 [1967]; Mann et al., Current Microbiol., 13:131-135 [1986]; and Holubova, Folia Microbiol., 30:97 [1985]; for B. subtilis, Chang et al., Mol. Gen. Genet., 168:11-115 [1979]; for B. megaterium, Vorobjeva et al., FEMS Microbiol. Lett., 7:261-263 [1980]; for B. amyloliquefaciens, Smith et al., Appl. Env. Microbiol., 51:634 (1986); for B. thuringiensis, Fisher et al., Arch. Microbiol., 139:213-217 [1981]; and for B. sphaericus, McDonald, J. Gen. Microbiol., 130:203 [1984]). Indeed, such methods as transformation including protoplast transformation and congression, transduction, and protoplast fusion are known and suited for use in the present invention. Methods of transformation are particularly preferred to introduce a DNA construct provided by the present invention into a host cell.

In addition to commonly used methods, in some embodiments, host cells are directly transformed (i.e., an intermediate cell is not used to amplify, or otherwise process, the DNA construct prior to introduction into the host cell). Introduction of the DNA construct into the host cell includes those physical and chemical methods known in the art to introduce DNA into a host cell, without insertion into a plasmid or vector. Such methods include but are not limited to calcium chloride precipitation, electroporation, naked DNA, liposomes and the like. In additional embodiments, DNA constructs are co-transformed with a plasmid without being inserted into the plasmid. In a further embodiments, a selective marker is deleted or substantially excised from the altered Bacillus strain by methods known in the art (See, Stahl et al., J. Bacteriol., 158:411-418 [1984]; and the conservative site-specific recombination [CSSR] method of Palmeros et al., described in Palmeros et al., Gene 247:255-264 [2000]). In some preferred embodiments, resolution of the vector from a host chromosome leaves the flanking regions in the chromosome while removing the indigenous chromosomal region.

In some embodiments, host cells are transformed with one or more DNA constructs according to the present invention to produce an altered Bacillus strain wherein two or more genes have been inactivated in the host cell. In some embodiments, two or more genes are deleted from the host cell chromosome. In alternative embodiments, two or more genes are inactivated by insertion of a DNA construct. In some embodiments, the inactivated genes are contiguous (whether inactivated by deletion and/or insertion), while in other embodiments, they are not contiguous genes.

As indicated above, there are various assays known to those of ordinary skill in the art for detecting and measuring activity of intracellularly and extracellularly expressed polypeptides. In particular, for proteases, there are assays based on the release of acid-soluble peptides from casein or hemoglobin measured as absorbance at 280 nm or calorimetrically using the Folin method (See e.g., Bergmeyer et al., “Methods of Enzymatic Analysis” vol. 5, Peptidases, Proteinases and their Inhibitors, Verlag Chemie, Weinheim [1984]). Other assays involve the solubilization of chromogenic substrates (See e.g., Ward, “Proteinases,” in Fogarty (ed.)., Microbial Enzymes and Biotechnology, Applied Science, London, [1983], pp 251-317). Other exemplary assays include succinyl-Ala-Ala-Pro-Phe-para nitroanilide assay (SAAPFpNA) and the 2,4,6-trinitrobenzene sulfonate sodium salt assay (TNBS assay). Numerous additional references known to those in the art provide suitable methods (See e.g., Wells et al., Nucleic Acids Res. 11:7911-7925 [1983]; Christianson et al., Anal. Biochem., 223:119-129 [1994]; and Hsia et al., Anal Biochem., 242:221-227 [1999]).

Also as indicated above, means for determining the levels of secretion of a protein of interest in a host cell and detecting expressed proteins include the use of immunoassays with either polyclonal or monoclonal antibodies specific for the protein. Examples include enzyme-linked immunosorbent assay (ELISA), radioimmunoassay (RIA), fluorescence immunoassay (FIA), and fluorescent activated cell sorting (FACS). However, other methods are known to those in the art and find use in assessing the protein of interest (See e.g., Hampton et al., Serological Methods, A Laboratory Manual, APS Press, St. Paul, Minn. [1990]; and Maddox et al., J. Exp. Med., 158:1211 [1983]). In some preferred embodiments, secretion of a protein of interest is higher in the altered strain obtained using the present invention than in a corresponding unaltered host. As known in the art, the altered Bacillus cells produced using the present invention are maintained and grown under conditions suitable for the expression and recovery of a polypeptide of interest from cell culture (See e.g., Hardwood and Cutting (eds.) Molecular Biological Methods for Bacillus, John Wiley & Sons [1990]).

As known in the art, bacteria utilize certain carbon sources for growth and synthesis of various proteins during incubation. In many cases, economics is a concern because the cost of the carbon source becomes a critical factor and the optimization of its use by the cells is a common target for strain improvement. As described herein, transcriptional arrays were utilized in order to analyze the fermentation of B. subtilis strains that produce a protease of interest. During these experiments, several metabolic reactions were identified (e.g., pckA), the modification of which were found to improve the efficiency of B. subtilis to utilize glucose and other carbon sources. Of particular interest was the anaplerotic reaction that converts oxaloacetate to phosphoenolpyruvate, which is catalyzed by the phosphoenolpyruvate carboxykinase (PckA) enzyme (EC 4.1.1.49). It is believed that this may be a futile cycle under certain growth conditions. In order to more completely analyze the role of PckA, B. subtilis constructs containing deletions in the pckA region were prepared using the PCR fusion method described herein. The effects of these deletions on cell growth, carbon yield and protease production were analyzed. Although the pckA-deletion mutant strain was more efficient in utilizing the carbon present in the complex medium (as indicated by exhibiting at least a 10% increase in carbon going to biomass), the protease production was not affected (on a per cell basis) in this medium. However, in minimal medium, it was determined that mutant pckA strains were able to produce more protease and more cells, as compared to the control strain. As discussed in greater detail in Example 6, the pckA-deletion strain, KHB5, was able to make larger halos than the control parental strain, FNA hyper1, on the LA+1.6% skin milk plate (i.e., more protease was produced by the mutant than the parent). In addition, the pckA-deletion strain, KHB5, was able to reach higher optical density than the control parental strain, FNA hyper1, in minimal medium (i.e., with glucose as the only carbon source). These results indicate that the mutant strain produced more cells from the same amount of carbon than the parental strain.

The manner and method of carrying out the present invention may be more fully understood by those of skill in the art by reference to the following examples, which examples are not intended in any manner to limit the scope of the present invention or of the claims directed thereto.

### EXPERIMENTAL

The following Examples are provided in order to demonstrate and further illustrate certain preferred embodiments and aspects of the present invention and are not to be construed as limiting the scope thereof.

In the experimental disclosure which follows, the following abbreviations apply: ° C. (degrees Centigrade); rpm (revolutions per minute); H₂O (water); dH₂O (deionized water); (HCl (hydrochloric acid); aa (amino acid); bp (base pair); kb (kilobase pair); kD (kilodaltons); gm (grams); μg (micrograms); mg (milligrams); ng (nanograms); μl (microliters); ml (milliliters); mm (millimeters); nm (nanometers); μm (micrometer); M (molar); mM (millimolar); μM (micromolar); U (units); V (volts); MW (molecular weight); sec (seconds); min(s) (minute/minutes); hr(s) (hour/hours); MgCl₂ (magnesium chloride); NaCl (sodium chloride); OD₂₈₀ (optical density at 280 nm); OD₆₀₀ (optical density at 600 nm); PAGE (polyacrylamide gel electrophoresis); PBS (phosphate buffered saline [150 mM NaCl, 10 mM sodium phosphate buffer, pH 7.2]); PEG (polyethylene glycol); ETF (elapsed fermentation time); PCR (polymerase chain reaction); RT-PCR (reverse transcription PCR); SDS (sodium dodecyl sulfate); Tris (tris(hydroxymethyl)aminomethane); w/v (weight to volume); v/v (volume to volume); LA medium (per liter: Difco Tryptone Peptone 20g, Difco Yeast Extract 10 g, EM Science NaCl 1 g, EM Science Agar 17.5 g, dH20 to 1 L); LA+1.6% Skim Milk plates contained the following compounds: Difco Tryptone 10 gm, Difco yeast extract 5 gm, NaCl 0.5 gm, 17.5 gm of agar, and distilled water to final volume of 1 liter); ATCC (American Type Culture Collection, Rockville, Md.); Clontech (CLONTECH Laboratories, Palo Alto, Calif.); Difco (Difco Laboratories, Detroit, Mich.); GIBCO BRL or Gibco BRL (Life Technologies, Inc., Gaithersburg, Md.); Invitrogen (Invitrogen Corp., San Diego, Calif.); NEB (New England Biolabs, Beverly, Mass.); Sigma (Sigma Chemical Co., St. Louis, Mo.); Takara (Takara Bio Inc. Otsu, Japan); Roche Diagnostics and Roche (Roche Diagnostics, a division of F. Hoffmann La Roche, Ltd., Basel, Switzerland); EM Science (EM Science, Gibbstown, N.J.); Qiagen (Qiagen, Inc., Valencia, Calif.); Stratagene (Stratagene Cloning Systems, La Jolla, Calif.); Affymetrix (Affymetrix, Santa Clara, Calif.).

### Example 1

### Creation of Deletion Strains

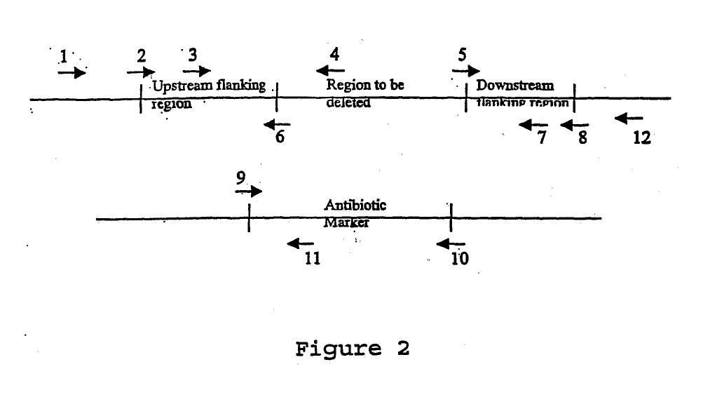

This Example describes “Method 1,” which is also depicted in FIG. 1. In this method, E. coli was used to produce a pJM102 plasmid vector carrying the DNA construct to be transformed into Bacillus strains. (See, Perego, supra). Regions immediately flanking the 5′ and 3′ ends of the deletion site were PCR amplified. PCR primers were designed to be approximately 37 base pairs in length, including 31 base pairs homologous to the Bacillus subtilis chromosome and a 6 base pair restriction enzyme site located 6 base pairs from the 5′ end of the primer. Primers were designed to engineer unique restriction sites at the upstream and downstream ends of the construct and a BamHI site between the two fragments for use in cloning. Primers for the antimicrobial markers contained BamHI sites at both ends of the fragment. Where possible, PCR primers were designed to remove promoters of deleted indigenous chromosomal regions, but to leave all terminators in the immediate area. The primary source of chromosome sequence, gene localization, and promoter and terminator information was obtained from Kunst et al., (1997) supra and also obtainable from the SubtiList World Wide Web Server known to those in the art (See e.g., Moszer et al., supra). Numerous deletions have been made using the present invention. A list of primer sequences from deletions created by this method is provided in Table 1. Reference is also made to FIG. 2 for an explanation of the primer naming system.

TABLE 1
Primers

| | | | |
|---|---|---|---|
| | Restriction | | |
| | Enzyme | | SEQ |
| Primer | Engineered | | ID |
| Name | Into Primer | Primer Sequence | NO |

| | | | |
|---|---|---|---|
| PBSX-UF | XbaI | CTACATTCTAGACGATTTGTTTGATCGATATGTGGAAGC | 60 |
| PBSX-UR | BamHI | GGCTGAGGATCCATTCCTCAGCCCAGAAGAGAACCTA | 61 |
| PBSX-DF | BamHI | TCCCTCGGATCCGAAATAGGTTCTGCTTATTGTATTCG | 62 |
| PBSX-DR | SacI | AGCGTTGAGCTCGCGCCATGCCATTATATTGGCTGCTG | 63 |
| Pphage 1- | EcoRI | GTGACGGAATTCCACGTGCGTCTTATATTGCTGAGCTT | 64 |
| Pphage 1- | BamHI | CGTTTTGGATCCAAAAACACCCCTTTAGATAATCTTAT | 65 |
| Pphage 1- | BamHI | ATCAAAGGATCCGCTATGCTCCAAATGTACACCTTTCCGT | 66 |
| Pphage 1- | PstI | ATATTTCTGCAGGCTGATATAAATAATACTGTGTGTTCC | 67 |
| Pphage 2- | SacI | CATCTTGAATTCAAAGGGTACAAGCACAGAGACAGAG | 68 |
| Pphage 2- | BamHI | TGACTTGGATCCGGTAAGTGGGCAGTTTGTGGGCAGT | 69 |
| Pphage 2- | BamHI | TAGATAGGATCCTATTGAAAACTGTTTAAGAAGAGGA | 70 |
| Pphage 2- | PstI | CTGATTCTGCAGGAGTGTTTTTGAAGGAAGCTTCATT | 71 |
| Pphage 4- | KpnI | CTCCGCGGTACCGTCACGAATGCGCCTCTTATTCTAT | 72 |
| Pphage 4- | BamHI | TCGCTGGGATCCTTGGCGCCGTGGAATCGATTTTGTCC | 73 |
| Pphage 4- | BamHI | GCAATGGGATCCTATATCAACGGTTATGAATTCACAA | 74 |
| Pphage 4- | PstI | CCAGAACTGCAGGAGCGAGGCGTCTCGCTGCCTGAAA | 75 |
| PPS-UF | SacI | GACAAGGAGCTCATGAAAAAAAGCATAAAGCTTTATGTTGC | 76 |
| PPS-UR | BamHI | GACAAGGGATCCCGGCATGTCCGTTATTACTTAATTTC | 77 |
| PPS-DF | BamHI | GACAAGGGATCCTGCCGCTTACCGGAAACGGA | 78 |
| PPS-DR | XbaI | GACAAGTCTAGATTATCGTTTGTGCAGTATTACTTG | 79 |
| SPβ-UF | SacI * | ACTGATGAGCTCTGCCTAAACAGCAAACAGCAGAAC | 80 |
| SPβ-UR | BamHI | ACGAATGGATCCATCATAAAGCCGCAGCAGATTAAATAT | 81 |
| SPβ-DF | BamHI | ACTGATGGATCCATCTTCGATAAATATGAAAGTGGC | 82 |
| SPβ-DR | XbaI | ACTGATTCTAGAGCCTTTTTCTCTTGATGCAATTCTTC | 83 |
| PKS-UF | XbaI | GAGCCTCTAGAGCCCATTGAATCATTTGTTT | 84 |
| PKS-UR | BamHI | GAGCCGGATCCTTAAGGATGTCGTTTTTGTGTCT | 85 |
| PKS-DF | BamHI | GAGCCGGATCCATTTCGGGGTTCTCAAAAAAA | 86 |
| PKS-DR | SacI | GAGCCGAGCTCATGCAAATGGAAAAATTGAT | 87 |
| Skin-UF | XbaI | GAAGTTCTAGAGATTGTAATTACAAAAGGGGGGTG | 88 |
| Skin-UR | BamHI | GAAGTGGATCCTTTCACCGATCATAAAAGCCC | 89 |
| Skin-DF | BamHI | TGAAAGGATCCATTTTTCATTGATTGTTAAGTC | 90 |
| Skin-DR | SacI | GAAGTTAGAGCTCGGGGGGGCATAAATTTCCCG | 91 |
| Phleo-UF | BamHI | GCTTATGGATCCGATACAAGAGAGGTCTCTCG | 92 |
| Phleo-DR | BamHI | GCTTATGGATCCCTGTCATGGCGCATTAACG | 93 |
| Spec-UF | BamHI | ACTGATGGATCCATCGATTTTCGTTCGTGAATACATG | 94 |
| Spec-DR | BamHI | ACTGATGGATCCCATATGCAAGGGTTTATTGTTTTC | 95 |
| CssS-UF | XbaI | GCACGTTCTAGACCACCGTCCCCTGTGTTGTATCCAC | 96 |
| CssS-UR | BamHI | AGGAAGGGATCCAGAGCGAGGAAGATGTAGGATGATC | 97 |
| CssS-DF | BamHI | TGACAAGGATCCTGTATCATACCGCATAGCAGTGCC | 98 |
| CssS-DR | SacI | TTCCGCGAGCTCGGCGAGAGCTTCAGACTCCGTCAGA | 99 |
| SBO- | XbaI | GAGCCTCTAGATCAGCGATTTGACGCGGCGC | 100 |
| SBO- | BamHI | TTATCTGGATCCCTGATGAGCAATGATGGTAAGATAGA | 101 |
| SBO- | BamHI | GGGTAA GGATCC CCCAAAAGGGCATAGTCATTCTACT | 102 |
| SBO- | Asp718 | GAGATCGGTACC CTTTTGGGCCATATCGTGGATTTC | 103 |
| PhrC-UF | HindIII | GAGCC AAGCTT CATTGACAGCAACCAGGCAGATCTC | 104 |
| PhrC-DF | PstI | GCTTATAAGCTTGATACAAGAGAGGTCTCTCG | 105 |
| PhrC-UR | PstI | GCTTATAAGCTTCTGTCATGGCGCATTAACG | 106 |
| PhrC-DR | SacI | GAGCCGAGCTC CATGCCGATGAAGTCATCGTCGAGC | 107 |
| PhrC-UF- | HindIII | CGTGAA AAGCTT TCGCGGGATGTATGAATTTGATAAG | 108 |
| PhrC-DR- | SacI | TGTAGGGAGCTC GATGCGCCACAATGTCGGTACAACG | 109 |

The restriction sites are designated as follows: XbaI is TCTAGA; BamHI is GGATCC; SacI is GAGCTC; Asp718 is GGTAGC; PstI is CTGCAG and HindIII is AAGCTT. Also prophage is designated as “Pphage.”

In this method, 100 μL PCR reactions were carried out in 150 μL Eppendorf tubes containing 84 μL water, 10 μL PCR buffer, 1 μL of each primer (i.e., PKS-UF and PKS-UR), 2 μL of dNTPs, 1 μL of wild type Bacillus chromosomal DNA template, and 1 μL of polymerase. DNA polymerases used included Taq Plus Precision polymerase and HERCULASE® polymerase (Stratagene). Reactions were carried out in a Hybaid PCRExpress thermocycler using the following program. The samples were first heated at 94° C. for 5 minutes, then cooled to a 50° hold. Polymerase was added at this point. Twenty-five cycles of amplification consisted of 1 minute at 95° C., 1 minute at 50° C. and 1 minute at 72° C. A final 10 minutes at 72° C. ensured complete elongation. Samples were held at 4° C. for analysis.

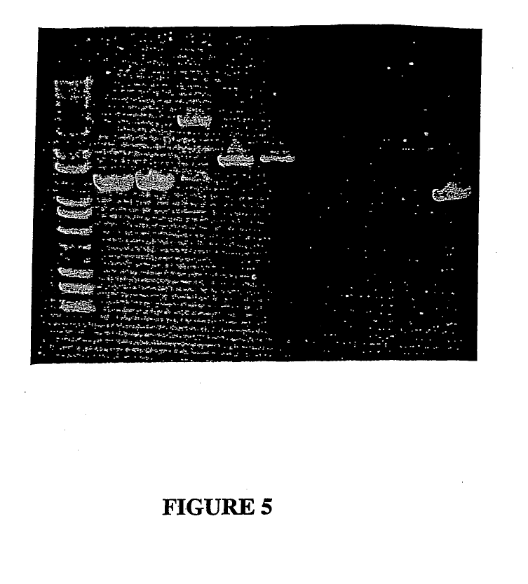

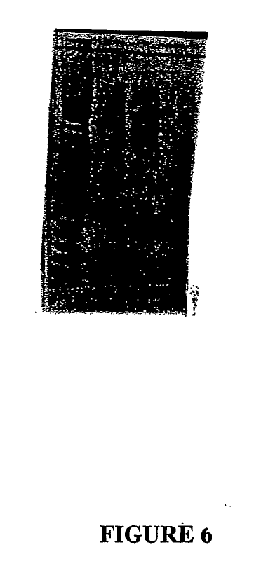

After completion of the PCR, 10 μL of each reaction were run on an Invitrogen 1.2% agarose E-gel at 60 volts for 30 minutes to check for the presence of a band at the correct size. All the gel electrophoresis methods described herein used these conditions. If a band was present, the remainder of the reaction tube was purified using the Qiagen QIAQUICK® PCR purification kit according to the manufacturer's instructions, then cut with the appropriate restriction enzyme pair. Digests were performed at 37° C. for 1 hour as a 20 μL reaction consisting of 9 μL of water, 2 μL of 10×BSA, 2 μL of an appropriate NEB restriction buffer (according to the 2000-01 NEB Catalog and Technical Reference), 5 μL of template, and 1 μL of each restriction enzyme. For example, the PBSX upstream fragment and CssS upstream fragments were cut with XbaI and BamHI in NEB (New England BioLabs) restriction buffer B. The digested fragments were purified by gel electrophoresis and extraction using the Qiagen QIAQUICK® gel extraction kit following the manufacturer's instructions. FIGS. 5 and 6 provide gels showing the results for various deletions.

Ligation of the fragments into a plasmid vector was done in two steps, using either the Takara ligation kit following the manufacturer's instructions or T4 DNA ligase (Reaction contents: 5 μL each insert fragment, 1 μL cut pJM102 plasmid, 3 μL T4 DNA ligase buffer, and 1 μL T4 DNA ligase). First, the cut upstream and downstream fragments were ligated overnight at 15° C. into unique restriction sites in the pJM102 plasmid polylinker, connecting at the common BamHI site to re-form a circular plasmid. The pJM102 plasmid was cut with the unique restriction enzyme sites appropriate for each deletion (See, Table 2; for cssS, XbaI and SacI were used) and purified as described above prior to ligation. This re-circularized plasmid was transformed into Invitrogen's “Top Ten” E. coli cells, using the manufacturers One Shot transformation protocol.

Transformants were selected on Luria-Bertani broth solidified with 1.5% agar (LA) plus 50 ppm carbanicillin containing X-gal for blue-white screening. Clones were picked and grown overnight at 37° C. in 5 mL of Luria Bertani broth (LB) plus 50 ppm carbanicillin and plasmids were isolated using Qiagen's QUIAQUICK® Mini-Prep kit. Restriction analysis confirmed the presence of the insert by cutting with the restriction sites at each end of the insert to drop an approximately 2 kb band out of the plasmid. Confirmed plasmids with the insert were cut with BamHI to linearize them in digestion reactions as described above (with an additional 1 μL of water in place of a second restriction enzyme), treated with 1 μL calf intestinal and shrimp phosphatases for 1 hour at 37° C. to prevent re-circularization, and ligated to the antimicrobial resistance marker as listed in Table 2. Antimicrobial markers were cut with BamHI and cleaned using the Qiagen Gel Extraction Kit following manufacturer's instructions prior to ligation. This plasmid was cloned into E. coli as before, using 5 ppm phleomycin (phl) or 100 ppm spectinomycin (spc) as appropriate for selection. Confirmation of marker insertion in isolated plasmids was done as described above by restriction analysis with BamHI. Prior to transformation into B. subtilis, the plasmid was linearized with ScaI to ensure a double crossover event.

TABLE 2
Unique Restriction Enzyme Pairs Used in Deletion Constructs

| Deletion Name | Unique Restriction Enzyme Pair | Antimicrobial Marker |
|---|---|---|
| Sbo | Xbal-Asp718 | spc |
| Slr | Xbal-Sacl | phleo |
| YbcO | Xbal-Sacl | spc |
| Csn | Xbal-Sall | phleo |
| PBSX | Xbal-Sacl | phl |
| PKS | Xbal-Sacl | phl |
| SPβ | Xbal-Sacl | spec |
| PPS | Xbal-Sacl | spec |
| Skin | Xbal-Sacl | phl |

### Example 2A

### Creation of DNA Constructs Using PCR Fusion to Bypass E. coli

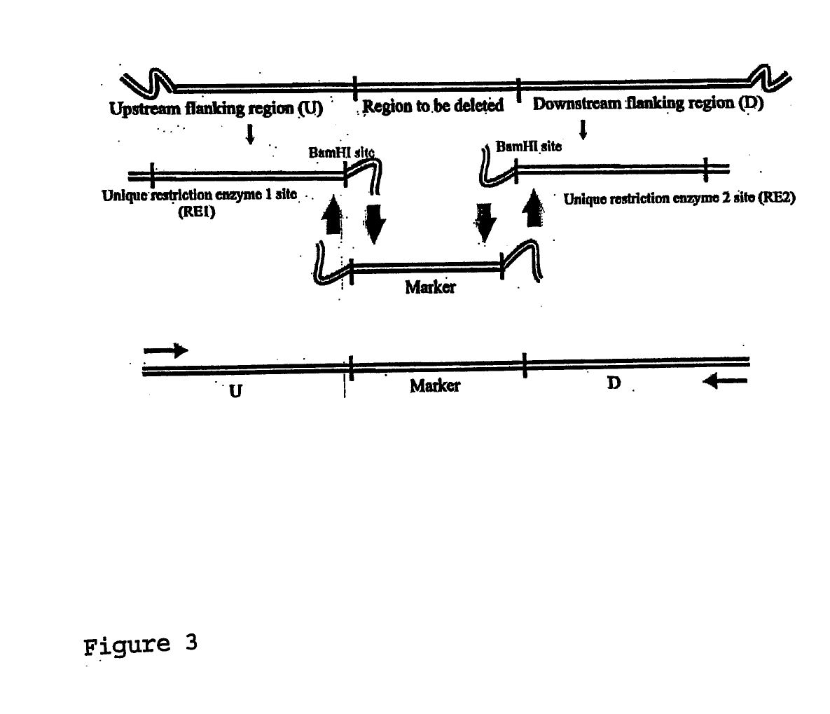

This Example describes “Method 2,” which is also depicted in FIG. 3. Upstream and downstream fragments were amplified as in Method 1, except the primers were designed with 25 bp “tails” complementary to the antimicrobial marker's primer sequences. A “tail” is defined herein as base pairs on the 5′ end of a primer that are not homologous to the sequence being directly amplified, but are complementary to another sequence of DNA. Similarly, the primers for amplifying the antimicrobial contain “tails” that are complementary to the fragments' primers. For any given deletion, the DeletionX-UFfus and DeletionX-URfus are direct complements of one another. This is also true for the DF-fus and DR-fus primer sets. In addition, in some embodiments, these primers contain restriction enzyme sites similar to those used in Method 1 for use in creating a plasmid vector (See, Table 3 and U.S. Pat. No. 5,023,171). Table 3 provides a list of primers useful for creation of deletion constructs by PCR fusion. Table 4 provides an additional list of primers useful for creation of deletion constructs by PCR fusion. However, in this Table, all deletion constructs would include the phleo^{R} marker.

TABLE 3
Primers

| | Restriction | | |
|---|---|---|---|
| | Enzyme | | SEQ |
| Primer | Engineered | | ID. |
| Name | Into Primer | Primer Sequence | NO. |
| DHB-UF | XbaI | CGAGAATCTAGAACAGGATGAATCATCTGTGGCGGG | 110 |
| DHB-UFfus-phleo | BamHI | CGACTGTCCAGCCGCTCGGCACATCGGATCCGCTTA | 111 |
| | | CCGAAAGCCAGACTCAGCAA | |
| DHB-URfus-phleo | BamHI | TTGCTGAGTCTGGCTTTCGGTAAGCGGATCCGATGT | 112 |
| | | GCCGAGCGGCTGGACAGTCG | |
| DHB-DFfus-phleo | BamHI | CGTTAATGCGCCATGACAGCCATGAGGATCCCACAA | 113 |
| | | GCCCGCACGCCTTGCCACAC | |
| DHB-DRfus-phleo | BamHI | GTGTGGCAAGGCGTGCGGGCTTGTGGGATCCTCATG | 114 |
| | | GCTGTCATGGCGCATTAACG | |
| DHB-DR | SacI | GACTTCGTCGACGAGTGCGGACGGCCAGCATCACCA | 115 |
| DHB-UF-nested | XbaI | GGCATATCTAGAGACATGAAGCGGGAAACAGATG | 116 |
| DHB-DR-nested | SacI | GGTGCGGAGCTCGACAGTATCACAGCCAGCGCTG | 117 |
| YvfF-yveK-UF | XbaI | AAGCGTTCTAGACTGCGGATGCAGATCGATCTCGGG | 118 |
| YvfF-yveK-UF- | BamHI | AACCTTCCGCTCACATGTGAGCAGGGGATCCGCTTA | 119 |
| phleo | | CCGAAAGCCAGACTCAGCAA | |
| YvfF-yveK-UR- | BamHI | TTGCTGAGTCTGGCTTTCGGTAAGCGGATCCCCTGC | 120 |
| phleo | | TCACATGTGAGCGGAAGGTT | |
| YvfF-yveK-DF- | BamHI | CGTTAATGCGCCATGACAGCCATGAGGATCCGCCTT | 121 |
| phleo | | CAGCCTTCCCGCGGCTGGCT | |
| YvfF-yveK-DR- | BamHI | AGCCAGCCGCGGGAAGGCTGAAGGCGGATCCTCATG | 122 |
| phleo | | GCTGTCATGGCGCATTAACG | |
| YvfF-yveK-DR | PstI | CAAGCACTGCAGCCCACACTTCAGGCGGCTCAGGTC | 123 |
| YvfF-yveK-UF- | XbaI | GAGATATCTAGAATGGTATGAAGCGGAATTCCCG | 124 |
| YvfF-yveK-DR- | KpnI | ATAAACGGTACCCCCCTATAGATGCGAACGTTAGCC | 125 |
| | | C | |
| Prophage7-UF | EcoRI | AAGGAGGAATTCCATCTTGAGGTATACAAACAGTCA | 126 |
| | | T | |
| Prophage 7-UF- | BamHI | TCTCCGAGAAAGACAGGCAGGATCGGGATCC | 127 |
| Prophage 7-UR- | BamHI | TTGCTGAGTCTGGCTTTCGGTAAGCGGATCC | 128 |
| Skin + prophage7- | Asp718 | AAGGACGGTACCGGCTCATTACCCTCTTTTCAAGGG | 129 |
| | | T | |
| Skin + pro7-UF- | BamHI | ACCAAAGCCGGACTCCCCCGCGAGAGGATCCGCTTA | 130 |
| phleo | | CCGAAAGCCAGACTCAGCAA | |
| Skin + pro7-UR- | BamHI | TTGCTGAGTCTGGCTTTCGGTAAGCGGATCCTCTCG | 131 |
| phleo | | CGGGGGAGTCCGGCTTTGGT | |
| Skin + pro7-DF- | BamHI | CGTTAATGCGCCATGACAGCCATGAGGATCCCATAC | 132 |
| phleo | | GGGGTACACAATGTACCATA | |
| Skin + pro7-DR- | BamHI | TATGGTACATTGTGTACCCCGTATGGGATCCTCATG | 133 |
| phleo | | GCTGTCATGGCGCATTAACG | |
| Skin + pro7-DR | PstI | GTCAACCTGCAGAGCGGCCCAGGTACAAGTTGGGGA | 134 |
| Skin + pro7-UF- | SacI | GGATCAGAGCTCGCTTGTCCTCCTGGGAACAGCCGG | 135 |
| Skin + pro7-DR- | PstI | TATATGCTGCAGGGCTCAGACGGTACCGGTTGTTCC | 136 |
| | | T | |

The restriction sites are designated as follows: XbaI is TCTAGA; BamHI is GGATCC; SacI is GAGCTC; Asp718 is GGTACC; PstI is CTGCAG and HindIII is AAGCTT.

TABLE 4

Additional Primers Used to Create Deletion Constructs
by PCR Fusion*.

| | Restriction | | |
|---|---|---|---|
| | Enzyme | | SEQ |
| Primer | Engineered | | ID |
| Name | Into Primer | Primer Sequence | NO: |
| Sir-UF | XbaI | CTGAACTCTAGACCTTCACCAGGCACAGAGGAGGTGA | 137 |
| Sir-Uffus | BamHI | GCCAATAAGTTCTCTTTAGAGAACAGGATCCGCTTAC | 138 |
| | | CGAAAGCCAGACTCAGCAA | |
| Sir-Urfus | BamHI | TTGCTGAGTCTGGCTTTCGGTAAGCGGATCCTTGTTC | 139 |
| | | TCTAAAGAGAACTTATTGGC | |
| Sir-Dffus | BamHI | CGTTAATGCGCCATGACAGCCATGAGGATCCGGGCTA | 140 |
| | | ACGTTCGCATCTATAGGGG | |
| Sir-Drfus | BamHI | CCCCTATAGATGCGAACGTTAGCCC GGATCC | 141 |
| | | TCATGGCTGTCATGGCGCATTAACG | |
| Sir-DR | SacI | TGAGACGAGCTCGATGCATAGGCGACGGCAGGGCGCC | 142 |
| Sir-UF-nested | XbaI | CGAAATTCTAGATCCCGCGATTCCGCCCTTTGTGG | 143 |
| Sir-DR-nested | SacI | TTCCAAGAGCTCGCGGAATACCGGAAGCAGCCCC | 144 |
| YbcO-UF | XbaI | CAATTCTCTAGAGCGGTCGGCGCAGGTATAGGAGGGG | 145 |
| YbcO-UF | BamHI | GAAAAGAAACCAAAAAGAATGGGAAGGATCCGCTTAC | 146 |
| | | CGAAAGCCAGACTCAGCAA | |
| YbcO-UR | BamHI | TTGCTGAGTCTGGCTTTCGGTAAGCGGATCCTTCCCA | 147 |
| | | TTCTTTTTGGTTTCTTTTC | |
| YbcO-DF | BamHI | CGTTAATGCGCCATGACAGCCATGAGGATCCGCTATT | 148 |
| | | TAACATTTGAGAATAGGGA | |
| YbcO-DR | BamHI | TCCCTATTCTCAAATGTTAAATAGCGGATCCTCATGG | 149 |
| | | CTGTCATGGCGCATTAACG | |
| YbcO-DR | SacI | CAGGCGGAGCTCCCATTTATGACGTGCTTCCCTAAGC | 150 |
| Csn-UF | XbaI | TACGAATCTAGAGATCATTGCGGAAGTAGAAGTGGAA | 151 |
| Csn-UF | BamHI | TTTAGATTGAGTTCATCTGCAGCGGGGATCCGCTTAC | 152 |
| | | CGAAAGCCAGACTCAGCAA | |
| Csn-UR | BamHI | TTGCTGAGTCTGGCTTTCGGTAAGCGGATCCCCGCTG | 153 |
| | | CAGATGAACTCAATCTAAA | |
| Csn-DF | BamHI | CGTTAATGCGCCATGACAGCCATGAGGATCCGCCAAT | 154 |
| | | CAGCCTTAGCCCCTCTCAC | |
| Csn-DR | BamHI | GTGAGAGGGGCTAAGGCTGATTGGCGGATCCTCATGG | 155 |
| | | CTGTCATGGCGCATTAACG | |
| Csn-DR | SalI | ATACTCGTCGACATACGTTGAATTGCCGAGAAGCCGC | 156 |
| Csn-UF- | NA | CTGGAGTACCTGGATCTGGATCTCC | 157 |
| Csn-DR- | NA | GCTCGGCTTGTTTCAGCTCATTTCC | 158 |
| SigB-UF | SacI | CGGTTTGAGCTCGCGTCCTGATCTGCAGAAGCTCATT | 159 |
| SigB-UF | BamHI | CTAAAGATGAAGTCGATCGGCTCATGGATCCGCTTAC | 160 |
| | | CGAAAGCCAGACTCAGCAA | |
| SigB-UR | BamHI | TTGCTGAGTCTGGCTTTCGGTAAGCGGATCCATGAGC | 161 |
| | | CGATCGACTTCATCTTTAG | |
| SigB-DF | BamHI | CGTTAATGCGCCATGACAGCCATGAGGATCCGAAGAT | 162 |
| | | CCCTCGATGGAGTTAATGT | |
| SigB-DR | BamHI | ACATTAACTCCATCGAGGGATCTTCGGATCCTCATGG | 163 |
| | | CTGTCATGGCGCATTAACG | |
| SigB-DR | SalI | GCTTCGGTCGACTTTGCCGTCTGGATATGCGTCTCTC | 164 |
| | | G | |
| SigB-UF- | SacI | GTCAAAGAGCTCTATGACAGCCTCCTCAAATTGCAGG | 165 |
| SigB-DR- | SalI | TTCCATGTCGACGCTGTGCAAAACCGCCGGCAGCGCC | 166 |
| SpoIISA-UF | EcoRI | ACATTCGAATTCAGCAGGTCAATCAGCTCGCTGACGC | 167 |
| SpoIISA-UF | BamHI | CCAGCACTGCGCTCCCTCACCCGAAGGATCCGCTTAC | 168 |
| | | CGAAAGCCAGACTCAGCAA | |
| SpoIISA-UR | BamHI | TTGCTGAGTCTGGCTTTCGGTAAGCGGATCCTTCGGG | 169 |
| | | TGAGGGAGCGCAGTGCTGG | |
| SpoIISA-DF | BamHI | CGTTAATGCGCCATGACAGCCATGAGGATCCTCGAGA | 170 |
| | | GATCCGGATGGTTTTCCTG | |
| SpoIISA-DR | BamHI | CAGGAAAACCATCCGGATCTCTCGAGGATCCTCATGG | 171 |
| | | CTGTCATGGCGCATTAACG | |
| SpoIISA-DR | HindIII | AGTCAT AAGCTTTCTGGCGTTTGATTTCATCAACGG | 172 |
| | | G | |
| SpoIISA-UF- | NA | CAGCGCGACTTGTTAAGGGACAATA | 173 |
| SpoIISA-DR- | NA | GGCTGCTGTGATGAACTTTGTCGGA | 174 |

*All deletion constructs include the phleo^{R} marker

The fragments listed in Tables 3 and 4 were size-verified by gel electrophoresis as described above. If correct, 1 μL each of the upstream, downstream, and antimicrobial resistance marker fragments were placed in a single reaction tube with the DeletionX-UF and DeletionX-DR primers or nested primers where listed. Nested primers are 25 base pairs of DNA homologous to an internal portion of the upstream or downstream fragment, usually about 100 base pairs from the outside end of the fragment (See, FIG. 2). The use of nested primers frequently enhances the success of fusion. The PCR reaction components were similar to those described above, except 82 μL of water was used to compensate for additional template volume. The PCR reaction conditions were similar to those described above, except the 72° C. extension was lengthened to 3 minutes. During extension, the antimicrobial resistance gene was fused in between the upstream and downstream pieces. This fusion fragment can be directly transformed into Bacillus without any purification steps or with a simple Qiagen QUIAQUICK® PRC purification done according to manufacturer's instructions.

### Example 2B

### Creation of pckA Deletion Construct Using PCR Fusion to Bypass E. coli

In addition to the above deletions, pckA was also modified. The PCR primers pckA UF, pckA-2Urfus, spc ffus, spc rfus, pckA Dffus and pckA DR, were used for PCR and PCR fusion reactions using the chromosomal DNA of a Bacillus subtilis 1168 derivative and pDG1726 (See, Guerout-Fleury et al., Gene 167(1-2):335-6 [1995]) as template. The primers are shown in Table 5. The method used in constructing these deletion mutants was the same as Method 1, described above.

TABLE 5
Primers Used for PckA Deletion

| | Restriction | | |
|---|---|---|---|
| | Enzyme | | Seq |
| Primer | Engineered | | ID |
| Name | Into Primer | Primer Sequence | NO: |
| pckA UF | None | TTTGCTTCCTCCTGCACAAGGCCTC | 199 |
| (PCK-1) | | | |
| pckA-2Urfus | None | CGTTATTGTGTGTGCATTTCCATTGT | 200 |
| (PCK-2) | | | |
| spc ffus | None | CAATGGAAATGCACACACAATAACGTGACTGGCAA | 201 |
| (PCK-3) | | GAGA | |
| pckA Difus | None | GTAATGGCCCTCTCGTATAAAAAAC | 202 |
| (PCK-4) | | | |
| spc rfus | None | GTTTTTTATACGAGAGGGCCATTACCAATTAGAAT | 203 |
| (PCK-5) | | GAATATTTCCC | |
| pckA DR | None | GACCAAAATGTTTCGATTCAGCATTCCT | 204 |
| (PCK-6) | | | |

### Example 3

### Creation of DNA Constructs Using Ligation of PCR Fragments and Direct Transformation of Bacillus subtilis to Bypass the E. coli Cloning Step

In this Example, a method (“Method 3”) for creating DNA constructs using ligation of PCR fragments and direct transformation of Bacillus are described. By way of example, modification of prpC, sigD and tdh/kbl are provided to demonstrate the method of ligation.

Indeed, sigD and tdh/kbl were constructed by one method and prpC by an alternate method.

A. Tdh/Kbl and SigD

The upstream and downstream fragments adjacent to the tdh/kbl region of the Bacillus subtilis chromosome were amplified by PCR similar to as described in Method 1, except that the inside primer of the flanking DNA was designed to contain type II s restriction sites. Primers for the loxP-spectinomycin-loxP cassette were designed with the same type II s restriction site as the flanks and complementary overhangs. Unique overhangs for the left flank and the right flank allowed directional ligation of the antimicrobial cassette between the upstream and downstream flanking DNA. All DNA fragments were digested with the appropriate restriction enzymes, and the fragments were purified with a Qiagen QIAQUICK® PCR purification kit using the manufacturer's instructions. This purification was followed by desalting in a 1 mL spin column containing BioRad P-6 gel and equilibrated with 2 mM Tris-HCl, pH 7.5. Fragments were concentrated to 124 to 250 ng/μL using a Savant Speed Vac SC110 system. Three piece ligations of 0.8 to 1 μg of each fragment were performed with 12U T4 ligase (Roche) in a 15 to 25 μL reaction volume at 14 to 16° C. for 16 hours. The total yield of the desired ligation product was >100 ng per reaction, as estimated by comparison to a standard DNA ladder on an agarose gel. The ligation mixture was used without purification for transformation reactions. Primers for this construction are shown in Table 6, below

TABLE 6
Primers for tdh/kbl Deletion

| | Restriction | | |
|---|---|---|---|
| | Enzyme | | SEQ |
| Primer | Engineered | | ID |
| Name | Into Primer | Primer Sequence | NO: |
| p70 DR | none | CTCAGTTCATCCATCAAATCACCAAGTCCG | 175 |
| P82 DF | BbsI | TACACGTTAGAAGACGGCTAGATGCGTCTGATTGTGACAGAC | 176 |
| | | GGCG | |
| p71 UF | none | AACCTTCCAGTCCGGTTTACTGTCGC | 177 |
| P83 UR | BbsI | GTACCATAAGAAGACGGAGCTTGCCGTGTCCACTCCGATTAT | 178 |
| | | AGCAG | |
| p98spc F | BbsI | CCTTGTCTTGAAGACGGAGCTGGATCCATAACTTCGTATAATG | 179 |
| p106 spc R | BbsI | GTACCATAAGAAGACGGCTAGAGGATGCATATGGCGGCCGC | 180 |
| p112 UF* | none | CATATGCTCCGGCTCTTCAAGCAAG (analytical primer) | 181 |
| p113 DR* | none | CCTGAGATTGATAAACATGAAGTCCTC (analytical primer) | 182 |

*primers for analytical PCR

The construct for the sigD deletion closely followed construction of tdh/kbl. The primers used for the sigD construction are provided in Table 7.

TABLE 7
Primers for sigD Construction

| | Restriction | | |
|---|---|---|---|
| | Enzyme | | SEQ |
| Primer | Engineered | | ID |
| Name | Into Primer | Primer Sequence | NO: |
| SigD UF | None | ATATTGAAGTCGGCTGGATTGTGG | 183 |
| SigD UR | BgIII | GCGGCAGATCTCGGCGCATTAAGTCGTCA | 184 |
| SigD DF | EcoRI | GCGGCGAATTCTCTGCTGGAAAAAGTGATACA | 185 |
| SigD DR | None | TTCGCTGGGATAACAACAT | 186 |
| Loxspc UF | BgIII | GCGGCAGATCTTAAGCTGGATCCATAACTTCG | 187 |
| Loxspc DR | EcoRI | GCGGCGAATTCATATGGCGGCCGCATAACTTC | 188 |
| SIgD UO | None | CAATTTACGCGGGGTGGTG | 189 |
| SigD DO | None | GAATAGGTTACGCAGTTGTTG | 190 |
| Spc UR | None | CTCCTGATCCAAACATGTAAG | 191 |
| Spc DF | None | AACCCTTGCATATGTCTAG | 192 |

B. PrpC

An additional example of creating a DNA molecule by ligation of PCR amplified DNA fragments for direct transformation of Bacillus involved a partial in-frame deletion of the gene prpC. A 3953 bp fragment of Bacillus subtilis chromosomal DNA containing the prpC gene was amplified by PCR using primers p95 and p96. The fragment was cleaved at unique restriction sites PflMI and BstXI. This yielded three fragments, an upstream, a downstream, and a central fragment. The latter is the fragment deleted and consists of 170 bp located internal to the prpC gene. The digestion mixture was purified with a Qiagen QUIAQUICK® PCR purification kit, followed by desalting in a 1 mL spin column containing BioRad P-6 gel and equilibrated with 2 mM Tris-HCl, pH 7.5. In a second PCR reaction, the antimicrobial cassette, loxP-spectinomycin-loxP, was amplified with the primer containing a BstXI site and the downstream primer containing a PflMI site both with cleavage sites complementary to the sites in the genomic DNA fragment. The fragment was digested with PflMI and BstXI and purified as described for the chromosomal fragment above. A three pieces ligation of the upstream, antimicrobial cassette, and the downstream fragments was carried out as for tdh/kbl, described above. The yield of desired ligation product was similar and the ligation product was used without further treatment for the transformation of xylRcomK competent Bacillus subtilis, as described in greater detail below.

TABLE 8
Primers for prpC Deletion

| | Restriction | | |
|---|---|---|---|
| | Enzyme | | SEQ |
| Primer | Engineered | | ID |
| Name | Into Primer | Primer Sequence | NO: |
| p95 | None | GCGCCCTTGATCCTAAGTCAGATGAAAC | 193 |
| DF | | | |
| p96 | None | CGGGTCCGATACTGACTGTAAGTTTGAC | 194 |
| UR | | | |
| p100 | PflMI | GTACCATAACCATGCCTTGGTTAGGATGCATATGGCGGCCGC | 195 |
| spc R | | | |
| p101 | BstXI | CCTTGTCTTCCATCTTGCTGGAGCTGGATCCATAACTTCGTATAATG | 196 |
| spc F | | | |
| p114 | None | GAGAGCAAGGACATGACATTGACGC | 197 |
| anal. | | | |
| p115 | None | GATCTTCACCCTCTTCAACTTGTAAAG | 198 |
| anal.,* | | | |

*anal., analytical PCR primer

C. Xylose-Induced Competence Host Cell Transformation with Ligated DNA

Cells of a host strain Bacillus subtilis with partial genotype xylRcomK, were rendered competent by growth for 2 hours in Luria-Bertani medium containing 1% xylose, as described in U.S. patent application Ser. No. 09/927,161, filed Aug. 10, 2001, herein incorporated by reference, to an OD₅₅₀ of 1. This culture was seeded from a 6 hour culture. All cultures were grown at 37° C., with shaking at 300 rpm. Aliquots of 0.3 mL of were frozen as 1:1 mixtures of culture and 30%. glycerol in round bottom 2 mL tubes and stored in liquid nitrogen for future use.

For transformation, frozen competent cells were thawed at 37° C. and immediately after thawing was completed, DNA from ligation reaction mixtures was added at a level of 5 to 15 μL per tube. Tubes were then shaken at 1400 rpm (Tekmar VXR S-10) for 60 min at 37° C. The transformation mixture was plated without dilution in 100 uL aliquots on 8 cm LA plates containing 100 ppm of spectinomycin. After growth over night, transformants were picked into Luria-Bertani (100 ppm spectinomycin) and grown at 37° C. for genomic DNA isolation performed as known in the art (See e.g., Harwood and Cuttings, Molecular Biological Methods for Bacillus, John Wiley and Son, New York, N.Y. [1990], at p. 23). Typically 400 to 1400 transformants were obtained from 100 uL transformation mix, when 5 uL of ligation reaction mix was used in the transformation.

When the antimicrobial marker was located between two loxP sites in the incoming DNA, the marker could be removed by transforming the strain with a plasmid containing the cre gene capable of expression the Cre protein. Cells were transformed with pCRM-TS-pleo (See below) cultured at 37° C. to 42° C., plated onto LA and after colonies formed patched onto LA containing 100 ppm spectinomycin. Patches which did not grow after overnight incubation were deemed to have lost the antimicrobial maker. Loss of maker was verified by PCR assay with primers appropriate for the given gene.

pCRM-TS-pleo has the following sequence (SEQ ID NO:205):

GGGGATCTCTGCAGTGAGATCTGGTAATGACTCTCTAGCTTGAGGCATCA
AATAAAACGAAAGGCTCAGTCGAAAGACTGGGCCTTTCGTTTTATCTGTT
GTTTGTCGGTGAACGCTCTCCTGAGTAGGACAAATCCGCCGCTCTAGCTA
AGCAGAAGGCCATCCTGACGGATGGCCTTTTTGCGTTTCTACAAACTCTT
GTTAACTCTAGAGCTGCCTGCCGCGTTTCGGTGATGAAGATCTTCCCGAT
GATTAATTAATTCAGAACGCTCGGTTGCCGCCGGGCGTTTTTTATGCAGC
AATGGCAAGAACGTTGCTCTAGAATAATTCTACACAGCCCAGTCCAGACT
ATTCGGCACTGAAATTATGGGTGAAGTGGTCAAGACCTCACTAGGCACCT
TAAAAATAGCGCACCCTGAAGAAGATTTATTTGAGGTAGCCCTTGCCTAC
CTAGCTTCCAAGAAAGATATCCTAACAGCACAAGAGCGGAAAGATGTTTT
GTTCTACATCCAGAACAACCTCTGCTAAAATTCCTGAAAAATTTTGCAAA
AAGTTGTTGACTTTATCTACAAGGTGTGGCATAATGTGTGGAATTGTGAG
CGGATAACAATTAAGCTTAGGAGGGAGTGTTAAATGTCCAATTTACTGAC
CGTACACCAAAATTTGCCTGCATTACCGGTCGATGCAACGAGTGATGAGG
TTCGCAAGAACCTGATGGACATGTTCAGGGATCGCCAGGCGTTTTCTGAG
CATACCTGGAAAATGCTTCTGTCCGTTTGCCGGTCGTGGGCGGCATGGTG
CAAGTTGAATAACCGGAAATGGTTTCCCGCAGAACCTGAAGATGTTCGCG
ATTATCTTCTATATCTTCAGGCGCGCGGTCTGGCAGTAAAAACTATCCAG
CAACATTTGGGCCAGCTAAACATGCTTCATCGTCGGTCCGGGCTGCCACG
ACCAAGTGACAGCAATGCTGTTTCACTGGTTATGCGGCGGATCCGAAAAG
AAAACGTTGATGCCGGTGAACGTGCAAAACAGGCTCTAGCGTTCGAACGC
ACTGATTTCGACCAGGTTCGTTCACTCATGGAAAATAGCGATCGCTGCCA
GGATATACGTAATCTGGCATTTCTGGGGATTGCTTATAACACCCTGTTAC
GTATAGCCGAAATTGCCAGGATCAGGGTTAAAGATATCTCACGTACTGAC
GGTGGGAGAATGTTAATCCATATTGGCAGAACGAAAACGCTGGTTAGCAC
CGCAGGTGTAGAGAAGGCACTTAGCCTGGGGGTAACTAAACTGGTCGAGC
GATGGATTTCCGTCTCTGGTGTAGCTGATGATCCGAATAACTACCTGTTT
TGCCGGGTCAGAAAAAATGGTGTTGCCGCGCCATCTGCCACCAGCCAGCT
ATCAACTCGCGCCCTGGAAGGGATTTTTGAAGCAACTCATCGATTGATTT
ACGGCGCTAAGGATGACTCTGGTCAGAGATACCTGGCCTGGTCTGGACAC
AGTGCCCGTGTCGGAGCCGCGCGAGATATGGCCCGCGCTGGAGTTTCAAT
ACCGGAGATCATGCAAGCTGGTGGCTGGACCAATGTAAATATTGTCATGA
ACTATATCCGTAACCTGGATAGTGAAACAGGGGCAATGGTGCGCCTGCTG
GAAGATGGCGATTAGGAGCTCGCATCACACGCAAAAAGGAAATTGGAATA
AATGCGAAATTTGAGATGTTAATTAAAGACCTTTTTGAGGTCTTTTTTTC
TTAGATTTTTGGGGTTATTTAGGGGAGAAAACATAGGGGGGTACTACGAC
CTCCCCCCTAGGTGTCCATTGTCCATTGTCCAAACAAATAAATAAATATT
GGGTTTTTAATGTTAAAAGGTTGTTTTTTATGTTAAAGTGAAAAAAACAG
ATGTTGGGAGGTACAGTGATAGTTGTAGATAGAAAAGAAGAGAAAAAAGT
TGCTGTTACTTTAAGACTTACAACAGAAGAAAATGAGATATTAAATAGAA
TCAAAGAAAAATATAATATTAGCAAATCAGATGCAACCGGTATTCTAATA
AAAAAATATGCAAAGGAGGAATACGGTGCATTTTAAACAAAAAAAGATAG
ACAGCACTGGCATGCTGCCTATCTATGACTAAATTTTGTTAAGTGTATTA
GCACCGTTATTATATCATGAGCGAAAATGTAATAAAAGAAACTGAAAACA
AGAAAAATTCAAGAGGACGTAATTGGACATTTGTTTTATATCCAGAATCA
GCAAAAGCCGAGTGGTTAGAGTATTTAAAAGAGTTACACATTCAATTTGT
AGTGTCTCCATTACATGATAGGGATACTGATACAGAAGGTAGGATGAAAA
AAGAGCATTATCATATTCTAGTGATGTATGAGGGTAATAAATCTTATGAA
CAGATAAAAATAATTAACAGAAGAATTGAATGCGACTATTCCGCAGATTG
CAGGAAGTGTGAAAGGTCTTGTGAGATATATGCTTCACATGGACGATCCT
AATAAATTTAAATATCAAAAAGAAGATATGATAGTTTATGGCGGTGTAGA
TGTTGATGAATTATTAAAGAAAACAACAACAGATAGATATAAATTAATTA
AAGAAATGATTGAGTTTATTGATGAACAAGGAATCGTAGAATTTAAGAGT
TTAATGGATTATGCAATGAAGTTTAAATTTGATGATTGGTTCCCGCTTTT
ATGTGATAACTCGGCGTATGTTATTCAAGAATATATAAAATCAAATCGGT
ATAAATCTGACCGATAGATTTTGAATTTAGGTGTCACAAGACACTCTTTT
TTCGCACCAGCGAAAACTGGTTTAAGCCGACTGGAGCTCCTGCACTGGAT
GGTGGCGCTGGATGGTAAGCCGCTGGCAAGCGGTGAAGTGCCTCTGGATG
TCGCTCCACAAGGTAAACAGTTGATTGAACTGCCTGAACTACCGCAGCCG
GAGAGCGCCGGGCAACTCTGGCTCACAGTACGCGTAGTGCAACCGAACGC
GACCGCATGGTCAGAAGCCGGGCACATCAGCGCCTGGCAGCAGTGGCGTC
TGGCGGAAAACCTCAGTGTGACGCTCCCCGCCGCGTCCCACGCCATCCCG
CATCTGACCACCAGCGAAATGGATTTTTGCATCGAGCTGGGTAATAAGCG
TTGGCAATTTAACCGCCAGTCAGGCTTTCTTTCACAGATGTGGATTGGCG
ATAAAAAACAACTGCTGACGCCGCTGCGCGATCAGTTCACCCGTGCACCG
CTGGATAACGACATTGGCGTAAGTGAAGCGACCCGCATTGACCCTAACGC
CTGGGTCGAACGCTGGAAGGCGGCGGGCCATTACCAGGCCGAAGCAGCGT
TGTTGCAGTGCACGGCAGATACACTTGCTGATGCGGTGCTGATTACGACC
GCTCACGCGTGGCAGCATCAGGGGAAAACCTTATTTATCAGCCGGAAAAC
CTACCGGATTGATGGTAGTGGTCAAATGGCGATTACCGTTGATGTTGAAG
TGGCGAGCGATACACCGCATCCGGCGCGGATTGGCCTGAACTGCCAGCTG
GCGCAGGTAGCAGAGCGGGTAAACTGGCTCGGATTAGGGCCGCAAGAAAA
CTATCCCGACCGCCTTACTGCCGCCTGTTTTGACCGCTGGGATCTGCCAT
TGTCAGACATGTATACCCCGTACGTCTTCCCGAGCGAAAACGGTCTGCGC
TGCGGGACGCGCGAATTGAATTATGGCCCACACCAGTGGCGCGGCGACTT
CCAGTTCAACATCAGCCGCTACAGTCAACAGCAACTGATGGAAACCAGCC
ATCGCCATCTGCTGCACGCGGAAGAAGGCACATGGCTGAATATCGACGGT
TTCCATATGGGGATTGGTGGCGACGACTCCTGGAGCCCGTCAGTATCGGC
GGAATTCCAGCTGAGCGCCGGTCGCTACCATTACCAGTTGGTCTGGTGTC
AAAAATAATAATAACCGGGCAGGCCATGTCTGCCCGTATTTCGCGTAAGG
AAATCCATTATGTACTATTTCAAGCTAATTCCGGTGGAAACGAGGTCATC
ATTTCCTTCCGAAAAAACGGTTGCATTTAAATCTTACATATGTAATACTT
TCAAAGACTACATTTGTAAGATTTGATGTTTGAGTCGGCTGAAAGATCGT
ACGTACCAATTATTGTTTCGTGATTGTTCAAGCCATAACACTGTAGGGAT
AGTGGAAAGAGTGCTTCATCTGGTTACGATCAATCAAATATTCAAACGGA
GGGAGACGATTTTGATGAAACCAGTAACGTTATACGATGTCGCAGAGTAT
GCCGGTGTCTCTTATCAGACCGTTTCCCGCGTGGTGAACCAGGCCAGCCA
CGTTTCTGCGAAAACGCGGGAAAAAGTGGAAGCGGCGATGGCGGAGCTGA
ATTACATTCCCAACCGCGTGGCACAACAACTGGCGGGCAAACAGTCGTTG
CTGATTGGCGTTGCCACCTCCAGTCTGGCCCTGCACGCGCCGTCGCAAAT
TGTCGCGGCGATTAAATCTCGCGCCGATCAACTGGGTGCCAGCGTGGTGG
TGTCGATGGTAGAACGAAGCGGCGTCGAAGCCTGTAAAGCGGCGGTGCAC
AATCTTCTCGCGCAACGCGTCAGTGGGCTGATCATTAACTATCCGCTGGA
TGACCAGGATGCCATTGCTGTGGAAGCTGCCTGCACTAATGTTCCGGCGT
TATTTCTTGATGTCTCTGACCAGACACCCATCAACAGTATTATTTTCTCC
CATGAAGACGGTACGCGACTGGGCGTGGAGCATCTGGTCGCATTGGGTCA
CCAGCAAATCGCGCTGTTAGCGGGCCCATTAAGTTCTGTCTCGGCGCGTC
TGCGTCTGGCTGGCTGGCATAAATATCTCACTCGCAATCAAATTCAGCCG
ATAGCGGAACGGGAAGGCGACTGGAGTGCCATGTCCGGTTTTCAACAAAC
CATGCAAATGCTGAATGAGGGCATCGTTCCCACTGCGATGCTGGTTGCCA
ACGATCAGATGGCGCTGGGCGCAATGCGCGCCATTACCGAGTCCGGGCTG
CGCGTTGGTGCGGATATCTCGGTAGTGGGATACGACGATACCGAAGACAG
CTCATGTTATATCCCGCCGTCAACCACCATCAAACAGGATTTTCGCCTGC
TGGGGCAAACCAGCGTGGACCGCTTGCTGCAACTCTCTCAGGGCCAGGCG
GTGAAGGGCAATCAGCTGTTGCCCGTCTCACTGGTGAAAAGAAAAACCAC
CCTGGCGCCCAATACGCAAACCGCCTCTCCCCGCGCGTTGGCCGATTCAT
TAATGCAGCTGGCACGACAGGTTTCCCGACTGGAAAGCGGGCAGTGAGCG
CAACGCAATTAATGTGAGTTAGGCATCGCATCCTGCCTCGCGCGTTTCGG
TGATGACGGTGAAAACCTCTGACACATGCAGCTCCCGGAGACGGTCACAG
CTTGTCTGTAAGCGGATGCCGGGAGCAGACAAGCCCGTCAGGGCGCGTCA
GCGGGTGTTGGCGGGTGTCGGGGCGCAGCCATGACCCAGTCACGTAGCGA
TAGCGGAGTGTATACTGGCTTAACTATGCGGCATCAGAGCAGATTGTACT
GAGAGTGCACCATATGCGGTGTGAAATACCGCACAGATGCGTAAGGAGAA
AATACCGCATCAGGCGCTCTTCCGCTTCCTCGCTCACTGACTCGCTGCGC
TCGGTCGTTCGGCTGCGGCGAGCGGTATCAGCTCACTCAAAGGCGGTAAT
ACGGTTATCCACAGAATCAGGGGATAACGCAGGAAAGAACATGTGAGCAA
AAGGCCAGCAAAAGGCCAGGAACCGTAAAAAGGCCGCGTTGCTGGCGTTT
TTCCATAGGCTCCGCCCCCCTGACGAGCATCACAAAAATCGACGCTCAAG
TCAGAGGTGGCGAAACCCGACAGGACTATAAAGATACCAGGCGTTTCCCC
CTGGAAGCTCCCTCGTGCGCTCTCCTGTTCCGACCCTGCCGCTTACCGGA
TACCTGTCCGCCTTTCTCCCTTCGGGAAGCGTGGCGCTTTCTCAATGCTC
ACGCTGTAGGTATCTCAGTTCGGTGTAGGTCGTTCGCTCCAAGCTGGGCT
GTGTGCACGAACCCCCCGTTCAGCCCGACCGCTGCGCCTTATCCGGTAAC
TATCGTCTTGAGTCCAACCCGGTAAGACACGACTTATCGCCACTGGCAGC
AGCCACTGGTAACAGGATTAGCAGAGCGAGGTATGTAGGCGGTGCTACAG
AGTTCTTGAAGTGGTGGCCTAACTACGGCTACACTAGAAGGACAGTATTT
GGTATCTGCGCTCTGCTGAAGCCAGTTACCTTCGGAAAAAGAGTTGGTAG
CTCTTGATCCGGCAAACAAACCACCGCTGGTAGCGGTGGTTTTTTTGTTT
GCAAGCAGCAGATTACGCGCAGAAAAAAAGGATCTCAAGAAGATCCTTTG
ATCTTTTCTACGGGGTCTGACGCTCAGTGGAACGAAAACTCACGTTAAGG
GATTTTGGTCATGAGATTATCAAAAAGGATCTTCACCTAGATCCTTTTAA
ATTAAAAATGAAGTTTTAAATCAATCTAAAGTATATATGAGTAAACTTGG
TCTGACAGTTACCAATGCTTAATCAGTGAGGCACCTATCTCAGCGATCTG
TCTATTTCGTTCATCCATAGTTGCCTGACTCCCCGTCGTGTAGATAACTA
CGATACGGGAGGGCTTACCATCTGGCCCCAGTGCTGCAATGATACCGCGA
GACCCACGCTCACCGGCTCCAGATTTATCAGCAATAAACCAGCCAGCCGG
AAGGGCCGAGCGCAGAAGTGGTCCTGCAACTTTATCCGCCTCCATCCAGT
CTATTAATTGTTGCCGGGAAGCTAGAGTAAGTAGTTCGCCAGTTAATAGT
TTGCGCAACGTTGTTGCCATTGCTACAGGCATCGTGGTGTCACGCTCGTC
GTTTGGTATGGCTTCATTCAGCTCCGGTTCCCAACGATCAAGGCGAGTTA
CATGATCCCCCATGTTGTGCAAAAAAGCGGTTAGCTCCTTCGGTCCTCCG
ATCGTTGTCAGAAGTAAGTTGGCCGCAGTGTTATCACTCATGGTTATGGC
AGCACTGCATAATTCTCTTACTGTCATGCCATCCGTAAGATGCTTTTCTG
TGACTGGTGAGTACTCAACCAAGTCATTCTGAGAATAGTGTATGCGGCGA
CCGAGTTGCTCTTGCCCGGCGTCAACACGGGATAATACCGCGCCACATAG
CAGAACTTTAAAAGTGCTCATCATTGGAAAACGTTCTTCGGGGCGAAAAC
TCTCAAGGATCTTACCGCTGTTGAGATCCAGTTCGATGTAACCCACTCGT
GCACCCAACTGATCTTCAGCATCTTTTACTTTCACCAGCGTTTCTGGGTG
AGCAAAAACAGGAAGGCAAAATGCCGCAAAAAAGGGAATAAGGGCGACAC
GGAAATGTTGAATACTCATACTCTTCCTTTTTCAATATTATTGAAGCATT
TATCAGGGTTATTGTCTCATGAGCGGATACATATTTGAATGTATTTAGAA
AAATAAACAAATAGGGGTTCCGCGCACATTTCCCCGAAAAGTGCCACCTG
ACGTCCAATAGACCAGTTGCAATCCAAACGAGAGTCTAATAGAATGAGGT
CGAAAAGTAAATCGCGTAATAAGGTAATAGATTTACATTAGAAAATGAAA
GGGGATTTTATGCGTGAGAATGTTACAGTCTATCCCGGCATTGCCAGTCG
GGGATATTAAAAAGAGTATAGGTTTTTATTGCGATAAACTAGGTTTCACT
TTGGTTCACCATGAAGATGGATTCGCAGTTCTAATGTGTAATGAGGTTCG
GATTCATCTATGGGAGGCAAGTGATGAAGGCTGGCGCTCTCGTAGTAATG
ATTCACCGGTTTGTACAGGTGCGGAGTCGTTTATTGCTGGTACTGCTAGT
TGCCGCATTGAAGTAGAGGGAATTGATGAATTATATCAACATATTAAGCC
TTTGGGCATTTTGCACCCCAATACATCATTAAAAGATCAGTGGTGGGATG
AACGAGACTTTGCAGTAATTGATCCCGACAACAATTTGATTACAAATAAA
AAGCTAAAATCTATTATTAATCTGTTCCTGCAGGAGAGACCG

D. Transcriptome DNA Array Methods

In addition to the above methods, transcriptome DNA array methods were used in the development of mutants of the present invention. First, target RNA was harvested from a Bacillus strain by guanidinium acid phenol extraction as known in the art (See e.g., Farrell, RNA Methodologies, (2nd Ed.). Academic Press, San Diego, at pp. 81] and time-point was reverse-transcribed into biotin-labeled cDNA by a method adopted from deSaizieu et al. (deSaizieu et al., J. Bacteriol., 182: 4696-4703 [2000]) and described herein. Total RNA (25 mg) was incubated 37° C. overnight in a 100-mL reaction: 1×GIBCO first-strand buffer (50 mM Tris-HCl pH 8.3, 75 mM KCl, 3 mM MgCl₂); 10 mM DTT; 40 mM random hexamer; 0.3 mM each dCTP, dGTP and dTTP; 0.12 mM dATP; 0.3 mM biotin-dATP (NEN0; 2500 units SuperScript II reverse-transcriptase (Roche). To remove RNA, the reaction was brought to 0.25 M NaOH and incubated at 65° C. for 30 minutes. The reaction was neutralized with HCl and the nucleic acid precipitated at −20° C. in ethanol with 2.5 M ammonium-acetate. The pellet was washed, air-dried, resuspended in water, and quantitated by UV spectroscopy. The reaction yield was approximately 20-25 mg biotin-labeled cDNA.

Twelve mg of this cDNA were fragmented in 33 mL 1× One-Phor-All buffer (Amersham-Pharmacia #27-0901-02) with 3.75 milliunits of DNaseI I at 37° C. for 10 minutes. After heat-killing the DNase, fragmentation was validated by running 2 mg of the fragmented cDNA on a 3% agarose gel. Biotin-containing cDNA routinely ranged in size from 25 to 125 nucleotides. The remaining 10 mg of cDNA were hybridized to an Affymetrix Bacillus GeneChip array.

Hybridizations were performed as described in the Affymetrix Expression Analysis Technical Manual (Affymetrix) using reagent suppliers as suggested. Briefly, 10 mg of fragmented biotin-labeled cDNA were added to a 220-mL hybridization cocktail containing: 100 mM MES (N-morpholinoethanesulfonic acid), 1M Na⁺, 20 mM EDTA, 0.01% Tween 20; 5 mg/mL total yeast RNA; 0.5 mg/mL BSA; 0.1 mg/mL herring-sperm DNA; 50 μM control oligonucleotide (AFFX-B1). The cocktails were heated to 95° C. for 5 minutes, cooled to 40° C. for 5 minutes, briefly centrifuged to remove particulates, and 200 mL was injected into each pre-warmed pre-rinsed (1×MES buffer+5 mg/ml yeast RNA) GeneChip cartridge. The arrays were rotated at 40° C. overnight.

The samples were removed and the arrays were filled with non-stringent wash buffer (6×SSPE, 0.01% TWEEN-20) and washed on the Affymetrix fluidics station with protocol Euk-GE-WS2, using non-stringent and stringent (0.1 M MES, 0.1 M [Na⁺], 0.01% Tween 20) wash buffers. Arrays were stained in three steps: (1) streptavidin; (2) anti-streptavidin antibody tagged with biotin; (3) streptavidin-phycoerythrin conjugate.

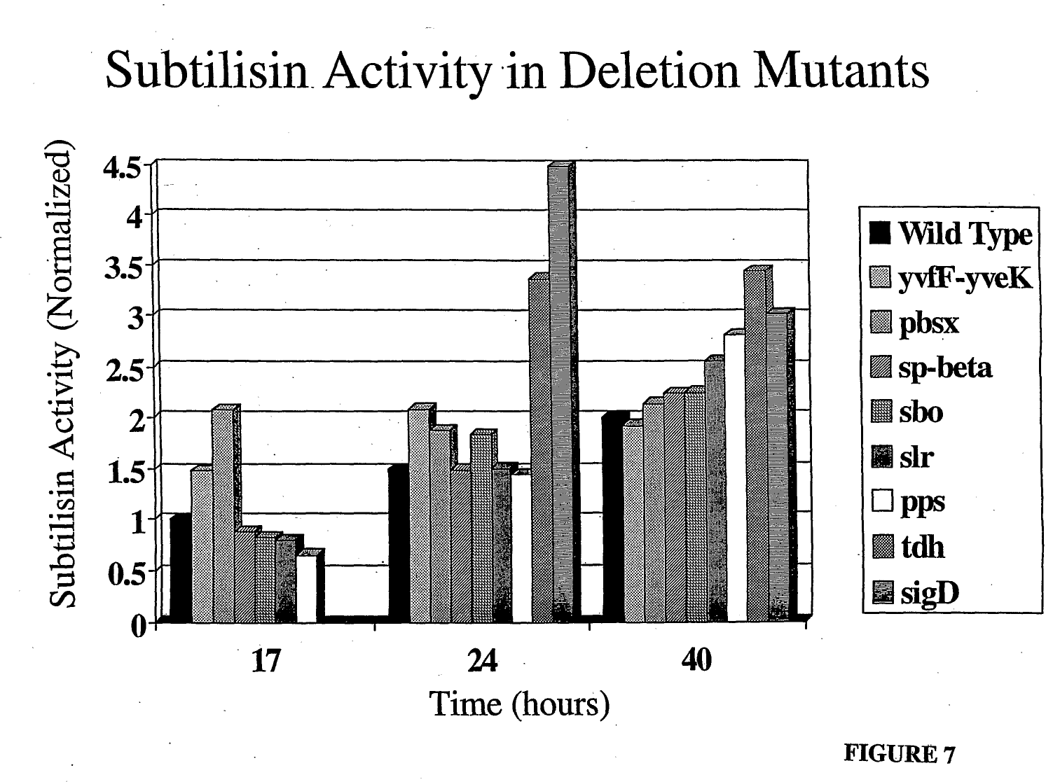

The signals in the arrays were detected with the Hewlett-Packard Gene Array Scanner using 570 nm laser light with 3-mm pixel resolution. The signal intensities of the 4351 ORF probe sets were scaled and normalized across all time points comprising a time course experiment. These signals were then compared to deduce the relative expression levels of genes under investigation. The threonine biosynthetic and degradative genes were simultaneous transcribed, indicating inefficient threonine utilization. Deletion of the degradative threonine pathway improved expression of the desired product (See, FIG. 7). The present invention provides means to modify pathways with transcription profiles that are similar to threonine biosynthetic and degradative profiles. Thus, the present invention also finds use in the modification of pathways with transcription profiles similar to threonine in order to optimize Bacillus strains. In some preferred embodiments, at least one gene selected from the group consisting of rocA, ycgN, ycgM rocF and rocD is deleted or otherwise modified. Using the present invention as described herein resulted in the surprising discovery that the sigD regulon was transcribed. Deletion of this gene resulted in better expression of the desired product (See, FIG. 7).

In these preliminary experiments, deletion of pckA in a histidine auxotropy host, “KH5,” did not result in improvement or detriment in the strain grown in minimal medium in shake flasks. However, the present invention provides means to improve strain protein production through use of the pckA deletion or modification and/or combination with deletion or modification of gapB and/or fbp. In addition, during the development of the present invention, it was observed that the tryptophan biosynthetic pathway genes showed unbalanced transcription. Thus, it is contemplated that the present invention will find use in producing strains that exhibit increased transcription of genes such as those selected from the group consisting of trpA, trpB, trpC, trpD, trpE, and/or trpF, such that the improved strains provide improved expression of the desired product, as compared to the parental (i.e., wild-type and/or originating strain). Indeed, it is contemplated that modifications of these genes in any combination will lead to improved expression of the desired product. Furthermore, additional experiments (described below in Example 6) indicated that inactivation of the pckA gene led to increased protein expression due to improved carbon utilization efficiency in the deletion strains developed.

E. Fermentations

Analysis of the strains produced using the above constructs were conducted following fermentation. Cultures at 14 L scale were conducted in BIOLAFITTE® fermenters. Media components per 7 liters are listed in Table 9.

TABLE 9
Media Components per 7L Fermentation

| | | | |
|---|---|---|---|
| NaH2PO4—H2O | 0.8% | 56 | g |
| KH2PO4 | 0.8% | 56 | g |
| MgSO4—7H2O | 0.28% | 19.6 | g |
| Antifoam | 0.1% | 7 | g |
| CaCl2—2H2O | 0.01% | 0.7 | g |
| ferrous sulfate-7H2O | 0.03% | 2.1 | g |
| MnCl2—4H2O | 0.02% | 1.4 | g |
| trace metals 100 x | 1% | 70 | g |
| stock* | | | |
| H2SO4 | 0.16% | 11.2 | g |

| | | |
|---|---|---|
| 60% glucose | 1.29% | 90 |

*See, Harwood and Cutting, supra, at p. 549

The tanks were stirred at 750 rpm and airflow was adjusted to 11 Liters per minute, the temperature was 37° C., and the pH was maintained at 6.8 using NH₄OH. A 60% glucose solution was fed starting at about 14 hours in a linear ramp from 0.5 to 2.1 grams per minute to the end of the fermentation. Off-gasses were monitored by mass spectrometry. Carbon balance and efficiency were calculated from glucose fed, yield of protein product, cell mass yield, other carbon in broth, and CO₂ evolved. A mutant strain was compared to parent strain to judge improvements. Indeed, as described in Example 6, below, modification of pckA in some strains has led to increased production of protein of interest. In some preferred embodiments, additional genes are selected from the group consisting of gapB, alsD, and/or fbp.

### Example 4

### Host Cell Transformation to Obtain an Altered Bacillus Strain

Once the DNA construct was created by Method 1 or 2 as described above, it was transformed into a suitable Bacillus subtilis lab strain (e.g., BG2036 or BG2097; any competent Bacillus immediate host cell may be used in the methods of the present invention). The cells were plated on a selective media of 0.5 ppm phleomycin or 100 ppm spectinomycin as appropriate (Ferrari and Miller, Bacillus Expression: A Gram-Positive Model in Gene Expression Systems: Using Nature for the Art of Expression, pgs 65-94 [1999]). The laboratory strains were used as a source of chromosomal DNA carrying the deletion that was transformed into a Bacillus subtilis production host strain twice or BG3594 and then MDT 98-113 once. Transformants were streaked to isolate a single colony, picked and grown overnight in 5 mL of LB plus the appropriate antimicrobial. Chromosomal DNA was isolated as known in the art (See e.g., Hardwood et al., supra).

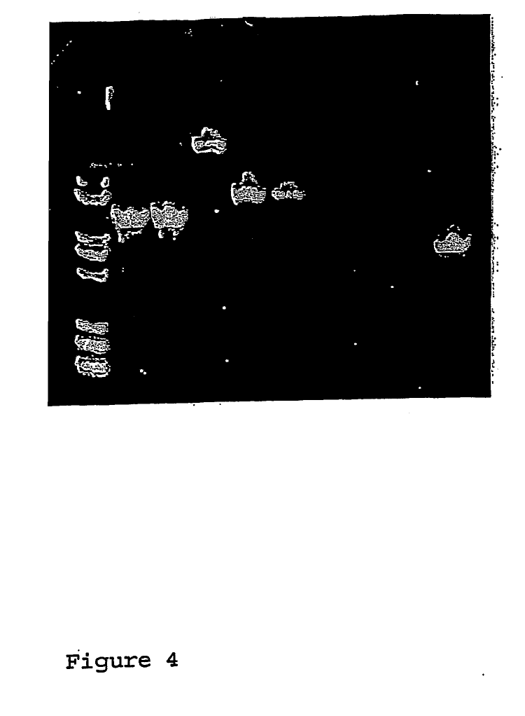

The presence of the integrated DNA construct was confirmed by three PCR reactions, with components and conditions as described above. For example, two reactions were designed to amplify a region from outside the deletion cassette into the antimicrobial gene in one case (primers 1 and 11) and through the entire insert in another (primers 1 and 12). A third check amplified a region from outside the deletion cassette into the deleted region (primers 1 and 4). FIG. 4 shows that a correct clone showed a band in the first two cases but not the third. Wild-type Bacillus subtilis chromosomal DNA was used as a negative control in all reactions, and should only amplify a band with the third primer set.

### Example 5

### Shake Flask Assays

### Measurement of Protease Activity

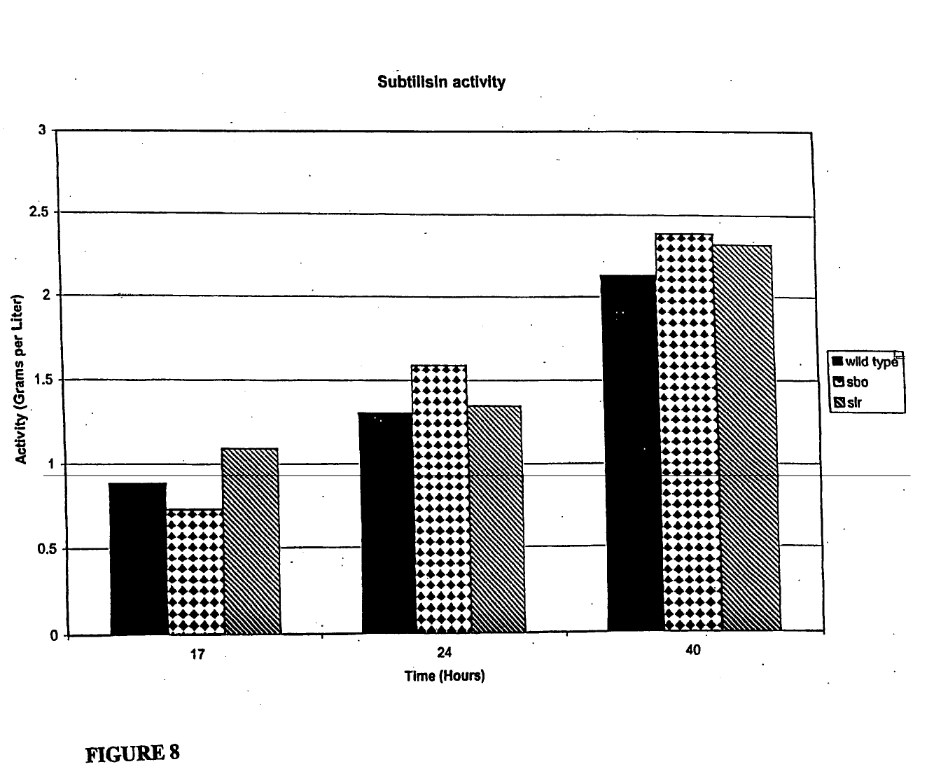

Once the DNA construct was stably integrated into a competent Bacillus subtilis strain, the subtilisin activity was measured by shake flask assays and the activity was compared to wild type levels. Assays were performed in 250 ml baffled flasks containing 50 mL of growth media suitable for subtilisin production as known in the art (See, Christianson et al., Anal. Biochem., 223:119-129 [1994]; and Hsia et al., Anal. Biochem. 242:221-227 [1996]). The media were inoculated with 50 μL of an 8 hour 5 mL culture and grown for 40 hours at 37° C. with shaking at 250 RPM. Then, 1 mL samples were taken at 17, 24 and 40 hours for protease activity assays. Protease activity was measured at 405 nM using the Monarch Automatic Analyser. Samples in duplicate were diluted 1:11 (3.131 g/L) in buffer. As a control to ensure correct machine calibration one sample was diluted 1:6 (5.585 g/L), 1:12 (2.793 g/L and 1:18 (1.862 g/L). FIG. 7 illustrates the protease activity in various altered Bacillus subtilis clones. FIG. 8 provides a graph showing improved protease secretion as measured from shake flask cultures in Bacillus subtilis wild-type strain (unaltered) and corresponding altered deletion strains (-sbo) and (-sir). Protease activity (g/L) was measured after 17, 24 and 40 hours.

Cell density was also determined using spectrophotometric measurement at an OD of 600. No significant differences were observed for the samples at the measured time (data not shown).

### Example 6

### pckA Deletion Construction

In this Example, additional descriptions of the pckA deletion mutants of the present invention are provided. As indicated in Example 2B, pckA deletion constructs were produced as described in Example 2B, using a PCR fusion method to bypass the requirement of using E. coli. In this Example, the deletion mutants produced according to Example were modified. The PCR primers PCKA-1, PCKA-2, PCKA-3, PCKA-4, PCKA-5, AND PCKA-6 (See, Table 5), were used for PCR and PCR fusion reactions using the chromosomal DNA of a Bacillus subtilis BG2816 and 1168 pDG1726 (See, Guerout-Fleury et al., Gene 167: 335-6 [1995]) as template. The methods used in constructing these deletion mutants was the same as Method 1, described above.

The PCR fusion was performed as described above on three different DNA fragments generated a DNA cassette of pckA upstream-spec-pckA down stream (1.998 kb). The PCR fusion strategy was as following:

pckA-1→ pckA-3→ pck-5→
-----pckA upstream----------------------spectinomycin--------------------pckA dowmstream---------
←pckA-2 ←pckA-4 ←pckA-6

Results showed the expected single fragment that indicated the 1.998 kb PCR fusion fragment had been correctly assembled. This cassette was then subcloned into pCR-script SK+ to generate “pKH5” (4.9 kb). The construction of pKH5 was confirmed by PCR and HindIII restriction digestion pattern results. The pKH5 was then digested by ScaI and used to transform BG2816 (ΔnprE, ΔaprE, his⁻) and a protease producing B. subtilis strain (a control strain named “FNA hyper1”: ΔaprE::subtilisin-Cm, ΔnprE, degUHy32, oppA, ΔspolIE350) to build KH5 and KHB5. The transformed cells were plated on LA plates containing 100 ug/ml spectinomycin and incubated overnight at 37° C. Transformants were selected for integration by antibiotic resistance and were further analyzed by PCR. All transformants of KH5 analyzed were identified as double crossover integrations. The chromosomal DNA of KH5 was isolated and used to transform FNA hyper1 to build KHB5.

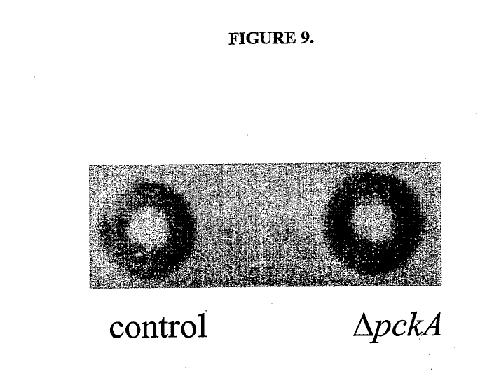

The genotype of KH5 was BG2816, ΔpckA::spec, and the genotype of KHB5 was FNA hyper1, ΔpckA::spec. In order to compare the pckA-deletion strain and a control pckA wild-type strain, several experiments were performed to characterize the pckA-deletion strain. FIG. 9 provides a photograph showing the clearing “halo” produced by the pckA-deletion strain and a control strain on LA+1.6% skim milk medium. As shown in this Figure, the pckA-deletion strain (KHB5) produced a slightly larger halo than the control strain (FNA hyper1).

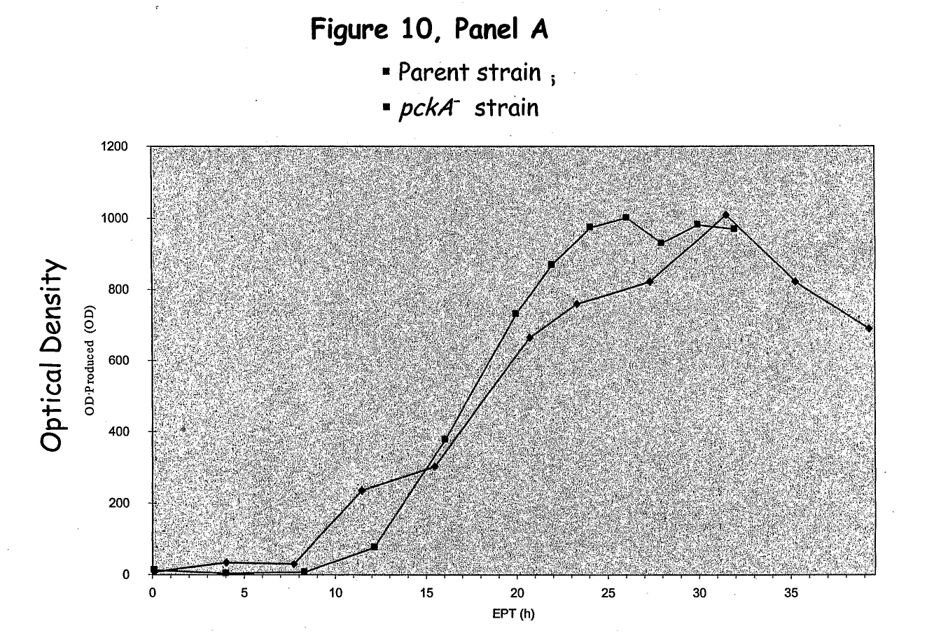

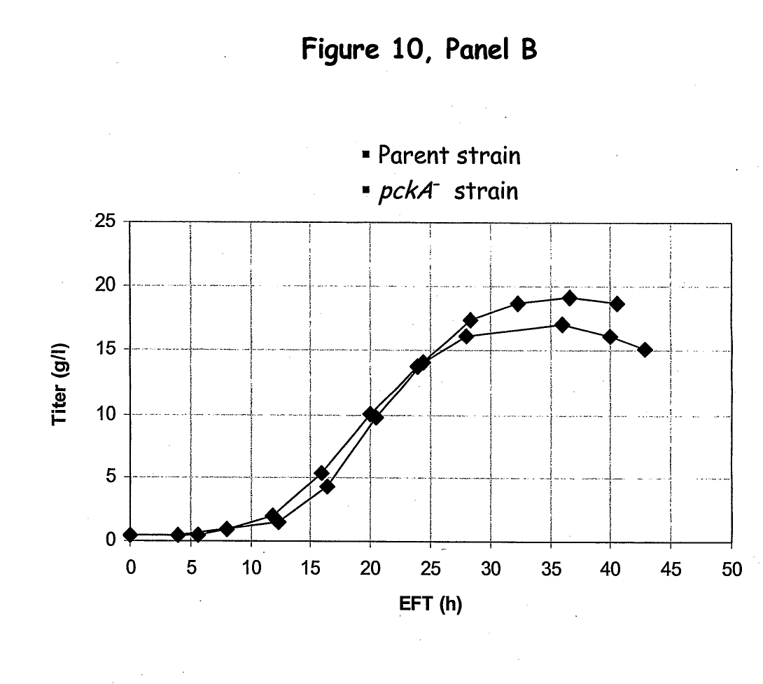

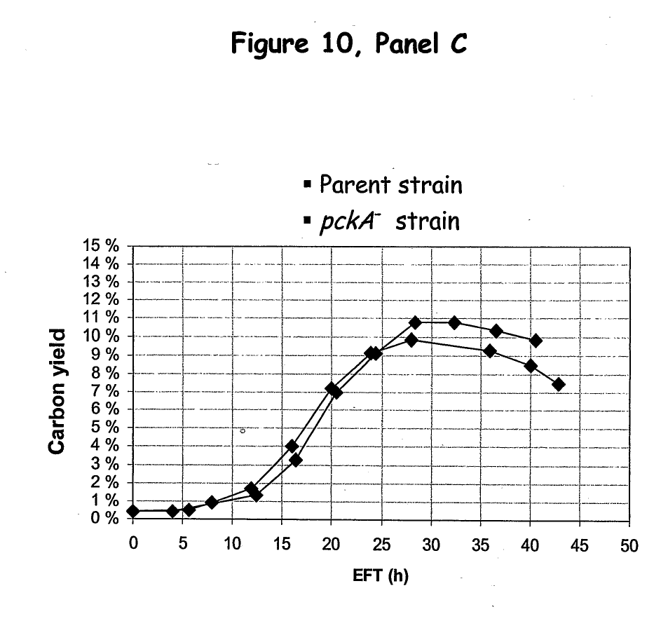

FIG. 10, Panel A provides a graph showing the optical density of the parent strain (FNA hyper1) and the pckA-deletion strain grown in a minimal medium as described in Example 3, Section E. As indicated by this graph, the pckA-deletion strain produced more growth in a shorter time period than the parent strain. FIG. 10, Panel B provides a graph showing the titer of the parent strain and the pckA-deletion strain grown in a soy meal-glucose based complex medium expressed in g/liter over time. FIG. 10, Panel C provides a graph showing the carbon yield of the parent strain and the pckA-deletion strain grown in a soy meal-glucose based complex medium. As indicated in this Panel, the pckA⁻-deletion strain was more efficient at carbon utilization, based on amount of protease produced on the based of gram of carbon.

The carbon yield was calculated based on the following formula:

$${Y}_{Product , C} ( t ) [ \% ] = \frac{\begin{matrix}{m}_{Broth} ( t ) [ kg ] \cdot \frac{1}{ρ} [ \frac{1}{kg} ] \cdot Activity ( t ) [ \frac{g Act}{1} ] \cdot \\ X [ \frac{g protein}{g Act} ] \cdot Y [ \frac{g C}{g protein} ]\end{matrix}}{{m}_{C , feed solids} ( t ) [ g C ] + {m}_{C , soy} ( t ) [ g C ]}$$

X=mass of protein/activity Unit, in the formula herein use “g protein/g activity”.
Y=mass % carbon in the molecule.
The index “(t)” means at a specific time point.
ρ=density.

Although the pckA-deletion mutant strain was more efficient in utilizing the carbon present in the complex medium (as indicated by exhibiting at least a 10% increase in carbon going to biomass), the protease production was not affected (on a per cell basis) in this medium. However, in minimal medium, it was determined that mutant pckA-deletion strains were able to produce more protease and more cells, as compared to the control strain. As discussed in greater detail herein, the pckA-deletion strain, KHB5, was able to make larger halo than the control parental strain (FNA hyper1), on the LA+1.6% skim milk plate (i.e., more protease was produced by the mutant than the parent). In addition, the pckA-deletion strain, KHB5, was able to reach higher optical density than the control parental strain (FNA hyper1) in minimal medium (i.e., with glucose as the only carbon source). These results indicate that the pckA mutant strain produced more cells from the same amount of carbon than the parental strain.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as should not be unduly limited to such specific embodiments. Indeed, various modifications of the described modes for carrying out the invention that are obvious to those skilled in the art and/or related fields are intended to be within the scope of the present invention.

We claim:

1. A microorganism comprising a modified pckA gene.

2. The microorganism of claim 1, wherein said pckA gene has been deleted.

3. The microorganism of claim 1, wherein said pckA gene has been inactivated.

4. The microorganism of claim 1, wherein said microorganism is a Gram-positive microorganism.

5. The microorganism of claim 4, wherein said Gram-positive microorganism is a Bacillus species.

6. The microorganism of claim 5, wherein said microorganism exhibits improved expression of at least one protein of interest, as compared to a microorganism having an unmodified pckA gene.

7. The microorganism of claim 6, wherein at least one indigenous chromosomal region within said Bacillus species has been modified to produce a variant Bacillus strain and wherein said variant Bacillus strain expresses a higher level of said protein of interest as compared to said Bacillus species.

8. The microorganism of claim 7, wherein said variant Bacillus strain comprises a heterologous polynucleotide encoding said protein of interest.

9. The variant Bacillus strain of claim 7, wherein said variant Bacillus strain is B. subtilis.

10. The microorganism of claim 7, wherein said at least one modified indigenous chromosomal region is selected from the group consisting of prophage regions, antimicrobial regions, regulator regions, multi-contiguous single gene regions, and operon regions.

11. The microorganism of claim 10, wherein said at least one modified indigenous chromosomal region is selected from the group consisting of PBSX regions, skin regions, prophage 7 regions, SPβ regions, prophage 1 regions, prophage 2 regions, prophage 3 regions, prophage 4 regions, prophage 5 regions, prophage 6 regions, PPS regions, PKS regions, yvfF-yveK regions, DHB regions, and fragments thereof.

12. The microorganism of claim 10, wherein said at least one modified indigenous chromosomal region is modified by deletion.

13. The microorganism of claim 12, wherein said deletion comprises a deletion of one or more indigenous chromosomal regions or fragments thereof, wherein the indigenous chromosomal region includes about 0.5 to 500 kilobases.

14. An altered Bacillus strain comprising deletion of the pckA gene.

15. The altered Bacillus strain of claim 14, wherein said altered Bacillus strain produces at least one protease.

16. The altered Bacillus strain of claim 14, wherein said protease is subtilisin.

17. The subtilisin of claim 16, wherein said subtilisin is selected from the group consisting of subtilisin 168, subtilisin BPN′, subtilisin Carlsberg, subtilisin DY, subtilisin 147, subtilisin 309, and variants thereof.

18. The altered Bacillus strain of claim 14, wherein said altered Bacillus strain further comprises at least one deletion in at least one chromosomal gene selected from the group of sbo, sir, ybcO, csn, spolISA, sigB, phrC, rapA, CssS, trpA, trpB, trpC, trpD, trpE, trpF, tdh/kbl, alsD, sigD, prpC, gapB, fbp, rocA, ycgN, ycgM, rocF, and rocD.

20. The altered Bacillus strain of claim 18, further comprising at least one mutation in at least one gene selected from the group consisting of degU, degQ, degS, scoC4, spolIE, and oppA.

21. The altered Bacillus strain of claim 20, wherein said mutation comprises degU(Hy)32.

22. The altered Bacillus strain of claim 14, further comprising at least one DNA construct comprising an incoming sequence.

23. The altered Bacillus strain of claim 22, wherein said incoming sequence comprises at least one selection marker and the pckA gene.

24. The altered Bacillus strain of claim 23, wherein said incoming sequence further comprises at least one gene selected from the group consisting of sbo, sir, ybcO, csn, spolISA, sigB, phrC, rapA, CssS, trpA, trpB, trpC, trpD, trpE, trpF, tdh/kbl, alsD, sigD, prpC, gapB, fbp, rocA, ycgN, ycgM, rocF, and rocD.

25. The altered Bacillus strain of claim 24, wherein said at least one selection marker is located in between two fragments of said gene.

26. The altered Bacillus strain of claim 22, wherein said incoming sequence comprises a selection marker and at least one homology box, wherein said homology box flanks the 5′ and/or 3′ end of said selection marker.

27. The altered Bacillus strain of claim 22, wherein said incoming sequence is incorporated into the genome of said altered Bacillus strain.

28. An isolated nucleic acid comprising at least one sequence set forth in a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:39, SEQ ID NO:40, SEQ ID NO:42, SEQ ID NO:44, SEQ ID NO:46, SEQ ID NO:48, SEQ ID NO:50, SEQ ID NO:37, SEQ ID NO:25, SEQ ID NO:21, SEQ ID NO:50, SEQ ID NO:23, SEQ ID NO:27, SEQ ID NO:19, SEQ ID NO:31, SEQ ID NO:48, SEQ ID NO:46, SEQ ID NO:35, and SEQ ID NO:33.

29. An isolated nucleic acid sequence encoding an amino acid, wherein said amino acid is selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:41, SEQ ID NO:43, SEQ ID NO:45, SEQ ID NO:47, SEQ ID NO:49, SEQ ID NO:51, SEQ ID NO:38, SEQ ID NO:26, SEQ ID NO:22, SEQ ID NO:57, SEQ ID NO:24, SEQ ID NO:28, SEQ ID NO:20, SEQ ID NO:32, SEQ ID NO:55, SEQ ID NO:53, SEQ ID NO:36, and SEQ ID NO:34.

30. A purified DNA construct comprising an incoming sequence, wherein said incoming sequence comprises a selection marker and either a cssS gene, a cssS gene fragment or a homologous sequence thereof.

31. The DNA construct of claim 30, wherein said selection marker is located between two fragments of said cssS gene.

32. The DNA construct of claim 30, wherein said incoming sequence further comprises at least one homology box wherein the homology box flanks the 5′ and/or 3′ end of said selection marker.

33. The DNA construct of claim 30, wherein said DNA sequence comprises at least one nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:39, SEQ ID NO:40, SEQ ID NO:42, SEQ ID NO:44, SEQ ID NO:46, SEQ ID NO:48, SEQ ID NO:50, SEQ ID NO:37, SEQ ID NO:25, SEQ ID NO:21, SEQ ID NO:50, SEQ ID NO:29; SEQ ID NO:23, SEQ ID NO:27, SEQ ID NO:19, SEQ ID NO:31, SEQ ID NO:48, SEQ ID NO:46, SEQ ID NO:35, and SEQ ID NO:33.

34. The DNA construct of claim 30, comprising at least one gene that encodes an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:41, SEQ ID NO:43, SEQ ID NO:45, SEQ ID NO:47, SEQ ID NO:49, SEQ ID NO:51, SEQ ID NO:38, SEQ ID NO:26, SEQ ID NO:22, SEQ ID NO:57, SEQ ID NO:30, SEQ ID NO:24, SEQ ID NO:28, SEQ ID NO:20, SEQ ID NO:32, SEQ ID NO:55, SEQ ID NO:53, SEQ ID NO:36, and SEQ ID NO:34.

35. A host cell transformed with the DNA construct of claim 30.

36. The host cell of claim 35, wherein said host cell is selected from the group consisting of Escherichia and Bacillus cells.

37. The host cell of claim 35, wherein said incoming sequence is chromosomally integrated in said host cell.

38. A method for enhancing production of at least one protein by a microorganism, comprising the steps:
a) providing a microorganism host cell;
b) inactivating the pckA gene in said host cell to produce an altered strain; and
c) growing said altered strain under growth conditions suitable for expression of said protein.

39. The method of claim 38, further comprising the step of collecting said protein expressed by said altered strain.

40. The method of claim 38, wherein said host cell is selected from the group consisting of Escherichia and Bacillus cells.

41. The method of claim 38, further comprising the step of inactivating at least one chromosomal gene selected from the group consisting of sbo, sir, ybcO, csn, spolISA, sigB, phrC, rapA, CssS trpA, trpB, trpC, trpD, trpE, trpF, tdh/kbl, alsD, sigD, prpC, gapB, fbp, rocA, ycgN, ycgM, rocF, and rocD.

42. The method of claim 38, wherein said protein is selected from the group consisting of homologous proteins and heterologous proteins.

43. The method of claim 38, wherein said at least one chromosomal gene is inactivated by insertional inactivation.

44. The method of claim 38, wherein said at least one protein is selected from the group consisting of proteases, cellulases, amylases, carbohydrases, lipases, isomerases, transferases, kinases, phosphatases, antibodies, hormones, and growth factors.

45. A method for obtaining an altered Bacillus strain expressing a protein of interest, comprising the steps of transforming a Bacillus host cell with a DNA construct comprising an incoming sequence which comprises the pckA gene, wherein said incoming sequence is integrated into the chromosome of said Bacillus host cell to produce an altered Bacillus strain, further in which one or more chromosomal genes have been inactivated; and growing said altered Bacillus strain under suitable growth conditions for the expression of a at least one protein of interest.

46. The method of claim 45, wherein said least one protein of interest is selected from proteases, cellulases, amylases, carbohydrases, lipases, isomerases, transferases, kinases, phosphatases, antibodies, hormones, and growth factors.

47. The method of claim 45, wherein said Bacillus host cell is selected from the group consisting of B. licheniformis, B. lentus, B. subtilis, B. amyloliquefaciens B. brevis, B. stearothermophilus, B. alkalophilus, B. coagulans, B. circulans, B. pumilus, B. thuringiensis, B. clausii, B. megaterium, and B. subtilis.

48. The method of claim 45, wherein said Bacillus host cell is a recombinant host cell.

49. The method of claim 45, wherein said protein of interest produced by said altered Bacillus strain is recovered.

50. The method of claim 45, wherein said DNA construct further comprises a selection marker.

51. The method of claim 50, wherein said selection marker is excised from said incoming sequence upon integration of said incoming sequence into said chromosome of said Bacillus host cell.

52. The method of claim 45, wherein the incoming sequence further comprises at least one gene selected from the group consisting of sbo, sir, ybcO, csn, spolISA, sigB, phrC, rapA, CssS, trpA, trpB, trpC, trpD, trpE, trpF, tdh/kbl, alsD, sigD, prpC, gapB, fbp, rocA, ycgN, ycgM, rocF, and rocD, wherein the DNA construct is integrated into the chromosome of the Bacillus host cell and results in the deletion of one or more genes present on said chromosome of said Bacillus host cell.

53. The method of claim 45, wherein said DNA construct comprises at least one nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:39, SEQ ID NO:40, SEQ ID NO:42, SEQ ID NO:44, SEQ ID NO:46, SEQ ID NO:48, SEQ ID NO:50, SEQ ID NO:37, SEQ ID NO:25, SEQ ID NO:21, SEQ ID NO:50, SEQ ID NO:29, SEQ ID NO:23, SEQ ID NO:27, SEQ ID NO:19, SEQ ID NO:31, SEQ ID NO:48, SEQ ID NO:46, SEQ ID NO:35, and SEQ ID NO:33.

54. The method of claim 45, wherein said DNA construct comprises at least one gene that encodes an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:41, SEQ ID NO:43, SEQ ID NO:45, SEQ ID NO:47, SEQ ID NO:49, SEQ ID NO:51, SEQ ID NO:38, SEQ ID NO:26, SEQ ID NO:22, SEQ ID NO:57, SEQ ID NO:30, SEQ ID NO:24, SEQ ID NO:28, SEQ ID NO:20, SEQ ID NO:32, SEQ ID NO:55, SEQ ID NO:53, SEQ ID NO:36, and SEQ ID NO:34.

55. A method for obtaining Bacillus subtilis strains having enhanced protease production comprising the steps of: transforming a B. subtilis host cell with a DNA construct; allowing homologous recombination of the DNA construct and a homologous region of the B. subtilis chromosome wherein pckA is deleted from the B. subtilis chromosome to produce an altered Bacillus subtilis strain; and growing the altered B. subtilis strain under conditions suitable for the expression of said protease.

56. The method of claim 55, wherein at least one gene selected from the group consisting of sbo, sir, ybcO, csn, spolISA, sigB, phrC, rapA, CssS, trpA, trpB, trpC, trpD, trpE, trpF, tdh/kbl, alsD, sigD, prpC, gapB, fbp, rocA, ycgN, ycgM, rocF, and rocD, is deleted from the chromosome of said B. subtilis.

57. The method of claim 55, wherein said protease is selected from the group consisting of homologous proteases and heterologous proteases.

58. The method of claim 57, wherein said protease is subtilisin.

59. The subtilisin of claim 58, wherein said subtilisin is selected from the group consisting of subtilisin 168, subtilisin BPN′, subtilisin Carlsberg, subtilisin DY, subtilisin 147, subtilisin 309, and variants thereof.

60. The method of claim 55, wherein said altered B. subtilis strain further comprises a mutation in at least one gene selected from the group consisting of degU, degQ, degS, scoC4, spolIE, and oppA.

61. The method of claim 60, wherein said mutation comprises degU(Hy)32.

62. The method of claim 55, wherein said altered B. subtilis strain comprises deletion of one or more indigenous chromosomal regions or fragments thereof, wherein the indigenous chromosomal region comprises about 0.5 to 500 kilobases.

63. The method of claim 55, wherein said DNA construct comprises at least one nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:39, SEQ ID NO:40, SEQ ID NO:42, SEQ ID NO:44, SEQ ID NO:46, SEQ ID NO:48, SEQ ID NO:50, SEQ ID NO:37, SEQ ID NO:25, SEQ ID NO:21, SEQ ID NO:50, SEQ ID NO:29, SEQ ID NO:23, SEQ ID NO:27, SEQ ID NO:19, SEQ ID NO:31, SEQ ID NO:48, SEQ ID NO:46, SEQ ID NO:35, and SEQ ID NO:33.

64. The method of claim 55, wherein said DNA construct comprises at least one gene that encodes an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:41, SEQ ID NO:43, SEQ ID NO:45, SEQ ID NO:47, SEQ ID NO:49, SEQ ID NO:51, SEQ ID NO:38, SEQ ID NO:26, SEQ ID NO:22, SEQ ID NO:57, SEQ ID NO:30, SEQ ID NO:24, SEQ ID NO:28, SEQ ID NO:20, SEQ ID NO:32, SEQ ID NO:55, SEQ ID NO:53, SEQ ID NO:36, and SEQ ID NO:34.

65. A method for enhancing the expression of a protein of interest in Bacillus comprising: introducing a DNA construct including a selective marker and an inactivating chromosomal segment into a Bacillus host strain, wherein said DNA construct is integrated into the chromosome of the Bacillus host strain, resulting in the deletion of an indigenous chromosomal region or fragment thereof from said Bacillus host strain to produce an altered Bacillus strain; and growing said altered Bacillus strain under suitable conditions, wherein expression of a protein of interest is greater in said altered Bacillus strain compared to the expression of the protein of interest in said Bacillus host strain.

66. The method of claim 65, further comprising the step of recovering said protein of interest.

67. The method of claim 65, further comprising the step of excising said selective marker from said altered Bacillus strain.

68. The method of claim 65, wherein said indigenous chromosomal region is selected from the group of regions consisting of PBSX, skin, prophage 7, SPβ, prophage 1, prophage 2, prophage 3, prophage 4, prophage 5, prophage 6, PPS, PKS, YVFF-YVEK, DHB, and fragments thereof.

69. The method of claim 65, wherein said altered Bacillus strain comprises deletion of at least two indigenous chromosomal regions.

70. The method of claim 65, wherein said protein of interest is selected from the group consisting of homologous proteins and heterologous proteins.

71. The method of claim 70, wherein said protein of interest is selected from proteases, cellulases, amylases, carbohydrases, lipases, isomerases, transferases, kinases phosphatases, antibodies, hormones, and growth factors.

72. The method of claim 65, wherein said Bacillus host strain is selected from the group consisting of B. licheniformis, B. lentus, B. subtilis, B. amyloliquefaciens, B. brevis, B. stearothermophilus, B. clausii, B. alkalophilus, B. coagulans, B. circulans, B. pumilus, and B. thuringiensis.

73. The method of claim 65, wherein said altered Bacillus strain further comprises at least one mutation in a gene selected from the group consisting of degU, degQ, degS, sco4, spolIE and oppA.

74. The method of claim 73, wherein said mutation comprises degU(Hy)32.

75. A method for enhancing the expression of a protein of interest in Bacillus comprising: obtaining nucleic acid from at least one Bacillus cell; performing transcriptome DNA array analysis on the nucleic acid from said Bacillus cell to identify at least one gene of interest; modifying said at least one gene of interest to produce a DNA construct; introducing said DNA construct into a Bacillus host cell to produce an altered Bacillus strain, wherein said altered Bacillus strain is capable of producing a protein of interest, under conditions such that expression of said protein of interest is enhanced as compared to the expression of the protein of interest in a Bacillus that has not been altered.

76. The method of claim 75, wherein said protein of interest is associated with at least one biochemical pathway selected from the group consisting of amino acid biosynthetic pathways and biodegradative pathways.

77. The method of claim 76, wherein at least one biodegradative pathway is disabled.

78. The method of claim 77, wherein said biodegradative pathway is disabled due to the transcription of a gene of interest.

79. The method of claim 75, wherein said Bacillus host cell is selected from the group consisting of B. licheniformis, B. lentus, B. subtilis, B. amyloliquefaciens, B. brevis, B. stearothermophilus, B. clausii, B. alkalophilus, B. coagulans, B. circulans, B. pumilus and B. thuringiensis.

80. The method of claim 75, wherein said protein of interest is selected from the group consisting of homologous proteins and heterologous proteins.

81. The method of claim 80, wherein said protein of interest is selected from proteases, cellulases, amylases, carbohydrases, lipases, isomerases, transferases, kinases, phosphatases, antibodies, hormones, and growth factors.

82. A method for enhancing the expression of a protein of interest in Bacillus, comprising: obtaining nucleic acid containing at least one gene of interest from at least one Bacillus cell; fragmenting said nucleic acid; amplifying said fragments to produce a pool of amplified fragments comprising said at least one gene of interest; ligating said amplified fragments to produce a DNA construct; directly transforming said DNA construct into a Bacillus host cell to produce an altered Bacillus strain, wherein said altered Bacillus strain comprises a modified gene selected from the group consisting of prpC, sigD and tdh/kbl; culturing said altered Bacillus strain under conditions such that expression of said protein of interest is enhanced as compared to the expression of said protein of interest in a Bacillus that has not been altered.

83. The method of claim 82, wherein said Bacillus host cell is selected from the group consisting of B. licheniformis, B. lentus, B. subtilis, B. amyloliquefaciens, B. brevis, B. stearothermophilus, B. clausii, B. alkalophilus, B. coagulans, B. circulans, B. pumilus and B. thuringiensis.

84. The method of claim 82, wherein said protein of interest is selected from the group consisting of homologous proteins and heterologous proteins.

85. The method of claim 84, wherein said protein of interest is selected from proteases, cellulases, amylases, carbohydrases, lipases, isomerases, transferases, kinases, phosphatases, antibodies, hormones, and growth factors.
